(19) United States
(12) Patent Application Publication
(10) Pub. No.: US 2024/0216572 A1
(43) Pub. Date: Jul. 4, 2024

(54) Biomaterials for the prevention and the treatment of tissue disorders

(71) Applicant: NOVADIP BIOSCIENCES, Mont-Saint-Guibert (BE)

(72) Inventors: DENIS DUFRANE, Lasne (BE); Nicolas THEYS, Dhuy (BE)

(73) Assignee: Novadip Biosciences, Mont-Saint-Guibert (BE)

(21) Appl. No.: 17/781,158

(22) PCT Filed: Nov. 27, 2020
(86) PCT No.: PCT/EP2020/083702

§ 371 Date: May 31, 2022

(30) Foreign Application Priority Data
Nov. 29, 2019 (EP) 19212634.0
Nov. 29, 2019 (EP) 19212654.8

(51) Int. Cl. A61L 27/12; A61L 27/10; A61L 27/26; A61L 27/36; A61L 27/38; C08L 5/04; C08L 5/08; C08L 5/12; C08L 29/04; C08L 31/06; C08L 39/06; C08L 67/04; C08L 89/06

(52) U.S. Cl. CPC A61L 27/12; A61L 27/10; A61L 27/26; A61L 27/3633; A61L 27/38; C08L 5/04; C08L 5/08; C08L 5/12; C08L 29/04; C08L 31/06; C08L 39/06; C08L 67/04; C08L 89/06; A61L 2300/414; A61L 2430/34

(57) ABSTRACT

The present invention relates to sterile and desiccated biomaterials comprising devitalized differentiated cells having tissue regenerating and/or repairing properties, and a particulate material, the cells and the particulate material being embedded in an extracellular matrix. The particulate material is preferably gelatin, a ceramic material, or a demineralized bone matrix (DBM).

### FIELD OF INVENTION

The present invention relates to the field of tissue regeneration and tissue repairing, including the prevention and/or the treatment of a skin disorder, a bone disorder and/or a cartilage disorder. More particularly, the invention relates to a sterile and desiccated biomaterial comprising devitalized differentiated cells having tissue regenerating and/or repairing properties, and a particulate material, the cells and the particulate material being embedded in an extracellular matrix.

### BACKGROUND OF INVENTION

Tissue reconstruction encompasses bone reconstruction, cartilage reconstruction, but also skin (including dermis and epidermis) reconstruction and muscle reconstruction. Bone defect is a lack of bone tissue in a body area, where bone should normally be. Bone defects can be treated by various surgical methods. However, often there are factors that impair bone healing, like diabetes mellitus, immunosuppressive therapy, poor locomotor status and others that one has to take into account when a procedure is planned. Surgical methods of bone defect reconstruction include inter alia decortication, excision and fixation, cancellous bone grafting and the Ilizarov intercalary bone transport method. However, patients commonly have prolonged ambulatory impairment with suboptimal functional and aesthetic results.

Tissue engineering involves the restoration of tissue structure or function through the use of living cells. The general process consists of cell isolation and proliferation, followed by a re-implantation procedure in which a scaffold material is used. Mesenchymal stem cells (MSCs) provide a good alternative to cells from mature tissue and have a number of advantages as a cell source for skin, bone and cartilage tissue regeneration, for example. By definition, a stem cell is characterized by its ability to undergo self-renewal and its ability to undergo multilineage differentiation and form terminally differentiated cells. Ideally, a stem cell for regenerative medicinal applications should meet the following set of criteria: (i) should be found in abundant quantities (millions to billions of cells); (ii) can be collected and harvested by a minimally invasive procedure; (iii) can be differentiated along multiple cell lineage pathways in a reproducible manner; (iv) can be safely and effectively transplanted to either an autologous or allogeneic host.

Studies have demonstrated that stem cells have the capacity to differentiate into cells of mesodermal, endodermal and ectodermal origins. The plasticity of MSCs most often refers to the inherent ability retained within stem cells to cross lineage barriers and to adopt the phenotypic, biochemical and functional properties of cells unique to other tissues. Adult mesenchymal stem cells can be isolated from bone marrow and adipose tissue, for example.

Adipose tissue-derived stem cells are multipotent and have profound regenerative capacities. Osteogenic differentiated ASCs were shown to have a great healing potential in various pre-clinical models when seeded on various scaffolds, such as β-tricalcium phosphate (β-TCP), hydroxyapatite (HA), type I collagen, poly-lactic-co-glycolic acid (PLGA) and alginate. The international patent application WO2013/059089 relates to a bone paste comprising stem cells and a mixture of calcium phosphate cement such as tricalcium phosphate and hydroxyapatite. US2011/104230 discloses a bone patch comprising scaffold material comprising synthetic ceramic material, mesenchymal stem cells and signaling molecules. US2016/287753 discloses a bone regeneration agent comprising a devitalized cell construct containing cells derived from stem cells a mineral and an extracellular matrix.

However, despite encouraging results in small animal models, critical-sized bone reconstruction using ASCs loaded on scaffolds remains limited by the large size of bone defect and consequently by the size of the implant to engineer. The cellular engraftment of the seeded cells is also limited by the poor diffusion of oxygen and nutrients. In addition, the cellular position within the scaffold is a major limitation for their in vitro and in vivo survival. Bioreactors with flow perfusion of scaffolds were designed to improve cell migration within the implant for a more homogenous cellular distribution, cell survival by delivering oxygen and nutrients to the core of the implant, and osteogenic cell differentiation (by the fluid shear force). Although these techniques are promising, relevant pre- and clinical data in large animal models are limited.

Recently, the publication WO2019/057861 disclosed a biomaterial comprising adipose-derived stem cells (ASCs), a biocompatible material and an extracellular matrix, wherein said biomaterial secreted OPG (osteoprotegerin).

Furthermore, the publication WO2019/057862 disclosed a biomaterial having a multi-dimensional structure comprising osteogenic differentiated adipose tissue-derived stem cells (ASCs), a ceramic material and an extracellular matrix, wherein the biomaterial secretes osteoprotegerin (OPG) and comprises insulin-like growth factor (IGF1) and stromal cell-derived factor 1-alpha (SDF-1α). It was shown that said biomaterial may be used for treating bone or cartilage defect.

In addition, the publication WO2020/058511 disclosed a biomaterial having a multi-dimensional structure comprising differentiated adipose tissue-derived stem cells (ASCs), an extracellular matrix and gelatin. It was shown that said biomaterial may be used for treating tissue disorders, such as bone, cartilage, muscle or skin disorders. Whereas both biomaterials may be suitable for autologous graft, on the other hand, allogenic or xenogeneic grafts cannot be however performed, because they may elicit an immune response hereby resulting in the rejection of the graft or they may carry adventitious pathogens resulting in infection of the recipient with the biomaterial. JP2004/305259 disclosed a biological tissue prosthesis which enables managing possible immunological rejection attributed to an implantation and is suitable for the long-time preservation.

Sterilization processes are often performed to alleviate these issues. However, these harsh conditions often deteriorate the biological properties of the sterilized material.

There is thus still a need in the art for tissue engineered materials for tissue reconstruction and/or regeneration that are fully biocompatible and provide appropriate mechanical features for the designated applications, although usable on a broad range of tissues.

There is also a need to provide a biomaterial for tissue reconstruction and/or regeneration, including for treating skin, bone or cartilage defects, that are adapted to allogenic or xenogeneic grafts. There is also a need to provide a sterile biomaterial that maintains the biological properties as compared to the fresh biomaterial, i.e., a biomaterial prior of being sterilized.

### SUMMARY

A first aspect of the invention pertains to a sterile and desiccated biomaterial comprising devitalized differentiated cells having tissue regenerating and/or repairing properties, and a particulate material, the cells and the particulate material being embedded in an extracellular matrix.

In certain embodiments, said cells are selected in a group comprising primary cells, stem cells, genetically modified cells, and a combination thereof. In some embodiments, at most 10% of said cells are viable, preferably at most 1%.

In certain embodiments, said particulate material is selected from the group comprising or consisting of:
an organic material, including demineralized bone matrix (DBM), gelatin, agar/agarose, alginates chitosan, chondroitin sulfate, collagen, elastin or elastin-like peptides (ELP), fibrinogen, fibrin, fibronectin, proteoglycans, heparan sulfate proteoglycans, hyaluronic acid, polysaccharides, laminins and cellulose derivatives;
a ceramic material, including particles of calcium phosphate (CaP), calcium carbonate (CaCO₃), calcium sulfate (CaSO₄), or calcium hydroxide (Ca(OH)₂), or combinations thereof,
a polymer, including polyanhydrides, polylactic acid (PLA), poly(lactic-co-glycolic acid) (PLGA), polyethylene oxide/polyethylene glycol (PEO/PEG), poly(vinyl alcohol) (PVA), fumarate-based polymers such as, for example poly(propylene fumarate) (PPF) or poly(propylene fumarate-co-ethylene glycol) (P(PF-co-EG)), oligo(poly(ethylene glycol) fumarate) (OPF), poly (n-isopropylacrylamide) (PNIPPAAm), poly(aldehyde guluronate) (PAG), poly(n-vinyl pyrrolidone) (PNVP), or combinations thereof;
a gel, including a self-assembling oligopeptide gel, a microgel, a nanogel, a particulate gel, a hydrogel, a thixotropic gel, a xerogel, a responsive gel, or combinations thereof,
a creamer;
and any combination thereof.

In some embodiments, said biomaterial comprises an altered factors content as compared to the factors content obtained from a corresponding fresh, non-sterile, non-desiccated biomaterial. In certain embodiments, the factors content includes growth factors and/or transcription factors. In some embodiments, the factors content comprises IGF-1 and/or VEGF and/or SDF-1α and/or OPG. In some embodiments, said factors content includes a RNAs content. In certain embodiments, the RNAs content comprises at least one miRNA selected in any one of Table 1, Table 2, Table 3, Table 4, Table 5, Table 6, Table 7, Table 8, Table 9, Table 10, Table 11 or Table 12. In some embodiments, said desiccated biomaterial is obtained by freeze-drying. In certain embodiments, said sterile biomaterial is obtained by gamma-irradiation, preferably at a dose of about 7 kGy to about 45 kGy, more preferably at room temperature.

Another aspect of the invention relates to a method for generating a sterile and desiccated biomaterial comprising devitalized differentiated cells and a particulate material, the cells and the particulate material being embedded in an extracellular matrix, said method comprising the steps of:
(1) contacting (i) viable cells capable to undergo differentiation with (ii) a particulate material, so as to obtain a first combination;
(2) culturing the first combination obtained in step (1) in a culture medium such that the cells secrete an extracellular matrix and synthesize a factors content, so as to acquire tissue regenerating and/or repairing properties, and wherein the cells and the particulate material are embedded in the extracellular matrix, so as to form a multidimensional structure;
(3) submitting the multidimensional structure obtained at step (2) to desiccation, so as to obtain a desiccated biomaterial;
(4) submitting the desiccated biomaterial obtained at step (3) to sterilization, preferably by gamma-irradiation, so as to obtain a sterile, desiccated biomaterial.

In some embodiments, the viable cells capable to undergo differentiation are selected in a group comprising primary cells; stem cells, in particular stems cells from adipose tissue, bone marrow, or umbilical cord blood; genetically modified cells; and a mixture thereof. A further aspect of the invention pertains to a desiccated and sterile biomaterial obtainable by the method according to the instant disclosure.

A still further aspect of the invention relates to a pharmaceutical composition comprising a biomaterial according to the instant disclosure, and a pharmaceutically acceptable vehicle. In certain embodiments, the composition is in the form of a paste or a film.

One aspect of the invention relates to a medical device comprising a biomaterial according to the instant disclosure, or a pharmaceutical composition according to the instant disclosure.

In one aspect, the invention also pertains to a biomaterial according to the instant disclosure, or a pharmaceutical composition according to the instant disclosure, for use as a medicament. In some embodiments, the biomaterial or the pharmaceutical composition is for use for preventing and/or treating a tissue disorder. In certain embodiments, said tissue is selected from the group comprising bone tissue, cartilage tissue, skin tissue, muscular tissue, epithelial tissue, endothelial tissue, neural tissue, connective tissue and adipose tissue. In some embodiments, the tissue disorder is selected in a group comprising aplasia cutis congenita; a burn; a cancer, including a breast cancer, a skin cancer and a bone cancer; a Compartment syndrome (CS); epidermolysis bulbosa; giant congenital nevi; an ischemic muscular injury of lower limbs; a muscle contusion, rupture or strain; a post-radiation lesion; and an ulcer, including a diabetic ulcer, preferably a diabetic foot ulcer; arthritis; bone fracture; bone frailty; Caffey's disease; congenital pseudarthrosis; cranial deformation; cranial malformation; delayed union; infiltrative disorders of bone; hyperostosis; loss of bone mineral density; metabolic bone loss; osteogenesis imperfecta; osteomalacia; osteonecrosis; osteopenia; osteoporosis; Paget's disease; pseudarthrosis; sclerotic lesions; spina bifida; spondylolisthesis; spondylolysis; chondrodysplasia; costochondritis; enchondroma; hallux rigidus; hip labral tear; osteochondritis dissecans; osteochondrodysplasia; polychondritis; and the likes. In certain embodiments, the biomaterial or the pharmaceutical composition is for use for preventing and/or treating a bone disorder and/or a cartilage disorder. In some embodiments, the biomaterial or the pharmaceutical composition is for use for tissue reconstruction. In certain embodiments, the biomaterial or the pharmaceutical composition is for use for compensating the side effects of a primary treatment of a tissue disorder, and/or for strengthening a primary treatment of a tissue disorder. In some embodiments, the biomaterial or the pharmaceutical composition is for use for compensating the side effects of a therapeutic treatment known to have a deleterious effect on tissues, in particular bone tissue, cartilage tissue, skin tissue, muscular tissue, epithelial tissue, endothelial tissue, neural tissue, connective tissue and adipose tissue.

### Definitions

Unless otherwise defined, all technical and scientific terms used herein have the same meaning as commonly understood by one of ordinary skill in the art to which this invention pertains. In case of conflict, the present definitions provided by the instant application are to be considered as authentic.

In the present invention, the following terms have the following meanings:
The term “about” preceding a value means plus or less 10% of the value of said value. It is to be understood that the value to which the term “about” refers to is itself also specifically, and preferably, disclosed.
The term “comprises” is intended to mean “contains”, “encompasses” and “includes”. In some embodiments, the term “comprises” also encompasses the term “consists of”.
The term “tissue disorder” is intended to refer to any disturbance, unbalance, of the physiological function of a tissue. Non-limitative examples of symptoms observed in tissue disorders include injury, strain, infection, sprain, trauma, fissure, swelling, redness, edema, aching, tenderness, soreness, wound, necrosis, or any combination thereof. As used herein, terms such as “tissue disorder”, “tissue disease”, “tissue medical condition” are intended to be equivalent.
The term “regeneration” or “tissue regeneration” includes, but is not limited to the growth, generation, or reconstruction of new cells types or tissues following a treatment with a biomaterial according to the invention. In one embodiment, these cells types or tissues include but are not limited to osteogenic cells (e.g. osteoblasts, osteocytes), chondrocytes, fibroblasts, keratinocytes, endothelial cells, cardiomyocytes, hematopoietic cells, hepatic cells, adipocytes, neural cells, and myotubes. As used herein, the term “regeneration” is envisioned as preventive and/or therapeutic treatment following injury, wounding, surgeries, congenital, degenerative, traumatic or non-traumatic symptoms or conditions, or other procedures that result in fissures, openings, depressions, wounds, and the like, of tissues.
The term “repair” or “tissue repair” includes but is not limited to the healing process to reconstruct a healthy tissue from a diseased or dysfunctional tissue. In one embodiment, tissue repair includes skin repair such as, e.g., would healing, scar formation and attenuation. In one embodiment, tissue repair includes bone repair, such as, e.g., fracture reduction. In one embodiment tissue repair includes cartilage repair. In one embodiment, tissue repair includes filling, bulking, supporting, enlarging, extending, or increasing the size, volume or mass of a body tissue.
The term “devitalized cells” as used herein refers to cells that do not possess an active metabolism and do not possess the ability to divide when contacted with a suitable culture medium.
The term “mesenchymal stem cells” as used herein refers to multipotent stem cells that can be differentiated into several types of cells belonging to skeletal tissues, such as cartilage, bone; skin tissues; muscular tissues; epithelium and/or endothelium tissues; neural tissue; connective tissue; and adipose tissue, such as fat.
The term “adipose tissue” refers to any fat tissue. The adipose tissue may be brown or white adipose tissue, derived from subcutaneous, omental/visceral, mammary, gonadal, or other adipose tissue site. Preferably, the adipose tissue is subcutaneous white adipose tissue. Such cells may comprise a primary cell culture or an immortalized cell line. The adipose tissue may be from any organism, living or deceased, having fat tissue. Preferably, the adipose tissue is originating from an animal, more preferably from a mammalian, most preferably the adipose tissue is from human origin. A convenient source of adipose tissue is from liposuction surgery, however, the source of adipose tissue or the method of isolation of adipose tissue is not critical to the invention.
The term “adipose tissue-derived stem cells” (also called “adipose tissue-derived stem cells”) as used herein refers to the “non-adipocyte” fraction of adipose tissue. The cells can be fresh, or in culture. “Adipose tissue-derived stem cells” (ASCs) refers to stromal cells that originate from adipose tissue which can serve as precursors to a variety of different cell types such as, but not limited to, adipocytes, osteocytes, chondrocytes, fibroblasts, myoblasts, epithelial cells, endothelial cells, connective cells, neural cells, the likes, and progenitor thereof.
The term “particulate material” as used herein refers to a solid material in the form of particles. Within the scope of the invention, “particulate material” includes organic materials, such as, e.g., demineralized bone matrix (DBM) and gelatin; ceramic materials; polymers, such as, e.g., polyanhydrides; gel, such as, e.g., hydrogel; and any combination thereof. Importantly, the particulate material may be also characterized by its mean diameter.
The term “ceramic material” as used herein refers to an inorganic, non-metallic, solid material. Ceramic material may comprise calcium phosphate (CaP), calcium carbonate (CaCO₃), calcium sulfate (CaSO₄), calcium hydroxide (Ca(OH)₂), or combinations thereof. Ceramic material may be in form of particles. The particulate material, preferably the ceramic material may be in form of powder, beads or granules. The particulate material, preferably the ceramic material may be porous.
The term “growth factors”, as used herein, encompass molecules that promote tissue growth, cellular proliferation, vascularization, and the like. In a particular embodiment, the term “growth factors” include molecules which promote skin, muscle, cartilage, endothelial, epithelial, neural, connective, adipose or bone tissue.
The term “transcription factors”, as used herein, are molecules that control whether a given gene is to be transcribed into its corresponding RNA.
The term “differentiated cell” as used herein refers to a precursor cell that has developed from an unspecialized phenotype to a specialized phenotype. For example, stem cells, in particular MSCs, such as ASCs, can differentiate into osteogenic, chondrogenic, adipose, epithelial, connective, neural or endothelial cells.
The term “differentiation medium” as used herein refers to one of a combination or collection of compounds that are used in culture systems of this invention to produce differentiated cells. No limitation is intended as to the mode of action of the compounds. For example, the agent may assist the differentiation process by inducing or assisting a change in phenotype, promoting growth of cells with a particular phenotype or retarding the growth of others. It may also act as an inhibitor to other factors that may be in the medium or synthesized by the cell population that would otherwise direct differentiation down the pathway to an unwanted cell type.
The term “miRNA” or “miR” refers to a non-coding RNA of about 18 to about 25 nucleotides in length. These miRNAs could originate from multiple origins including: an individual gene encoding for a miRNA, from introns of protein coding gene, or from poly-cistronic transcript that often encode multiple, closely related miRNAs. In the following disclosure, the standard nomenclature system is applied, in which uncapitalized “mir-X” refers to the pre-miRNA (precursor), and capitalized “miR-X” refers to the mature form. When two mature miRNAs originate from opposite arms of the same pre-miRNA, they are denoted with a −3p or −5p suffix. In the following disclosure, unless otherwise specified, the use of the expression miR-X refers to the mature miRNA including both forms −3p and −5p, if any. Within the scope of the invention, the expressions microRNA, miRNA and miR designate the same compound.
The term “exosome” is intended to refer to the extracellular vesicles that are released from cells upon fusion of an intermediate endocytic compartment, the multivesicular body (MVB), with the plasma membrane. In other words, exosomes correspond to the intraluminal vesicles that are released into the extracellular milieu.
The term “secretion” refers to a physiologically active substance transported out of the cell in which it is synthesized. In one embodiment, the physiologically active substance may be any molecule, in particular a protein (such as a growth factor or a transcription factor) or a nucleic acid (such as a miRNA). As used herein, the term “secretion” includes both active and passive secretion. In this application, the term “active secretion” refers to the secretion of physiologically active substances by living cells, in particular mesenchymal stem cells and preferably adipose tissue-derived stem cells, out of the cells as a response to a stimulus, thereby diffusing into the environment of the cells, such as, e.g., the extracellular matrix. By “living cells”, it is meant herein cells presenting at least one of the following characteristics: growth and development, reproduction, homeostasis, response to stimuli, consumption, metabolism, excretion. In this application, the term “passive secretion” refers to physiologically active substances released by non-living cells or fragments or extracts thereof, out of the cells or fragments or extracts thereof in the absence of a stimulus, thereby diffusing into the environment of the originating cells or fragments or extracts thereof, such as, e.g., the extracellular matrix. By “non-living cells”, it is meant herein cells presenting none of the following characteristics: growth and development, reproduction, homeostasis, response to stimuli, consumption, metabolism, excretion (non-living cells or fragments or extracts thereof are, for example, dead cells or cellular extracts). Physiologically active substances that are actively or passively secreted may then diffuse into the tissue or organ in which the biomaterial comprising such extracellular matrix is administered.
The terms “treatment”, “treating” or “alleviation” refer to therapeutic treatments wherein the object is to prevent or slow down (lessen) a tissue disorder, including skin disorder, bone disorder and/or a cartilage disorder. Those in need of treatment include those already with the disorder as well as those prone to have the disorder or those in whom the tissue disorder, including the bone or cartilage defect, is to be prevented. A subject is successfully “treated” for a tissue disorder, including a skin disorder, bone disorder and/or a cartilage disorder if, after receiving a therapeutic amount of a biomaterial according to the methods of the present invention, the individual shows observable and/or measurable reduction in, or absence of, one or more of the following: reduction in the tissue disorder, including the skin disorder, bone disorder and/or cartilage disorder; and/or relief to some extent, one or more of the symptoms associated with the tissue disorder, including the skin disorder, bone disorder and/or cartilage disorder; reduced morbidity and mortality, and improvement in quality of life issues. The above parameters for assessing successful treatment and improvement in the disorder are readily measurable by routine procedures familiar to a physician. In the context of therapeutic use of the disclosed biomaterials, in “allogeneic” (or “allogenic”) therapy, the donor and the recipient are different individuals of the same species, whereas in “autologous” therapy, the donor and the recipient is the same individual, and in “xenogeneic” therapy, the donor derived from an animal of a different species than the recipient.
The term “prevention” refers to preventing or avoiding the occurrence of symptom of a tissue disorder, including a skin disorder, bone disorder and/or cartilage disorder. In the present invention, the term “prevention” may refer to a secondary prevention, i.e. to the prevention of the re-occurrence of a symptom or a relapse of a tissue disorder, including a skin disorder, bone disorder and/or cartilage disorder. It may also refer, when the disease is cancer, such as, e.g., a bone cancer, to the occurrence of metastases after the treatment and/or the removal of a tumor.
The term “effective amount” refers to an amount sufficient to effect beneficial or desired results including clinical results. An effective amount can be administered in one or more administrations.
“Pharmaceutically acceptable vehicle” refers to a vehicle that does not produce any adverse, allergic or other unwanted reactions when administered to an animal individual, preferably a human individual. It includes any and all solvents, dispersion media, coatings, antibacterial and antifungal agents, isotonic and absorption delaying agents and the like. For human administration, preparations should meet sterility, pyrogenicity, general safety, quality and purity standards as required by regulatory Offices, such as, e.g., the Food and Drug Administration (FDA) in the United States or the European Medicines Agency (EMA) in the European Union.
The term “individual” refers to a vertebrate animal, preferably a mammal, more preferably a human. Examples of individuals include humans, non-human primates, dogs, cats, mice, rats, horses, cows, sheep and transgenic species thereof. In one embodiment, an individual may be a “patient”, i.e. a warm-blooded animal, more preferably a human, who/which is awaiting the receipt of, or is receiving medical care or was/is/will be the object of a medical procedure, or is monitored for the development of a disease. In one embodiment, the individual is an adult (for example a human subject above the age of 18). In another embodiment, the individual is a child (for example a human subject below the age of 18). In one embodiment, the individual is a male. In another embodiment, the individual is a female.

Other definitions may appear in context throughout this disclosure.

### DETAILED DESCRIPTION

The inventors have further characterized the biomaterials disclosed in the publications WO2019/057862 and WO2020/058511. It emerged from this further characterization that the cellular content and/or the secreted content is/are of primary importance for promoting tissue repair, including skin repair, bone repair or cartilage repair. Noticeably, it was shown that growth factors, transcription factors, and factors that are involved in tissue formation, including skin formation, bone formation or cartilage formation, together with various micro RNAs (miRNAs), may represent the active agents, as they promote, e.g., the osteogenic and/or chondrogenic properties of the biomaterial.

In addition, the inventors have observed that said biomaterials may be advantageously freeze-dried and gamma-irradiated and, against all odds, may maintain their tissue regenerating and/or tissue repairing properties, including their osteogenic and/or chondrogenic properties. The fresh multidimensional biomaterials have been characterized and comprise numerous factors, such as growth factors, transcription factors, including polypeptides and miRNAs. However, the inventors were surprised by the fact that the freeze-drying and the gamma-irradiation, which are known to alter biological properties, in particular miRNAs properties, did not alter tissue regenerating and/or tissue repairing properties, including osteogenic and/or chondrogenic properties of the treated biomaterials, despite a significant variation of the relative amounts of various factors as compared to the untreated original biomaterials.

The recitation of an embodiment below includes that embodiment as any single embodiment or in combination with any other embodiments or portions thereof and the recited embodiments are applicable to one or more of the aspects recited below. Other features and advantages of the invention will be apparent from the detailed description and from the claims. Thus, other aspects and embodiments of the invention are described in the following disclosure and are within the ambit of the invention.

This invention relates to a sterile and desiccated biomaterial comprising devitalized differentiated cells having tissue regenerating and/or repairing properties, and a particulate material, the cells and the particulate material being embedded in an extracellular matrix.

As used herein, the expression “differentiated cells having tissue regenerating and/or repairing properties” is intended to refer to a cell population that possesses the ability to promote tissue regenerating and/or repairing, and/or to maintain existing tissues in a healthy physiological condition.

This invention also relates to a sterile and desiccated biomaterial comprising devitalized differentiated cells having tissue regenerating and/or repairing properties, and gelatin, the cells and the gelatin being embedded in an extracellular matrix.

This invention further relates to a sterile and desiccated biomaterial comprising devitalized osteo-differentiated and/or chondro-differentiated cells, and a particulate material, the cells and the particulate material being embedded in an extracellular matrix.

As used herein, the expression “osteo-differentiated and/or chondro-differentiated cells” is intended to refer to a cell population that possesses the ability to promote bone and/or cartilage formation, and/or to maintain existing bone and/or cartilage in a healthy physiological condition.

In certain embodiments, said cells are selected in a group comprising primary cells, stem cells, genetically modified cells, and a combination thereof.

In practice, the cells according to the instant invention may be animal cells, preferably mammal cells, more preferably human cells.

In some embodiments, the primary cells are selected in the group comprising, or consisting of, bone cells, brain cells, skin cells, breast cells, neural cells, cervix cells, cells of the upper aero digestive tract, colorectal cells, endometrial cells, germ cells, bladder cells, kidney cells, laryngeal cells, liver cells, lung cells, esophageal cells, ovarian cells, pancreatic cells, pleural cells, prostate cells, ocular cells, small intestine cells, stomach cells, testicular cells, thyroid cells, the likes, and progenitors thereof.

In some embodiments, primary cells may be selected in a group comprising or consisting of osteocytes, osteoblasts, osteoclasts, chondroblasts, chondrocytes, keratinocytes, dermal fibroblasts, fibroblasts, epithelial cells, hematopoietic cells, hepatic cells, neural cells, myofibroblasts, endothelial cells, adipocytes, and a combination thereof.

As used herein the term “neural cells” encompasses cells of the central nervous system, such as, e.g., neurons and glial cells.

In some embodiments, primary cells may be selected in a group comprising osteocytes, osteoblasts, osteoclasts, chondroblasts, chondrocytes and a combination thereof. Because primary cells are differentiated cells, they can be cultured in any suitable culture medium for maintenance or proliferation purposes. In some embodiments, the primary cells may be cultured in a culture medium suitable for allowing proliferation or maintenance of the cells (also referred to as proliferation medium (MP)).

In one embodiment, proliferation medium may be any culture medium designed to support the growth of the cells known to one of ordinary skill in the art. As used herein, the proliferation medium is also called “growth medium”. Examples of growth medium include, without limitation, RPMI, MEM, DMEM, IMDM, RPMI 1640, FGM or FGM-2, 199/109 medium, HamF10/HamF12 or McCoy's 5A. In a preferred embodiment, the proliferation medium is DMEM.

In certain embodiments, stem cells may be selected in a group comprising osteoprogenitors, embryonic stem cells (ESCs), mesenchymal stem cells (MSCs), pluripotent stem cells (pSCs) and induced pluripotent stem cells (ipSCs).

As used herein, “embryonic stem cells” (ESCs) generally refer to embryonic cells, which are capable of differentiating into cells of any one of the three embryonic germ layers, namely endoderm, ectoderm or mesoderm, or capable of being maintained in an undifferentiated state. Such cells may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation and other methods with non-fertilized eggs, such as parthenogenesis method or nuclear transfer.

In certain embodiments, the ESCs according to the invention are animal ESCs, preferably mammal ESCs, more preferably human ESCs (hESCs).

In practice, suitable EScs may be obtained using well-known cell-culture methods. For example, ESCs can be isolated from blastocysts. Blastocysts are typically obtained from in vivo preimplantation embryos or from in vitro fertilized (IVF) embryos. Alternatively, a single cell embryo can be expanded to the blastocyst stage. Further details on methods of preparation ESCs may be found in U.S. Pat. No. 5,843,780.

In some embodiments, hESCs may advantageously be obtained without embryo destruction, as described by Chung et al. (2008)). In some embodiments, hESCs may be advantageously obtained from embryo collected or isolated less than 14 days upon fertilization. In some embodiments, the ESCs are not human ESCs.

As used herein, “mesenchymal stem cells” (MSCs) generally refer to stromal cells from a specialized tissue (also named differentiated tissue) and capable of self-renewal (i.e. making identical copies of themselves) for the lifetime of the organism and have multipotent differentiation potential.

In some embodiments, the MSCs according to the invention are animal MSCs, preferably mammal MSCs, more preferably human MSCs (hMSCs). In practice, hMSCs suitable for implementing the instant invention thus encompass any suitable human multipotent stem cells derived from any suitable tissue, using any appropriate isolation method.

Illustratively, hMSCs encompass, but are not limited to, adult multilineage inducible (MIAMI) cells (D'Ippolito et al.; 2004), cord blood derived stem cells (Kögler et al.; 2004), mesoangioblasts (Sampaolesi et al.; 2006; Dellavalle et al.; 2007), and amniotic stem cells (De Coppi et al.; 2007). Furthermore, umbilical cord blood banks (e.g., Etablissement Français du Sang, France) provide secure and easily available sources of such cells for transplantation.

In some embodiments, the MSCs are pre-osteoblasts, pre-chondroblasts, pre-keratinocytes, pre-fibroblasts. In some embodiments, the MSCs according to the invention are pre-osteoblasts or pre-chondroblasts.

In some embodiments, the mesenchymal stem cells are adipose tissue-derived stem cells (ASCs). As used herein, the following terms are considered to refer to ASCs: Adipose-derived Stem/Stromal Cells (ASCs); Adipose Derived Adult Stem (ADAS) Cells, Adipose Derived Adult Stromal Cells, Adipose Derived Stromal Cells (ADSC), Adipose Stromal Cells (ASC), Adipose Mesenchymal Stem Cells (AdMSC), Lipoblasts, Pericytes, Pre-Adipocytes, Processed Lipoaspirate (PLA) Cells.

In one embodiment, ASCs are of animal origin, preferably of mammal origin, more preferably of human origin. Accordingly, in one embodiment, ASCs are animal ASCs, preferably mammal ASCs, more preferably human ASCs. In a preferred embodiment, ASCs are human ASCs.

Methods of isolating stem cells from adipose tissue are known in the art and are disclosed for example in Zuk et al. (Tissue Engineering. 2001, 7:211-228). In one embodiment, ASCs are isolated from adipose tissue by liposuction.

As an illustration, adipose tissue may be collected by needle biopsy or liposuction aspiration. ASCs may be isolated from adipose tissue by first washing the tissue sample extensively with phosphate-buffered saline (PBS), optionally containing antibiotics, for example 1% Penicillin/Streptomycin (P/S). Then the sample may be placed in a sterile tissue culture plate, or a sterile tube, with collagenase for tissue digestion (for example, Collagenase Type I prepared in PBS containing 2% P/S), and incubated for 60 min at 37° C., 5% CO₂, in a water bath, with manual shaking every 20 min. The collagenase activity may be neutralized by adding culture medium (for example DMEM containing 10% human platelet lysate (hPL)). Upon disintegration, the sample may be transferred to a tube. The stromal vascular fraction (SVF), containing the ASCs, is obtained by centrifuging the sample (for example at 2,000 rpm for 5 min). To complete the separation of the stromal cells from the primary adipocytes, the sample may be shaken vigorously to thoroughly disrupt the pellet and to mix the cells. The centrifugation step may be repeated. After spinning and the collagenase solution aspirate, the pellet may be resuspended in lysis buffer, incubated on ice (for example for 10 min), washed (for example with PBS/2% P/S) and centrifuged (for example at 2,000 rpm for 5 min). The supernatant may be then aspirated, the cell pellet resuspended in medium (for example, stromal medium, i.e. α-MEM, supplemented with 20% FBS, 1% L-glutamine, and 1% P/S), and the cell suspension filtered (for example, through 70 μm cell strainer). The sample containing the cells may be finally plated in culture plates and incubated at 37° C., 5% Co₂.

In one embodiment, ASCs of the invention are isolated from the stromal vascular fraction of adipose tissue. In one embodiment, the lipoaspirate may be kept several hours at room temperature, or at +4° C. for 24-72 hours prior to use, or below 0° C., for example −18° C. or −80° C., for long-term conservation.

In one embodiment, ASCs may be fresh ASCs or refrigerated ASCs. Fresh ASCs are isolated ASCs which have not undergone a refrigerating treatment. Refrigerated ASCs are isolated ASCs which have undergone a refrigerating treatment. In one embodiment, a refrigerating treatment means any treatment below 0° C. In one embodiment, the refrigerating treatment may be performed at about −18° C., at −80° C. or at −180° C. In a specific embodiment, the refrigerating treatment may be cryopreservation.

As an illustration of refrigerating treatment, ASCs may be harvested at 80-90% confluence. After steps of washing and detachment from the dish, cells may be pelleted at 20° C. with a refrigerating preservation medium and placed in vials. In one embodiment, the refrigerating preservation medium comprises 80% fetal bovine serum or human serum, 10% dimethylsulfoxide (DMSO) and 10% DMEM/Ham's F-12. Then, vials may be stored at −80° C. overnight. For example, vials may be placed in an alcohol freezing container which cools the vials slowly, at approximately 1° C. every minute, until reaching −80° C. Finally, frozen vials may be transferred to a liquid nitrogen container for long-term storage.

In one embodiment, ASCs are differentiated ASCs. In some embodiments, said differentiated cells are differentiated adipose tissue-derived stem cells (ASCs), preferably ASCs differentiated into cells selected from the group comprising or consisting of osteoblasts, chondrocytes, keratinocytes, myofibroblasts, epithelial, endothelial, connective, or neural cells and adipocytes.

In a preferred embodiment, ASCs are osteogenic differentiated ACSs. In other words, in a preferred embodiment, ASCs are differentiated into osteogenic cells. In a particular embodiment, ASCs are differentiated into osteoblasts.

In another embodiment, ASCs are chondrogenic differentiated ACSs. In other words, in one embodiment, ASCs are differentiated into chondrogenic cells. In a particular embodiment, ASCs are differentiated into chondrocytes.

In another embodiment, ASCs are keratinic differentiated ACSs. In other words, in one embodiment, ASCs are differentiated into keratinic cells. In a particular embodiment, ASCs are differentiated into keratinocytes.

In another embodiment, ASCs are myofibroblastic differentiated ACSs. In other words, in one embodiment, ASCs are differentiated into myofibroblastic cells. In a particular embodiment, ASCs are differentiated into myofibroblasts.

In another embodiment, ASCs are endothelial differentiated ACSs. In other words, in one embodiment, ASCs are differentiated into endothelial cells. In a particular embodiment, ASCs are differentiated into endothelial cells.

In another embodiment, ASCs are epithelial differentiated ACSs. In other words, in one embodiment, ASCs are differentiated into epithelial cells. In a particular embodiment, ASCs are differentiated into epithelial cells.

In another embodiment, ASCs are adipogenic differentiated ACSs. In other words, in one embodiment, ASCs are differentiated into adipogenic cells. In a particular embodiment, ASCs are differentiated into adipocytes.

In another embodiment, ASCs are neural differentiated ACSs. In other words, in one embodiment, ASCs are differentiated into neural cells.

As used herein, the term “pluripotent” refers to cells having the capacity to generate a cellular progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8 to 12 weeks-old SCID mice, can be used to establish the pluripotency of a cell population. However, identification of various pluripotent stem cell characteristics can also be used to identify pluripotent cells. In some embodiments of the invention, the pluripotent stem cells are animal pluripotent stem cells, preferably mammal pluripotent stem cells, more preferably human pluripotent stem cells.

As used herein, an “induced pluripotent stem cell” (iPSC) refers to a pluripotent stem cell artificially derived from a non-pluripotent cell. A non-pluripotent cell may be a cell of lesser ability (or potency) to self-renew and to differentiate as compared to a pluripotent stem cell. Cells of lesser potency may be, but are not limited to, somatic stem cells, tissue specific progenitor cells, primary or secondary cells. In some embodiments, the iPSCs are human iPSCs (hiPSCs).

On the contrary, because stem cells and genetically modified cells are not differentiated cells, they may undergo a differentiation process, such as, e.g., an osteogenic and/or chondrogenic differentiation process.

In some embodiments, the cells are genetically modified cells. In practice genetically modified cells are engineered so as to synthesize the factors and the nucleic acids that promote tissue regeneration and/or tissue repairing, including osteogenic and/or chondrogenic properties.

Within the scope of the instant invention, the expression “genetically modified” is intended to refer to a cell that possesses one or more nucleotide substitution, addition or deletion in its genome and/or comprises one or more additional extra chromosomic nucleic acids encoding one or more factors interfering with the physiological outcome of the cell's fate. In certain embodiments, the genetically modified cells are of animal origin, preferably of mammal origin, more preferably of human origin.

In some embodiments, the genetically modified cells are engineered so as to allow the synthesis of one or more growth factor, transcription factor or RNAs involved in tissue regeneration and/or tissue repairing, including osteogenesis and/or chondrogenesis.

In one embodiment, osteogenic differentiation of stem cells or genetically modified cells, in particular ASCs, is performed by culture of cells in osteogenic differentiation medium (MD). In one embodiment, the osteogenic differentiation medium comprises human serum. In a particular embodiment, the osteogenic differentiation medium comprises human platelet lysate (hPL). In one embodiment, the osteogenic differentiation medium does not comprise any other animal serum, preferably it comprises no other serum than human serum.

Methods to control and assess the osteogenic differentiation are known in the art. For example, the osteo-differentiation of the cells or tissues of the invention may be assessed by staining of osteocalcin and/or phosphate (e.g., with von Kossa); by staining calcium phosphate (e.g., with Alizarin red); by magnetic resonance imaging (MRI); by measurement of mineralized matrix formation; or by measurement of alkaline phosphatase activity.

In one embodiment, the osteogenic differentiation medium comprises or consists of proliferation medium supplemented with dexamethasone, ascorbic acid and sodium phosphate. In one embodiment, the osteogenic differentiation medium further comprises antibiotics, such as penicillin, streptomycin, gentamycin and/or amphotericin B. In one embodiment, all media are free of animal proteins.

In one embodiment, the osteogenic differentiation medium comprises or consists of DMEM supplemented with L-alanyl-L-glutamine (Ala-Gln, also called ‘Glutamax®’ or ‘Ultraglutamine®’), hPL, dexamethasone, ascorbic acid and sodium phosphate. In one embodiment, the osteogenic differentiation medium comprises or consists of DMEM supplemented with L-alanyl-L-glutamine, hPL, dexamethasone, ascorbic and sodium phosphate, and antibiotics, preferably penicillin, streptomycin, gentamycin and/or amphotericin B.

In one embodiment, the osteogenic differentiation medium comprises or consists of DMEM supplemented with L-alanyl-L-glutamine, hPL (about 5%, v/v), dexamethasone (about 1 μM), ascorbic acid (about 0.25 mM) and sodium phosphate (about 2.93 mM). In one embodiment, the osteogenic differentiation medium comprises or consists of DMEM supplemented with L-alanyl-L-glutamine, hPL (about 5%, v/v), dexamethasone (about 1 μM), ascorbic acid (about 0.25 mM) and sodium phosphate (about 2.93 mM), penicillin (about 100 U/mL) and streptomycin (about 100 μg/mL). In one embodiment, the osteogenic differentiation medium further comprises amphotericin B (about 0.1%).

In one embodiment, the osteogenic differentiation medium consists of DMEM supplemented with L-alanyl-L-glutamine, hPL (about 5%, v/v), dexamethasone (about 1 μM), ascorbic acid (about 0.25 mM) and sodium phosphate (about 2.93 mM). In one embodiment, the osteogenic differentiation medium comprises or consists of DMEM supplemented with L-alanyl-L-glutamine, hPL (about 5%, v/v), dexamethasone (about 1 μM), ascorbic acid (about 0.25 mM) and sodium phosphate (about 2.93 mM), penicillin (about 100 U/mL), streptomycin (about 100 μg/mL) and amphotericin B (about 0.1%).

In another embodiment, the cells, in particular ASCs, are chondrogenic differentiated. In other words, in a preferred embodiment, the cells, in particular ASCs, are differentiated into chondrogenic cells. In still other words, in a preferred embodiment, the cells, in particular ASCs, are differentiated in chondrogenic medium. In a particular embodiment, the cells, in particular ASCs, are differentiated into chondrocytes.

Methods to control and assess the chondrogenic differentiation are known in the art. For example, the chondro-differentiation of the cells or tissues of the invention may be assessed by staining of Alcian Blue, by measurement of the expression level of chondrocyte-specific genes such as aggrecan, collagen II and SOX-9. Methods include, but are not limited to, real-time PCR or histological analysis.

In one embodiment, chondrogenic differentiation is performed by culture of the cells, in particular ASCs, in chondrogenic differentiation medium.

In one embodiment, the chondrogenic differentiation medium comprises or consists of DMEM, hPL, sodium pyruvate, ITS, proline, TGF-β1 and dexamethazone. In one embodiment, the chondrogenic differentiation medium further comprises antibiotics, such as penicillin, streptomycin, gentamycin and/or amphotericin B.

In one embodiment, the chondrogenic differentiation medium comprises or consists of proliferation medium supplemented with sodium pyruvate, ascorbic acid and dexamethasone. In one embodiment, the chondrogenic differentiation medium further comprises antibiotics, such as penicillin, streptomycin, gentamycin and/or amphotericin B. In one embodiment, the chondrogenic differentiation medium further comprises growth factors, such as IGF and TGF-β. In one embodiment, all media are free of animal proteins.

In one embodiment, the chondrogenic differentiation medium comprises or consists of DMEM supplemented with hPL, dexamethasone, ascorbic acid and sodium pyruvate. In one embodiment, the chondrogenic differentiation medium may further comprise proline and/or growth factors and/or antibiotics.

In one embodiment, the chondrogenic differentiation medium comprises or consists of DMEM, hPL (about 5%, v/v), dexamethasone (about 1 μM), sodium pyruvate (about 100 μg/mL), ITS (about 1×), proline (about 40 μg/mL) and TGF-β1 (about 10 ng/mL). In another embodiment, the cells, in particular ASCs, are keratinogenic differentiated. In other words, in a preferred embodiment, the cells, in particular ASCs, are differentiated into keratinogenic cells. In still other words, in a preferred embodiment, the cells, in particular ASCs, are differentiated in keratinogenic medium. In a particular embodiment, the cells, in particular ASCs, are differentiated into keratinocytes.

Methods to control and assess the keratinogenic differentiation are known in the art. For example, the keratinogenic differentiation of the cells or tissues of the invention may be assessed by staining of Pankeratin or CD34.

In one embodiment, differentiation into keratinocytes are performed by culture of the cells, in particular ASCs, in keratinogenic differentiation medium.

In one embodiment, the keratinogenic differentiation medium comprises or consists of DMEM, hPL, insulin, KGF, hEGF, hydrocortisone and CaCl₂). In one embodiment, the keratinogenic differentiation medium further comprises antibiotics, such as penicillin, streptomycin, gentamycin and/or amphotericin B.

In one embodiment, the keratinogenic differentiation medium comprises or consists of DMEM, hPL (about 5%, v/v), insulin (about 5 μg/mL), KGF (about 10 ng/mL), hEGF (about 10 ng/mL), hydrocortisone (about 0.5 μg/mL) and CaCl₂) (about 1.5 mM).

In another embodiment, the cells, in particular ASCs, are endothelial differentiated. In still other words, in a preferred embodiment, the cells, in particular ASCs, are differentiated in endothelial medium. In a particular embodiment, the cells, in particular ASCs, are differentiated into endothelial cells.

Methods to control and assess the endothelial differentiation are known in the art. For example, the endothelial differentiation of the cells or tissues of the invention may be assessed by staining of CD34.

In one embodiment, differentiation into endothelial cells are performed by culture of ASCs in endothelial differentiation medium.

In one embodiment, the endothelial differentiation medium comprises or consists of EBM™-2 medium, hPL, hEGF, VEGF, R3-IGF-1, ascorbic acid, hydrocortisone and hFGFb. In one embodiment, the endothelial differentiation medium further comprises antibiotics, such as penicillin, streptomycin, gentamycin and/or amphotericin B.

In one embodiment, the endothelial differentiation medium comprises or consists of EBM™-2 medium, hPL (about 5%, v/v), hEGF (about 0.5 mL), VEGF (about 0.5 mL), R3-IGF-1 (about 0.5 mL), ascorbic acid (about 0.5 mL), hydrocortisone (about 0.2 mL) and hFGFb (about 2 mL), reagents of the kit Clonetics™ EGM™-2MV BulletKit™ CC-3202 (Lonza).

In another embodiment, the cells, in particular ASCs, are myofibrogenic differentiated. In other words, in a preferred embodiment, the cells, in particular ASCs, are differentiated into myofibrogenic cells. In still other words, in a preferred embodiment, the cells, in particular ASCs, are differentiated in myofibrogenic medium. In a particular embodiment, the cells, in particular ASCs, are differentiated into myofibroblasts.

Methods to control and assess the myofibrogenic differentiation are known in the art. For example, the myofibrogenic differentiation of the cells or tissues of the invention may be assessed by staining of α-SMA.

In one embodiment, differentiation into myofibrogenic cells are performed by culture of the cells, in particular ASCs, in myofibrogenic differentiation medium.

In one embodiment, the myofibrogenic differentiation medium comprises or consists of DMEM:F12, sodium pyruvate, ITS, RPMI 1640 vitamin, TGF-β1, Glutathione, MEM. In one embodiment, the myofibrogenic differentiation medium further comprises antibiotics, such as penicillin, streptomycin, gentamycin and/or amphotericin B.

In one embodiment, the myofibrogenic differentiation medium comprises or consists of DMEM:F12, sodium pyruvate (about 100 μg/mL), ITS (about 1×), RPMI 1640 vitamin (about 1×), TGF-β1 (about 1 ng/mL), Glutathione (about 1 μg/mL), MEM (about 0.1 mM).

In another embodiment, the cells, in particular ASCs, are adipogenic differentiated. In other words, in a preferred embodiment, the cells, in particular ASCs, are differentiated into adipogenic cells. In still other words, in a preferred embodiment, the cells, in particular ASCs, are differentiated in adipogenic medium. In a particular embodiment, the cells, in particular ASCs, are differentiated into adipocytes.

Methods to control and assess the adipogenic differentiation are known in the art. For example, the adipogenic differentiation of the cells or tissues of the invention may be assessed by staining by Oil-Red.

In one embodiment, differentiation into adipocytes are performed by culture of the cells, in particular ASCs, in adipogenic differentiation medium.

In one embodiment, the adipogenic differentiation medium comprises or consists of DMEM, hPL, Dexamethazone, insulin, Indomethacin and IBMX. In one embodiment, the adipogenic differentiation medium further comprises antibiotics, such as penicillin, streptomycin, gentamycin and/or amphotericin B.

In one embodiment, the adipogenic differentiation medium comprises or consists of DMEM, hPL (about 5%), Dexamethazone (about 1 μM), insulin (about 5 μg/mL), Indomethacin (about 50 μM) and IBMX (about 0.5 mM).

In another embodiment, the cells, in particular ASCs, are neural differentiated. In other words, in a preferred embodiment, the cells, in particular ASCs, are differentiated into neural cells. In a particular embodiment, the cells, in particular ASCs, are differentiated into neural cells. In a specific embodiment, the cells, in particular ASCs, are differentiated into neurons. In another specific embodiment, the cells, in particular ASCs, are differentiated into glial cells.

In one embodiment, differentiation into neural cells are performed by culture of the cells, in particular ASCs, in neurons or glial cells differentiation medium.

Methods to control and assess the neural differentiation are known in the art. For example, the neural differentiation of the cells or tissues of the invention may be assessed according to the morphology, physiology, or global gene expression pattern. For instance, the neural differentiation of the cells or tissues of the invention may be assessed by the cell growth in length, by the development of a growth cone, and/or by staining of neuroectodermal stem cell markers including NESTIN, PAX6, and SOX2. Another method to control and assess the neural differentiation is to assess the electrophysiological profile of the differentiated cells.

In one embodiment, the cells, in particular ASCs, are late passaged adipose tissue-derived stem cells. As used herein, the term “late passages” means adipose tissue-derived stem cells differentiated at least after passage 4. As used herein, the passage 4 refers to the fourth passage, i.e. the fourth act of splitting cells by detaching them from the surface of the culture vessel before they are resuspended in fresh medium. In one embodiment, late passaged adipose tissue-derived stem cells are differentiated after passage 4, passage 5, passage 6 or more. In a preferred embodiment, cells, in particular ASCs, are differentiated after passage 4.

As used herein, the term “vessel” means any cell culture surface, such as for example a flask or a well-plate.

The initial passage of the primary cells was referred to as passage 0 (P0). According to the present invention, passage P0 refers to the seeding of cell suspension from the pelleted Stromal Vascular Fraction (SVF) on culture vessels. Therefore, passage P4 means that cells were detached 4 times (at P1, P2, P3 and P4) from the surface of the culture vessel (for example by digestion with trypsin) and resuspended in fresh medium.

In one embodiment, the cells of the invention, in particular ASCs, are cultured in proliferation medium up to the fourth passage. In one embodiment, the cells of the invention, in particular ASCs, are cultured in differentiation medium after the fourth passage. Accordingly, in one embodiment, at passages P1, P2 and P3, the cells of the invention, in particular ASCs are detached from the surface of the culture vessel and then diluted to the appropriate cell density in proliferation medium. Still according to this embodiment, at passage P4, cells, in particular ASCs, are detached from the surface of the culture vessel and then diluted to the appropriate cell density in differentiation medium. Therefore, according to this embodiment, at P4 the cells of the invention, in particular ASCs, are not resuspended and cultured in proliferation medium until they reach confluence before being differentiated (i.e. before being cultured in differentiation medium) but are directly resuspended and cultured in differentiation medium.

In one embodiment, cells are maintained in differentiation medium at least until they reach confluence, preferably between 70% and 100% confluence, more preferably between 80% and 95% confluence. In one embodiment, cells are maintained in differentiation medium for at least 5 days, preferably at least 10 days, more preferably at least 15 days. In one embodiment, cells are maintained in differentiation medium from 5 days to 30 days, preferably from 10 days to 25 days, more preferably from 15 days to 20 days. In one embodiment, differentiation medium is replaced every 2 days. However, as it is known in the art, the cell growth rate from one donor to another could slightly differ.

Thus, the duration of the differentiation and the number of medium changes may vary from one donor to another.

In one embodiment, cells are maintained in differentiation medium at least until formation of distinctive tissue depending on the differentiation medium used.

In one embodiment, cells are maintained in osteogenic differentiation medium at least until formation of osteoid, i.e. the unmineralized, organic portion of the bone matrix that forms prior to the maturation of bone tissue.

In one embodiment, cells are maintained in chondrogenic differentiation medium at least until formation of cartilage, immature or mature, with viscoelastic properties.

In some embodiments, at most 10% of said cells are viable, preferably at most 1%.

Within the scope of the invention, the expression “at most 10%” includes 10%, 9%, 8%, 7%, 6%, 5% 4%, 3%, 2%, 1%, 0.5%, 0.1%, 0.05%, 0.01%, 0.005%, 0.001% and 0%.

Viability of the cells according to the invention may be assessed by any suitable methods known in the state of the art, or adapted therefrom. One may refer to, e.g., “Mammalian cell viability: Methods and Protocols” (2011; Editor: M J Stoddart). Illustratively, cells may be recovered upon hydration of the desiccated biomaterial and contacted with a suitable culturing medium in adapted culturing conditions. Viability of the cells may be assessed upon trypan blue dye exclusion staining. Alternatively, viability of the cells may be assessed upon measurement of the consumption of a carbon source, in particular glucose, in the culture medium.

In some embodiment, the biomaterial comprises substantially non-viable cells. In said embodiment, the biomaterial comprises an undetectable level of viable cells. In said embodiment, the biomaterial may be referred to as being devitalized.

In one embodiment, the particulate material of the invention is in form of particles. In one embodiment, particles may be beads, powder, spheres, microspheres, and the like.

In some embodiments, the particulate material of the invention is formed by a material that provides a structural support for the growth and propagation of cells. In one embodiment, particulate material is biocompatible, and comprises a natural or synthetic material, or a chemical-derivative thereof.

Within the scope of the instant invention, “biocompatible” refers to the quality of not having toxic or injurious effects on the body.

In one embodiment, the particulate material of the invention is not structured to form a predefined 3D shape or scaffold, such as for example a cube. In one embodiment, the particulate material of the invention has not a predefined shape or scaffold. In one embodiment, the particulate material of the invention has not the form of a cube. In one embodiment, the particulate material is not a 3D scaffold. In one embodiment, the biomaterial of the invention is scaffold-free.

In certain embodiments, the particulate material is selected from the group comprising or consisting of:
an organic material, including demineralized bone matrix (DBM), gelatin, agar/agarose, alginates chitosan, chondroitin sulfate, collagen, elastin or elastin-like peptides (ELP), fibrinogen, fibrin, fibronectin, proteoglycans, heparan sulfate proteoglycans, hyaluronic acid, polysaccharides, laminins and cellulose derivatives;
a ceramic material, including particles of calcium phosphate (CaP), calcium carbonate (CaCO₃), calcium sulfate (CaSO₄), or calcium hydroxide (Ca(OH)₂), or combinations thereof,
a polymer, including polyanhydrides, polylactic acid (PLA), poly(lactic-co-glycolic acid) (PLGA), polyethylene oxide/polyethylene glycol (PEO/PEG), poly(vinyl alcohol) (PVA), fumarate-based polymers such as, for example poly(propylene fumarate) (PPF) or poly(propylene fumarate-co-ethylene glycol) (P(PF-co-EG)), oligo(poly(ethylene glycol) fumarate) (OPF), poly (n-isopropylacrylamide) (PNIPPAAm), poly(aldehyde guluronate) (PAG), poly(n-vinyl pyrrolidone) (PNVP), or combinations thereof,
a gel, including a self-assembling oligopeptide gel, a microgel, a nanogel, a particulate gel, a hydrogel, a thixotropic gel, a xerogel, a responsive gel, or combinations thereof,
a creamer;
and any combination thereof.

In some preferred embodiments, the particulate material is gelatin.

In one embodiment, the gelatin of the invention is animal gelatin, preferably mammal gelatin, more preferably porcine gelatin. As used herein, the term “porcine gelatin” may be replaced by “pork gelatin” or “pig gelatin”. In one embodiment, the gelatin is porcine skin gelatin.

In certain embodiments, said gelatin is in the form of particles, preferably particles having a mean diameter ranging from about 50 μm to about 1,000 μm.

Within the scope of the invention, the expression “from about 50 μm to about 1,000 μm” encompasses 50 μm, 60 μm, 70 μm, 80 μm, 90 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm and 1,000 μm.

In one embodiment, the gelatin of the invention is in form of particles, beads, spheres, microspheres, and the like.

In one embodiment, the gelatin of the invention is not structured to form a predefined 3D shape or scaffold, such as for example a cube. In one embodiment, the gelatin of the invention has not a predefined shape or scaffold. In one embodiment, the gelatin of the invention has not the form of a cube. In one embodiment, the gelatin, preferably the porcine gelatin, is not a 3D scaffold. In one embodiment, the gelatin of the invention is a macroporous microcarrier.

Examples of porcine gelatin particles include, but are not limited to, Cultispher® G, Cultispher® S, Spongostan and Cutanplast. In one embodiment, the gelatin of the invention is Cultispher® G or Cultispher® S.

In one embodiment, the gelatin, preferably the porcine gelatin, of the invention have a mean diameter of at least about 50 μm, preferably of at least about 75 μm, more preferably of at least about 100 μm, more preferably of at least about 130 μm. In one embodiment, the gelatin of the invention, preferably the porcine gelatin, have a mean diameter of at most about 1,000 μm, preferably of at most about 750 μm, more preferably of at most about 500 μm. In another embodiment, the gelatin of the invention, preferably the porcine gelatin, have a mean diameter of at most about 450 μm, preferably of at most about 400 μm, more preferably of at least most about 380 μm.

In one embodiment, the gelatin of the invention, preferably the porcine gelatin, has a mean diameter ranging from about 50 μm to about 1,000 μm, preferably from about 75 μm to about 750 μm, more preferably from about 100 μm to about 500 μm. In another embodiment, the gelatin of the invention, preferably the porcine gelatin, has a mean diameter ranging from about 50 μm to about 500 μm, preferably from about 75 μm to about 450 μm, more preferably from about 100 μm to about 400 μm. In another embodiment, the gelatin of the invention, preferably the porcine gelatin, have a mean diameter ranging from about 130 μm to about 380 μm.

Methods to assess the mean diameter of gelatin particles according to the invention are known in the art. Examples of such methods include, but are not limited to, granulometry, in particular using suitable sieves; sedimentometry; centrifugation techniques; laser diffraction; and images analysis, in particular by the means of a high-performance camera with telecentric lenses; and the like.

In one embodiment, gelatin is added at a concentration ranging from about 0.1 cm³ to about 5 cm³ for a 150 cm² vessel, preferably from about 0.5 cm³ to about 4 cm³, more preferably from about 0.75 cm³ to about 3 cm³. In one embodiment, gelatin is added at a concentration ranging from about 1 cm³ to about 2 cm³ for a 150 cm² vessel. In one embodiment, gelatin is added at a concentration of about 1 cm³, 1.5 cm³ or 2 cm³ for a 150 cm² vessel. Within the scope of the invention, the expression “0.1 cm³ to about 5 cm³” encompasses 0.1 cm³, 0.2 cm³, 0.3 cm³, 0.4 cm³, 0.5 cm³, 0.6 cm³, 0.7 cm³, 0.8 cm³, 0.9 cm³, 1.0 cm³, 1.5 cm³, 2.0 cm³, 2.5 cm³, 3.0 cm³, 3.5 cm³, 4.0 cm³, 4.5 cm³ and 5.0 cm³.

In one embodiment, gelatin is added at a concentration ranging from about 0.1 g to about 5 g for a 150 cm² vessel, preferably from about 0.5 g to about 4 g, more preferably from about 0.75 g to about 3 g. In one embodiment, gelatin is added at a concentration ranging from about 1 g to about 2 g for a 150 cm² vessel. In one embodiment, gelatin is added at a concentration of about 1 g, 1.5 g or 2 g for a 150 cm² vessel. Within the scope of the invention, the expression “0.1 g to about 5 g” encompasses 0.1 g, 0.2 g, 0.3 g, 0.4 g, 0.5 g, 0.6 g, 0.7 g, 0.8 g, 0.9 g, 1.0 g, 1.5 g, 2.0 g, 2.5 g, 3.0 g, 3.5 g, 4.0 g, 4.5 g and 5.0 g.

In one embodiment, the gelatin of the invention is added to the culture medium after differentiation of the cells. In one embodiment, the gelatin of the invention is added to the culture medium when cells are sub-confluent. In one embodiment, the gelatin of the invention is added to the culture medium when cells are overconfluent. In one embodiment, the gelatin of the invention is added to the culture medium when cells have reached confluence after differentiation. In others words, in one embodiment, the gelatin of the invention is added to the culture medium when cells have reached confluence in differentiation medium. In one embodiment, the gelatin of the invention is added to the culture medium at least 5 days after P4, preferably 10 days after P4, more preferably 15 days after P4. In one embodiment, the gelatin of the invention is added to the culture medium from 5 days to 30 days after P4, preferably from 10 days to 25 days after P4, more preferably from 15 days to 20 days after P4.

In some preferred embodiments, the particulate material is a ceramic material.

In one embodiment, the ceramic material of the invention are particles of calcium phosphate (CaP), calcium carbonate (CaCO₃), calcium sulfate (CaSO₄), or calcium hydroxide (Ca(OH)₂), or combinations thereof.

Examples of calcium phosphate particles include, but are not limited to, hydroxyapatite (HA, Ca₁₀(P0₄)₆(OH)₂), tricalcium phosphate (TCP, Ca₃(P0₄)₂), α-tricalcium phosphate (α-TCP, (α-Ca₃(P0₄)₂), β-tricalcium phosphate (β-TCP, β-Ca₃(P0₄)₂), tetracalcium phosphate (TTCP, Ca₄(P0₄)₂O), octacalcium phosphate (CasH₂(P0₄)₆·5H₂O), amorphous calcium phosphate (Ca₃(P0₄)₂), hydroxyapatite/β-tricalcium phosphate (HA/β-TCP), hydroxyapatite/tetracalcium phosphate (HA/TTCP), and the like.

In one embodiment, the ceramic material of the invention comprises or consists of hydroxyapatite (HA), tricalcium phosphate (TCP), hydroxyapatite/β-tricalcium phosphate (HA/β-TCP), calcium sulfate (CaSO₄), or combinations thereof. In one embodiment, the ceramic material of the invention comprises or consists of hydroxyapatite (HA), β-tricalcium phosphate (β-TCP), hydroxyapatite/β-tricalcium phosphate (HA/β-TCP), α-tricalcium phosphate (α-TCP), calcium sulfate (CaSO₄), or combinations thereof.

In some embodiments, said particulate material comprises a ceramic material, preferably comprising calcium phosphate, preferably hydroxyapatite (HA) and/or β-tricalcium phosphate (β-TCP), more preferably particles of calcium phosphate.

In certain embodiments, said ceramic material comprises calcium phosphate, preferably hydroxyapatite (HA) and/or β-tricalcium phosphate (β-TCP), more preferably particles of calcium phosphate.

In one embodiment, the ceramic particles of the invention are particles of hydroxyapatite (HA). In another embodiment, the ceramic particles of the invention are particles of β-tricalcium phosphate (β-TCP). In another embodiment, the ceramic particles of the invention are particles of hydroxyapatite/β-tricalcium phosphate (HA/β-TCP). In other words, in one embodiment, the ceramic particles of the invention are a mixture of hydroxyapatite and β-tricalcium phosphate particles (called HA/β-TCP particles). In one embodiment, the ceramic particles of the invention consist of hydroxyapatite particles and β-tricalcium phosphate particles (called HA/β-TCP particles).

In one embodiment, the particulate material, preferably the ceramic particles, more preferably HA, β-TCP and/or HA/β-TCP particles, are in form of granules, powder or beads. In one embodiment, the particulate material, preferably the ceramic particles, more preferably HA, β-TCP and/or HA/β-TCP particles, are in form of porous granules, powder or beads. In one embodiment, the particulate material, preferably the ceramic particles, more preferably HA, 3-TCP and/or HA/β-TCP particles, are porous ceramic material. In one embodiment, the particulate material, preferably the ceramic particles, more preferably HA, β-TCP and/or HA/β-TCP particles, are powder particles. In a particular embodiment, the particulate material, preferably the ceramic particles, more preferably HA, β-TCP and/or HA/β-TCP particles, are in form of porous granules. In another particular embodiment, the particulate material, preferably the ceramic particles, more preferably HA, β-TCP and/or HA/β-TCP particles, are in form of powder.

In one embodiment, the particulate material, preferably the ceramic particles, more preferably HA, β-TCP and/or HA/β-TCP particles, are not structured to form a predefined 3D shape or scaffold, such as for example a cube. In one embodiment, the particulate material, preferably the ceramic material of the invention is not a 3D scaffold. In one embodiment, the particulate material, preferably the ceramic material has not a predefined shape or scaffold. In one embodiment, the particulate material, preferably the ceramic material of the invention has not the form of a cube.

In one embodiment, the particulate material, preferably the ceramic particles of the invention, more preferably HA, β-TCP and/or HA/β-TCP particles, are larger than about 50 μm, preferably larger than about 100 μm. In one embodiment, the particulate material, preferably the ceramic particles of the invention, more preferably HA, β-TCP and/or HA/β-TCP particles, have a mean diameter larger than about 50 μm, preferably larger than about 100 μm.

In one embodiment, the particulate material, preferably the ceramic particles of the invention, more preferably HA, β-TCP and/or HA/β-TCP particles, have a mean diameter of at least about 50 μm, preferably of at least about 100 μm, more preferably of at least about 150 μm. In another embodiment, the particulate material, preferably the ceramic particles of the invention, more preferably HA, β-TCP and/or HA/β-TCP particles, have a mean diameter of at least about 200 μm, preferably of at least about 250 μm, more preferably of at least about 300 μm.

In another embodiment, the particulate material, preferably the ceramic particles of the invention, more preferably HA, β-TCP and/or HA/β-TCP particles, have a mean diameter of at most about 2,500 μm, preferably of at most about 2,000 μm, more preferably of at most about 1,500 μm. In one embodiment, the particulate material, preferably the ceramic particles of the invention, more preferably HA, β-TCP and/or HA/β-TCP particles, have a mean diameter of at most about 1,000 μm, 900 μm, 800 μm, 700 μm or 600 μm.

In one embodiment, the particulate material, preferably the ceramic particles of the invention, more preferably HA, β-TCP and/or HA/β-TCP particles, have a mean diameter ranging from about 50 μm to about 1,500 μm, preferably from about 50 μm to about 1,250 μm, more preferably from about 100 μm to about 1,000 μm. In one embodiment, the particulate material, preferably the ceramic particles of the invention, more preferably HA, β-TCP and/or HA/β-TCP particles, have a mean diameter ranging from about 100 μm to about 800 μm, preferably from about 150 μm to about 700 μm, more preferably from about 200 μm to about 600 μm.

In one embodiment, the HA/β-TCP particles have a mean diameter ranging from about 50 μm to about 1,500 μm, preferably from about 50 μm to about 1,250 μm, more preferably from about 100 μm to about 1,000 μm. In one embodiment, the HA and 0-TCP particles have a mean diameter ranging from about 100 μm to about 800 μm, preferably from about 150 μm to about 700 μm, more preferably from about 200 μm to about 600 μm.

In practice, the measure of the mean sizes and diameters of particles may be performed by any suitable methods known in the state of the art, or a method adapted therefrom. Non-limiting examples of such methods include atomic force microscopy (AFM), transmission electron microscopy (TEM), scanning electron microscopy (SEM), dynamic light scattering (DLS).

In one embodiment, the ratio between HA and β-TCP (HA/β-TCP ratio) in the particles ranges from about 0/100 to about 100/0, preferably from about 10/90 to about 90/10, more preferably from about 20/80 to about 80/20. In one embodiment, the ratio HA/β-TCP in the particles ranges from about 30/70 to about 70/30, from about 35/65 to about 65/35, or from about 40/60 to about 60/40.

In one embodiment, the HA/β-TCP ratio in the particles is 0/100, i.e. the particles are particles of β-tricalcium phosphate. In another embodiment, the HA/β-TCP ratio in the particles is 100/0, i.e. the particles are particles of hydroxyapatite. In one embodiment, the HA/β-TCP ratio in the particles is about 10/90. In another embodiment, the HA/β-TCP ratio in the particles is about 90/10. In one embodiment, the HA/β-TCP ratio in the particles is about 20/80. In another embodiment, the HA/β-TCP ratio in the particles is about 80/20. In one embodiment, the HA/β-TCP ratio in the particles is about 30/70. In another embodiment, the HA/β-TCP ratio in the particles is about 70/30. In another embodiment, the HA/β-TCP ratio in the particles is about 35/65. In another embodiment, the HA/β-TCP ratio in the particles is about 65/35. In one embodiment, the HA/β-TCP ratio in the particles is about 40/60. In another embodiment, the HA/β-TCP ratio in the particles is about 60/40. In another embodiment, the HA/β-TCP ratio in the particles is about 50/50.

In one embodiment, the HA/β-TCP ratio in the particles is 100/0, 99/1, 98/2, 97/3, 96/4, 95/5, 94/6, 93/7, 92/8, 91/9, 90/10, 89/11, 88/12, 87/13, 86/14, 85/15, 84/16, 83/17, 82/18, 81/19, 80/20, 79/21, 78/22, 77/23, 76/24, 75/25, 74/26, 73/27, 72/28, 71/29, 70/30, 69/31, 68/32, 67/33, 66/34, 65/35, 64/36, 63/37, 62/38, 61/39, 60/40, 59/41, 58/42, 57/43, 56/44, 55/45, 54/46, 53/47, 52/48, 51/49, 50/50, 49/51, 48/52, 47/53, 46/54, 45/55, 44/56, 43/57, 42/58, 41/59, 40/60, 39/61, 38/62, 37/63, 36/64, 35/65, 34/66, 33/67, 32/68, 31/69, 30/70, 29/71, 28/72, 27/73, 26/74, 25/75, 24/76, 23/77, 22/78, 21/79, 20/80, 19/81, 18/82, 17/83, 16/84, 15/85, 14/86, 13/87, 12/88, 11/89, 10/90, 9/91, 8/92, 7/93, 6/94, 5/95, 4/96, 3/97, 2/98, 1/99, or 0/100.

According to one embodiment, the quantity of particulate material, preferably ceramic particles, more preferably HA, β-TCP and/or HA/β-TCP particles, is optimal for providing a 3D structure to the biomaterial. In one embodiment, the particulate material, preferably the ceramic particles, more preferably HA, β-TCP and/or HA/β-TCP particles, are added at a concentration ranging from about 0.1 cm³ to about 5 cm³ for a 150 cm² vessel, preferably from about 0.5 cm³ to about 3 cm³, more preferably from about 1 cm³ to about 3 cm³. In a preferred embodiment, the particulate material, preferably the ceramic particles, more preferably HA, β-TCP and/or HA/β-TCP particles, are added at a concentration of about 1.5 cm³ to about 3 cm³ for a 150 cm² vessel.

In one embodiment, the particulate material, preferably the ceramic particles, more preferably HA, β-TCP and/or HA/β-TCP particles, are added at a concentration ranging from about 7×10⁻³ to 7×10⁻² cm³ per mL of medium. In one embodiment, the particulate material, preferably the ceramic particles, more preferably HA, β-TCP and/or HA/β-TCP particles, are added at a concentration ranging from about 3.3×10-3 to 3.3×10⁻² cm³ per cm² of vessel.

In one embodiment, the particulate material, preferably the ceramic material, of the invention is added to the culture medium after differentiation of the cells. In one embodiment, the particulate material, preferably the ceramic material, of the invention is added when cells are subconfluent. In one embodiment, the particulate material, preferably the ceramic material, of the invention is added when cells are overconfluent. In one embodiment, the particulate material, preferably the ceramic material, of the invention is added when cells have reached confluence after differentiation. In others words, in one embodiment, the particulate material, preferably the ceramic material, of the invention is added when cells have reached confluence in differentiation medium. In one embodiment, the particulate material, preferably the ceramic material, of the invention is added at least 5 days after P4, preferably 10 days after P4, more preferably 15 days after P4. In one embodiment, the particulate material, preferably the ceramic material, of the invention is added from 5 days to 30 days after P4, preferably from 10 days to 25 days after P4, more preferably from 15 days to 20 days after P4.

In another embodiment, the particulate material of the invention is demineralized bone matrix (DBM).

In one embodiment, DBM is of animal origin, preferably of mammal origin, more preferably of human origin. In a particular embodiment, human DBM is obtained by grinding cortical bones from human donors.

Methods to obtain DBM are known in the art. For example, human bone tissue may first be defatted by acetone (e.g., at about 99%) bath during an overnight and then be washed in demineralized water during about 2 hours. Decalcification may be performed by immersion in HCL (e.g., at about 0.6 N) during about 3 hours (20 mL solution per gram of bone) under agitation at room temperature. Then, demineralized bone powder may be rinsed with demineralized water during about 2 hours and the pH is controlled. If the pH is too acid, DBM may be buffered with a phosphate solution (e.g., at about 0.1 M) under agitation. Finally, DBM may be dried and weighted. The DBM may be sterilized by Gamma irradiation following techniques known in the field, for example at about 25 kGray.

In one embodiment, the DBM is allogenic. In one embodiment, the DBM is homogenous.

In another embodiment, the DBM is heterogeneous.

In one embodiment, DBM is in the form of particles, herein referred to as demineralized bone matrix particles or DBM particles. In one embodiment, the DBM particles have a mean diameter ranging from about 50 to about 2,500 μm, preferably from about 50 μm to about 1500 μm, more preferably from about 50 μm to about 1,000 μm. In one embodiment, the DBM particles have a mean diameter ranging from about 100 μm to about 1,500 μm, more preferably from about 150 μm to about 1,000 μm. In one embodiment, the DBM particles have a mean diameter ranging from about 200 to about 1,000 μm, preferably from about 200 μm to about 800 μm, more preferably from about 300 μm to about 700 μm.

In one embodiment, the biomaterial of the invention comprises an extracellular matrix.

In one embodiment, the extracellular matrix of the invention derives from the cells, preferably ASCs. In one embodiment, the extracellular matrix of the invention is produced by the cells, preferably ASCs. In one embodiment, the extracellular matrix of the biomaterial of the invention derived from the differentiated cells, preferably differentiated ASCs.

As used herein, the term “extracellular matrix” (ECM) means a non-cellular three-dimensional macromolecular network. Matrix components of ECM bind each other as well as cell adhesion receptors, thereby forming a complex network into which cells reside in tissues or in biomaterials of the invention.

In one embodiment, the extracellular matrix of the invention comprises collagen, proteoglycans/glycosaminoglycans, elastin, fibronectin, laminin, and/or other glycoproteins. In a particular embodiment, the extracellular matrix of the invention comprises collagen. In another particular embodiment, the extracellular matrix of the invention comprises proteoglycans. In another particular embodiment, the extracellular matrix of the invention comprises collagen and proteoglycans. In one embodiment, the extracellular matrix of the invention comprises growth factors, proteoglycans, secreting factors, extracellular matrix regulators, and glycoproteins.

In one embodiment, the cells, preferably ASCs, and the particulate material, preferably the gelatin, the DBM or the ceramic material of the invention are embedded into the extracellular matrix.

In certain embodiments, said biomaterial comprises an altered factors content as compared to the factors content obtained from a corresponding fresh, non-sterile, non-desiccated biomaterial.

Within the scope of the instant invention, the term “altered factors content” refers to a content of factors within the biomaterial that is distinct when compared to a reference. As used herein, when a specific factor is considered, an altered content for this factor is intended to refer to either an increased relative amount of said factor or a decreased relative amount of said factor, as compared to a reference.

In some embodiments, the reference is a factors content obtained from a corresponding fresh, non-sterile, non-desiccated biomaterial.

As used herein, the term “altered” is intended to mean “of a substantially distinct relative amount”. In other words, an “altered content of ingredients” means that the relative amount of the ingredients from the biomaterial according to the invention varies of at least about 10% with respect to the relative amount of the ingredients from a reference biomaterial. Within the scope of the invention, the expression “at least about 10%” encompasses 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1,000%, 1,250%, 1,500%, 1,750%, 2,000%, 2,250%, 2,500%, 2,750%, 3,000%, 3,250%, 3,500%, 3,750%, 4,000%, 4,250%, 4,500%, 4,750%, 5,000%, 6,000%, 7,000%, 8,000%, 9,000% and 10,000%.

Within the scope of the instant invention, the term “varies” is intended to mean “decreases” or “increases”.

In some embodiments, the average relative amount of the content comprised in the biomaterial according to the invention varies of at least about 10% with respect to the average relative amount of said content obtained from a corresponding fresh, non-sterile, non-desiccated biomaterial.

In some embodiments, the average relative amount of the content comprised in the biomaterial according to the invention varies of at least about 10% with respect to the average relative amount of said content obtained from the viable differentiated cells, including osteo-differentiated and/or chondro-differentiated cells, before being submitted to the steps for generating a sterile and desiccated biomaterial.

In some embodiments, the factors content includes proteins, such as growth factors, transcription factors, osteogenic factors and chondrogenic factors, and nucleic acids, such as RNAs and in particular miRNAs.

In some embodiments, the factors content includes growth factors and/or transcription factors.

In some embodiments, the factors content includes growth factors and/or transcription factors, and/or osteogenic factors, and/or chondrogenic factors.

As used herein, growth factors are intended to refer to polypeptides that regulate many aspects of cellular function, including survival, proliferation, migration and differentiation. Non-limitative examples of growth factors according to the invention include BMPs, EGF, FGFs, HGF, IGF-1, OPG (osteoprotegerin), SDF-1α, TGFB-1, TGFB-3, VEGF, including VEGFA and VEGFB.

As used herein, transcription factors are intended to refer to polypeptides that control whether a given gene is to be transcribed into its corresponding RNA. In some embodiments, transcription factors according to the invention include, but are not limited to SMAD-2, SMAD-3, SMAD-4, SMAD-5. In certain embodiments, transcription factors according to the invention include, but are not limited to, AKT, ANG, ANGPT1, ANGPTL4, ANPEP, COL18A1, CTGF, CXCL1, EDN1, EFNA1, EFNB2, ENG, EPHB4, F3, FGF1, FGF2, FN1, HIF1A, ID1, IL6, ITGAV, JAG1, LEP, MMP14, MMP2, NRP1, PTGS1, SERPINEl, SERPINF1, TGFB1, TGFBR1, THBS1, THBS2, TIMP1, TIMP2, TIMP3, VEGFA, VEGFB, VEGFC.

As used herein, osteogenic factors are intended to refer to polypeptides that promote osteogenesis and/or impair osteoclasia. In some embodiments, the osteogenic factors according to the invention are involve in the skeletal development. Non-limitative examples of osteogenic factors according to the invention include OPG, SDF-1α, BMPR-1A, BMPR-2, FGFR-1, FGFR-2, TWIST-1, CSF-1, IGFR, RUNX2, TGFBR-1.

In some embodiments, the factors content comprises VEGF and/or IGF-1 and/or SDF-1α. Without wishing to be bound to a particular theory, the inventors considers that the factors such as VEGF and/or IGF-1 and/or SDF-1α participate in the healing process; VEGF, by promoting angiogenesis; IGF-1, by being positively correlated with the wound healing process, including myofibroblasts recruitment, promotion of collagen synthesis and stimulation of fibroblasts and keratinocytes; and SDF-1α, by promoting stem cells recruitment for wound healing.

In some embodiments, the factors content comprises IGF-1 and/or VEGF and/or SDF-1α and/or (OPG).

As can be seen from the example section hereunder, the average relative amounts of the factors OPG, SDF-1α, BMPR-1A, BMPR-2, CSF-1, IGF1R, TWIST-1, SMAD-2, SMAD-3, SMAD-4, SMAD-5 in the desiccated and sterile biomaterial according to the invention are increased by above 10% with respect to the average relative amount of said content obtained from a corresponding fresh, non-sterile, non-desiccated biomaterial.

In some embodiments, the biomaterial according to the instant invention comprises from about 0.1 ng to about 200 ng of IGF-1 per g (w/w) of the biomaterial, preferably from about 1 ng to about 150 ng per g of the biomaterial, more preferably from about 10 ng to about 80 ng per g of the biomaterial. Within the scope of the invention, the expression “from about 0.1 ng to about 200 ng per g” includes about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 110, 120, 130, 140, 150, 160, 170, 180, 190 and 200 ng per g.

In certain embodiments, the biomaterial according to the instant invention comprises from about 0.1 ng to about 100 ng of OPG per g (w/w) of the biomaterial, preferably from about 1 ng to about 50 ng per g of the biomaterial. Within the scope of the invention, the expression “from about 0.1 ng to about 100 ng per g” includes about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95 and 100 ng per g.

In some embodiments, the biomaterial according to the instant invention comprises from about 0.1 ng to about 200 ng of VEGF per g (w/w) of the biomaterial, preferably from about 1 ng to about 150 ng per g of the biomaterial, more preferably from about 20 ng to about 100 ng per g of the biomaterial. Within the scope of the invention, the expression “from about 0.1 ng to about 200 ng per g” includes about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190 and 200 ng per g.

In some embodiments, the biomaterial according to the instant invention comprises from about 0.1 ng to about 20 ng of VEGF per g (w/w) of the biomaterial, preferably from about 1 ng to about 15 ng per g of the biomaterial. Within the scope of the invention, the expression “from about 0.1 ng to about 20 ng per g” includes about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 and 20 ng per g.

In certain embodiments, the biomaterial according to the instant invention comprises from about 0.1 ng to about 400 ng of SDF-1α per g (w/w) of the biomaterial, preferably from about 1 ng to about 250 ng per g of the biomaterial, more preferably from about 10 ng to about 200 ng per g of the biomaterial. Within the scope of the invention, the expression “from about 0.1 ng to about 400 ng per g” includes about 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390 and 400 ng per g.

In practice, under the suitable culture conditions, the cells are secreting an extracellular matrix and synthesize factors, including polypeptides and nucleic acids, that promote tissue regeneration and/or tissue repair. Said factors, including polypeptides and nucleic acids, may be considered as being biomarkers for the tissue regeneration and/or tissue repair.

In practice, the content of factors, as polypeptides, of the biomaterial according to the instant invention may be assessed by any suitable method known in the art, or any method adapted therefrom. Illustratively, the expression or absence of expression (non-expression) of these biomarkers may be monitored at the nucleic acid level or the polypeptide level. Non-limitative example of methods for monitoring biomarkers at the nucleic acid level encompasses RT-PCR (qPCR) analysis of RNA extracted from cultured cells with specific primers. Non-limitative examples of methods for monitoring biomarkers at the polypeptide level encompass immunofluorescence analysis with markers-specific antibodies, such as Western blotting or ELISA; Fluorescent activated cell sorting (FACS); mass spectrometry, and enzymatic assays.

In certain embodiments, said factors content includes a RNAs content.

In some embodiments, the average relative expression of the RNAs comprised in the biomaterial according to the invention varies of at least about 10% with respect to the average relative expression of said RNAs from a corresponding fresh, non-sterile, non-desiccated biomaterial.

In certain embodiments, the RNAs content comprises one or more miRNA(s).

Within the scope of the invention, criteria and conventions for miRNAs identification and naming have been described in Ambros et al. (A uniform system for microRNA annotation. RNA 2003 9(3):277-279). The miRNAs sequences may be easily retrieved from the miRbase database (http://www.mirbase.org/) or the miRDB database (http://www.mirdb.org/).

In certain embodiments, the RNAs content comprises at least one miRNA selected in any one of Table 1, Table 2, Table 3, Table 4, Table 5, Table 6, Table 7, Table 8, Table 9, Table 10, Table 11 or Table 12.

As used herein, the term “at least one” include, 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50 or more.

TABLE 1
miRNAs according to the invention

| | | | |
|---|---|---|---|
| hsa-let-7a-5p | hsa-miR-199a-3p | hsa-miR-10a-5p | hsa-miR-411-5p |
| hsa-let-7b-5p | hsa-miR-145-5p | hsa-miR-495-3p | hsa-miR-505-5p |
| hsa-let-7f-5p | hsa-miR-30a-3p | hsa-miR-425-5p | hsa-miR-664a-3p |
| hsa-miR-24-3p | hsa-miR-382-5p | hsa-miR-2053 | hsa-miR-26a-5p |
| hsa-miR-21-5p | hsa-miR-19b-3p | hsa-miR-5096 | hsa-miR-377-3p |
| hsa-miR-23b-3p | hsa-miR-210-3p | hsa-miR-494-3p | hsa-miR-485-3p |
| hsa-miR-1273g-3p | hsa-miR-619-5p | hsa-miR-27a-3p | hsa-miR-590-3p |
| hsa-miR-574-3p | hsa-miR-17-5p | hsa-miR-4449 | hsa-miR-99a-3p |
| hsa-miR-25-3p | hsa-miR-193a-5p | hsa-miR-532-3p | hsa-miR-143-3p |
| hsa-let-7e-5p | hsa-miR-320b | hsa-miR-532-5p | hsa-miR-26b-3p |
| hsa-miR-214-3p | hsa-miR-193b-5p | hsa-miR-126-5p | hsa-miR-3607-5p |
| hsa-miR-199a-5p | hsa-miR-320a | hsa-miR-30c-5p | hsa-miR-3651 |
| hsa-miR-196a-5p | hsa-miR-151a-3p | hsa-miR-130b-3p | hsa-miR-374a-3p |
| hsa-miR-199b-5p | hsa-let-7a-3p | hsa-miR-136-3p | hsa-miR-376a-3p |
| hsa-miR-221-3p | hsa-miR-30e-3p | hsa-miR-15b-3p | hsa-miR-485-5p |
| hsa-miR-424-5p | hsa-miR-22-3p | hsa-miR-29b-1-5p | hsa-miR-103b |
| hsa-miR-23a-3p | hsa-miR-99b-5p | hsa-miR-99b-3p | hsa-miR-126-3p |
| hsa-let-7c-5p | hsa-miR-625-3p | hsa-miR-127-3p | hsa-miR-149-5p |
| hsa-miR-199b-3p | hsa-miR-4668-5p | hsa-miR-134-5p | hsa-miR-193b-3p |
| hsa-miR-191-5p | hsa-miR-29b-3p | hsa-miR-324-5p | hsa-miR-223-3p |
| hsa-miR-574-5p | hsa-miR-423-3p | hsa-miR-3605-3p | hsa-miR-340-3p |
| hsa-miR-424-3p | hsa-miR-376c-3p | hsa-miR-101-3p | hsa-miR-369-5p |
| hsa-miR-423-5p | hsa-let-7b-3p | hsa-miR-103a-3p | hsa-miR-6724-5p |
| hsa-miR-342-3p | hsa-miR-3074-5p | hsa-miR-1246 | hsa-miR-7847-3p |
| hsa-let-7d-3p | hsa-miR-98-5p | hsa-miR-138-5p | hsa-miR-874-3p |
| hsa-miR-130a-3p | hsa-miR-185-5p | hsa-miR-190a-5p | hsa-miR-3653-5p |
| hsa-miR-3184-3p | hsa-miR-19a-3p | hsa-miR-24-2-5p | hsa-miR-664b-3p |
| hsa-miR-222-3p | hsa-miR-34a-5p | hsa-miR-26a-2-3p | hsa-miR-664b-5p |
| hsa-let-7g-5p | hsa-miR-374c-3p | hsa-miR-301a-3p | hsa-miR-6516-3p |
| hsa-miR-125a-5p | hsa-miR-181a-5p | hsa-miR-98-3p | hsa-let-7i-3p |
| hsa-let-7d-5p | hsa-miR-328-3p | hsa-miR-1273a | hsa-miR-154-5p |
| hsa-miR-29a-3p | hsa-miR-92b-3p | hsa-miR-28-5p | hsa-miR-664a-5p |
| hsa-let-7i-5p | hsa-miR-335-5p | hsa-miR-34a-3p | hsa-miR-1291 |
| hsa-miR-146b-5p | hsa-let-7f-1-3p | hsa-miR-425-3p | hsa-miR-140-5p |
| hsa-miR-4454 | hsa-miR-196b-5p | hsa-miR-505-3p | hsa-miR-3609 |
| hsa-miR-28-3p | hsa-miR-3613-3p | hsa-miR-34b-3p | hsa-miR-4461 |
| hsa-miR-92a-3p | hsa-miR-23a-5p | hsa-miR-361-3p | hsa-miR-3613-5p |
| hsa-miR-125b-5p | hsa-miR-374b-5p | hsa-miR-10b-5p | hsa-miR-663b |
| hsa-miR-337-3p | hsa-miR-660-5p | hsa-miR-1306-5p | hsa-miR-378a-3p |
| hsa-miR-93-5p | hsa-miR-186-5p | hsa-miR-22-5p | hsa-miR-454-3p |
| hsa-miR-409-3p | | | |

In some embodiments, the at least one miRNA is selected in a group comprising hsa-let-7a-5p, hsa-miR-199a-3p, hsa-miR-10a-5p, hsa-miR-411-5p, hsa-let-7b-5p, hsa-miR-145-5p, hsa-miR-495-3p, hsa-miR-505-5p, hsa-let-7f-5p, hsa-miR-30a-3p, hsa-miR-425-5p, hsa-miR-664a-3p, hsa-miR-24-3p, hsa-miR-382-5p, hsa-miR-2053, hsa-miR-26a-5p, hsa-miR-21-5p, hsa-miR-19b-3p, hsa-miR-5096, hsa-miR-377-3p, hsa-miR-23b-3p, hsa-miR-210-3p, hsa-miR-494-3p, hsa-miR-485-3p, hsa-miR-1273g-3p, hsa-miR-619-5p, hsa-miR-27a-3p, hsa-miR-590-3p, hsa-miR-574-3p, hsa-miR-17-5p, hsa-miR-4449, hsa-miR-99a-3p, hsa-miR-25-3p, hsa-miR-193a-5p, hsa-miR-532-3p, hsa-miR-143-3p, hsa-let-7e-5p, hsa-miR-320b, hsa-miR-532-5p, hsa-miR-26b-3p, hsa-miR-214-3p, hsa-miR-193b-5p, hsa-miR-126-5p, hsa-miR-3607-5p, hsa-miR-199a-5p, hsa-miR-320a, hsa-miR-30c-5p, hsa-miR-3651, hsa-miR-196a-5p, hsa-miR-151a-3p, hsa-miR-130b-3p, hsa-miR-374a-3p, hsa-miR-199b-5p, hsa-let-7a-3p, hsa-miR-136-3p, hsa-miR-376a-3p, hsa-miR-221-3p, hsa-miR-30e-3p, hsa-miR-15b-3p, hsa-miR-485-5p, hsa-miR-424-5p, hsa-miR-22-3p, hsa-miR-29b-1-5p, hsa-miR-103b, hsa-miR-23a-3p, hsa-miR-99b-5p, hsa-miR-99b-3p, hsa-miR-126-3p, hsa-let-7c-5p, hsa-miR-625-3p, hsa-miR-127-3p, hsa-miR-149-5p, hsa-miR-199b-3p, hsa-miR-4668-5p, hsa-miR-134-5p, hsa-miR-193b-3p, hsa-miR-191-5p, hsa-miR-29b-3p, hsa-miR-324-5p, hsa-miR-223-3p, hsa-miR-574-5p, hsa-miR-423-3p, hsa-miR-3605-3p, hsa-miR-340-3p, hsa-miR-424-3p, hsa-miR-376c-3p, hsa-miR-101-3p, hsa-miR-369-5p, hsa-miR-423-5p, hsa-let-7b-3p, hsa-miR-103a-3p, hsa-miR-6724-5p, hsa-miR-342-3p, hsa-miR-3074-5p, hsa-miR-1246, hsa-miR-7847-3p, hsa-let-7d-3p, hsa-miR-98-5p, hsa-miR-138-5p, hsa-miR-874-3p, hsa-miR-130a-3p, hsa-miR-185-5p, hsa-miR-190a-5p, hsa-miR-3653-5p, hsa-miR-3184-3p, hsa-miR-19a-3p, hsa-miR-24-2-5p, hsa-miR-664b-3p, hsa-miR-222-3p, hsa-miR-34a-5p, hsa-miR-26a-2-3p, hsa-miR-664b-5p, hsa-let-7g-5p, hsa-miR-374c-3p, hsa-miR-301a-3p, hsa-miR-6516-3p, hsa-miR-125a-5p, hsa-miR-181a-5p, hsa-miR-98-3p, hsa-let-7i-3p, hsa-let-7d-5p, hsa-miR-328-3p, hsa-miR-1273a, hsa-miR-154-5p, hsa-miR-29a-3p, hsa-miR-92b-3p, hsa-miR-28-5p, hsa-miR-664a-5p, hsa-let-7i-5p, hsa-miR-335-5p, hsa-miR-34a-3p, hsa-miR-1291, hsa-miR-146b-5p, hsa-let-7f-1-3p, hsa-miR-425-3p, hsa-miR-140-5p, hsa-miR-4454, hsa-miR-196b-5p, hsa-miR-505-3p, hsa-miR-3609, hsa-miR-28-3p, hsa-miR-3613-3p, hsa-miR-34b-3p, hsa-miR-4461, hsa-miR-92a-3p, hsa-miR-23a-5p, hsa-miR-361-3p, hsa-miR-3613-5p, hsa-miR-125b-5p, hsa-miR-374b-5p, hsa-miR-10b-5p, hsa-miR-663b, hsa-miR-337-3p, hsa-miR-660-5p, hsa-miR-1306-5p, hsa-miR-378a-3p, hsa-miR-93-5p, hsa-miR-186-5p, hsa-miR-22-5p, hsa-miR-454-3p, hsa-miR-409-3p, and a combination thereof.

TABLE 2
miRNAs according to the invention

| | | | |
|---|---|---|---|
| hsa-let-7a-5p | hsa-miR-30a-3p | hsa-miR-103a-3p | hsa-miR-542-3p |
| hsa-let-7b-5p | hsa-miR-320b | hsa-miR-19a-3p | hsa-miR-663a |
| hsa-miR-24-3p | hsa-miR-193a-5p | hsa-miR-126-5p | hsa-miR-101-3p |
| hsa-miR-21-5p | hsa-miR-382-5p | hsa-miR-2053 | hsa-miR-143-3p |
| hsa-let-7f-5p | hsa-miR-423-3p | hsa-miR-29b-1-5p | hsa-miR-21-3p |
| hsa-miR-574-3p | hsa-miR-17-5p | hsa-miR-3648 | hsa-miR-224-5p |
| hsa-miR-23b-3p | hsa-miR-19b-3p | hsa-miR-374a-3p | hsa-miR-26a-5p |
| hsa-miR-1273g-3p | hsa-miR-92b-3p | hsa-miR-454-3p | hsa-miR-27a-5p |
| hsa-miR-25-3p | hsa-miR-320a | hsa-miR-532-3p | hsa-miR-324-5p |
| hsa-miR-199a-5p | hsa-miR-3074-5p | hsa-miR-136-3p | hsa-miR-340-3p |
| hsa-miR-196a-5p | hsa-miR-376c-3p | hsa-miR-361-3p | hsa-miR-379-5p |
| hsa-miR-214-3p | hsa-let-7b-3p | hsa-miR-1246 | hsa-miR-409-5p |
| hsa-miR-125a-5p | hsa-miR-625-3p | hsa-miR-130b-3p | hsa-miR-543 |
| hsa-miR-221-3p | hsa-miR-99b-5p | hsa-miR-134-5p | hsa-miR-5787 |
| hsa-miR-222-3p | hsa-miR-34a-5p | hsa-miR-154-5p | hsa-miR-6089 |
| hsa-let-7e-5p | hsa-miR-5096 | hsa-miR-34a-3p | hsa-miR-127-3p |
| hsa-miR-191-5p | hsa-miR-30e-3p | hsa-miR-576-5p | hsa-miR-149-5p |
| hsa-miR-199b-3p | hsa-miR-22-3p | hsa-miR-874-3p | hsa-miR-181c-5p |
| hsa-miR-342-3p | hsa-miR-151a-3p | hsa-miR-100-5p | hsa-miR-193b-3p |
| hsa-miR-23a-3p | hsa-miR-186-5p | hsa-miR-103b | hsa-miR-222-5p |
| hsa-miR-424-3p | hsa-miR-193b-5p | hsa-miR-1273a | hsa-miR-3613-5p |
| hsa-miR-28-3p | hsa-miR-328-3p | hsa-miR-1306-5p | hsa-miR-365b-3p |
| hsa-let-7g-5p | hsa-miR-4449 | hsa-miR-138-5p | hsa-miR-3960 |
| hsa-miR-92a-3p | hsa-miR-27a-3p | hsa-miR-15b-3p | hsa-miR-485-3p |
| hsa-miR-424-5p | hsa-miR-30c-5p | hsa-miR-26b-3p | hsa-miR-6087 |
| hsa-let-7d-3p | hsa-miR-494-3p | hsa-miR-10b-5p | hsa-miR-92a-1-5p |
| hsa-miR-4454 | hsa-miR-98-5p | hsa-miR-22-5p | hsa-miR-3607-5p |
| hsa-miR-146b-5p | hsa-miR-10a-5p | hsa-miR-3613-3p | hsa-miR-3653-5p |
| hsa-miR-423-5p | hsa-miR-29b-3p | hsa-miR-655-3p | hsa-miR-664b-5p |
| hsa-miR-29a-3p | hsa-miR-374b-5p | hsa-miR-7-1-3p | hsa-miR-664b-3p |
| hsa-miR-574-5p | hsa-miR-335-5p | hsa-miR-23a-5p | hsa-miR-6516-3p |
| hsa-miR-199b-5p | hsa-miR-374c-3p | hsa-miR-24-2-5p | hsa-miR-1291 |
| hsa-miR-125b-5p | hsa-miR-425-5p | hsa-miR-3605-3p | hsa-let-7i-3p |
| hsa-miR-3184-3p | hsa-miR-181a-5p | hsa-miR-6832-3p | hsa-miR-455-3p |
| hsa-let-7c-5p | hsa-miR-196b-5p | hsa-miR-146a-5p | hsa-miR-671-5p |
| hsa-miR-337-3p | hsa-let-7f-1-3p | hsa-miR-16-2-3p | hsa-miR-1271-5p |
| hsa-let-7d-5p | hsa-miR-4668-5p | hsa-miR-181b-5p | hsa-miR-4461 |
| hsa-miR-145-5p | hsa-miR-660-5p | hsa-miR-26a-2-3p | hsa-miR-6724-5p |
| hsa-miR-93-5p | hsa-miR-664a-3p | hsa-miR-376a-3p | hsa-miR-190a-5p |
| hsa-miR-619-5p | hsa-miR-185-5p | hsa-miR-539-5p | hsa-miR-3609 |
| hsa-miR-130a-3p | hsa-miR-3651 | hsa-miR-708-5p | hsa-miR-411-5p |
| hsa-let-7i-5p | hsa-miR-495-3p | hsa-miR-98-3p | hsa-miR-425-3p |
| hsa-miR-409-3p | hsa-let-7a-3p | hsa-miR-1237-5p | hsa-miR-4485-3p |
| hsa-miR-210-3p | hsa-miR-28-5p | hsa-miR-223-3p | hsa-miR-532-5p |
| hsa-miR-199a-3p | hsa-miR-99b-3p | | |

In some embodiments, at least one miRNA is selected in a group comprising or consisting of hsa-let-7a-5p, hsa-miR-30a-3p, hsa-miR-103a-3p, hsa-miR-542-3p, hsa-let-7b-5p, hsa-miR-320b, hsa-miR-19a-3p, hsa-miR-663a, hsa-miR-24-3p, hsa-miR-193a-5p, hsa-miR-126-5p, hsa-miR-101-3p, hsa-miR-21-5p, hsa-miR-382-5p, hsa-miR-2053, hsa-miR-143-3p, hsa-let-7f-5p, hsa-miR-423-3p, hsa-miR-29b-1-5p, hsa-miR-21-3p, hsa-miR-574-3p, hsa-miR-17-5p, hsa-miR-3648, hsa-miR-224-5p, hsa-miR-23b-3p, hsa-miR-19b-3p, hsa-miR-374a-3p, hsa-miR-26a-5p, hsa-miR-1273g-3p, hsa-miR-92b-3p, hsa-miR-454-3p, hsa-miR-27a-5p, hsa-miR-25-3p, hsa-miR-320a, hsa-miR-532-3p, hsa-miR-324-5p, hsa-miR-199a-5p, hsa-miR-3074-5p, hsa-miR-136-3p, hsa-miR-340-3p, hsa-miR-196a-5p, hsa-miR-376c-3p, hsa-miR-361-3p, hsa-miR-379-5p, hsa-miR-214-3p, hsa-let-7b-3p, hsa-miR-1246, hsa-miR-409-5p, hsa-miR-125a-5p, hsa-miR-625-3p, hsa-miR-130b-3p, hsa-miR-543, hsa-miR-221-3p, hsa-miR-99b-5p, hsa-miR-134-5p, hsa-miR-5787, hsa-miR-222-3p, hsa-miR-34a-5p, hsa-miR-154-5p, hsa-miR-6089, hsa-let-7e-5p, hsa-miR-5096, hsa-miR-34a-3p, hsa-miR-127-3p, hsa-miR-191-5p, hsa-miR-30e-3p, hsa-miR-576-5p, hsa-miR-149-5p, hsa-miR-199b-3p, hsa-miR-22-3p, hsa-miR-874-3p, hsa-miR-181c-5p, hsa-miR-342-3p, hsa-miR-151a-3p, hsa-miR-100-5p, hsa-miR-193b-3p, hsa-miR-23a-3p, hsa-miR-186-5p, hsa-miR-103b, hsa-miR-222-5p, hsa-miR-424-3p, hsa-miR-193b-5p, hsa-miR-1273a, hsa-miR-3613-5p, hsa-miR-28-3p, hsa-miR-328-3p, hsa-miR-1306-5p, hsa-miR-365b-3p, hsa-let-7g-5p, hsa-miR-4449, hsa-miR-138-5p, hsa-miR-3960, hsa-miR-92a-3p, hsa-miR-27a-3p, hsa-miR-15b-3p, hsa-miR-485-3p, hsa-miR-424-5p, hsa-miR-30c-5p, hsa-miR-26b-3p, hsa-miR-6087, hsa-let-7d-3p, hsa-miR-494-3p, hsa-miR-10b-5p, hsa-miR-92a-1-5p, hsa-miR-4454, hsa-miR-98-5p, hsa-miR-22-5p, hsa-miR-3607-5p, hsa-miR-146b-5p, hsa-miR-10a-5p, hsa-miR-3613-3p, hsa-miR-3653-5p, hsa-miR-423-5p, hsa-miR-29b-3p, hsa-miR-655-3p, hsa-miR-664b-5p, hsa-miR-29a-3p, hsa-miR-374b-5p, hsa-miR-7-1-3p, hsa-miR-664b-3p, hsa-miR-574-5p, hsa-miR-335-5p, hsa-miR-23a-5p, hsa-miR-6516-3p, hsa-miR-199b-5p, hsa-miR-374c-3p, hsa-miR-24-2-5p, hsa-miR-1291, hsa-miR-125b-5p, hsa-miR-425-5p, hsa-miR-3605-3p, hsa-let-7i-3p, hsa-miR-3184-3p, hsa-miR-181a-5p, hsa-miR-6832-3p, hsa-miR-455-3p, hsa-let-7c-5p, hsa-miR-196b-5p, hsa-miR-146a-5p, hsa-miR-671-5p, hsa-miR-337-3p, hsa-let-7f-1-3p, hsa-miR-16-2-3p, hsa-miR-1271-5p, hsa-let-7d-5p, hsa-miR-4668-5p, hsa-miR-181b-5p, hsa-miR-4461, hsa-miR-145-5p, hsa-miR-660-5p, hsa-miR-26a-2-3p, hsa-miR-6724-5p, hsa-miR-93-5p, hsa-miR-664a-3p, hsa-miR-376a-3p, hsa-miR-190a-5p, hsa-miR-619-5p, hsa-miR-185-5p, hsa-miR-539-5p, hsa-miR-3609, hsa-miR-130a-3p, hsa-miR-3651, hsa-miR-708-5p, hsa-miR-411-5p, hsa-let-7i-5p, hsa-miR-495-3p, hsa-miR-98-3p, hsa-miR-425-3p, hsa-miR-409-3p, hsa-let-7a-3p, hsa-miR-1237-5p, hsa-miR-4485-3p, hsa-miR-210-3p, hsa-miR-28-5p, hsa-miR-223-3p, hsa-miR-532-5p, hsa-miR-199a-3p, hsa-miR-99b-3p and a combination thereof.

TABLE 3
miRNAs according to the invention

| | | | |
|---|---|---|---|
| hsa-let-7a-5p | hsa-miR-92a-3p | hsa-miR-92b-3p | hsa-miR-24-2-5p |
| hsa-let-7b-5p | hsa-miR-125b-5p | hsa-miR-335-5p | hsa-miR-26a-2-3p |
| hsa-let-7f-5p | hsa-miR-337-3p | hsa-let-7f-1-3p | hsa-miR-301a-3p |
| hsa-miR-24-3p | hsa-miR-93-5p | hsa-miR-196b-5p | hsa-miR-98-3p |
| hsa-miR-21-5p | hsa-miR-409-3p | hsa-miR-3613-3p | hsa-miR-1273a |
| hsa-miR-23b-3p | hsa-miR-199a-3p | hsa-miR-23a-5p | hsa-miR-28-5p |
| hsa-miR-1273g-3p | hsa-miR-145-5p | hsa-miR-374b-5p | hsa-miR-34a-3p |
| hsa-miR-574-3p | hsa-miR-30a-3p | hsa-miR-660-5p | hsa-miR-425-3p |
| hsa-miR-25-3p | hsa-miR-382-5p | hsa-miR-186-5p | hsa-miR-505-3p |
| hsa-let-7e-5p | hsa-miR-19b-3p | hsa-miR-454-3p | hsa-miR-34b-3p |
| hsa-miR-214-3p | hsa-miR-210-3p | hsa-miR-10a-5p | hsa-miR-361-3p |
| hsa-miR-199a-5p | hsa-miR-619-5p | hsa-miR-495-3p | hsa-miR-10b-5p |
| hsa-miR-196a-5p | hsa-miR-17-5p | hsa-miR-425-5p | hsa-miR-1306-5p |
| hsa-miR-199b-5p | hsa-miR-193a-5p | hsa-miR-2053 | hsa-miR-22-5p |
| hsa-miR-221-3p | hsa-miR-320b | hsa-miR-5096 | hsa-miR-378a-3p |
| hsa-miR-424-5p | hsa-miR-193b-5p | hsa-miR-494-3p | hsa-miR-411-5p |
| hsa-miR-23a-3p | hsa-miR-320a | hsa-miR-27a-3p | hsa-miR-505-5p |
| hsa-let-7c-5p | hsa-miR-151a-3p | hsa-miR-4449 | hsa-miR-664a-3p |
| hsa-miR-199b-3p | hsa-let-7a-3p | hsa-miR-532-3p | hsa-miR-26a-5p |
| hsa-miR-191-5p | hsa-miR-30e-3p | hsa-miR-532-5p | hsa-miR-377-3p |
| hsa-miR-574-5p | hsa-miR-22-3p | hsa-miR-126-5p | hsa-miR-485-3p |
| hsa-miR-424-3p | hsa-miR-99b-5p | hsa-miR-30c-5p | hsa-miR-590-3p |
| hsa-miR-423-5p | hsa-miR-625-3p | hsa-miR-130b-3p | hsa-miR-99a-3p |
| hsa-miR-342-3p | hsa-miR-4668-5p | hsa-miR-136-3p | hsa-miR-143-3p |
| hsa-let-7d-3p | hsa-miR-29b-3p | hsa-miR-15b-3p | hsa-miR-26b-3p |
| hsa-miR-130a-3p | hsa-miR-423-3p | hsa-miR-29b-1-5p | hsa-miR-3607-5p |
| hsa-miR-3184-3p | hsa-miR-376c-3p | hsa-miR-99b-3p | hsa-miR-3651 |
| hsa-miR-222-3p | hsa-let-7b-3p | hsa-miR-127-3p | hsa-miR-374a-3p |
| hsa-let-7g-5p | hsa-miR-3074-5p | hsa-miR-134-5p | hsa-miR-376a-3p |
| hsa-miR-125a-5p | hsa-miR-98-5p | hsa-miR-324-5p | hsa-miR-485-5p |
| hsa-let-7d-5p | hsa-miR-185-5p | hsa-miR-3605-3p | hsa-miR-103b |
| hsa-miR-29a-3p | hsa-miR-19a-3p | hsa-miR-101-3p | hsa-miR-126-3p |
| hsa-let-7i-5p | hsa-miR-34a-5p | hsa-miR-103a-3p | hsa-miR-149-5p |
| hsa-miR-146b-5p | hsa-miR-374c-3p | hsa-miR-1246 | hsa-miR-193b-3p |
| hsa-miR-4454 | hsa-miR-181a-5p | hsa-miR-138-5p | hsa-miR-223-3p |
| hsa-miR-28-3p | hsa-miR-328-3p | hsa-miR-190a-5p | hsa-miR-340-3p |
| hsa-miR-874-3p | hsa-miR-7847-3p | hsa-miR-6724-5p | hsa-miR-369-5p |

In some embodiments, the at least one miRNA is selected in a group comprising hsa-let-7a-5p, hsa-miR-92a-3p, hsa-miR-92b-3p, hsa-miR-24-2-5p, hsa-let-7b-5p, hsa-miR-125b-5p, hsa-miR-335-5p, hsa-miR-26a-2-3p, hsa-let-7f-5p, hsa-miR-337-3p, hsa-let-7f-1-3p, hsa-miR-301a-3p, hsa-miR-24-3p, hsa-miR-93-5p, hsa-miR-196b-5p, hsa-miR-98-3p, hsa-miR-21-5p, hsa-miR-409-3p, hsa-miR-3613-3p, hsa-miR-1273a, hsa-miR-23b-3p, hsa-miR-199a-3p, hsa-miR-23a-5p, hsa-miR-28-5p, hsa-miR-1273g-3p, hsa-miR-145-5p, hsa-miR-374b-5p, hsa-miR-34a-3p, hsa-miR-574-3p, hsa-miR-30a-3p, hsa-miR-660-5p, hsa-miR-425-3p, hsa-miR-25-3p, hsa-miR-382-5p, hsa-miR-186-5p, hsa-miR-505-3p, hsa-let-7e-5p, hsa-miR-19b-3p, hsa-miR-454-3p, hsa-miR-34b-3p, hsa-miR-214-3p, hsa-miR-210-3p, hsa-miR-10a-5p, hsa-miR-361-3p, hsa-miR-199a-5p, hsa-miR-619-5p, hsa-miR-495-3p, hsa-miR-10b-5p, hsa-miR-196a-5p, hsa-miR-17-5p, hsa-miR-425-5p, hsa-miR-1306-5p, hsa-miR-199b-5p, hsa-miR-193a-5p, hsa-miR-2053, hsa-miR-22-5p, hsa-miR-221-3p, hsa-miR-320b, hsa-miR-5096, hsa-miR-378a-3p, hsa-miR-424-5p, hsa-miR-193b-5p, hsa-miR-494-3p, hsa-miR-411-5p, hsa-miR-23a-3p, hsa-miR-320a, hsa-miR-27a-3p, hsa-miR-505-5p, hsa-let-7c-5p, hsa-miR-151a-3p, hsa-miR-4449, hsa-miR-664a-3p, hsa-miR-199b-3p, hsa-let-7a-3p, hsa-miR-532-3p, hsa-miR-26a-5p, hsa-miR-191-5p, hsa-miR-30e-3p, hsa-miR-532-5p, hsa-miR-377-3p, hsa-miR-574-5p, hsa-miR-22-3p, hsa-miR-126-5p, hsa-miR-485-3p, hsa-miR-424-3p, hsa-miR-99b-5p, hsa-miR-30c-5p, hsa-miR-590-3p, hsa-miR-423-5p, hsa-miR-625-3p, hsa-miR-130b-3p, hsa-miR-99a-3p, hsa-miR-342-3p, hsa-miR-4668-5p, hsa-miR-136-3p, hsa-miR-143-3p, hsa-let-7d-3p, hsa-miR-29b-3p, hsa-miR-15b-3p, hsa-miR-26b-3p, hsa-miR-130a-3p, hsa-miR-423-3p, hsa-miR-29b-1-5p, hsa-miR-3607-5p, hsa-miR-3184-3p, hsa-miR-376c-3p, hsa-miR-99b-3p, hsa-miR-3651, hsa-miR-222-3p, hsa-let-7b-3p, hsa-miR-127-3p, hsa-miR-374a-3p, hsa-let-7g-5p, hsa-miR-3074-5p, hsa-miR-134-5p, hsa-miR-376a-3p, hsa-miR-125a-5p, hsa-miR-98-5p, hsa-miR-324-5p, hsa-miR-485-5p, hsa-let-7d-5p, hsa-miR-185-5p, hsa-miR-3605-3p, hsa-miR-103b, hsa-miR-29a-3p, hsa-miR-19a-3p, hsa-miR-101-3p, hsa-miR-126-3p, hsa-let-7i-5p, hsa-miR-34a-5p, hsa-miR-103a-3p, hsa-miR-149-5p, hsa-miR-146b-5p, hsa-miR-374c-3p, hsa-miR-1246, hsa-miR-193b-3p, hsa-miR-4454, hsa-miR-181a-5p, hsa-miR-138-5p, hsa-miR-223-3p, hsa-miR-28-3p, hsa-miR-328-3p, hsa-miR-190a-5p, hsa-miR-340-3p, hsa-miR-874-3p, hsa-miR-7847-3p, hsa-miR-6724-5p, hsa-miR-369-5p, and a combination thereof.

TABLE 4
miRNAs according to the invention

| | | | |
|---|---|---|---|
| hsa-let-7a-5p | hsa-let-7i-5p | hsa-miR-660-5p | hsa-miR-6832-3p |
| hsa-let-7b-5p | hsa-miR-409-3p | hsa-miR-664a-3p | hsa-miR-146a-5p |
| hsa-miR-24-3p | hsa-miR-210-3p | hsa-miR-185-5p | hsa-miR-16-2-3p |
| hsa-miR-21-5p | hsa-miR-199a-3p | hsa-miR-3651 | hsa-miR-181b-5p |
| hsa-let-7f-5p | hsa-miR-30a-3p | hsa-miR-495-3p | hsa-miR-26a-2-3p |
| hsa-miR-574-3p | hsa-miR-320b | hsa-let-7a-3p | hsa-miR-376a-3p |
| hsa-miR-23b-3p | hsa-miR-193a-5p | hsa-miR-28-5p | hsa-miR-539-5p |
| hsa-miR-1273g-3p | hsa-miR-382-5p | hsa-miR-99b-3p | hsa-miR-708-5p |
| hsa-miR-25-3p | hsa-miR-423-3p | hsa-miR-103a-3p | hsa-miR-98-3p |
| hsa-miR-199a-5p | hsa-miR-17-5p | hsa-miR-19a-3p | hsa-miR-1237-5p |
| hsa-miR-196a-5p | hsa-miR-19b-3p | hsa-miR-126-5p | hsa-miR-223-3p |
| hsa-miR-214-3p | hsa-miR-92b-3p | hsa-miR-2053 | hsa-miR-532-5p |
| hsa-miR-125a-5p | hsa-miR-320a | hsa-miR-29b-1-5p | hsa-miR-542-3p |
| hsa-miR-221-3p | hsa-miR-3074-5p | hsa-miR-3648 | hsa-miR-663a |
| hsa-miR-222-3p | hsa-miR-376c-3p | hsa-miR-374a-3p | hsa-miR-101-3p |
| hsa-let-7e-5p | hsa-let-7b-3p | hsa-miR-454-3p | hsa-miR-143-3p |
| hsa-miR-191 -5p | hsa-miR-625-3p | hsa-miR-532-3p | hsa-miR-21-3p |
| hsa-miR-199b-3p | hsa-miR-99b-5p | hsa-miR-136-3p | hsa-miR-224-5p |
| hsa-miR-342-3p | hsa-miR-34a-5p | hsa-miR-361-3p | hsa-miR-26a-5p |
| hsa-miR-23a-3p | hsa-miR-5096 | hsa-miR-1246 | hsa-miR-27a-5p |
| hsa-miR-424-3p | hsa-miR-30e-3p | hsa-miR-130b-3p | hsa-miR-324-5p |
| hsa-miR-28-3p | hsa-miR-22-3p | hsa-miR-134-5p | hsa-miR-340-3p |
| hsa-let-7g-5p | hsa-miR-151a-3p | hsa-miR-154-5p | hsa-miR-379-5p |
| hsa-miR-92a-3p | hsa-miR-186-5p | hsa-miR-34a-3p | hsa-miR-409-5p |
| hsa-miR-424-5p | hsa-miR-193b-5p | hsa-miR-576-5p | hsa-miR-543 |
| hsa-let-7d-3p | hsa-miR-328-3p | hsa-miR-874-3p | hsa-miR-5787 |
| hsa-miR-4454 | hsa-miR-4449 | hsa-miR-100-5p | hsa-miR-6089 |
| hsa-miR-146b-5p | hsa-miR-27a-3p | hsa-miR-103b | hsa-miR-127-3p |
| hsa-miR-423-5p | hsa-miR-30c-5p | hsa-miR-1273a | hsa-miR-149-5p |
| hsa-miR-29a-3p | hsa-miR-494-3p | hsa-miR-1306-5p | hsa-miR-181c-5p |
| hsa-miR-574-5p | hsa-miR-98-5p | hsa-miR-138-5p | hsa-miR-193b-3p |
| hsa-miR-199b-5p | hsa-miR-10a-5p | hsa-miR-15b-3p | hsa-miR-222-5p |
| hsa-miR-125b-5p | hsa-miR-29b-3p | hsa-miR-26b-3p | hsa-miR-3613-5p |
| hsa-miR-3184-3p | hsa-miR-374b-5p | hsa-miR-10b-5p | hsa-miR-365b-3p |
| hsa-let-7c-5p | hsa-miR-335-5p | hsa-miR-22-5p | hsa-miR-3960 |
| hsa-miR-337-3p | hsa-miR-374c-3p | hsa-miR-3613-3p | hsa-miR-485-3p |
| hsa-let-7d-5p | hsa-miR-425-5p | hsa-miR-655-3p | hsa-miR-6087 |
| hsa-miR-145-5p | hsa-miR-181a-5p | hsa-miR-7-1-3p | hsa-miR-92a-1-5p |
| hsa-miR-93-5p | hsa-miR-196b-5p | hsa-miR-23a-5p | hsa-miR-4668-5p |
| hsa-miR-619-5p | hsa-let-7f-1-3p | hsa-miR-24-2-5p | hsa-miR-3605-3p |
| hsa-miR-130a-3p | | | |

In certain embodiments, at least one miRNA is selected in a group comprising or consisting of hsa-let-7a-5p, hsa-let-7i-5p, hsa-miR-660-5p, hsa-miR-6832-3p, hsa-let-7b-5p, hsa-miR-409-3p, hsa-miR-664a-3p, hsa-miR-146a-5p, hsa-miR-24-3p, hsa-miR-210-3p, hsa-miR-185-5p, hsa-miR-16-2-3p, hsa-miR-21-5p, hsa-miR-199a-3p, hsa-miR-3651, hsa-miR-181b-5p, hsa-let-7f-5p, hsa-miR-30a-3p, hsa-miR-495-3p, hsa-miR-26a-2-3p, hsa-miR-574-3p, hsa-miR-320b, hsa-let-7a-3p, hsa-miR-376a-3p, hsa-miR-23b-3p, hsa-miR-193a-5p, hsa-miR-28-5p, hsa-miR-539-5p, hsa-miR-1273g-3p, hsa-miR-382-5p, hsa-miR-99b-3p, hsa-miR-708-5p, hsa-miR-25-3p, hsa-miR-423-3p, hsa-miR-103a-3p, hsa-miR-98-3p, hsa-miR-199a-5p, hsa-miR-17-5p, hsa-miR-19a-3p, hsa-miR-1237-5p, hsa-miR-196a-5p, hsa-miR-19b-3p, hsa-miR-126-5p, hsa-miR-223-3p, hsa-miR-214-3p, hsa-miR-92b-3p, hsa-miR-2053, hsa-miR-532-5p, hsa-miR-125a-5p, hsa-miR-320a, hsa-miR-29b-1-5p, hsa-miR-542-3p, hsa-miR-221-3p, hsa-miR-3074-5p, hsa-miR-3648, hsa-miR-663a, hsa-miR-222-3p, hsa-miR-376c-3p, hsa-miR-374a-3p, hsa-miR-101-3p, hsa-let-7e-5p, hsa-let-7b-3p, hsa-miR-454-3p, hsa-miR-143-3p, hsa-miR-191-5p, hsa-miR-625-3p, hsa-miR-532-3p, hsa-miR-21-3p, hsa-miR-199b-3p, hsa-miR-99b-5p, hsa-miR-136-3p, hsa-miR-224-5p, hsa-miR-342-3p, hsa-miR-34a-5p, hsa-miR-361-3p, hsa-miR-26a-5p, hsa-miR-23a-3p, hsa-miR-5096, hsa-miR-1246, hsa-miR-27a-5p, hsa-miR-424-3p, hsa-miR-30e-3p, hsa-miR-130b-3p, hsa-miR-324-5p, hsa-miR-28-3p, hsa-miR-22-3p, hsa-miR-134-5p, hsa-miR-340-3p, hsa-let-7g-5p, hsa-miR-151a-3p, hsa-miR-154-5p, hsa-miR-379-5p, hsa-miR-92a-3p, hsa-miR-186-5p, hsa-miR-34a-3p, hsa-miR-409-5p, hsa-miR-424-5p, hsa-miR-193b-5p, hsa-miR-576-5p, hsa-miR-543, hsa-let-7d-3p, hsa-miR-328-3p, hsa-miR-874-3p, hsa-miR-5787, hsa-miR-4454, hsa-miR-4449, hsa-miR-100-5p, hsa-miR-6089, hsa-miR-146b-5p, hsa-miR-27a-3p, hsa-miR-103b, hsa-miR-127-3p, hsa-miR-423-5p, hsa-miR-30c-5p, hsa-miR-1273a, hsa-miR-149-5p, hsa-miR-29a-3p, hsa-miR-494-3p, hsa-miR-1306-5p, hsa-miR-181c-5p, hsa-miR-574-5p, hsa-miR-98-5p, hsa-miR-138-5p, hsa-miR-193b-3p, hsa-miR-199b-5p, hsa-miR-10a-5p, hsa-miR-15b-3p, hsa-miR-222-5p, hsa-miR-125b-5p, hsa-miR-29b-3p, hsa-miR-26b-3p, hsa-miR-3613-5p, hsa-miR-3184-3p, hsa-miR-374b-5p, hsa-miR-10b-5p, hsa-miR-365b-3p, hsa-let-7c-5p, hsa-miR-335-5p, hsa-miR-22-5p, hsa-miR-3960, hsa-miR-337-3p, hsa-miR-374c-3p, hsa-miR-3613-3p, hsa-miR-485-3p, hsa-let-7d-5p, hsa-miR-425-5p, hsa-miR-655-3p, hsa-miR-6087, hsa-miR-145-5p, hsa-miR-181a-5p, hsa-miR-7-1-3p, hsa-miR-92a-1-5p, hsa-miR-93-5p, hsa-miR-196b-5p, hsa-miR-23a-5p, hsa-miR-4668-5p, hsa-miR-619-5p, hsa-let-7f-1-3p, hsa-miR-24-2-5p, hsa-miR-3605-3p, hsa-miR-130a-3p and a combination thereof.

TABLE 5
miRNAs according to the invention

| | | | |
|---|---|---|---|
| hsa-let-7a-5p | hsa-miR-210-3p | hsa-miR-29b-3p | hsa-miR-30e-3p |
| hsa-let-7b-5p | hsa-miR-3184-3p | hsa-miR-92a-3p | hsa-miR-320a |
| hsa-miR-24-3p | hsa-let-7d-5p | hsa-miR-193b-5p | hsa-miR-361-3p |
| hsa-miR-199a-5p | hsa-miR-25-3p | hsa-miR-181a-5p | hsa-miR-151a-3p |
| hsa-miR-214-3p | hsa-miR-193a-5p | hsa-miR-30c-5p | hsa-miR-154-5p |
| hsa-let-7f-5p | hsa-miR-199a-3p | hsa-miR-664b-3p | hsa-miR-664a-5p |
| hsa-miR-3607-5p | hsa-miR-29a-3p | hsa-miR-27a-3p | hsa-miR-92b-3p |
| hsa-miR-199b-3p | hsa-miR-342-3p | hsa-miR-320b | hsa-miR-1291 |
| hsa-let-7e-5p | hsa-miR-130a-3p | hsa-miR-3651 | hsa-miR-103b |
| hsa-miR-1273g-3p | hsa-miR-30a-3p | hsa-miR-664b-5p | hsa-miR-34a-3p |
| hsa-miR-125a-5p | hsa-miR-145-5p | hsa-miR-664a-3p | hsa-miR-140-5p |
| hsa-miR-21-5p | hsa-miR-28-3p | hsa-miR-98-5p | hsa-miR-3609 |
| hsa-let-7i-5p | hsa-miR-93-5p | hsa-miR-146b-5p | hsa-miR-374c-3p |
| hsa-miR-125b-5p | hsa-miR-34a-5p | hsa-miR-337-3p | hsa-miR-10a-5p |
| hsa-let-7g-5p | hsa-miR-222-3p | hsa-miR-4449 | hsa-miR-22-3p |
| hsa-miR-191-5p | hsa-miR-3074-5p | hsa-miR-6516-3p | hsa-miR-4668-5p |
| hsa-miR-574-3p | hsa-miR-424-5p | hsa-let-7i-3p | hsa-miR-24-2-5p |
| hsa-miR-199b-5p | hsa-miR-424-3p | hsa-miR-103a-3p | hsa-miR-29b-1-5p |
| hsa-miR-423-5p | hsa-miR-328-3p | hsa-miR-324-5p | hsa-miR-335-5p |
| hsa-miR-574-5p | hsa-miR-17-5p | hsa-miR-660-5p | hsa-miR-425-5p |
| hsa-miR-23b-3p | hsa-miR-23a-3p | hsa-miR-185-5p | hsa-miR-4461 |
| hsa-miR-196a-5p | hsa-let-7d-3p | hsa-miR-374b-5p | hsa-miR-127-3p |
| hsa-let-7c-5p | hsa-miR-423-3p | hsa-miR-409-3p | hsa-miR-196b-5p |
| hsa-miR-221-3p | hsa-miR-382-5p | hsa-miR-619-5p | hsa-miR-3613-5p |
| hsa-miR-3653-5p | hsa-miR-19b-3p | hsa-miR-99b-5p | hsa-miR-376c-3p |
| hsa-miR-99b-3p | hsa-miR-663b | hsa-miR-495-3p | hsa-miR-454-3p |

In some embodiments, the at least one miRNA is selected in a group comprising hsa-let-7a-5p, hsa-miR-210-3p, hsa-miR-29b-3p, hsa-miR-30e-3p, hsa-let-7b-5p, hsa-miR-3184-3p, hsa-miR-92a-3p, hsa-miR-320a, hsa-miR-24-3p, hsa-let-7d-5p, hsa-miR-193b-5p, hsa-miR-361-3p, hsa-miR-199a-5p, hsa-miR-25-3p, hsa-miR-181a-5p, hsa-miR-151a-3p, hsa-miR-214-3p, hsa-miR-193a-5p, hsa-miR-30c-5p, hsa-miR-154-5p, hsa-let-7f-5p, hsa-miR-199a-3p, hsa-miR-664b-3p, hsa-miR-664a-5p, hsa-miR-3607-5p, hsa-miR-29a-3p, hsa-miR-27a-3p, hsa-miR-92b-3p, hsa-miR-199b-3p, hsa-miR-342-3p, hsa-miR-320b, hsa-miR-1291, hsa-let-7e-5p, hsa-miR-130a-3p, hsa-miR-3651, hsa-miR-103b, hsa-miR-1273g-3p, hsa-miR-30a-3p, hsa-miR-664b-5p, hsa-miR-34a-3p, hsa-miR-125a-5p, hsa-miR-145-5p, hsa-miR-664a-3p, hsa-miR-140-5p, hsa-miR-21-5p, hsa-miR-28-3p, hsa-miR-98-5p, hsa-miR-3609, hsa-let-7i-5p, hsa-miR-93-5p, hsa-miR-146b-5p, hsa-miR-374c-3p, hsa-miR-125b-5p, hsa-miR-34a-5p, hsa-miR-337-3p, hsa-miR-10a-5p, hsa-let-7g-5p, hsa-miR-222-3p, hsa-miR-4449, hsa-miR-22-3p, hsa-miR-191-5p, hsa-miR-3074-5p, hsa-miR-6516-3p, hsa-miR-4668-5p, hsa-miR-574-3p, hsa-miR-424-5p, hsa-let-7i-3p, hsa-miR-24-2-5p, hsa-miR-199b-5p, hsa-miR-424-3p, hsa-miR-103a-3p, hsa-miR-29b-1-5p, hsa-miR-423-5p, hsa-miR-328-3p, hsa-miR-324-5p, hsa-miR-335-5p, hsa-miR-574-5p, hsa-miR-17-5p, hsa-miR-660-5p, hsa-miR-425-5p, hsa-miR-23b-3p, hsa-miR-23a-3p, hsa-miR-185-5p, hsa-miR-4461, hsa-miR-196a-5p, hsa-let-7d-3p, hsa-miR-374b-5p, hsa-miR-127-3p, hsa-let-7c-5p, hsa-miR-423-3p, hsa-miR-409-3p, hsa-miR-196b-5p, hsa-miR-221-3p, hsa-miR-382-5p, hsa-miR-619-5p, hsa-miR-3613-5p, hsa-miR-3653-5p, hsa-miR-19b-3p, hsa-miR-99b-5p, hsa-miR-376c-3p, hsa-miR-99b-3p, hsa-miR-663b, hsa-miR-495-3p, hsa-miR-454-3p, and a combination thereof.

TABLE 6
miRNAs according to the invention

| | | | |
|---|---|---|---|
| hsa-let-7a-5p | hsa-miR-3653-5p | hsa-miR-98-5p | hsa-miR-28-5p |
| hsa-let-7b-5p | hsa-miR-342-3p | hsa-miR-664a-3p | hsa-miR-10a-5p |
| hsa-miR-24-3p | hsa-miR-28-3p | hsa-miR-92b-3p | hsa-miR-151a-3p |
| hsa-let-7f-5p | hsa-miR-23b-3p | hsa-miR-4449 | hsa-miR-30e-3p |
| hsa-miR-199a-5p | hsa-let-7c-5p | hsa-miR-320a | hsa-miR-324-5p |
| hsa-miR-214-3p | hsa-miR-222-3p | hsa-miR-181a-5p | hsa-miR-495-3p |
| hsa-miR-3607-5p | hsa-miR-29a-3p | hsa-miR-3651 | hsa-miR-576-5p |
| hsa-miR-125a-5p | hsa-miR-92a-3p | hsa-miR-185-5p | hsa-miR-625-3p |
| hsa-miR-199b-3p | hsa-miR-30a-3p | hsa-miR-664b-5p | hsa-miR-671-5p |
| hsa-miR-125b-5p | hsa-miR-424-3p | hsa-miR-196b-5p | hsa-miR-1271-5p |
| hsa-miR-21-5p | hsa-miR-423-3p | hsa-miR-27a-3p | hsa-miR-186-5p |
| hsa-let-7e-5p | hsa-miR-34a-5p | hsa-miR-29b-3p | hsa-miR-23a-5p |
| hsa-let-7i-5p | hsa-miR-424-5p | hsa-miR-664b-3p | hsa-miR-3613-5p |
| hsa-let-7g-5p | hsa-miR-145-5p | hsa-miR-99b-5p | hsa-miR-376c-3p |
| hsa-miR-574-3p | hsa-miR-328-3p | hsa-miR-103a-3p | hsa-miR-409-3p |
| hsa-miR-574-5p | hsa-miR-3074-5p | hsa-miR-6516-3p | hsa-miR-4461 |
| hsa-miR-191-5p | hsa-let-7d-3p | hsa-miR-22-3p | hsa-miR-454-3p |
| hsa-miR-196a-5p | hsa-miR-93-5p | hsa-miR-26a-5p | hsa-miR-6724-5p |
| hsa-miR-221-3p | hsa-miR-23a-3p | hsa-miR-103b | hsa-let-7b-3p |
| hsa-miR-25-3p | hsa-miR-19b-3p | hsa-miR-1291 | hsa-miR-190a-5p |
| hsa-miR-423-5p | hsa-miR-146b-5p | hsa-miR-425-5p | hsa-miR-26b-3p |
| hsa-miR-210-3p | hsa-miR-320b | hsa-miR-22-5p | hsa-miR-3609 |
| hsa-miR-1273g-3p | hsa-miR-337-3p | hsa-miR-374c-3p | hsa-miR-411-5p |
| hsa-let-7d-5p | hsa-miR-17-5p | hsa-let-7i-3p | hsa-miR-425-3p |
| hsa-miR-199b-5p | hsa-miR-130a-3p | hsa-miR-374b-5p | hsa-miR-4485-3p |
| hsa-miR-199a-3p | hsa-miR-193b-5p | hsa-miR-455-3p | hsa-miR-30c-5p |
| hsa-miR-193a-5p | hsa-miR-382-5p | hsa-miR-532-3p | hsa-miR-619-5p |
| hsa-miR-3184-3p | | | |

In some embodiments, at least one miRNA is selected in a group comprising or consisting of hsa-let-7a-5p, hsa-miR-3653-5p, hsa-miR-98-5p, hsa-miR-28-5p, hsa-let-7b-5p, hsa-miR-342-3p, hsa-miR-664a-3p, hsa-miR-10a-5p, hsa-miR-24-3p, hsa-miR-28-3p, hsa-miR-92b-3p, hsa-miR-151a-3p, hsa-let-7f-5p, hsa-miR-23b-3p, hsa-miR-4449, hsa-miR-30e-3p, hsa-miR-199a-5p, hsa-let-7c-5p, hsa-miR-320a, hsa-miR-324-5p, hsa-miR-214-3p, hsa-miR-222-3p, hsa-miR-181a-5p, hsa-miR-495-3p, hsa-miR-3607-5p, hsa-miR-29a-3p, hsa-miR-3651, hsa-miR-576-5p, hsa-miR-125a-5p, hsa-miR-92a-3p, hsa-miR-185-5p, hsa-miR-625-3p, hsa-miR-199b-3p, hsa-miR-30a-3p, hsa-miR-664b-5p, hsa-miR-671-5p, hsa-miR-125b-5p, hsa-miR-424-3p, hsa-miR-196b-5p, hsa-miR-1271-5p, hsa-miR-21-5p, hsa-miR-423-3p, hsa-miR-27a-3p, hsa-miR-186-5p, hsa-let-7e-5p, hsa-miR-34a-5p, hsa-miR-29b-3p, hsa-miR-23a-5p, hsa-let-7i-5p, hsa-miR-424-5p, hsa-miR-664b-3p, hsa-miR-3613-5p, hsa-let-7g-5p, hsa-miR-145-5p, hsa-miR-99b-5p, hsa-miR-376c-3p, hsa-miR-574-3p, hsa-miR-328-3p, hsa-miR-103a-3p, hsa-miR-409-3p, hsa-miR-574-5p, hsa-miR-3074-5p, hsa-miR-6516-3p, hsa-miR-4461, hsa-miR-191-5p, hsa-let-7d-3p, hsa-miR-22-3p, hsa-miR-454-3p, hsa-miR-196a-5p, hsa-miR-93-5p, hsa-miR-26a-5p, hsa-miR-6724-5p, hsa-miR-221-3p, hsa-miR-23a-3p, hsa-miR-103b, hsa-let-7b-3p, hsa-miR-25-3p, hsa-miR-19b-3p, hsa-miR-1291, hsa-miR-190a-5p, hsa-miR-423-5p, hsa-miR-146b-5p, hsa-miR-425-5p, hsa-miR-26b-3p, hsa-miR-210-3p, hsa-miR-320b, hsa-miR-22-5p, hsa-miR-3609, hsa-miR-1273g-3p, hsa-miR-337-3p, hsa-miR-374c-3p, hsa-miR-411-5p, hsa-let-7d-5p, hsa-miR-17-5p, hsa-let-7i-3p, hsa-miR-425-3p, hsa-miR-199b-5p, hsa-miR-130a-3p, hsa-miR-374b-5p, hsa-miR-4485-3p, hsa-miR-199a-3p, hsa-miR-193b-5p, hsa-miR-455-3p, hsa-miR-30c-5p, hsa-miR-193a-5p, hsa-miR-382-5p, hsa-miR-532-3p, hsa-miR-619-5p, hsa-miR-3184-3p and a combination thereof.

TABLE 7
miRNAs according to the invention

| | | | |
|---|---|---|---|
| hsa-miR-3687 | hsa-miR-619-5p | hsa-let-7e-5p | hsa-miR-24-3p |
| hsa-miR-664b-5p | hsa-miR-181a-5p | hsa-miR-25-3p | hsa-miR-382-5p |
| hsa-miR-210-3p | hsa-miR-409-3p | hsa-miR-374c-3p | hsa-miR-214-3p |
| hsa-miR-4449 | hsa-let-7a-3p | hsa-miR-29b-3p | hsa-miR-199b-5p |
| hsa-miR-3651 | hsa-miR-4454 | hsa-let-7b-3p | hsa-miR-199a-5p |
| hsa-miR-663a | hsa-let-7i-5p | hsa-miR-23b-3p | hsa-miR-3074-5p |
| hsa-miR-664b-3p | hsa-miR-335-5p | hsa-miR-3613-3p | hsa-miR-361-3p |
| hsa-miR-3653-5p | hsa-miR-1246 | hsa-miR-138-5p | hsa-miR-6723-5p |
| hsa-miR-664a-3p | hsa-miR-6516-5p | hsa-miR-6516-3p | hsa-miR-130a-3p |
| hsa-miR-3648 | hsa-miR-3607-5p | hsa-miR-4485-3p | hsa-miR-660-5p |
| hsa-miR-196b-5p | hsa-miR-342-3p | hsa-miR-221-3p | |

In some embodiments, the at least one miRNA is selected in a group comprising hsa-miR-3687, hsa-miR-619-5p, hsa-let-7e-5p, hsa-miR-24-3p, hsa-miR-664b-5p, hsa-miR-181a-5p, hsa-miR-25-3p, hsa-miR-382-5p, hsa-miR-210-3p, hsa-miR-409-3p, hsa-miR-374c-3p, hsa-miR-214-3p, hsa-miR-4449, hsa-let-7a-3p, hsa-miR-29b-3p, hsa-miR-199b-5p, hsa-miR-3651, hsa-miR-4454, hsa-let-7b-3p, hsa-miR-199a-5p, hsa-miR-663a, hsa-let-7i-5p, hsa-miR-23b-3p, hsa-miR-3874-5p, hsa-miR-664b-3p, hsa-miR-334-3p, hsa-miR-3613-3p, hsa-miR-361-3p, hsa-miR-363-p, hsa-miR-1246, hsa-miR-138-5p, hsa-miR-6723-5p, hsa-miR-664a-3p, hsa-miR-656-5p, hsa-miR-656-3p, hsa-miR-130a-3p, hsa-miR-3648, hsa-miR-3607-5p, hsa-miR-4485-3p, hsa-miR-660-5p, hsa-miR-196b-5p, hsa-miR-342-3p, hsa-miR-221-3p, and a combination thereof.

TABLE 8
miRNAs according to the invention

| | | | |
|---|---|---|---|
| hsa-miR-210-3p | hsa-miR-409-3p | hsa-miR-219 | hsa-miR-29b |
| hsa-miR-4454 | hsa-miR-3607-5p | hsa-miR-299-5p | has-miR-140-5p |
| hsa-miR-619-5p | hsa-miR-3609 | hsa-miR-302b | hsa-miR-31 |
| hsa-miR-1246 | hsa-miR-663a | has-miR-221 | hsa-miR-30 |
| hsa-miR-222-3p | hsa-miR-19a-3p | hsa-miR-155 | hsa-miR-30e |
| hsa-miR-181a-5p | hsa-miR-3651 | hsa-miR-885-5p | hsa-miR-17 |
| hsa-miR-6832-3p | hsa-miR-4668-5p | hsa-miR-181a | hsa-miR-433 |
| hsa-miR-335-5p | hsa-miR-301a-3p | hsa-miR-320c | hsa-miR-486-5p |
| hsa-let-7a-3p | hsa-miR-664a-3p | hsa-miR-548d-5p | hsa-miR-335 |
| hsa-miR-28-3p | hsa-miR-485-5p | hsa-miR-34a | hsa-miR-106a |
| hsa-miR-125a-5p | hsa-miR-382-5p | hsa-miR-378 | hsa-miR-21-3p |
| hsa-miR-374c-3p | hsa-miR-4449 | hsa-346 | hsa-miR-26a-5p |
| hsa-miR-181c-5p | hsa-miR-138-5p | hsa-10a | let-7a-5p |
| hsa-miR-374b-5p | let-7a | hsa-125b | hsa-miR-10a |
| hsa-miR-3687 | hsa-miR-199b | hsa-miR-322 | hsa-miR-148-a |
| hsa-miR-3653-5p | hsa-miR-218 | hsa-miR-21 | hsa-miR-31-5p |
| hsa-miR-664b-5p | hsa-miR-148a | hsa-miR-96 | hsa-miR-486-5p |
| hsa-miR-664b-3p | hsa-miR-135b | hsa-miR-22 | hsa-miR-24-3p |
| hsa-miR-3613-3p | hsa-miR-203 | hsa-miR-27 | hsa-let-7i-5p |
| hsa-miR-3074-5p | hsa-miR-4485-3p | hsa-let-7c-5p | hsa-miR-6723-5p |
| hsa-miR-671-5p | hsa-miR-93-5p | hsa-miR-154-5p | |

In certain embodiments, at least one miRNA is selected in a group comprising or consisting of hsa-miR-210-3p, hsa-miR-409-3p, hsa-miR-219, hsa-miR-29b, hsa-miR-4454, hsa-miR-3607-5p, hsa-miR-299-5p, has-miR-140-5p, hsa-miR-619-5p, hsa-miR-3609, hsa-miR-302b, hsa-miR-31, hsa-miR-1246, hsa-miR-663a, has-miR-221, hsa-miR-30, hsa-miR-222-3p, hsa-miR-19a-3p, hsa-miR-155, hsa-miR-30e, hsa-miR-181a-5p, hsa-miR-3651, hsa-miR-885-5p, hsa-miR-17, hsa-miR-6832-3p, hsa-miR-4668-5p, hsa-miR-181a, hsa-miR-433, hsa-miR-335-5p, hsa-miR-301a-3p, hsa-miR-320c, hsa-miR-486-5p, hsa-let-7a-3p, hsa-miR-664a-3p, hsa-miR-548d-5p, hsa-miR-335, hsa-miR-28-3p, hsa-miR-485-5p, hsa-miR-34a, hsa-miR-106a, hsa-miR-125a-5p, hsa-miR-382-5p, hsa-miR-378, hsa-miR-21-3p, hsa-miR-374c-3p, hsa-miR-4449, hsa-346, hsa-miR-26a-5p, hsa-miR-181c-5p, hsa-miR-138-5p, hsa-10a, let-7a-5p, hsa-miR-374b-5p, let-7a, hsa-125b, hsa-miR-10a, hsa-miR-3687, hsa-miR-199b, hsa-miR-322, hsa-miR-148-a, hsa-miR-3653-5p, hsa-miR-218, hsa-miR-21, hsa-miR-31-5p, hsa-miR-664b-5p, hsa-miR-148a, hsa-miR-96, hsa-miR-486-5p, hsa-miR-664b-3p, hsa-miR-135b, hsa-miR-22, hsa-miR-24-3p, hsa-miR-3613-3p, hsa-miR-203, hsa-miR-27, hsa-let-7i-5p, hsa-miR-3074-5p, hsa-miR-4485-3p, hsa-let-7c-5p, hsa-miR-6723-5p, hsa-miR-671-5p, hsa-miR-93-5p, hsa-miR-154-5p and a combination thereof.

TABLE 9
miRNAs according to the invention

| | | | |
|---|---|---|---|
| hsa-miR-210-3p | hsa-let-7i-5p | hsa-miR-29b-3p | hsa-miR-199a-5p |
| hsa-miR-619-5p | hsa-miR-335-5p | hsa-miR-23b-3p | hsa-miR-3074-5p |
| hsa-miR-181a-5p | hsa-miR-1246 | hsa-miR-24-3p | hsa-miR-361-3p |
| hsa-let-7a-3p | hsa-let-7e-5p | hsa-miR-214-3p | hsa-miR-130a-3p |
| hsa-miR-4454 | hsa-miR-374c-3p | hsa-miR-199b-5p | hsa-miR-3607-5p |
| hsa-miR-660-5p | hsa-miR-342-3p | | |

In some embodiments, the at least one miRNA is selected in a group comprising hsa-miR-210-3p, hsa-let-7i-5p, hsa-miR-29b-3p, hsa-miR-199a-5p, hsa-miR-619-5p, hsa-miR-335-5p, hsa-miR-23b-3p, hsa-miR-3074-5p, hsa-miR-181a-5p, hsa-miR-1246, hsa-miR-24-3p, hsa-miR-361-3p, hsa-let-7a-3p, hsa-let-7e-5p, hsa-miR-214-3p, hsa-miR-130a-3p, hsa-miR-4454, hsa-miR-374c-3p, hsa-miR-199b-5p, hsa-miR-3607-5p, hsa-miR-660-5p, hsa-miR-342-3p, and a combination thereof.

TABLE 10
miRNAs according to the invention

| | | | |
|---|---|---|---|
| hsa-miR-210-3p | hsa-miR-125a-5p | hsa-miR-219 | hsa-miR-21 |
| hsa-miR-4454 | hsa-miR-374c-3p | hsa-miR-299-5p | hsa-miR-96 |
| hsa-miR-619-5p | hsa-miR-181c-5p | hsa-miR-302b | hsa-miR-22 |
| hsa-miR-1246 | hsa-miR-374b-5p | hsa-miR-548d-5p | hsa-miR-27 |
| hsa-miR-222-3p | let-7a | hsa-miR-34a | hsa-miR-29b |
| hsa-miR-181a-5p | hsa-miR-199b | hsa-miR-378 | hsa-miR-24-3p |
| hsa-miR-6832-3p | hsa-miR-218 | hsa-346 | hsa-let-7i-5p |
| hsa-miR-335-5p | hsa-miR-148a | hsa-10a | hsa-miR-3074-5p |
| hsa-let-7a-3p | hsa-miR-135b | hsa-125b | hsa-miR-671-5p |
| hsa-miR-28-3p | hsa-miR-203 | hsa-miR-322 | |

In certain embodiments, at least one miRNA is selected in a group comprising or consisting of hsa-miR-210-3p, hsa-miR-125a-5p, hsa-miR-219, hsa-miR-21, hsa-miR-4454, hsa-miR-374c-3p, hsa-miR-299-5p, hsa-miR-96, hsa-miR-619-5p, hsa-miR-181c-5p, hsa-miR-302b, hsa-miR-22, hsa-miR-1246, hsa-miR-374b-5p, hsa-miR-548d-5p, hsa-miR-27, hsa-miR-222-3p, let-7a, hsa-miR-34a, hsa-miR-29b, hsa-miR-181a-5p, hsa-miR-199b, hsa-miR-378, hsa-miR-24-3p, hsa-miR-6832-3p, hsa-miR-218, hsa-346, hsa-let-7i-5p, hsa-miR-335-5p, hsa-miR-148a, hsa-10a, hsa-miR-3074-5p, hsa-let-7a-3p, hsa-miR-135b, hsa-125b, hsa-miR-671-5p, hsa-miR-28-3p, hsa-miR-203, hsa-miR-322 and a combination thereof.

TABLE 11
miRNAs according to the invention

| | | | |
|---|---|---|---|
| hsa-miR-3687 | hsa-miR-664b-3p | hsa-miR-6516-5p | hsa-miR-138-5p |
| hsa-miR-664b-5p | hsa-miR-3653-5p | hsa-miR-3607-5p | hsa-miR-6516-3p |
| hsa-miR-4449 | hsa-miR-664a-3p | hsa-miR-25-3p | hsa-miR-4485-3p |
| hsa-miR-3651 | hsa-miR-3648 | hsa-let-7b-3p | hsa-miR-382-5p |
| hsa-miR-663a | hsa-miR-409-3p | hsa-miR-3613-3p | hsa-miR-6723-5p |
| hsa-miR-3687 | hsa-miR-664b-3p | hsa-miR-6516-5p | hsa-miR-138-5p |
| hsa-miR-196b-5p | hsa-miR-221-3p | | |

In some embodiments, the at least one miRNA is selected in a group comprising hsa-miR-3687, hsa-miR-664b-3p, hsa-miR-6516-5p, hsa-miR-138-5p, hsa-miR-664b-5p, hsa-miR-3653-5p, hsa-miR-3607-5p, hsa-miR-6516-3p, hsa-miR-4449, hsa-miR-664a-3p, hsa-miR-25-3p, hsa-miR-4485-3p, hsa-miR-3651, hsa-miR-3648, hsa-let-7b-3p, hsa-miR-382-5p, hsa-miR-663a, hsa-miR-409-3p, hsa-miR-3613-3p, hsa-miR-6723-5p, hsa-miR-3687, hsa-miR-664b-3p, hsa-miR-6516-5p, hsa-miR-138-5p, hsa-miR-196b-5p, hsa-miR-221-3p, and a combination thereof

TABLE 12
miRNAs according to the invention

| | | | |
|---|---|---|---|
| hsa-miR-3687 | hsa-miR-19a-3p | has-miR-221 | hsa-miR-17 |
| hsa-miR-3653-5p | hsa-miR-3651 | hsa-miR-155 | hsa-miR-433 |
| hsa-miR-664b-5p | hsa-miR-4668-5p | hsa-miR-885-5p | hsa-miR-486-5p |
| hsa-miR-664b-3p | hsa-miR-301a-3p | hsa-miR-181a | hsa-miR-335 |
| hsa-miR-3613-3p | hsa-miR-664a-3p | hsa-miR-320c | hsa-miR-106a |
| hsa-miR-409-3p | hsa-miR-485-5p | has-miR-140-5p | hsa-miR-4485-3p |
| hsa-miR-3607-5p | hsa-miR-382-5p | hsa-miR-31 | hsa-miR-93-5p |
| hsa-miR-3609 | hsa-miR-4449 | hsa-miR-30 | hsa-let-7c-5p |
| hsa-miR-663a | hsa-miR-138-5p | hsa-miR-30e | hsa-miR-154-5p |
| hsa-miR-6723-5p | | | |

In certain embodiments, at least one miRNA is selected in a group comprising or consisting of hsa-miR-3687, hsa-miR-19a-3p, has-miR-221, hsa-miR-17, hsa-miR-3653-5p, hsa-miR-3651, hsa-miR-155, hsa-miR-433, hsa-miR-664b-5p, hsa-miR-4668-5p, hsa-miR-885-5p, hsa-miR-486-5p, hsa-miR-664b-3p, hsa-miR-301a-3p, hsa-miR-181a, hsa-miR-335, hsa-miR-3613-3p, hsa-miR-664a-3p, hsa-miR-320c, hsa-miR-106a, hsa-miR-409-3p, hsa-miR-485-5p, has-miR-140-5p, hsa-miR-4485-3p, hsa-miR-3607-5p, hsa-miR-382-5p, hsa-miR-31, hsa-miR-93-5p, hsa-miR-3609, hsa-miR-4449, hsa-miR-30, hsa-let-7c-5p, hsa-miR-663a, hsa-miR-138-5p, hsa-miR-30e, hsa-miR-154-5p, hsa-miR-6723-5p and a combination thereof.

In certain embodiments, the at least one miRNA is selected in a group comprising hsa-miR-210-3p, hsa-miR-409-3p, hsa-miR-361-3p, hsa-miR-130a-3p, hsa-miR-660-5p, hsa-miR-199b-5p, hsa-miR-3074-5p, hsa-let-7i-5p, hsa-miR-24-3p, hsa-miR-342-3p, hsa-miR-214-3p, hsa-miR-199a-5p, hsa-miR-3607-5p, hsa-miR-221-3p, hsa-miR-4449, hsa-miR-382-5p, hsa-miR-196b-5p, hsa-miR-663a, hsa-miR-4485-3p, hsa-miR-6723-5p and a combination thereof.

In some embodiments, said at least one miRNA is selected in a group comprising hsa-miR210-3p, hsa-miR-409-3p, hsa-let-7i-5p, hsa-miR-93-5p, hsa-miR-382-5p, hsa-miR-4485-3p, and a combination thereof. In certain embodiments, said at least one miRNA is selected in a group comprising hsa-miR210-3p, hsa-let-7i-5p, hsa-miR-93-5p, hsa-miR-382-5p, and a combination thereof.

In certain embodiments, said at least one miRNA is hsa-miR210-3p and/or hsa-miR-409-3p.

In one embodiment, the pharmaceutical composition of the invention comprises a therapeutically effective amount of at least two miRNAs selected in Table 1, Table 2, Table 3, Table 4, Table 5, Table 6, Table 7, Table 8, Table 9, Table 10, Table 11 or Table 12. In another embodiment, the pharmaceutical composition of the invention comprises a therapeutically effective amount of at least 3, 4, 5, 6, 7, 8, 9, 10, 12, 14, 16, 18, 20 miRNAs selected in Table 1, Table 2, Table 3, Table 4, Table 5, Table 6, Table 7, Table 8, Table 9, Table 10, Table 11 or Table 12.

In some embodiments, said composition comprises a combination of hsa-miR-210-3p, hsa-miR-361-3p, hsa-miR-130a-3p, hsa-miR-660-5p, hsa-miR-199b-5p, hsa-miR-3074-5p, hsa-let-7i-5p, hsa-miR-24-3p, hsa-miR-342-3p, hsa-miR-214-3p, hsa-miR-199a-5p, hsa-miR-3607-5p.

In some embodiments, said RNAs content comprises or consists of hsa-miR210-3p, hsa-miR-409-3p, hsa-let-7i-5p, hsa-miR-93-5p, hsa-miR-382-5p, and hsa-miR-4485-3p. In some preferred embodiments, said RNAs content comprises or consists of hsa-miR210-3p, hsa-let-7i-5p, hsa-miR-93-5p and hsa-miR-382-5p.

In some embodiments, said composition comprises a combination of hsa-miR210-3p, hsa-miR-409-3p, hsa-let-7i-5p, hsa-miR-93-5p, hsa-miR-382-5p, and hsa-miR-4485-3p.

In some preferred embodiments, said composition comprises a combination of hsa-miR210-3p, hsa-let-7i-5p, hsa-miR-93-5p and hsa-miR-382-5p. In some preferred embodiments, said composition comprises at least hsa-miR210-3p.

In certain embodiments, a combination of miRNAs may be referred to as a miRNAs cocktail.

In practice, the RNAs content of the biomaterial according to the instant invention may be assessed by any suitable method known in the art, or any method adapted therefrom.

Illustratively, RNA may be extracted, e.g. by the mean of commercial kit (such as miRNeasy kit from Qiagen®); and further sequenced, e.g. by the mean of a high-throughput sequencing system (such as NextSeq 500 system from Illumina®). Illustratively, one may use the Qiazol lysis reagent (Qiagen®, Hilden, Germany) and a Precellys homogenizer (Bertin® instruments, Montigny-le-Bretonneux, France). RNAs may be purified using Rneasy mini kit (Qiagen®, Hilden, Germany) with an additional on column DNase digestion according to the manufacturer's instruction. Quality and quantity of RNA may be determined using a spectrophotometer (Spectramax 190, Molecular Devices, California, USA). cDNA may be synthesized from 0.5 μg of total RNA using RT² RNA first strand kit (Qiagen®, Hilden, Germany) for genes expression profiles though customized PCR arrays (Customized Human Osteogenic and angiogenic RT² Profiler Assay—Qiagen®, Hilden, Germany). The ABI Quantstudio 5 system (Applied Biosystems®) and SYBR Green ROX Mastermix (Qiagen®, Hilden, Germany) may be used for detection of the amplification product. Quantification may be obtained according to the ΔΔCT method. The final result of each sample may be normalized to the means of expression level of three housekeeping genes (e.g. ACTB, B2M and GAPDH). In some embodiments, the factors content, including proteins and nucleic acids, is originating from the cells.

In some embodiments, at least part of the factors content is cellular. In some embodiments, at least part of the factors content is cellular miRNAs.

In practice, cellular miRNAs may be isolated by any suitable method known from the state of the art, or a method adapted therefrom. One may refer, e.g., to Chapter 7: Extraction, Purification, and Analysis of mRNA from Eukaryotic Cells of Molecular Cloning: a laboratory manual (Russell and Sambrook; 2001; Cold Spring Harbor Laboratory). Illustratively, miRNAs may be isolated by a commercial kit, such as, e.g., RNeasy Mini kit (Qiagen®) or MagMax mirVana Total RNA isolation kit (Applied Biosystems®), following the manufacturer's instructions. RNA concentration may be determined by Nanodrop (ThermoFisher®, Waltham, Massachusetts, USA).

In some embodiments, the at least one miRNA is selected in a group comprising hsa-let-7a-5p, hsa-miR-210-3p, hsa-miR-29b-3p, hsa-miR-30e-3p, hsa-let-7b-5p, hsa-miR-3184-3p, hsa-miR-92a-3p, hsa-miR-320a, hsa-miR-24-3p, hsa-let-7d-5p, hsa-miR-193b-5p, hsa-miR-361-3p, hsa-miR-199a-5p, hsa-miR-25-3p, hsa-miR-181a-5p, hsa-miR-151a-3p, hsa-miR-214-3p, hsa-miR-193a-5p, hsa-miR-30c-5p, hsa-miR-154-5p, hsa-let-7f-5p, hsa-miR-199a-3p, hsa-miR-664b-3p, hsa-miR-664a-5p, hsa-miR-3607-5p, hsa-miR-29a-3p, hsa-miR-27a-3p, hsa-miR-92b-3p, hsa-miR-199b-3p, hsa-miR-342-3p, hsa-miR-320b, hsa-miR-1291, hsa-let-7e-5p, hsa-miR-130a-3p, hsa-miR-3651, hsa-miR-103b, hsa-miR-1273g-3p, hsa-miR-30a-3p, hsa-miR-664b-5p, hsa-miR-34a-3p, hsa-miR-125a-5p, hsa-miR-145-5p, hsa-miR-664a-3p, hsa-miR-140-5p, hsa-miR-21-5p, hsa-miR-28-3p, hsa-miR-98-5p, hsa-miR-3609, hsa-let-7i-5p, hsa-miR-93-5p, hsa-miR-146b-5p, hsa-miR-374c-3p, hsa-miR-125b-5p, hsa-miR-34a-5p, hsa-miR-337-3p, hsa-miR-10a-5p, hsa-let-7g-5p, hsa-miR-222-3p, hsa-miR-4449, hsa-miR-22-3p, hsa-miR-191-5p, hsa-miR-3074-5p, hsa-miR-6516-3p, hsa-miR-4668-5p, hsa-miR-574-3p, hsa-miR-424-5p, hsa-let-7i-3p, hsa-miR-24-2-5p, hsa-miR-199b-5p, hsa-miR-424-3p, hsa-miR-103a-3p, hsa-miR-29b-1-5p, hsa-miR-423-5p, hsa-miR-328-3p, hsa-miR-324-5p, hsa-miR-335-5p, hsa-miR-574-5p, hsa-miR-17-5p, hsa-miR-660-5p, hsa-miR-425-5p, hsa-miR-23b-3p, hsa-miR-23a-3p, hsa-miR-185-5p, hsa-miR-4461, hsa-miR-196a-5p, hsa-let-7d-3p, hsa-miR-374b-5p, hsa-miR-127-3p, hsa-let-7c-5p, hsa-miR-423-3p, hsa-miR-409-3p, hsa-miR-196b-5p, hsa-miR-221-3p, hsa-miR-382-5p, hsa-miR-619-5p, hsa-miR-3613-5p, hsa-miR-3653-5p, hsa-miR-19b-3p, hsa-miR-99b-5p, hsa-miR-376c-3p, hsa-miR-99b-3p, hsa-miR-663b, hsa-miR-495-3p, hsa-miR-454-3p, and a combination thereof.

In certain embodiments, the cellular miRNAs are selected in a group comprising hsa-miR-210-3p, hsa-miR-409-3p, hsa-miR-361-3p, hsa-miR-130a-3p, hsa-miR-660-5p, hsa-miR-199b-5p, hsa-miR-3074-5p, hsa-let-7i-5p, hsa-miR-24-3p, hsa-miR-342-3p, hsa-miR-214-3p, hsa-miR-199a-5p, hsa-miR-3607-5p, hsa-miR-221-3p, hsa-miR-4449, hsa-miR-382-5p, hsa-miR-196b-5p, hsa-miR-663a, hsa-miR-4485-3p, hsa-miR-6723-5p and a combination thereof.

In one embodiment, the cellular miRNAs are selected in a group comprising hsa-let-7a-5p, hsa-let-7b-5p, hsa-miR-24-3p, hsa-let-7f-5p, hsa-miR-199a-5p, hsa-miR-214-3p, hsa-miR-3607-5p, hsa-miR-125a-5p, hsa-miR-199b-3p, hsa-miR-125b-5p, hsa-miR-21-5p, hsa-let-7e-5p, hsa-let-7i-5p, hsa-let-7g-5p, hsa-miR-574-3p, hsa-miR-574-5p, hsa-miR-191-5p, hsa-miR-196a-5p, hsa-miR-221-3p, hsa-miR-25-3p, hsa-miR-423-5p, hsa-miR-210-3p, hsa-miR-1273g-3p, hsa-let-7d-5p, hsa-miR-199b-5p, hsa-miR-199a-3p, hsa-miR-193a-5p, hsa-miR-3184-3p, hsa-miR-3653-5p, hsa-miR-342-3p, hsa-miR-28-3p, hsa-miR-23b-3p, hsa-let-7c-5p, hsa-miR-222-3p, hsa-miR-29a-3p, hsa-miR-92a-3p, hsa-miR-30a-3p, hsa-miR-424-3p, hsa-miR-423-3p, hsa-miR-34a-5p, hsa-miR-424-5p, hsa-miR-145-5p, hsa-miR-328-3p, hsa-miR-3074-5p, hsa-let-7d-3p, hsa-miR-93-5p, hsa-miR-23a-3p, hsa-miR-19b-3p, hsa-miR-146b-5p, hsa-miR-320b, hsa-miR-337-3p, hsa-miR-17-5p, hsa-miR-130a-3p, hsa-miR-193b-5p, hsa-miR-382-5p, hsa-miR-30c-5p, hsa-miR-98-5p, hsa-miR-664a-3p, hsa-miR-92b-3p, hsa-miR-4449, hsa-miR-320a, hsa-miR-181a-5p, hsa-miR-3651, hsa-miR-185-5p, hsa-miR-664b-5p, hsa-miR-196b-5p, hsa-miR-27a-3p, hsa-miR-29b-3p, hsa-miR-664b-3p, hsa-miR-99b-5p, hsa-miR-103a-3p, hsa-miR-6516-3p, hsa-miR-22-3p, hsa-miR-26a-5p, hsa-miR-103b, hsa-miR-1291, hsa-miR-425-5p, hsa-miR-22-5p, hsa-miR-374c-3p, hsa-let-7i-3p, hsa-miR-374b-5p, hsa-miR-455-3p, hsa-miR-532-3p, hsa-miR-619-5p, hsa-miR-28-5p, hsa-miR-10a-5p, hsa-miR-151a-3p, hsa-miR-30e-3p, hsa-miR-324-5p, hsa-miR-495-3p, hsa-miR-576-5p, hsa-miR-625-3p, hsa-miR-671-5p, hsa-miR-1271-5p, hsa-miR-186-5p, hsa-miR-23a-5p, hsa-miR-3613-5p, hsa-miR-376c-3p, hsa-miR-409-3p, hsa-miR-4461, hsa-miR-454-3p, hsa-miR-6724-5p, hsa-let-7b-3p, hsa-miR-190a-5p, hsa-miR-26b-3p, hsa-miR-3609, hsa-miR-411-5p, hsa-miR-425-3p, hsa-miR-4485-3p and a mixture thereof.

In certain embodiments, the cellular miRNAs are selected in a group comprising hsa-miR210-3p, hsa-miR-409-3p, hsa-let-7i-5p, hsa-miR-24-3p, hsa-miR-93-5p, hsa-miR-382-5p, hsa-miR-4485-3p, and a combination thereof.

In certain embodiments, at least part of the factors content is secreted by the cells, preferably in the form of exosomes or exosome-like vesicles. In said embodiments, at least part of the factors content is comprised in exosomes or exosome-like vesicles. In some embodiments, at least part of the factors content is exosomal miRNAs.

As used herein, the term “exosome” refers to endocytic-derived nanovesicles that are secreted by nearly all cell types in the body. The exosomes comprise proteins, nucleic acids, in particular miRNAs, and lipids. In practice, the exosomes may be isolated and/or purified according to any suitable method known in the state of the art, or a method adapted therefrom. Illustratively, the exosome fraction may be isolated by differential centrifugation from culture medium; by polymer precipitation; by high-performance liquid chromatography (HPLC). Non-limitative example of differential centrifugation method from culture medium may include the following steps:
Centrifugation for 10-20 min at a speed of about 300×g to about 500×g, so as to remove cells;
Centrifugation for 10-20 min at a speed of about 1,500×g to about 3,000×g, so as to remove dead cells;
Centrifugation for 20-45 min at a speed of about 7,500×g to about 15,000×g, so as to remove cell debris;
One or more ultracentrifugation for 30-120 min at a speed of about 100,000×g to about 200,000×g, so as to pellet the exosomes.

Alternative methods to isolate exosomes may take advantage of commercial kits, such as, e.g., the exoEasy Maxi Kit (Qiagen®) or the Total Exosome Isolation Kit (ThermoFisher Scientific®).

In some embodiments, the exosomes or the exosome-like vesicles have an average diameter ranging from about 25 nm to about 150 nm, preferably from about 30 nm to 120 nm. Within the scope of the instant invention, the expression “from about 25 nm to about 150 nm” includes 25, 30, 35, 40, 45, 50, 55, 60, 65, 70, 75, 80, 85, 90, 95, 100, 105, 110, 115, 120, 125, 130, 135, 140, 145 and 150 nm.

In one embodiment, the exosomal miRNAs are selected in a group comprising hsa-let-7a-5p, hsa-miR-92a-3p, hsa-miR-92b-3p, hsa-miR-24-2-5p, hsa-let-7b-5p, hsa-miR-125b-5p, hsa-miR-335-5p, hsa-miR-26a-2-3p, hsa-let-7f-5p, hsa-miR-337-3p, hsa-let-7f-1-3p, hsa-miR-301a-3p, hsa-miR-24-3p, hsa-miR-93-5p, hsa-miR-196b-5p, hsa-miR-98-3p, hsa-miR-21-5p, hsa-miR-409-3p, hsa-miR-3613-3p, hsa-miR-1273a, hsa-miR-23b-3p, hsa-miR-199a-3p, hsa-miR-23a-5p, hsa-miR-28-5p, hsa-miR-1273g-3p, hsa-miR-145-5p, hsa-miR-374b-5p, hsa-miR-34a-3p, hsa-miR-574-3p, hsa-miR-30a-3p, hsa-miR-660-5p, hsa-miR-425-3p, hsa-miR-25-3p, hsa-miR-382-5p, hsa-miR-186-5p, hsa-miR-505-3p, hsa-let-7e-5p, hsa-miR-19b-3p, hsa-miR-454-3p, hsa-miR-34b-3p, hsa-miR-214-3p, hsa-miR-210-3p, hsa-miR-10a-5p, hsa-miR-361-3p, hsa-miR-199a-5p, hsa-miR-619-5p, hsa-miR-495-3p, hsa-miR-10b-5p, hsa-miR-196a-5p, hsa-miR-17-5p, hsa-miR-425-5p, hsa-miR-1306-5p, hsa-miR-199b-5p, hsa-miR-193a-5p, hsa-miR-2053, hsa-miR-22-5p, hsa-miR-221-3p, hsa-miR-320b, hsa-miR-5096, hsa-miR-378a-3p, hsa-miR-424-5p, hsa-miR-193b-5p, hsa-miR-494-3p, hsa-miR-411-5p, hsa-miR-23a-3p, hsa-miR-320a, hsa-miR-27a-3p, hsa-miR-505-5p, hsa-let-7c-5p, hsa-miR-151a-3p, hsa-miR-4449, hsa-miR-664a-3p, hsa-miR-199b-3p, hsa-let-7a-3p, hsa-miR-532-3p, hsa-miR-26a-5p, hsa-miR-191-5p, hsa-miR-30e-3p, hsa-miR-532-5p, hsa-miR-377-3p, hsa-miR-574-5p, hsa-miR-22-3p, hsa-miR-126-5p, hsa-miR-485-3p, hsa-miR-424-3p, hsa-miR-99b-5p, hsa-miR-30c-5p, hsa-miR-590-3p, hsa-miR-423-5p, hsa-miR-625-3p, hsa-miR-130b-3p, hsa-miR-99a-3p, hsa-miR-342-3p, hsa-miR-4668-5p, hsa-miR-136-3p, hsa-miR-143-3p, hsa-let-7d-3p, hsa-miR-29b-3p, hsa-miR-15b-3p, hsa-miR-26b-3p, hsa-miR-130a-3p, hsa-miR-423-3p, hsa-miR-29b-1-5p, hsa-miR-3607-5p, hsa-miR-3184-3p, hsa-miR-376c-3p, hsa-miR-99b-3p, hsa-miR-3651, hsa-miR-222-3p, hsa-let-7b-3p, hsa-miR-127-3p, hsa-miR-374a-3p, hsa-let-7g-5p, hsa-miR-3074-5p, hsa-miR-134-5p, hsa-miR-376a-3p, hsa-miR-125a-5p, hsa-miR-98-5p, hsa-miR-324-5p, hsa-miR-485-5p, hsa-let-7d-5p, hsa-miR-185-5p, hsa-miR-3605-3p, hsa-miR-103b, hsa-miR-29a-3p, hsa-miR-19a-3p, hsa-miR-101-3p, hsa-miR-126-3p, hsa-let-7i-5p, hsa-miR-34a-5p, hsa-miR-103a-3p, hsa-miR-149-5p, hsa-miR-146b-5p, hsa-miR-374c-3p, hsa-miR-1246, hsa-miR-193b-3p, hsa-miR-4454, hsa-miR-181a-5p, hsa-miR-138-5p, hsa-miR-223-3p, hsa-miR-28-3p, hsa-miR-328-3p, hsa-miR-190a-5p, hsa-miR-340-3p, hsa-miR-874-3p, hsa-miR-7847-3p, hsa-miR-6724-5p, hsa-miR-369-5p, and a combination thereof.

In some embodiments, the exosomal miRNAs are selected in a group comprising hsa-miR-210-3p, hsa-miR-409-3p, hsa-let-7i-5p, hsa-miR-3607-5p, hsa-let-7a-3p, hsa-miR-1246, hsa-miR-335-5p, hsa-miR-4454, hsa-miR-181a-5p, hsa-miR-374c-3p, hsa-miR-619-5p, hsa-miR-29b-3p, hsa-let7e-5p, hsa-miR-23b-3p, hsa-miR-4449, hsa-miR-663a, hsa-miR-25-3p, hsa-let-7b-3p, hsa-miR-138-5p, hsa-miR-3613-3p, hsa-miR-6516-3p, hsa-miR-664a-3p, hsa-miR-3648, hsa-miR-3653-5p, hsa-miR-6516-5p, hsa-miR-3651, hsa-miR-3687, hsa-miR-664-5p, hsa-miR-664-3p, and a combination thereof.

In one embodiment, the exosomal miRNAs are selected in a group comprising hsa-let-7a-5p, hsa-let-7b-5p, hsa-miR-24-3p, hsa-miR-21-5p, hsa-let-7f-5p, hsa-miR-574-3p, hsa-miR-23b-3p, hsa-miR-1273g-3p, hsa-miR-25-3p, hsa-miR-199a-5p, hsa-miR-196a-5p, hsa-miR-214-3p, hsa-miR-125a-5p, hsa-miR-221-3p, hsa-miR-222-3p, hsa-let-7e-5p, hsa-miR-191-5p, hsa-miR-199b-3p, hsa-miR-342-3p, hsa-miR-23a-3p, hsa-miR-424-3p, hsa-miR-28-3p, hsa-let-7g-5p, hsa-miR-92a-3p, hsa-miR-424-5p, hsa-let-7d-3p, hsa-miR-4454, hsa-miR-146b-5p, hsa-miR-423-5p, hsa-miR-29a-3p, hsa-miR-574-5p, hsa-miR-199b-5p, hsa-miR-125b-5p, hsa-miR-3184-3p, hsa-let-7c-5p, hsa-miR-337-3p, hsa-let-7d-5p, hsa-miR-145-5p, hsa-miR-93-5p, hsa-miR-619-5p, hsa-miR-130a-3p, hsa-let-7i-5p, hsa-miR-409-3p, hsa-miR-210-3p, hsa-miR-199a-3p, hsa-miR-30a-3p, hsa-miR-320b, hsa-miR-193a-5p, hsa-miR-382-5p, hsa-miR-423-3p, hsa-miR-17-5p, hsa-miR-19b-3p, hsa-miR-92b-3p, hsa-miR-320a, hsa-miR-3074-5p, hsa-miR-376c-3p, hsa-let-7b-3p, hsa-miR-625-3p, hsa-miR-99b-5p, hsa-miR-34a-5p, hsa-miR-5096, hsa-miR-30e-3p, hsa-miR-22-3p, hsa-miR-151a-3p, hsa-miR-186-5p, hsa-miR-193b-5p, hsa-miR-328-3p, hsa-miR-4449, hsa-miR-27a-3p, hsa-miR-30c-5p, hsa-miR-494-3p, hsa-miR-98-5p, hsa-miR-10a-5p, hsa-miR-29b-3p, hsa-miR-374b-5p, hsa-miR-335-5p, hsa-miR-374c-3p, hsa-miR-425-5p, hsa-miR-181a-5p, hsa-miR-196b-5p, hsa-let-7f-1-3p, hsa-miR-4668-5p, hsa-miR-660-5p, hsa-miR-664a-3p, hsa-miR-185-5p, hsa-miR-3651, hsa-miR-495-3p, hsa-let-7a-3p, hsa-miR-28-5p, hsa-miR-99b-3p, hsa-miR-103a-3p, hsa-miR-19a-3p, hsa-miR-126-5p, hsa-miR-2053, hsa-miR-29b-1-5p, hsa-miR-3648, hsa-miR-374a-3p, hsa-miR-454-3p, hsa-miR-532-3p, hsa-miR-136-3p, hsa-miR-361-3p, hsa-miR-1246, hsa-miR-130b-3p, hsa-miR-134-5p, hsa-miR-154-5p, hsa-miR-34a-3p, hsa-miR-576-5p, hsa-miR-874-3p, hsa-miR-100-5p, hsa-miR-103b, hsa-miR-1273a, hsa-miR-1306-5p, hsa-miR-138-5p, hsa-miR-15b-3p, hsa-miR-26b-3p, hsa-miR-10b-5p, hsa-miR-22-5p, hsa-miR-3613-3p, hsa-miR-655-3p, hsa-miR-7-1-3p, hsa-miR-23a-5p, hsa-miR-24-2-5p, hsa-miR-3605-3p, hsa-miR-6832-3p, hsa-miR-146a-5p, hsa-miR-16-2-3p, hsa-miR-181b-5p, hsa-miR-26a-2-3p, hsa-miR-376a-3p, hsa-miR-539-5p, hsa-miR-708-5p, hsa-miR-98-3p, hsa-miR-1237-5p, hsa-miR-223-3p, hsa-miR-532-5p, hsa-miR-542-3p, hsa-miR-663a, hsa-miR-101-3p, hsa-miR-143-3p, hsa-miR-21-3p, hsa-miR-224-5p, hsa-miR-26a-5p, hsa-miR-27a-5p, hsa-miR-324-5p, hsa-miR-340-3p, hsa-miR-379-5p, hsa-miR-409-5p, hsa-miR-543, hsa-miR-5787, hsa-miR-6089, hsa-miR-127-3p, hsa-miR-149-5p, hsa-miR-181c-5p, hsa-miR-193b-3p, hsa-miR-222-5p, hsa-miR-3613-5p, hsa-miR-365b-3p, hsa-miR-3960, hsa-miR-485-3p, hsa-miR-6087, hsa-miR-92a-1-5p and a mixture thereof.

In some embodiments, the exosomal miRNAs are selected in a group comprising hsa-miR210-3p, hsa-miR-409-3p, hsa-miR-4454, hsa-miR-619-5p, hsa-miR-3607-5p, hsa-miR-3613-3p, hsa-miR-664b-5p, hsa-miR-3687, hsa-miR-3653-5p, hsa-miR-664b-3p, and a combination thereof.

In certain embodiments, said biomaterial comprises an altered factors and/or RNAs content as compared to the factors and RNAs content obtained from the corresponding viable differentiated cells, including osteo-differentiated and/or chondro-differentiated cells.

In certain embodiments, the biomaterial according to the invention is a multi-dimensional biomaterial, in particular in the form of a particulate composition, a powder, a bead. In some embodiments, the multi-dimensional biomaterial comprises or consists of particles, preferably gelatin, DBM or ceramic particles, which particles are coated with cells and the extracellular matrix.

In some embodiments, the multi-dimensional biomaterial according to the invention is added onto, or contained within, a predefined 3D shape or scaffold, such as for example a piece of lyophilized human bone tissue, by the means of techniques available to the ordinary person skilled in the art. In some embodiments, the multi-dimensional biomaterial according to the invention is structured to form a predefined 3D shape or scaffold, such as for example a cube, with techniques available to the ordinary person skilled in the art.

In some embodiments, the multi-dimensional biomaterial is in the form of particles having an average mean diameter of from about 100 μm to about 1.5 mm, preferably from about 500 μm to about 1 mm. Within the scope of the instant invention, the expression “from about 100 μm to about 1.5 mm” encompasses 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1.0 mm, 1.1 mm, 1.2 mm, 1.3 mm, 1.4 mm and 1.5 mm.

In practice, the measure of the average size of particles may be performed by any suitable methods known in the state of the art, or a method adapted therefrom. Non-limiting examples of such methods include atomic force microscopy (AFM), transmission electron microscopy (TEM), scanning electron microscopy (SEM), dynamic light scattering (DLS).

In some embodiments, said desiccated biomaterial is obtained by freeze-drying. Freeze-drying, otherwise referred to as lyophilization, may be performed accordingly any one of the protocols disclosed in the state of the art, or a protocol adapted therefrom.

In some embodiments, the freeze-drying of the biomaterial is performed at a temperature of about −80° C., under vacuum.

In practice, sterilization may be performed by any suitable method known from the state of the art, or a method adapted therefrom. Non-limitative examples of suitable methods include irradiation such as electron beam irradiation, X-ray irradiation, gamma-irradiation, or ultraviolet irradiation.

In certain embodiments, said sterile biomaterial is obtained by gamma-irradiation, preferably at a dose of about 7 kGy to about 45 kGy, more preferably at room temperature. Within the scope of the invention, the expression “about 7 kGy to about 45 KGy” encompasses 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 and 45 kGy.

In some embodiments, the biomaterial is obtained by gamma-irradiation at a dose of about 10 kGy to about 40 kGy.

Within the scope of the invention, the term “room temperature” is intended to refer to a temperature comprised from about 18° C. to about 22° C., which encompasses 18° C., 19° C., 20° C., 21° C. and 22° C. In some embodiments, room temperature is a temperature of about 20° C.

The inventors observed that, in spite of the fact that sample undergoing gamma-irradiation have a general tendency to overheat and to potentially destroy valuable ingredients, gamma-irradiation of the biomaterial of the invention could be performed at room temperature without being substantially affected by overheating.

In some embodiments, the gamma-irradiation may be performed at a temperature below about 10° C., preferably on ice (about 0° C.). Within the scope of the invention, a temperature below about 10° C. encompasses 9.5° C., 8° C., 8.5° C., 8° C., 7.5° C., 7° C., 6.5° C., 6° C., 5° C., 4° C., 3° C., 2° C., 1° C., 0° C., −1° C., −2° C., −3° C., −4° C., −5° C., −10° C., −20° C., −30° C., −40° C., −50° C., −60° C., −70° C. and −80° C.

In practice, the gamma-irradiation may be performed for a duration that would depend from the size (e.g. expressed in mm³ or cm³) and/or the amount (e.g. expressed in mg or g) of biomaterial to be sterilized and/or the dose to be administered.

In certain embodiments, the gamma-irradiation may be performed from about 10 sec to about 24 h, preferably from about 5 min (300 sec) to about 12h, more preferably, from about 10 min (600 sec) to about 3 h (10,800 sec). Within the scope of the invention, the expression “from about 10 sec to about 24 h” encompasses 10 sec, 12 sec, 14 sec, 16 sec, 18 sec 20 sec, 25 sec, 30 sec, 35 sec, 40 sec, 45 sec, 50 sec, 55 sec, 1 min, 1 min 30, 2 min, 2 min 30, 3 min, 3 min 30, 4 min, 4 min 30, 5 min, 6 min, 7 min, 8 min, 9 min, 10 min, 12 min, 14 min, 16 min, 18 min, 20 min, 22 min, 24 min, 26 min, 28 min, 30 min, 35 min, 40 min, 45 min, 50 min, 55 min, 1 h, 1 h 30, 2 h, 2 h 30, 3 h, 3 h 30, 4 h, 4 h 30, 5 h, 5 h 30, 6 h, 7 h, 8 h, 9 h, 10 h, 11 h, 12h, 13 h, 14h, 15 h, 16h, 17h, 18 h, 19 h, 20 h, 21 h, 22 h, 23 h and 24 h.

In certain embodiments, said desiccated and sterile biomaterial is obtained by freeze-drying and by gamma-irradiation.

In some embodiments, said biomaterial is autologous. In some embodiments, said biomaterial is allogenic.

In some embodiments, said biomaterial is xenogeneic. In one embodiment, the biomaterial originates from an animal, such as a vertebrate animal, a non-human mammal animal or a human.

The invention relates to a method for generating a sterile and desiccated biomaterial comprising devitalized differentiated cells and a particulate material, the cells and the particulate material being embedded in an extracellular matrix, said method comprising the steps of:
(1) contacting (i) viable cells capable to undergo differentiation with (ii) a particulate material, so as to obtain a first combination;
(2) culturing the first combination obtained in step (1) in a culture medium such that the cells secrete an extracellular matrix and synthesize a factors content, so as to acquire tissue regenerating and/or repairing properties, and wherein the cells and the particulate material are embedded in the extracellular matrix, so as to form a multidimensional structure;
(3) submitting the multidimensional structure obtained at step (2) to desiccation, so as to obtain a desiccated biomaterial;
(4) submitting the desiccated biomaterial obtained at step (3) to sterilization, preferably by gamma-irradiation, so as to obtain a sterile, desiccated biomaterial.

In some embodiments, devitalized differentiated cells have regenerating and/or repairing properties.

The invention also pertains to a method for generating a sterile and desiccated biomaterial comprising devitalized differentiated cells having tissue regenerating and/or repairing properties, and gelatin, the cells and the gelatin being embedded in an extracellular matrix, said method comprising the steps of:
(1) contacting (i) viable cells capable to undergo differentiation with (ii) gelatin, so as to obtain a first combination;
(2) culturing the first combination obtained in step (1) in a culture medium, such that the cells secrete an extracellular matrix and synthesize a factors content, so as to acquire tissue regenerating and/or repairing properties, wherein the cells and the gelatin are embedded in the extracellular matrix, so as to form a multidimensional structure;
(3) submitting the multidimensional structure obtained at step (2) to desiccation, so as to obtain a desiccated biomaterial;
(4) submitting the desiccated biomaterial obtained at step (3) to sterilization, preferably by the means of gamma-irradiation, so as to obtain a sterile, desiccated biomaterial.

As used herein, the expression “viable cells capable to undergo differentiation” is intended to refer to a population of cells that can be differentiated in cells that possess tissue regenerating and/or repairing properties.

The invention also pertains to a method for generating a sterile and desiccated biomaterial comprising devitalized osteo-differentiated and/or chondro-differentiated cells and a particulate material, the cells and the particulate material being embedded in an extracellular matrix, said method comprising the steps of:
(1) contacting (i) viable cells capable to undergo osteogenic and/or chondrogenic differentiation (ii) with a particulate material, so as to obtain a first combination;
(2) culturing the first combination obtained in step (1) in a culture medium such that the cells secrete an extracellular matrix and synthesize a factor content, so as to acquire osteogenic and/or chondrogenic properties, and wherein the cells and the particulate material are embedded in the extracellular matrix, so as to form a multidimensional structure;
(3) submitting the multidimensional structure obtained at step (2) to desiccation, so as to obtain a desiccated biomaterial;
(4) submitting the desiccated biomaterial obtained at step (3) to sterilization, preferably by gamma-irradiation, so as to obtain a sterile, desiccated biomaterial.

As used herein, the expression “viable cells capable to undergo osteogenic and/or chondrogenic differentiation” is intended to refer to a population of cells that can be differentiated in cells that possess osteogenic and/or chondrogenic properties.

As used herein, the term “embedded in” is intended to mean “enclosed closely in” or “being an integral part of”. In other words, by “cells and the particulate material are embedded in the extracellular matrix”, one may understand that the cells, the particulate material and the extracellular matrix are intimately linked one to another and that the three ingredients make one unique structure.

In some embodiments, the viable cells capable to undergo differentiation are selected in a group comprising primary cells; stem cells, in particular stems cells from adipose tissue, or bone marrow, umbilical cord blood; genetically modified cells; and a mixture thereof.

In some embodiments, the primary cells may be cultured in a culture medium suitable for allowing proliferation or maintenance of the cells.

In certain embodiments, the stem cells and/or the genetically modified cells may be cultured in a culture medium that allows the cellular differentiation into a cell population that possesses tissue regenerating and/or repairing properties.

In certain embodiments, the stem cells and/or the genetically modified cells may be cultured in a culture medium that allow the cellular differentiation into a cell population that possesses osteogenic and/or chondrogenic properties.

In certain embodiments, the biomaterial comprises from about 10² to about 10¹⁶ cells per gram of the biomaterial, preferably from about 10⁶ to about 10¹² cells per gram of the biomaterial. Within the scope of the instant invention, the expression “from about 10² to about 10¹⁶ cells” encompasses 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², 5×10¹², 10¹³, 5×10¹³, 10¹⁴, 5×10¹⁴, 10¹⁵, 5×10¹⁵ and 10¹⁶ cells.

As used herein, a “culture medium” refers to the generally accepted definition in the field of cellular biology, i.e. any medium suitable for promoting the growth of the cells of interest.

In some embodiments, a suitable culture medium may include a chemically defined medium, i.e. a nutritive medium only containing specified components, preferably components of known chemical structure.

In some embodiments, a chemically defined medium may be a serum-free and/or feeder-free medium. As used herein, a “serum-free” medium refers to a culture medium containing no added serum. As used herein, a “feeder-free” medium refers to a culture medium containing no added feeder cells.

A culture medium for use according to the invention may be an aqueous medium that may include a combination of substances such as one or more salts, carbon sources, amino acids, vitamins, minerals, reducing agents, buffering agents, lipids, nucleosides, antibiotics, cytokines, and growth factors.

Examples of suitable culture media include, without being limited to, RPMI medium, William's E medium, Basal Medium Eagle (BME), Eagle's Minimum Essential Medium (EMEM), Minimum Essential Medium (MEM), Dulbecco's Modified Eagles Medium (DMEM), Ham's F-10, Ham's F-12 medium, Kaighn's modified Ham's F-12 medium, DMEM/F-12 medium, and McCoy's 5A medium, which may be further supplemented with any one of the above mentioned substances.

In some embodiments, a culture medium according to the invention may be a synthetic culture medium such as the RPMI (Roswell Park Memorial Institute medium) or the CMRL-1066 (Connaught Medical Research Laboratory).

In practice, both media may be supplemented with additional additives, commonly used in the field. In some embodiments, the additional additives may be intended to promote osteogenesis, chondrogenesis, myogenesis, angiogenesis, neurogenesis, epitheliogenesis, endotheliogenesis, adipogenesis. In some embodiments, the additional additives may be intended to promote osteogenesis and/or chondrogenesis. Non-limitative examples of suitable additional additives encompass growth factors, transcription factors, osteocytes activators, osteoblasts activators, osteoclasts inhibitors, chondrocytes activators, the likes and a mixture thereof.

In practice, the culture parameters such as the temperature, the pH, the salinity, and the levels of O₂ and CO₂ are adjusted accordingly to the standards established in the state of the art. Illustratively, the temperature for culturing the cells according to the invention may range from about 30° C. to about 42° C., preferably from about 35° C. to about 40° C., and more preferably from about 36° C. to about 38° C. Within the scope of the invention, the expression “from about 30° C. to about 42° C.” encompasses 30° C., 31° C., 32° C., 33° C., 34° C., 35° C., 36° C., 37° C., 38° C., 39° C., 40° C., 41° C. and 42° C.

In some embodiments, the level of CO₂ during the course of culture is maintained constant and ranges from about 1% to about 10%, preferably from about 2.5% to about 7.5%. Within the scope of the invention, the expression “from about 1% to about 10%” encompasses 1, 2, 3, 4, 5, 6, 7, 8, 9 and 10%.

In certain embodiments, step (2) is performed in the presence of one or more exogenous factors selected in the group comprising growth factors, transcription factors, osteogenic and/or chondrogenic factors, and a mixture thereof.

In practice, under the suitable culture conditions, the cells are secreting an extracellular matrix and synthesize polypeptides and nucleic acids that promote osteogenesis and/or chondrogenesis. Said polypeptides and nucleic acids may be considered as being biomarkers for the osteogenesis and/or chondrogenesis properties and may be monitored at the polypeptide level and/or at the nucleic acids level, by the means of methods mentioned hereinabove.

In some embodiments, said biomaterial further comprises one or more exogenous factors having osteogenic and/or chondrogenic properties selected in the group comprising growth factors, transcription factors, osteogenic and/or chondrogenic factors, osteogeny-inducing or chondrogeny-inducing nucleic acids, and a mixture thereof.

As used herein, the biomaterial according to the invention possesses osteogenic and/or chondrogenic properties. In practice, the osteogenic and/or chondrogenic properties of the biomaterial may be assessed by any suitable method available in the art, after administration in an individual. Illustratively, upon administration of the biomaterial according to the invention to an individual, one may measure biomarkers of osteoinduction. Non-limitative examples of such biomarkers include BMPR-1A, BMPR-2, CSF-1, IGF-1R, RUNX2, SMAD-2, SMAD-3, SMAD-4, SMAD-5 and TWIST-1.

One aspect of the invention relates to a sterile and desiccated biomaterial obtainable by the method according to the invention.

Another aspect of the invention relates to a pharmaceutical composition comprising a biomaterial according to the invention, and a pharmaceutically acceptable vehicle.

As used herein, “pharmaceutically acceptable vehicle” refers to any solvent, dispersion medium, coating, antibacterial and/or antifungal agent, isotonic and absorption delaying agent and the like.

In practice, the pharmaceutically acceptable vehicle may comprise one or more ingredient(s) selected in a group of additives polypeptides; amino acids; lipids; and carbohydrates. Among carbohydrates, one may cite sugars, including monosaccharides, di-, tri-, tetra-, and oligosaccharides; derivatized sugars such as alditols, aldonic acids, esterified sugars and the like; and polysaccharides or sugar polymers.

Examples of suitable pharmaceutically acceptable vehicles may include polypeptides such as, e.g., gelatin, casein, and the like.

In some embodiments, the pharmaceutical composition is in the form of a paste or a film.

In certain embodiments, the paste is a moldable paste. In some embodiment the moldable paste or the film can be easily handled, manipulated and grafted.

A further aspect of the invention relates to a medicament comprising a biomaterial according to the invention.

Another aspect of the invention relates to a medical device comprising a biomaterial or a pharmaceutical composition according to the invention.

In certain embodiments, the medical device is a dressing for local application. In some embodiments, the dressing may comprise woven or non-woven fabrics. In some embodiments, the medical device is coated by or with the composition according to the present invention. In certain embodiments, the medical device according to the invention is configured to allow the controlled release of the pharmaceutical composition. In some embodiments, the medical device is in the form of a patch.

In some embodiments, the medical device is an implant. In some embodiments, the implant may be in the form of an organic or inorganic scaffold. In certain embodiments, the implant is resorbable.

The invention further pertains to an implant comprising a multi-dimensional biomaterial according to the instant disclosure. In some embodiments, the implant is allogeneic. In certain embodiments, the implant is autologous. In some embodiments, the implant is xenogeneic. In certain embodiments, the implant is lyophilized and sterilized, preferably sterilized by gamma-irradiation.

Uses and methods may be performed in vivo or ex vivo.

In one aspect, the invention pertains to a biomaterial or a pharmaceutical composition according to the invention, for use as a medicament.

In some embodiments, the invention relates to the biomaterial or the pharmaceutical composition according to the instant invention for use for preventing and/or treating a tissue disorder.

In some embodiments, the biomaterial or the pharmaceutical composition for use according to the instant invention is for preventing and/or the treating a bone disorder and/or a cartilage disorder.

The invention further relates to the use of a biomaterial or a pharmaceutical composition according to the invention, for the manufacture or the preparation of a medicament, in particular for the prevention and/or the treatment of a tissue disorder.

The invention further relates to the use of a biomaterial or a pharmaceutical composition according to the invention, for the manufacture or the preparation of a medicament and to the use of a biomaterial or a pharmaceutical composition according to the invention, for preventing and/or treating a bone disorder and/or a cartilage disorder.

The invention also pertains to a method for preventing and/or treating a tissue disorder in an individual in need thereof, comprising the administration of a therapeutically effective amount of a biomaterial or a pharmaceutical composition according to the invention.

The invention also pertains to a method for preventing and/or treating a bone disorder and/or a cartilage disorder in an individual in need thereof, comprising the administration of a therapeutically effective amount of a biomaterial or a pharmaceutical composition according to the invention.

In certain embodiments, said tissue is selected from the group comprising bone tissue, cartilage tissue, skin tissue, muscular tissue, epithelial tissue, endothelial tissue, neural tissue, connective tissue and adipose tissue.

In one embodiment, the term “tissue” comprises or consists of bone, cartilage, skin, muscle, endothelium, epithelium, neural, connective and adipose tissue defect.

In certain embodiments, the tissue disorder is selected in a group comprising aplasia cutis congenita; a burn; a cancer, including a breast cancer, a skin cancer and a bone cancer; a Compartment syndrome (CS); epidermolysis bulbosa; giant congenital nevi; an ischemic muscular injury of lower limbs; a muscle contusion, rupture or strain; a post-radiation lesion; and an ulcer, including a diabetic ulcer, preferably a diabetic foot ulcer; arthritis; bone fracture; bone frailty; Caffey's disease; congenital pseudarthrosis; cranial deformation; cranial malformation; delayed union; infiltrative disorders of bone; hyperostosis; loss of bone mineral density; metabolic bone loss; osteogenesis imperfecta; osteomalacia; osteonecrosis; osteopenia; osteoporosis; Paget's disease; pseudarthrosis; sclerotic lesions; spina bifida; spondylolisthesis; spondylolysis; chondrodysplasia; costochondritis; enchondroma; hallux rigidus; hip labral tear; osteochondritis dissecans; osteochondrodysplasia; polychondritis; and the likes.

In some embodiments, the tissue disorder is selected from the group comprising aplasia cutis congenita; a burn; a cancer, including a breast cancer, a skin cancer; a Compartment syndrome (CS); epidermolysis bulbosa; giant congenital nevi; an ischemic muscular injury of lower limbs; a muscle contusion, rupture or strain; a post-radiation lesion; and an ulcer, including a diabetic ulcer, preferably a diabetic foot ulcer.

As used herein, the term “cancer” encompasses a solid cancer, in particular, a cancer selected in the group comprising, or consisting of, a bone cancer, a brain cancer, a skin cancer, a breast cancer, a cancer of the central nervous system, a cancer of the cervix, a cancer of the upper aero digestive tract, a colorectal cancer, an endometrial cancer, a germ cell cancer, a bladder cancer, a kidney cancer, a laryngeal cancer, a liver cancer, a lung cancer, a neuroblastoma, an esophageal cancer, an ovarian cancer, a pancreatic cancer, a pleural cancer, a prostate cancer, a retinoblastoma, a small intestine cancer, a soft tissue sarcoma, a stomach cancer, a testicular cancer and a thyroid cancer.

In some embodiments, the bone disorder is selected in a group comprising arthritis, bone cancer, bone fracture, bone frailty, Caffey's disease, congenital pseudarthrosis, cranial deformation, cranial malformation, delayed union, infiltrative disorders of bone, hyperostosis, loss of bone mineral density, metabolic bone loss, osteogenesis imperfecta, osteomalacia, osteonecrosis, osteopenia, osteoporosis, Paget's disease, pseudarthrosis, sclerotic lesions, spina bifida, spondylolisthesis and spondylolysis.

In certain embodiments, the cartilage disorder is selected in a group comprising arthritis, chondrodysplasia, costochondritis, enchondroma, hallux rigidus, hip labral tear, osteochondritis dissecans, osteochondrodysplasia and polychondritis.

In one embodiment, the biomaterial or the pharmaceutical composition is for use for tissue reconstruction.

In one embodiment, tissue reconstruction is selected from the group comprising or consisting of bone reconstruction, cartilage reconstruction, dermis reconstruction, muscle or myogenic reconstruction, endothelial reconstruction, epithelial reconstruction, connective tissue reconstruction, neural reconstruction, and adipogenic reconstruction.

Examples of bone and skin reconstruction include, but are not limited to, dermal and/or epidermal reconstruction, wound healing, diabetic ulcer treatment such as diabetic foot ulcer, post-burn lesions reconstruction, post-radiation lesions reconstruction, reconstruction after breast cancer or breast deformities.

Examples of cartilage reconstruction include, but are not limited to, knee chondroplasty, nose or ear reconstruction, costal or sternal reconstruction.

Examples of myogenic reconstruction include, but are not limited to, skeletal muscle reconstruction, reconstruction after break of the abdominal wall, reconstruction after ischemic muscular injury of lower limbs, reconstruction associated with compartment syndrome (CS).

Examples of endothelial reconstruction include, but are not limited to, recellularization of vascular patchs for vascular anastomosis such as venous arteriosclerosis shunt.

Examples of adipogenic reconstruction include, but are not limited to, esthetic surgery, rejuvenation, lipofilling reconstruction.

In some aspect, the invention relates to the biomaterial or the pharmaceutical composition for use according to the invention, for skin reconstruction, preferably for treating a skin wound.

The invention also pertains to a method for skin reconstruction, preferably for treating a skin wound, in an individual in need thereof, comprising the administration of a therapeutically effective amount of a biomaterial or a pharmaceutical composition according to the invention.

In one embodiment, the biomaterial, pharmaceutical composition or medical device of the invention is for use in treating skin tissue disorders. In one embodiment, the biomaterial, pharmaceutical composition or medical device of the invention is for use for skin reconstruction, including dermis and/or epidermis reconstruction. In one embodiment, the biomaterial, pharmaceutical composition or medical device of the invention is for dermal and/or epidermal reconstruction, wound healing, diabetic ulcer treatment such as diabetic foot ulcer, post-burn lesions reconstruction, post-radiation lesions reconstruction, reconstruction after breast cancer or breast deformities. In a particular embodiment, the biomaterial, pharmaceutical composition or medical device of the invention is for use for, or for use in treating, skin wound, preferably diabetic skin wound. In one embodiment, the biomaterial, pharmaceutical composition or medical device of the invention is for promoting the closure of wound. In one embodiment, the biomaterial, pharmaceutical composition or medical device of the invention is for reducing the thickness of wound, in particular during wound healing.

In a particular embodiment, the biomaterial, pharmaceutical composition or medical device of the invention is for use for, or for use in treating, epidermolysis bulbosa, giant congenital nevi, and/or aplasia cutis congenita.

In still another aspect, the invention relates to the biomaterial, pharmaceutical composition or medical device of the invention for use for reconstructive and/or aesthetic surgery.

In one embodiment, the biomaterial, pharmaceutical composition or medical device of the invention may be used as an allogeneic implant or as an autologous implant. In one embodiment, the biomaterial, pharmaceutical composition or medical device of the invention may be used in tissue grafting.

In one embodiment, the subject has already been treated for tissue defect. In another embodiment, the subject has not already been treated for a tissue defect.

In one embodiment, the subject was non-responsive to at least one other treatment for a tissue defect.

In one embodiment, the subject is diabetic. In one embodiment, the subject is suffering from a diabetic wound.

In another aspect, the invention relates to the biomaterial or the pharmaceutical composition for use according to the instant invention, for compensating the side effects of a primary treatment of a tissue disorder, and/or for strengthening a primary treatment of a tissue disorder.

The invention further pertains to a method for compensating the side effects of a primary treatment of a tissue disorder, and/or for strengthening a primary treatment of a tissue disorder, in an individual in need thereof, comprising the administration of a therapeutically effective amount of a biomaterial or a pharmaceutical composition according to the invention.

In certain embodiments, the primary treatment may be selected in a group comprising an anti-inflammatory treatment, a cancer treatment, an ulcer treatment, a burn treatment, the like and a combination thereof.

In practice, the biomaterial or the pharmaceutical composition according to the invention may be administered prior, during or upon the primary treatment.

In one aspect, the invention relates to the biomaterial or the pharmaceutical composition for use according to the instant invention, for compensating the side effects of a therapeutic treatment known to have a deleterious effect on tissues, in particular bone tissue, cartilage tissue, skin tissue, muscular tissue, epithelial tissue, endothelial tissue, neural tissue, connective tissue and adipose tissue.

In certain embodiments, the said therapeutic treatment may be selected in a group comprising an anti-inflammatory treatment, a cancer treatment, an antibiotic treatment, an immunotherapy, a chemotherapy, the like and a combination thereof.

In another aspect, the invention also relates to a biomaterial or a pharmaceutical composition for use according to the invention for promoting osteogenesis, and/or reducing osteoclastogenesis and/or promoting chondrogenesis, and/or reducing chondroclastogenesis.

In some embodiments, the biomaterial may be for further use for promoting angiogenesis. The invention further relates to a method for promoting osteogenesis, and/or reducing osteoclastogenesis, and/or promoting chondrogenesis and/or reducing chondroclastogenesis, in an individual in need thereof, comprising the administration of a therapeutically effective amount of a biomaterial or a pharmaceutical composition according to the invention.

Another aspect of the invention also relates to a biomaterial or a pharmaceutical composition for use according to the invention for demoting abnormal or dysfunctional osteogenesis and/or chondrogenesis. In some embodiments, the biomaterial or pharmaceutical composition of the invention is for use for restoring abnormal or dysfunctional osteogenesis and/or chondrogenesis.

In some embodiments, an individual in need thereof is an individual having or susceptible to develop a bone disorder selected in a group comprising arthritis, bone cancer, bone fracture, bone frailty, Caffey's diseases, congenital pseudarthrosis, cranial deformation, cranial malformation, delayed union, infiltrative disorders of bone, hyperostosis, loss of bone mineral density, metabolic bone loss, osteogenesis imperfecta, osteomalacia, osteonecrosis, osteopenia, osteoporosis, Paget's disease, pseudarthrosis, sclerotic lesions, spina bifida, spondylolisthesis and spondylolysis.

In certain embodiments, an individual in need thereof is an individual having or susceptible to develop a cartilage disorder selected in a group comprising arthritis, chondrodysplasia, costochondritis, enchondroma, hallux rigidus, hip labral tear, osteochondritis dissecans, osteochondrodysplasia and polychondritis.

Another aspect of the invention also relates to a biomaterial or a pharmaceutical composition for use according to the invention, for compensating the side effects of a primary treatment of a bone disorder and/or a cartilage disorder, and/or for strengthening a primary treatment of a bone disorder and/or a cartilage disorder.

The invention further pertains to a method for compensating the side effects of a primary treatment of a bone disorder and/or a cartilage disorder, and/or for strengthening a primary treatment of a bone disorder and/or a cartilage disorder, in an individual in need thereof, comprising the administration of a therapeutically effective amount of a biomaterial or a pharmaceutical composition according to the invention.

In certain embodiments, the primary treatment may be selected in a group comprising an anti-inflammatory treatment, a bone cancer treatment, the like and a combination thereof.

In practice, the biomaterial or the pharmaceutical composition according to the invention may be administered prior, during or upon the primary treatment.

Another aspect of the invention also relates to a biomaterial or a pharmaceutical composition for use according to the invention for compensating the side effects of a therapeutic treatment known to have a deleterious effect on bones and/or cartilages.

In certain embodiments, the said therapeutic treatment may be selected in a group comprising an anti-inflammatory treatment, a cancer treatment, an antibiotic treatment, an immunotherapy, a chemotherapy, the like and a combination thereof.

In certain embodiments, the biomaterial or the pharmaceutical composition according to the invention is combined before use with any one of an isotonic aqueous solution; a scaffold material; another pharmaceutical composition; medical device; a material of biological origin; and any combination thereof.

In some embodiments, the biomaterial or the pharmaceutical composition according to the invention may be formulated in any suitable form encompassed by the state in the art, e.g., in the form of an injectable solution or suspension, a tablet, a coated tablet, a capsule, a syrup, a suppository, a cream, an ointment, a lotion, a gel and the like.

In some embodiments, the biomaterial of the instant invention may be rehydrated before administration. Illustratively, the biomaterial of the instant invention may be rehydrated with a sterile saline composition, in particular a sterile saline composition comprising from about 0.75% to about 1.25% NaCl, more preferably a sterile saline composition comprising from about 0.90% NaCl.

In some embodiments, the biomaterial or the pharmaceutical composition according to the invention is to be formulated as a putty, an emollient, a cream, an ointment, a lotion, a gel, a salve, a controlled-release matrix, a liposomal or a lipid particle preparation, a microcapsule, or a nanocapsule, a suppository, a transdermal delivery system, or any combination thereof.

In some embodiments, the biomaterial or the pharmaceutical composition is in the form of a semi solid. In some embodiments, the pharmaceutical composition is in the form of a paste, an ointment, a cream, a plaster or a gel. In some embodiments, the pharmaceutical composition may be in the form of a moldable paste or a film that can be manipulated and grafted.

In certain embodiments, the biomaterial or the pharmaceutical composition of the invention can be processed together with suitable excipients to the semi solid form, preferably the paste. Suitable excipients are, in particular, those excipients normally used to produce paste bases. Particularly suitable according to the invention are excipients normally used to produce gel-like paste bases, such as gel formers. Gel formers are substances which form gels with a dispersant such as water. Examples of gel formers of the invention are sheet silicates, carrageenan, xanthan, gum acacia, alginates, alginic acids, pectins, modified celluloses or poloxamers.

In some embodiments, the biomaterial or the pharmaceutical composition in a semi solid form, preferably in the form of a paste, is ready for use. In another embodiment, the pharmaceutical composition in a semi solid form, preferably in the form of a paste, has to be extemporaneously produced.

In some embodiments, the factor's content, including the miRNAs, comprised in the biomaterials of the invention are encapsulated, i.e., are immobilized in a vesicular system.

In one embodiment, the encapsulation is a bilayer encapsulation. In another embodiment, the encapsulation is a single layer encapsulation. In still another embodiment, the encapsulation is a matrix encapsulation.

In certain embodiments, the vesicles encapsulating the factor's content, including the miRNAs, are made of a biopolymer. In another embodiment, the vesicles encapsulating the factor's content, including the miRNAs, are extracellular vesicles. In a particular embodiment, the vesicles encapsulating the factor's content, including the miRNAs, are exosomes. In some embodiments, the exosomes are cells-derived exosomes, preferably exosomes from which the factor's content, including the miRNAs, are derived. In another specific embodiment, the exosomes are engineered exosomes.

Exosome engineering may be performed by any suitable methods known in the state of the art, or adapted therefrom. One may refer to, e.g., “Exosome engineering: Current progress in cargo loading and targeted delivery” (Fu et al., NanoImplant, 2020, Volume 20, 100261).

According to one embodiment, the biomaterial, pharmaceutical composition, medicament or medical device of the invention is administered by any suitable route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical, mucosal, nasal, buccal, sublingual; by intratracheal instillation, bronchial instillation, and/or inhalation; and/or as an oral spray, nasal spray, and/or aerosol.

According to one embodiment, the biomaterial, pharmaceutical composition, medicament or medical device of the invention is administered topically or by surgical implantation.

In one embodiment, the biomaterial, pharmaceutical composition, medicament or medical device is administered at the site of the tissue disorder.

In one embodiment, the biomaterial, pharmaceutical composition, medicament or medical device is administered at the site of the bone and/or cartilage disorder.

In certain embodiments, the biomaterial or medical device according to the invention is combined to one or more ingredients for bioprinting said biomaterial or medical device.

As used herein, the term “bioprinting” refers to the technic allowing the fabrication of three-dimensional constructs mimicking natural tissue and/or organs. In some embodiments, the one or more ingredients include natural polymers, such as, e.g., cellulose, gelatin, alginate, and chitosan; and synthetic polymers, such as, polyvinyl polymers, polyethylene glycol polymers.

For examples of suitable bioprinting processes, one may refer to Aljohani et al. (Internat. J. Biol. Macromol., Volume 107, Part A, 2018, p 261-275), Daly et al. (Adv Healthc Mater. 2017 November; 6(22)); Gu et al. (Adv Exp Med Biol. 2018; 1078:15-28), Matai et al. (Biomaterials. 2020 January; 226:119536).

The invention further pertains to a multi-dimensional biomaterial comprising differentiated human mesenchymal stem cells (MSCs), a particulate material, an extracellular matrix, vascular endothelial growth factor (VEGF), and insulin-like growth factor 1 (IGF-1), wherein the differentiated MSCs are devitalized, wherein the devitalized MSCs and particulate material are embedded in the extracellular matrix, and wherein the multi-dimensional biomaterial is enriched for exosomes or exosome-like vesicles comprising one or more nucleic acids which promote normal tissue differentiation and/or inhibit abnormal tissue differentiation.

In some embodiments, the biomaterial is three-dimensional. In certain embodiments, the mesenchymal stem cells are adipose tissue-derived stem cells (ASCs). In some embodiments, the ASCs are late-passage ASCs. In certain embodiments, the MSCs are devitalized by lyophilization. In some embodiments, the biomaterial is sterilized, optionally sterilized by gamma-irradiation. In certain embodiments, the lyophilized and sterilized biomaterial retains its three-dimensional structure. In some embodiments, the VEGF and IGF-1 are biologically active. In certain embodiments, the biomaterial comprises at least 10 ng of VEGF per g of biomaterial. In some embodiments, the biomaterial comprises at least 60 ng of VEGF per g of biomaterial. In certain embodiments, the biomaterial comprises at least 20 ng of IGF-1 per g of biomaterial. In some embodiments, the biomaterial comprises at least 40 ng of IGF-1 per g of biomaterial. In certain embodiments, the extracellular matrix is secreted by the differentiated MSCs and comprises one or more matrisomal proteins specific to soft tissue or calcified tissue. In some embodiments, the soft tissue is dermal tissue. In certain embodiments, the calcified tissue is bone. In certain embodiments, the extracellular matrix is secreted by the differentiated MSCs and comprises collagen. In some embodiments, the particulate material is selected from the group consisting of demineralized bone particles, gelatin particles, and ceramic particles. In certain embodiments, the particulate material is demineralized bone particles. In some embodiments, the particulate material is gelatin particles. In certain embodiments, the particulate material is ceramic particles. In some embodiments, the ceramic particles are particles of calcium phosphate. In certain embodiments, the particles of calcium phosphate are particles of hydroxyapatite (HA) and/or β-tricalcium phosphate (β-TCP).

In some embodiments, the one or more nucleic acids are one or more micro-RNAs (miRs). In certain embodiments, the one or more miRs are selected from the group consisting of miR-210-3p and hsa-miR-361-3p. In some embodiments, the one or more miRs is miR-210-3p. In certain embodiments, miR-210-3p promotes osteogenesis. In some embodiments, the one or more miRs is hsa-miR-361-3p.

Another aspect of the invention also relates to a method for producing a multi-dimensional biomaterial according to the instant disclosure, said method comprising:
(a) culturing isolated human MSCs with a proliferation medium;
(b) differentiating the MSCs with a differentiation medium;
(c) adding a particulate material to the differentiating MSCs and culturing the MSCs with differentiation medium for a further duration to generate a multi-dimensional tissue; and
(d) lyophilizing the multi-dimensional tissue to produce the multi-dimensional biomaterial.

In some embodiments, the method further comprises (e) sterilizing the multi-dimensional biomaterial, optionally by gamma-irradiation. In certain embodiments, the MSCs are adipose tissue-derived stem cells (ASCs). In some embodiments, step (c) differentiates the MSCs into a cell-type selected from the group consisting of osteoblasts, chondrocytes, keratinocytes, myofibroblasts, endothelial cells, and adipocytes. In certain embodiments, the particulate material is selected from the group consisting of demineralized bone particles, gelatin particles, and ceramic particles.

The invention further pertains to a multi-dimensional biomaterial obtainable by the method according to the instant disclosure.

Another aspect of the invention also pertains to a medical device comprising the multi-dimensional biomaterial according to the instant disclosure or the implant according to the instant disclosure.

The invention also pertains to a kit comprising the multi-dimensional biomaterial according to the instant disclosure or the implant according to the instant disclosure, and a suitable fixation means.

Another aspect of the invention relates to a pharmaceutical composition comprising the multi-dimensional biomaterial according to the instant disclosure, and a pharmaceutically acceptable carrier.

### BRIEF DESCRIPTION OF THE DRAWINGS

FIG. 1A-B is a set of graphs showing the cell viability (expressed as luminescence (RLU)) for NVDX2 (A) and NVDX3 (B) in comparison to ASCs viability at different cellular concentrations, 1%, 10%, 50% and 100% (Tukey test, n=3-6). Mean RLU 3.5 for NVDX2, −91.9 for NVDX3, 6820.3 for 10% ASCs and 2235 for 1% ASCs. ** p<0.01 and *** p<0.001; n.s=not significant.

FIG. 2A-B is a set of graphs showing the glucose consumption (expressed as mmol) by NVDX2 (A) and NVDX3 (B) in comparison to glucose consumption by ASCs at different cellular concentrations, 1%, 10%, 50% and 100%. (Fisher's LSD test, n=3-6). Mean −0.0031 mmol for NVDX2 and 0.0020 mmol for 1% ASCs. Mean −0.0011 mmol for NVDX3, 0.0018 mmol for 10% ASCs and 0.0020 mmol for 1% ASCs. * p<0.05; ** p<0.01 and *** p<0.001.

FIG. 3A-B is a set of graphs showing the lactate production (expressed as mmol) by NVDX2 (A) and NVDX3 (B) in comparison to lactate production by ASCs at different cellular concentrations, 1%, 10%, 50% and 100%. (Fisher's LSD test, n=3-6). *** p<0.001; **** p<0.0001; n.s=not significant.

FIG. 4A-D is a set of plots showing the moisture content (expressed as %) of NVD002 (A), NVDX2 (A and B), NVD003 (C), NVDX3 (C and D).

FIG. 5 is a graph showing the amount of VEFG (expressed in ng/g of biomaterial) in biomaterials NVD002 (fresh), NVD002 lyo (lyophilized) and NVDX2 (lyophilized and sterilized), obtained from 3D-induction in the presence of gelatin.

FIG. 6 is a graph showing the amount of IGF-1 (expressed in ng/g of biomaterial) in biomaterials NVD002 (fresh), NVD002 lyo (lyophilized) and NVDX2 (lyophilized and sterilized), obtained from 3D-induction in the presence of gelatin.

FIG. 7 is a graph showing the amount of SDF-1α (expressed in ng/g of biomaterial) in biomaterials NVD002 (fresh), NVD002 lyo (lyophilized) and NVDX2 (lyophilized and sterilized), obtained from 3D-induction in the presence of gelatin.

FIG. 8 is a graph showing the total protein amounts (expressed in arbitrary units) in biomaterials NVD002 (fresh), NVD002 lyo (lyophilized) and NVDX2 (lyophilized and sterilized), obtained from 3D-induction in the presence of gelatin.

FIG. 9A-B is a set of graphs showing the relative expression of miR-199-5p (A) and miR-361-3p (B) in NVD00X2 (lyophilized NVD002) and NVDX2 (lyophilized and gamma-irradiated NVD002).

FIG. 10A-B is a set of graphs showing the CD3 recruitment in ischemic and non-ischemic legs. Mean±SD including globally 1-time and 2-times NVDX2 treated rats (no difference made between the two “treatment” groups). A: CD3 recruitment in ischemic leg. B: CD3 recruitment in non-ischemic leg. These means were obtained by several counts, on HE stained histological slides, performed at the periphery and at the core of the wound area and mixed together (CD3+ cells/mm²).

FIG. 11A-B is a set of graphs showing the CD68 recruitment in ischemic and non-ischemic legs. Mean±SD including globally 1-time and 2-times NVDX2 treated rats (no difference made between the two “treatment” groups). A: CD68 recruitment in ischemic leg. B: CD68 recruitment in non-ischemic leg. These means were obtained by several counts, on HE stained histological slides, performed at the periphery and at the core of the wound area and mixed together (CD68+ cells/mm²).

FIG. 12A-B is a set of photographs obtained by scanning electron microscopy showing the microscopic structures of fresh (NVD003) (A) and freeze-dried, non-irradiated (NVDX3) (B) biomaterials obtained as detailed in example 1, after 3D-induction in the presence of HA//β-TCP. Upper panels represent a microscopic view obtained with a ×25 zoom. Lower panels represent a microscopic view obtained with a ×1,200-1,300 zoom.

FIG. 13A-B is a set of graphs showing the expression profile of genes VEGFA (A), VEGFB (B) in the biomaterials NVD003 and NVD0031yo, as compared to HA/β-TCP. * p<0.05.

FIG. 14A-D is a set of graphs showing the expression profile of genes SMAD2 (A), SMAD3 (B), SMAD4 (C) and SMAD5 (D) in the biomaterials NVD003 and NVD0031yo, as compared to HA/β-TCP. * p<0.05; ** p<0.01.

FIG. 15A-C is a set of graphs showing the expression profile of genes ITGAV (A), ITGB1 (B) and VCAM1 (C) in the biomaterials NVD003 and NCD0031yo, as compared to HA/β-TCP. * p<0.05.

FIG. 16A-K is a set of graphs showing the expression profile of genes ACVR1 (A), BMPR1A (B), BMPR1B (C), BMPR2 (D), CSF1 (E), EGFR (F), FGFR1 (G), IGF1R (H), RUNX2 (I), TGFBR1 (J) and TWIST1 (K) in the biomaterials NVD003 and NVD0031yo obtained from 3D-induction in the presence of HA//β-TCP.

FIG. 17A-B is a graph showing the relative miRNA expression profile of a subset of miRNAs (A: hsa-miR-4485-3p; Let-7i-5p; hsa-miR-24-3p; hsa-miR-210-3p; B: miR-4454; miR-619-5p; miR-3607-5p; miR-3653-5p) in fresh NVD003 (a) and lyophilized NVD003 (b) biomaterials.

FIG. 18A-B s a set of graphs showing the relative expression of miR-210-3p (A) and miR-24-3p (B) of fresh NVD003 and lyophilized and gamma irradiated NVD003 (NVDX3).

FIG. 19 is a graph showing the content of OPG, IGF1 and VEGF (expressed in ng/g of biomaterial) in the lyophilized NVD003 biomaterials upon irradiation at 12 kGy or 25 kGy, at room temperature (RT) or at −80° C. (−80).

FIG. 20 is a graph showing the relative expression of hsa-miR-210-3p in the NVD003 lyo and the NVDX3 biomaterials (gamma-irradiated lyophilized NVD003 biomaterial).

FIG. 21A-B is a set of graphs showing the level of inhibition of osteoclastogenesis (A) or of inhibition of mature osteoclasts (B) obtained with respect to the doses (mg) of the NVD003 biomaterial, obtained from 3D-induction in the presence of HA//β-TCP.

FIG. 22A-B is a set of graphs showing the level of inhibition of osteoclastogenesis (A) or of inhibition of mature osteoclasts (B) obtained with respect to the doses (mg) of HA/β-TCP.

FIG. 23A-B is a set of graphs showing the level of inhibition of osteoclastogenesis (A) or of inhibition of mature osteoclasts (B) obtained with respect to the doses (mg) of the NVDX3 biomaterial, obtained from 3D-induction in the presence of HA//β-TCP.

FIG. 24A-B is a set of graphs showing the relative induction of BGLAP (osteocalcin) (A) or SPP-1 (osteopontin) (B) obtained with adipose stem cells in osteogenic differentiation medium (‘MD’; control), in the presence of sclerotin (SCL) for 10 days with or without NVDX3 biomaterial.

FIG. 25 is a graph showing the viability of adipose stem cells in osteogenic differentiation medium in the presence or absence of sclerotin at 10 ng/ml (SCL10) or 100 ng/ml (SCL100), and in the presence or absence of the NVDX3 biomaterial. The viability is express as a percentage compared to the viability of adipose stem cells in osteogenic differentiation medium supplemented with 0.5% of human platelet lysate (% vs MD 0.5%).

FIG. 26A-X is a set of graphs showing the relative levels (expressed as a fold of induction) of RUNX-2 (A), BGLAP (B), BMPR1A (C), SMAD5 (D), SMAD2 (E), SPP-1 (F), CSF-1 (G), EGFR (H), TWIST1 (I), TGFB-1 (J), TGFB2 (K), SMAD4 (L), ITGA1 (M), ITGA3 (N), ICAM1 (O), HIFla (P), THBS1 (Q), Leptine (R), MMP-2 (S), EDN1 (T), ENG (U), EFNA1 (V), VEGFA (W), and EFNB2 (X), as a variation of time (7 days (7J) or 14 days (14J)) and of doses (5 mg, 20 mg or 100 mg) of the biomaterial NVDX3. Control conditions (C or CTL) are performed without the NVDX3 biomaterial but with the differentiation medium).

FIG. 27A-D is a set of plots showing the cytotoxicity (A), viability (B), LDH content (C), and DNA content (D) expressed as percentage, of 10, 20, 40, 100, 200 mg NVDX3 or 200 mg HA/PTCP, triton or differentiation medium (MD).

FIG. 28A-H is a set of graphs showing the relative levels (expressed as a fold of induction) of BMPR1A (A), CSF-1 (B), IGF1R (C), TWIST1 (D), SMAD2 (E), SMAD3 (F), SMAD4 (G) and SMAD5 (H), 1 month post transplantation of HA/TCP, NVD003 or NVDX3 biomaterials, obtained from 3D-induction in the presence of HA//β-TCP.

FIG. 29A-B is a set of graphs showing the evolution of the median anti-HLA IgM (A) or IgG (B) antibodies levels in sera of implanted female Wistar with respect of time (1, 3, 7, 15 and 30 days post implantation). Implantation is performed with HA/β-TCP, NVD003 or NVDX3 biomaterials, obtained from 3D-induction in the presence of HA//β-TCP.

FIG. 30A-B is a set of plots showing the cell viability (expressed as percentage) of HDFa (A) and ASCs (B) incubated for 48h in the presence of 10 μM of dexamethasone (GC, for glucocorticoid), with 3 different doses (20 or 50 mg) of NVDX2 or without NVDX2 and measured by metabolic activity (CCK-8 assay), n=2, t-test.

FIG. 31A-B is a set of plots showing the DNA quantification (expressed as percentage) of HDFa (A) and ASCs (B) incubated for 48h in the presence of 10 μM of dexamethasone (GC, for glucocorticoid), with 2 different doses (20 or 50 mg) of NVDX2 or without NVDX2, n=2, t-test.

FIG. 32A-B is a set of plots showing the proliferation (A) and the linear regression of the loss of proliferation (B) of human osteosarcoma cells H143B in the absence (black curve) or in the presence of 2.5 μg/ml (dark grey curve) and 25 μg/ml (light grey curve) of NVD002-Exosomes. (A) The proliferation is expressed as viability (DO) with respect of the time of the co-culture of the cells and the exosomes. (B) Results are expressed as the % of viable cells vs negative control (without exosomes) at each time point. **: p<0.01; ***: p<0.005; ****: p<0.0001; -: no statistical difference.

FIG. 33A-B is a set of plots showing the proliferation (A) and the linear regression of the loss of proliferation (B) of human osteosarcoma cells H143B in the absence (black curve) or in the presence of 2.5 μg/ml (dark grey curve) and 25 μg/ml (light grey curve) of NVD003-Exosomes. (A) The proliferation is expressed as viability (DO) with respect of the time of the co-culture of the cells and the exosomes. (B) Results are expressed as the % of viable cells vs negative control (without exosomes) at each time point. (*: p<0.05; **: p<0.01; -: no statistical difference.

FIG. 34A-B is a set of plots showing the proliferation (A) and the linear regression of the loss of proliferation (B) of human melanoma cells A375 in the absence (black curve) or in the presence of 2.5 μg/ml (dark grey curve) and 25 μg/ml (light grey curve) of NVD002-Exosomes. (A) The proliferation is expressed as viability (DO) with respect of the time of the co-culture of the cells and the exosomes. (B) Results are expressed as the % of viable cells vs negative control (without exosomes) at each time point. *: p<0.05; **: p<0.01; ****: p<0.0001; 00: p<0.01; ^{∘∘∘∘}: p<0.0001; -: no statistical difference.

FIG. 35A-B is a set of plots showing the proliferation (A) and the linear regression of the loss of proliferation (B) of human melanoma cells A375 in the absence (black curve) or in the presence of 2.5 μg/ml (dark grey curve) and 25 μg/ml (light grey curve) of NVD003-Exosomes. (A) The proliferation is expressed as viability (DO) with respect of the time of the co-culture of the cells and the exosomes. (B) Results are expressed as the % of viable cells vs negative control (without exosomes) at each time point. ***: p<0.005; ****: p<0.0001; ^{∘∘∘∘}: p<0.0001; -: no statistical difference.

FIG. 36A-B is a set of plots showing the proliferation (A) and the linear regression of the loss of proliferation (B) of human glioblastoma cells U87 in the absence (black curve) or in the presence of 2.5 μg/ml (dark grey curve) and 25 μg/ml (light grey curve) of NVD002-Exosomes. (A) The proliferation is expressed as viability (DO) with respect of the time of the co-culture of the cells and the exosomes. (B) Results are expressed as the % of viable cells vs negative control (without exosomes) at each time point. *: p<0.05; ****: p<0.0001; ^{∘∘∘∘}: p<0.0001; -: no statistical difference.

FIG. 37A-B is a set of plots showing the proliferation (A) and the linear regression of the loss of proliferation (B) of human glioblastoma cells U87 in the absence (black curve) or in the presence of 2.5 μg/ml (dark grey curve) and 25 μg/ml (light grey curve) of NVD003-Exosomes. (A) The proliferation is expressed as viability (DO) with respect of the time of the co-culture of the cells and the exosomes. (B) Results are expressed as the % of viable cells vs negative control (without exosomes) at each time point. ***: p<0.005; ****: p<0.0001; ^{∘∘∘∘}: p<0.0001; -: no statistical difference.

### EXAMPLES

The present invention is further illustrated by the following examples.

### Example 1: Production of Biomaterials According to the Invention

a) Isolation of hASCs

Human subcutaneous adipose tissues were harvested by lipo-aspiration following Coleman technique in the abdominal region and after informed consent and serologic screening.

Human adipose tissue-derived stem cells (hASCs) were promptly isolated from the incoming adipose tissue. Lipoaspirate can be stored at +4° C. for 24 hours or for a longer time at −80° C.

First, a fraction of the lipoaspirate was isolated for quality control purposes and the remaining volume of the lipoaspirate was measured. Then, the lipoaspirate was digested by a collagenase solution (NB 1, Serva Electrophoresis® GmbH, Heidelberg, Germany) prepared in HBSS (with a final concentration of −8 U/mL). The volume of the enzyme solution used for the digestion was the double of the volume of the adipose tissue. The digestion was performed during 50-70 min at 37° C.±1° C. A first intermittent shaking was performed after 15-25 min and a second one after 35-45 min. The digestion was stopped by the addition of MP medium (proliferation medium, or growth medium). The MP medium comprised DMEM medium (4.5 g/L glucose and 4 mM Ala-Gln; Sartorius Stedim Biotech®, Gottingen, Germany) supplemented with 5% human platelet lysate (hPL) (v/v). DMEM is a standard culture medium containing salts, amino acids, vitamins, pyruvate and glucose, buffered with a carbonate buffer and has a physiological pH (7.2-7.4). The DMEM used contained Ala-Gln. Human platelet lysate (hPL) is a rich source of growth factor used to stimulate in vitro growth of mesenchymal stem cells (such as hASCs).

The digested adipose tissue was centrifuged (500×g, 10 min, 20° C.) and the supernatant was removed. The pelleted Stromal Vascular Fraction (SVF) was re-suspended into MP medium and passed through a 200-500 μm mesh filter. The filtered cell suspension was centrifuged a second time (500×g, 10 min, 20° C.). The pellet containing the hASCs was re-suspended into MP medium. A small fraction of the cell suspension can be kept for cell counting and the entire remaining cell suspension was used to seed one 75 cm² T-flask (referred as Passage P0). Cell counting was performed (for information only) in order to estimate the number of seeded cells.

The day after the isolation step (day 1), the growth medium was removed from the 75 cm² T-flask. Cells were rinsed three times with phosphate buffer and freshly prepared MP medium was then added to the flask.

### b) Growth and Expansion of Human Adipose Tissue-Derived Stem Cells

During the proliferation phase, hASCs were passaged 4 times (P1, P2, P3 and P4) in order to obtain a sufficient amount of cells for the subsequent steps of the process.

Between P0 and the fourth passage (P4), cells were cultivated on T-flasks and fed with fresh MP medium. Cells were passaged when reaching a confluence ≥70% and ≤100% (target confluence: 80-90%). All the cell culture recipients from 1 batch were passaged at the same time. At each passage, cells were detached from their culture vessel with TrypLE (Select 1X; 9 mL for 75 cm² flasks or 12 mL for 150 cm² flasks), a recombinant animal-free cell-dissociation enzyme. TrypLe digestion was performed for 5-15 min at 37° C.±2° C. and stopped by the addition of MP medium.

Cells were then centrifuged (500×g, 5 min, 20° C.), and re-suspended in MP medium. Harvested cells were pooled in order to guaranty a homogenous cell suspension. After resuspension, cells were counted.

At passages P1, P2 and P3, the remaining cell suspension was then diluted to the appropriate cell density in MP medium and seeded on larger tissue culture surfaces. At these steps, 75 cm² flasks were seeded with a cell suspension volume of 15 mL, while 150 cm² flasks were seeded with a cell suspension volume of 30 mL. At each passage, cells were seeded between 0.5×104 and 0.8×10⁴ cells/cm². Between the different passages, culture medium was exchanged every 3-4 days. The cell behavior and growth rate from one donor to another could slightly differ. Hence the duration between two passages and the number of medium exchanges between passages may vary from one donor to another.

### c) Osteogenic Differentiation

At passage P4 (i.e., the fourth passage), cells were centrifuged a second time, and re-suspended in MD medium (differentiation medium). After resuspension, cells were counted a second time before being diluted to the appropriate cell density in MD medium, and a cell suspension volume of 70 mL was seeded on 150 cm² flasks and fed with osteogenic MD medium. According to this method, cells were directly cultured in osteogenic MD medium after the fourth passage. Therefore, osteogenic MD medium was added while cells have not reached confluence.

The osteogenic MD medium was composed of proliferation medium (DMEM, Ala-Gln, hPL 5%) supplemented with dexamethasone (1 μM), ascorbic acid (0.25 mM) and sodium phosphate (2.93 mM).

The cell behavior and growth rate from one donor to another could slightly differ. Hence the duration of the osteogenic differentiation step and the number of medium exchanges between passages may vary from one donor to another.

### d) Multi-Dimensional Induction of Cells

The multi-dimensional induction of ASCs was launched when cells reach a confluence and if a morphologic change appears and if at least one osteoid nodule (i.e., the un-mineralized, organic portion of the bone matrix that forms prior to the maturation of bone tissue) was observed in the flasks.

3D-Induction with Gelatin Particles

After being exposed to the osteogenic MD medium, the culture vessels containing the confluent monolayer of adherent osteogenic cells were slowly and homogeneously sprinkled with gelatin particles (Cultispher-S, Percell Biolytica, Astorp, Sweden) at a concentration of 1.5 cm³ for a 150 cm² vessel.

Cells were maintained in MD medium. Regular medium exchanges were performed every 3 to 4 days during the multi-dimensional induction. Those medium exchanges were performed by carefully preventing removal of gelatin particles and developing structure(s).

After about 15 days, the scaffold-free 3D culture (NVD-002 biomaterial) is developed and detached from the T-flasks. Cultures are maintained during 5 to 8 weeks after the addition of particles with medium change every 3-4 days.

3D-Induction with HA ZA-TCP Particles (Ceramic Particles)

After being exposed to the osteogenic MD medium, the culture vessels containing the confluent monolayer of adherent osteogenic cells were slowly and homogeneously sprinkled with HA/β-TCP particles (ratio of 60/40), 3 cc/cm² for a 150 cm² flask (Biomatlante®, France).

Cells were maintained in MD medium. Regular medium exchanges were performed every 3 to 4 days during the multi-dimensional induction. Those medium exchanges were performed by carefully preventing removal of ceramic material particles and developing structure(s).

After about 15 days, the scaffold-free 3D culture (NVD-003 biomaterial) is developed and detached from the T-flasks. Cultures are maintained during 5 to 8 weeks after the addition of particles with medium change every 3-4 days.

### e) Freeze-Drying and Gamma-Irradiation

The obtained biomaterials, hereunder referred to as NVD002 (gelatin) and NVD003 (HA/TCP), is further freeze-dried (NVD002 lyo and NVD003 lyo)) or freeze-dried and further sterilized so as to obtain a desiccated, sterile biomaterial, hereunder referred to as NVDX2 and NVDX3, respectively.

Freeze-drying is performed by sublimation of the fresh biomaterial at −80° C. and under vacuum for at least 24 hours (<0.05 mBar, −50° c., 24-36 hours).

Sterilization is performed by submitting the freeze-dried biomaterial to a dose comprised from about 12 kGy to about 25 kGy (for 730 sec) at a temperature of about 20° C. to about −80° C.

### Example 2: Viability of the Cells in NVDX2 and NVDX3 Biomaterials

### 1. Materials and Methods

To determine the content of viable cells in the product, biopsies of NVDX2 or NVDX3 were compared with known concentrations of ASCs, which corresponded to 100%, 50%, 10% and 1% of viable cells.

The NVDX2 and NVDX3 were placed in 2 ml of differentiated medium (MD) for 24 hours. The cellularity of NVD003 and NVD002 biopsies was calculated, for 300 mg of each tissue, estimated to be 2.1×10⁶ cells, this corresponding to 100% of viable cells. The proliferation doubling time for the ASCs is about 30 hours.

The viable cells were determined by 2 different methods:

1) A cell viability test (CellTiter-Glo Cell® viability assay) was carried out after 24 hours in culture, to estimate the viable cells in the ASCs and in the NVDX2 and NVDX3 biopsies. The Cell Titer-Glo® luminescent cell viability assay is a homogeneous method to determine the number of viable cells in culture based on quantitation of the ATP present inside the cells, which signals the presence of metabolically active cells.

2) The quantitative determination of glucose or lactate in differentiation medium after 24 hours in culture using a Cedex Bio® analyzer. The determination of glucose is based on the rate of NADPH formation and is directly proportional to the glucose concentration in the medium. The determination of lactate is based on the production of a dye and is directly proportional to the L-lactate concentration in the medium.

The statistical analysis was performed by Prism GraphPad 2, using multiple comparisons with Tukey test (all pairwise comparisons) and Fisher's LSD test (stand-alone comparisons). * pvalue <0.05; ** pvalue<0.01 and *** pvalue<0.001; n.s=not significant.

### 2. Results

### 2.1 Viability

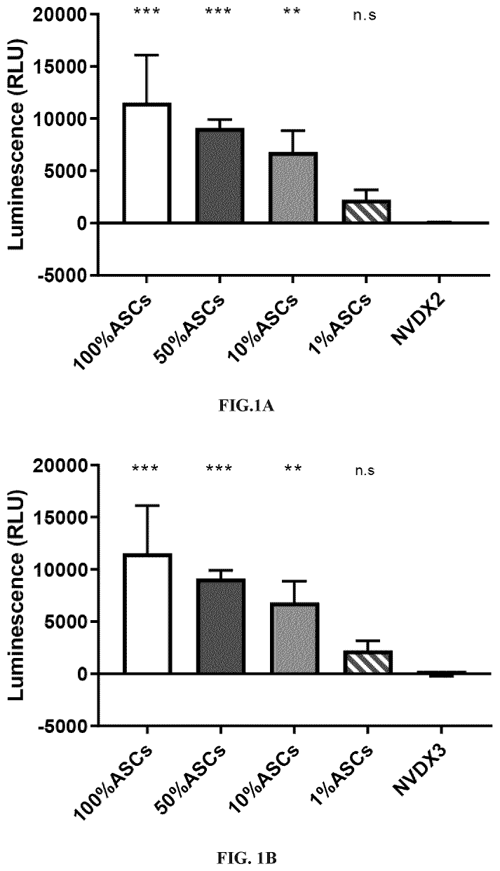

After freeze drying and gamma-irradiation, the presence of viable cells was determined by a Cell Titer-Glo Cell® viability assay. The technique is based on the estimation of the number of viable cells in a culture, based on quantitation of the ATP present inside the cells, which indicates the presence of metabolically active cells (=viable cells). The cell is the source of ATP and the luminescence produced is proportional to the number of viable cells. As seen in FIG. 1A-B, the percentage of viable cells in NVDX2 and NVDX3 was significantly less than 10%.

### 2.2 Glucose Consumption

After freeze drying and gamma-irradiation, the glucose consumption in the culture medium, which reflected the viable cells consumption, was determined using a Cedex Bio analyzer. The technique is based on the determination of glucose in the medium consumed by the viable cells.

In the presence of hexokinase (HK), the glucose is phosphorylated by ATP into glucose 6-phosphate (G-6-P), which is oxidized by NADPH in the presence of glucose-6 phosphate dehydrogenase (G-6-PDH). The rate of NADPH formation is measured by UV photometry and is directly proportional to the glucose concentration.

$$Glucose + ATP \to Glucose - 6 P + ADP$$

$$Glucose - 6 P + {NADP}^{+} \to Gluconate - 6 P + NADPH + {H}^{+}$$

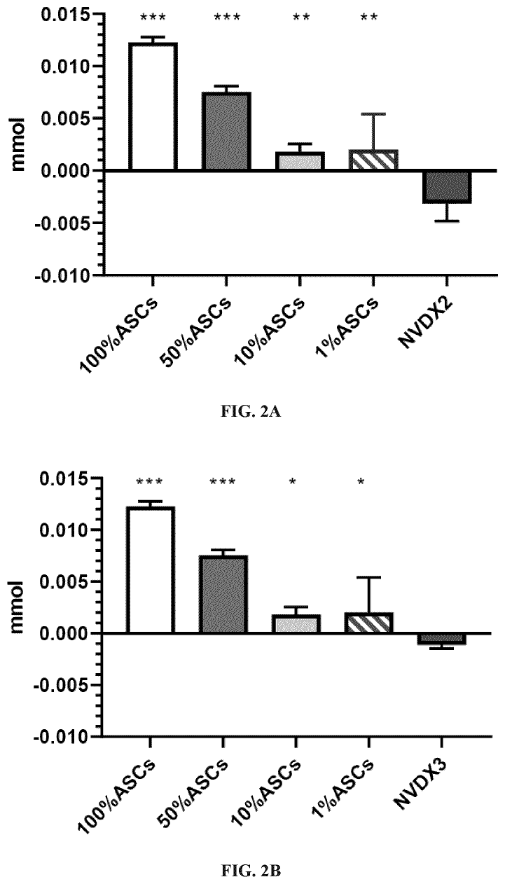

In NVDX2, the glucose consumption was less than the glucose consumed by 1% of viable cells (FIG. 2A), whereas it was less than the amount of glucose consumed by 10% of viable cells in NVDX3 (FIG. 2B).

### 2.3 Lactate Production

After freeze drying and gamma-irradiation, the lactate production in the culture medium, which reflected part of the viable cellular metabolism, was determined using a Cedex Bio Analyzer. The principle of this technique is based on the determination of the lactate production in the medium by the viable cells. The L-lactate is oxidized by lactate oxidase (LaOD) into pyruvate and H₂O₂, which generates a dye in the presence of peroxidase (POD). The photometrically measured absorbance of the dye is directly proportional to the L-lactate concentration in the medium.

$$L - Lactate + {O}_{2} \to Pyruvate + {H}_{2} {O}_{2}$$

$${H}_{2} {O}_{2} + H - donor + 4 - AAP \to {H}_{2} O + chromogen$$

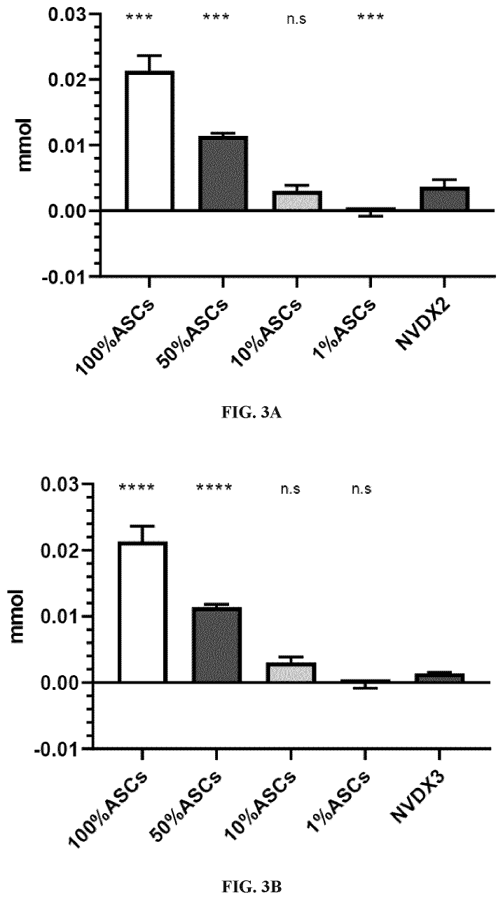

The percentage of viable cells in NVDX2 is shown to be closed to 10% ASCs (FIG. 3A). However, this value may not reflect the actual production of lactate as the value is related to the volume put in the well. NVDX2, with its constitutive gelatin beads, is very porous and absorbs liquid present in the medium, leading to a volume of remaining liquid available of less than 2 ml. This decrease in the volume of available liquid has an impact on the estimation of the presence of lactate in the medium in comparison to the presence of lactate in medium+ASCs. The lactate is concentrated, leading to an overestimation of the remaining viable cells. It can therefore be postulated that the percentage of remaining viable cells is less than 10% in NVDX2. We obtained respectively (0.0037±0.0010 mmol of lactate) for NVDX2 and (0.0030±0.0008 mmol of lactate) for 10% ASCs (FIG. 3A). The lactate production from 10% of ASCs was estimated at 0.0030 mmol (±0.0008 mmol) while it was only 0.0014 mmol (±0.001 mmol) (FIG. 3B). However, the difference was not significant due to the small sample size.

### 2.4 Conclusion

NVD002 and NVD003 are large moldable structures compound of ASCs and Cultispher-S or HA/PTCP particles respectively entrapped in the extracellular matrix. After freeze drying and gamma-irradiation, the structure of both 3D grafts (NVDX2 and NVDX3, respectively) was strongly modified.

Taken into account a viability study and a balance of key cellular metabolites, our study showed a viable cells content in the freeze-dried products of less than 10%, probably at most 1% in both NVDX2 and NVDX3 products.

### Example 3: Impact of Lyophilization on Moisture Content of NVD002 and NVD003 Biomaterials

The aim of this study was to assess the impact of freeze-drying on moisture content of NVD002 and NVD003.

### 1. Materials and Methods

### a) Biopsies

Biopsies of NVD002 and NVD003 were compared to biopsies of lyophilized NVD002 (=NVD00X2) and lyophilized NVD003 (NVD00X3), respectively, to assess the residual moisture content with a moisture analyzer. The analysis was performed on NVD002 and NVD00X2 from one donor, on NVD003 from one donor and NVD00X3 from 2 donor.

### b) Moisture Analysis

To determine the moisture content of a sample, we were used a moisture analyzer (Ohaus®, moisture analyzer MB120). The principle of this technique is to determine the weight of the sample at the beginning. The sample is quickly heated by the dryer unit and moisture vaporizes. During the drying operation, instrument continuously determines the weight of the sample. On completion of drying, result is displayed as percentage of moisture content.

NVD002, NVD003, NVD00X2 and NVD00X3 were weighted, afterwards, the products were heated by infrared until the sample no longer lost weight and the moisture content was calculated. The total loss in weight was used to calculate the moisture.

### 2. Results

NVD002 is a scaffold-free, malleable and translucid 3D sheet-like structure; NVD003 is a scaffold-free, mouldable, white-yellow 3D structure. After lyophilization NVD00X2 is pink brown like skin and dry like a flake; NVD00X3 is a white-yellow dry powder.

After lyophilization, moisture content was determined by moisture analyzer, MB120 OHAUS®. The principle of this technique is to determine the weight of the sample at the beginning. The sample is quickly heated by the dryer unit and moisture vaporizes. During the drying operation, instrument continuously determines the weight of the sample. On completion of drying, result is displayed as % moisture content.

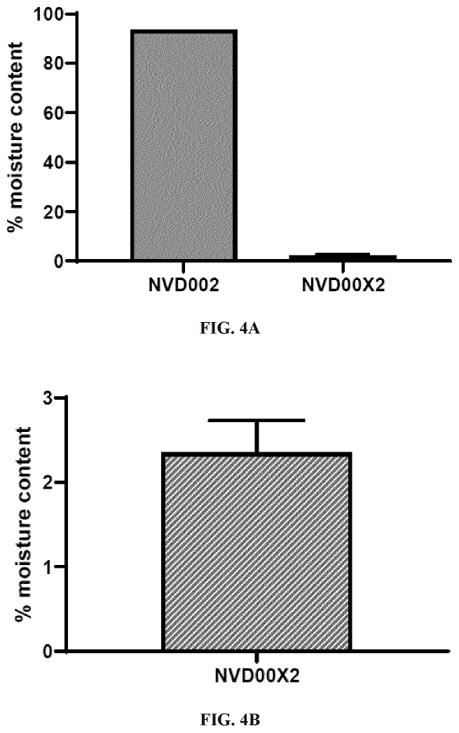

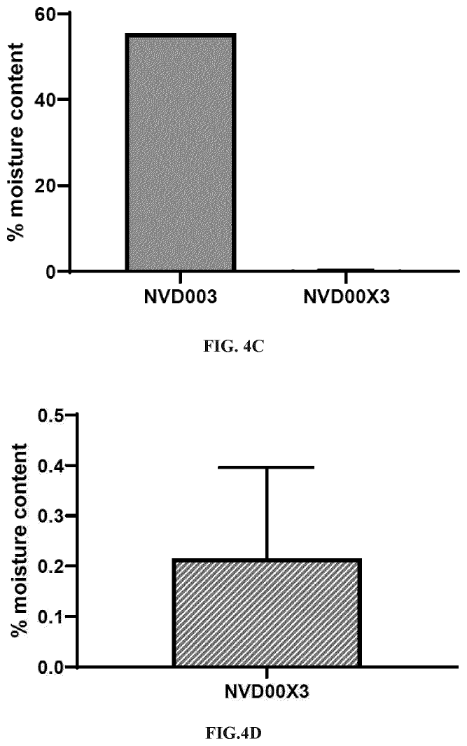

The moisture content was about 90% for fresh NVD002 biomaterial (FIG. 4A) and about 55% for fresh NVD003 biomaterial (FIG. 4C). After lyophilization, the moisture content was less 5%, i.e., less than 2.5% for NVD00X2 (FIG. 4A-B); less than 0.25% for NVD00X3 (FIG. 4C-D). We were observed a good freeze-dried product.

### Example 4: Characterization of the Biomaterials NVD002, NVD002 Lyo and NVDX2; Obtained after 3D-Induction in the Presence of Gelatin

### 1. Materials and Methods

a) mRNAs isolation was performed from biopsies. mRNAs were extracted using miRNeasy kit Mastermix (Qiagen®, Hilden, Germany) following the manufacturer's protocol. RNA concentration was determined by Nanodrop (ThermoFisher®, Waltham, Massachusetts, USA).

b) For quantification of miRNA expression, 50 ng RNA was reverse transcribed into cDNA using qScript miRNA cDNA Synthesis kit (Quanta Biosciences®), and qRT-PCR was conducted in triplicate using Perfecta SYBR Green Super Mix (Quanta Biosciences®). Thermal cycling was performed on an Applied Biosystems 7900 HT detection system (Applied Biosystems®). Data was normalized to miR-16-5p and U6 small nuclear RNA using the Delta-Delta Ct method.

c) Exosomes have been isolated by differential centrifugation from culture medium whereby larger “contaminants” are first excluded by pelleting out through increasing speeds of centrifugation before exosomes, small extracellular vesicles and even protein aggregates are pelleted at very high speeds (˜100,000×g).

### 2. Results

TABLE 13
identification of miRNAs from purified exosomes

| | | | |
|---|---|---|---|
| hsa-let-7a-5p | hsa-miR-92a-3p | hsa-miR-92b-3p | hsa-miR-24-2-5p |
| hsa-let-7b-5p | hsa-miR-125b-5p | hsa-miR-335-5p | hsa-miR-26a-2-3p |
| hsa-let-7f-5p | hsa-miR-337-3p | hsa-let-7f-1-3p | hsa-miR-301a-3p |
| hsa-miR-24-3p | hsa-miR-93-5p | hsa-miR-196b-5p | hsa-miR-98-3p |
| hsa-miR-21-5p | hsa-miR-409-3p | hsa-miR-3613-3p | hsa-miR-1273a |
| hsa-miR-23b-3p | hsa-miR-199a-3p | hsa-miR-23a-5p | hsa-miR-28-5p |
| hsa-miR-1273g-3p | hsa-miR-145-5p | hsa-miR-374b-5p | hsa-miR-34a-3p |
| hsa-miR-574-3p | hsa-miR-30a-3p | hsa-miR-660-5p | hsa-miR-425-3p |
| hsa-miR-25-3p | hsa-miR-382-5p | hsa-miR-186-5p | hsa-miR-505-3p |
| hsa-let-7e-5p | hsa-miR-19b-3p | hsa-miR-454-3p | hsa-miR-34b-3p |
| hsa-miR-214-3p | hsa-miR-210-3p | hsa-miR-10a-5p | hsa-miR-361-3p |
| hsa-miR-199a-5p | hsa-miR-619-5p | hsa-miR-495-3p | hsa-miR-10b-5p |
| hsa-miR-196a-5p | hsa-miR-17-5p | hsa-miR-425-5p | hsa-miR-1306-5p |
| hsa-miR-199b-5p | hsa-miR-193a-5p | hsa-miR-2053 | hsa-miR-22-5p |
| hsa-miR-221-3p | hsa-miR-320b | hsa-miR-5096 | hsa-miR-378a-3p |
| hsa-miR-424-5p | hsa-miR-193b-5p | hsa-miR-494-3p | hsa-miR-411-5p |
| hsa-miR-23a-3p | hsa-miR-320a | hsa-miR-27a-3p | hsa-miR-505-5p |
| hsa-let-7c-5p | hsa-miR-151a-3p | hsa-miR-4449 | hsa-miR-664a-3p |
| hsa-miR-199b-3p | hsa-let-7a-3p | hsa-miR-532-3p | hsa-miR-26a-5p |
| hsa-miR-191-5p | hsa-miR-30e-3p | hsa-miR-532-5p | hsa-miR-377-3p |
| hsa-miR-574-5p | hsa-miR-22-3p | hsa-miR-126-5p | hsa-miR-485-3p |
| hsa-miR-424-3p | hsa-miR-99b-5p | hsa-miR-30c-5p | hsa-miR-590-3p |
| hsa-miR-423-5p | hsa-miR-625-3p | hsa-miR-130b-3p | hsa-miR-99a-3p |
| hsa-miR-342-3p | hsa-miR-4668-5p | hsa-miR-136-3p | hsa-miR-143-3p |
| hsa-let-7d-3p | hsa-miR-29b-3p | hsa-miR-15b-3p | hsa-miR-26b-3p |
| hsa-miR-130a-3p | hsa-miR-423-3p | hsa-miR-29b-1-5p | hsa-miR-3607-5p |
| hsa-miR-3184-3p | hsa-miR-376c-3p | hsa-miR-99b-3p | hsa-miR-3651 |
| hsa-miR-222-3p | hsa-let-7b-3p | hsa-miR-127-3p | hsa-miR-374a-3p |
| hsa-let-7g-5p | hsa-miR-3074-5p | hsa-miR-134-5p | hsa-miR-376a-3p |
| hsa-miR-125a-5p | hsa-miR-98-5p | hsa-miR-324-5p | hsa-miR-485-5p |
| hsa-let-7d-5p | hsa-miR-185-5p | hsa-miR-3605-3p | hsa-miR-103b |
| hsa-miR-29a-3p | hsa-miR-19a-3p | hsa-miR-101-3p | hsa-miR-126-3p |
| hsa-let-7i-5p | hsa-miR-34a-5p | hsa-miR-103a-3p | hsa-miR-149-5p |
| hsa-miR-146b-5p | hsa-miR-374c-3p | hsa-miR-1246 | hsa-miR-193b-3p |
| hsa-miR-4454 | hsa-miR-181a-5p | hsa-miR-138-5p | hsa-miR-223-3p |
| hsa-miR-28-3p | hsa-miR-328-3p | hsa-miR-190a-5p | hsa-miR-340-3p |
| hsa-miR-874-3p | hsa-miR-7847-3p | hsa-miR-6724-5p | hsa-miR-369-5p |

TABLE 14
identification of cellular miRNAs

| | | | |
|---|---|---|---|
| hsa-let-7a-5p | hsa-miR-210-3p | hsa-miR-29b-3p | hsa-miR-30e-3p |
| hsa-let-7b-5p | hsa-miR-3184-3p | hsa-miR-92a-3p | hsa-miR-320a |
| hsa-miR-24-3p | hsa-let-7d-5p | hsa-miR-193b-5p | hsa-miR-361-3p |
| hsa-miR-199a-5p | hsa-miR-25-3p | hsa-miR-181a-5p | hsa-miR-151a-3p |
| hsa-miR-214-3p | hsa-miR-193a-5p | hsa-miR-30c-5p | hsa-miR-154-5p |
| hsa-let-7f-5p | hsa-miR-199a-3p | hsa-miR-664b-3p | hsa-miR-664a-5p |
| hsa-miR-3607-5p | hsa-miR-29a-3p | hsa-miR-27a-3p | hsa-miR-92b-3p |
| hsa-miR-199b-3p | hsa-miR-342-3p | hsa-miR-320b | hsa-miR-1291 |
| hsa-let-7e-5p | hsa-miR-130a-3p | hsa-miR-3651 | hsa-miR-103b |
| hsa-miR-1273g-3p | hsa-miR-30a-3p | hsa-miR-664b-5p | hsa-miR-34a-3p |
| hsa-miR-125a-5p | hsa-miR-145-5p | hsa-miR-664a-3p | hsa-miR-140-5p |
| hsa-miR-21-5p | hsa-miR-28-3p | hsa-miR-98-5p | hsa-miR-3609 |
| hsa-let-7i-5p | hsa-miR-93-5p | hsa-miR-146b-5p | hsa-miR-374c-3p |
| hsa-miR-125b-5p | hsa-miR-34a-5p | hsa-miR-337-3p | hsa-miR-10a-5p |
| hsa-let-7g-5p | hsa-miR-222-3p | hsa-miR-4449 | hsa-miR-22-3p |
| hsa-miR-191-5p | hsa-miR-3074-5p | hsa-miR-6516-3p | hsa-miR-4668-5p |
| hsa-miR-574-3p | hsa-miR-424-5p | hsa-let-7i-3p | hsa-miR-24-2-5p |
| hsa-miR-199b-5p | hsa-miR-424-3p | hsa-miR-103a-3p | hsa-miR-29b-1-5p |
| hsa-miR-423-5p | hsa-miR-328-3p | hsa-miR-324-5p | hsa-miR-335-5p |
| hsa-miR-574-5p | hsa-miR-17-5p | hsa-miR-660-5p | hsa-miR-425-5p |
| hsa-miR-23b-3p | hsa-miR-23a-3p | hsa-miR-185-5p | hsa-miR-4461 |
| hsa-miR-196a-5p | hsa-let-7d-3p | hsa-miR-374b-5p | hsa-miR-127-3p |
| hsa-let-7c-5p | hsa-miR-423-3p | hsa-miR-409-3p | hsa-miR-196b-5p |
| hsa-miR-221-3p | hsa-miR-382-5p | hsa-miR-619-5p | hsa-miR-3613-5p |
| hsa-miR-3653-5p | hsa-miR-19b-3p | hsa-miR-99b-5p | hsa-miR-376c-3p |
| hsa-miR-99b-3p | hsa-miR-663b | hsa-miR-495-3p | hsa-miR-454-3p |

TABLE 15
level of miRNAs from purified exosomes as compared to 2D culture

| miRNA | logFC | FC | logCPM | PValue | FDR |
|---|---|---|---|---|---|
| hsa-miR-3687 | −5.4340 | −43.2330 | 9.986598816 | 6.14E−08 | 3.38E−05 |
| hsa-miR-664b-5p | −6.7300 | −106.1558 | 9.571690269 | 1.19E−06 | 0.00032614 |
| hsa-miR-210-3p | 3.3558 | 10.2381 | 10.48338867 | 2.04E−06 | 0.00035487 |
| hsa-miR-4449 | −2.9142 | −7.5382 | 11.07025445 | 2.58E−06 | 0.00035487 |
| hsa-miR-3651 | −4.4165 | −21.3551 | 11.30176655 | 1.47E−05 | 0.00140376 |
| hsa-miR-663a | −5.5373 | −46.4407 | 10.73936527 | 1.53E−05 | 0.00140376 |
| hsa-miR-664b-3p | −7.2564 | −152.9025 | 10.0417161 | 2.26E−05 | 0.00177881 |
| hsa-miR-3653-5p | −3.7408 | −13.3696 | 9.73359759 | 5.05E−05 | 0.00347481 |
| hsa-miR-664a-3p | −2.8640 | −7.28064 | 10.34200935 | 0.000189789 | 0.01159819 |
| hsa-miR-3648 | −3.5109 | −11.4000 | 9.789761883 | 0.000273373 | 0.01503553 |
| hsa-miR-619-5p | 1.9219 | 3.7892 | 10.62269524 | 0.002222957 | 0.11114785 |
| hsa-miR-181a-5p | 2.5395 | 5.8140 | 9.387142262 | 0.002792503 | 0.12798972 |
| hsa-miR-409-3p | −1.3868 | −2.6151 | 12.81792844 | 0.00335429 | 0.14191227 |
| hsa-let-7a-3p | 6.5703 | 95.0297 | 9.606951776 | 0.004329701 | 0.17009541 |
| hsa-miR-4454 | 2.6272 | 6.1784 | 11.53331453 | 0.004846818 | 0.17771667 |
| hsa-let-7i-5p | 1.4660 | 2.7626 | 11.86103511 | 0.006780515 | 0.23308021 |
| hsa-miR-335-5p | 3.5260 | 11.5196 | 9.315665201 | 0.007991541 | 0.25854987 |
| hsa-miR-1246 | 5.0336 | 32.7554 | 8.539445396 | 0.009781646 | 0.29888364 |
| hsa-miR-6516-5p | −4.1176 | −17.3593 | 9.073414111 | 0.011915311 | 0.34491691 |
| hsa-miR-3607-5p | −2.0844 | −4.2410 | 9.626131484 | 0.012915156 | 0.35516678 |
| hsa-let-7e-5p | 1.3793 | 2.6014 | 13.88843995 | 0.016482404 | 0.43168201 |
| hsa-miR-25-3p | −1.0699 | −2.0993 | 14.99237678 | 0.020116155 | 0.50290388 |
| hsa-miR-374c-3p | 2.1608 | 4.4719 | 9.494278145 | 0.0222919 | 0.53306717 |
| hsa-miR-29b-3p | 1.5768 | 2.9832 | 9.845249887 | 0.025174313 | 0.56328252 |
| hsa-let-7b-3p | −1.4233 | −2.6820 | 10.95150956 | 0.025603751 | 0.56328252 |
| hsa-miR-23b-3p | 1.2910 | 2.4470 | 14.73760912 | 0.028045493 | 0.5796539 |
| hsa-miR-3613-3p | −2.0169 | −4.0472 | 10.92938585 | 0.028455737 | 0.5796539 |
| hsa-miR-138-5p | −1.5674 | −2.9637 | 9.777277597 | 0.040019769 | 0.78610261 |
| hsa-miR-6516-3p | −2.0847 | −4.2420 | 8.826423155 | 0.042870909 | 0.81306896 |

TABLE 16
Level of cellular miRNAs in the biomaterial as compared to a 2D culture

| miRNA | logFC | FC | logCPM | PValue | FDR |
|---|---|---|---|---|---|
| hsa-miR-210-3p | 6.4208 | 85.6764 | 12.48420101 | 6.80E−18 | 2.42E−15 |
| hsa-miR-4485-3p | −4.0409 | −16.4603 | 11.3547262 | 8.18E−11 | 1.46E−08 |
| hsa-miR-24-3p | 1.1078 | 2.1551 | 16.03988644 | 2.58E−05 | 0.0030666 |
| hsa-miR-409-3p | −2.2145 | −4.6413 | 11.39958036 | 0.000257967 | 0.02295903 |
| hsa-let-7i-5p | 1.1353 | 2.1966 | 13.67239011 | 0.000499245 | 0.03554627 |
| hsa-miR-382-5p | −1.2517 | −2.3813 | 11.92251263 | 0.00225027 | 0.133516 |
| hsa-miR-214-3p | 0.8790 | 1.8391 | 15.62352032 | 0.003282859 | 0.16695684 |
| hsa-miR-199b-5p | 1.2889 | 2.4435 | 13.44753159 | 0.006034794 | 0.24998011 |
| hsa-miR-199a-5p | 0.8790 | 1.8391 | 15.68100144 | 0.006319722 | 0.24998011 |
| hsa-miR-3074-5p | 1.2659 | 2.4048 | 11.37814998 | 0.009651574 | 0.34359603 |
| hsa-miR-361-3p | 2.8526 | 7.2232 | 9.80277522 | 0.013351705 | 0.43210973 |
| hsa-miR-6723-5p | −4.6621 | −25.3195 | 9.311990262 | 0.015695848 | 0.46564349 |
| hsa-miR-130a-3p | 1.7314 | 3.3205 | 12.0290808 | 0.017837723 | 0.48847918 |
| hsa-miR-663a | −2.7768 | −6.8533 | 9.692314667 | 0.019868045 | 0.505216 |
| hsa-miR-3607-5p | 0.6038 | 1.5197 | 14.71277969 | 0.021552995 | 0.5115244 |
| hsa-miR-660-5p | 1.5747 | 2.9787 | 10.15389819 | 0.025273272 | 0.5623303 |
| hsa-miR-196b-5p | −1.6406 | −3.1181 | 10.12403868 | 0.028246436 | 0.5915136 |
| hsa-miR-4449 | −1.1855 | −2.2744 | 11.22700361 | 0.031038083 | 0.61386431 |
| hsa-miR-342-3p | 0.9434 | 1.9230 | 12.22137602 | 0.038570501 | 0.70780213 |
| hsa-miR-221-3p | −0.7419 | −1.6723 | 13.67334394 | 0.039764165 | 0.70780213 |

As compared to a 2D culture, the miRNAs obtained from the biomaterial according to the invention has an altered miRNAs content. For example, both exosomal and cellular hsa-miR-210-3p and hsa-let-7i-5p are up-regulated, whereas exosomal hsa-miR-664b-3p and hsa-miR-664b-5p, and cellular hsa-miR-4485-3p and hsa-miR-6723-5p, are down-regulated. This suggests that lyophilization and sterilization affects the nature of the biomaterial NVDX2 as compared to a fresh, non-desiccated, non-lyophilized biomaterial.

### Example 5: Characterization of the Composition NVD002, NVD002 lyo and NVDX2, Obtained from 3D-Induction in the Presence of Gelatin

### 1. Materials and Methods

Samples of the ‘fresh’ biomaterial in the presence of gelatin obtained in example 1 (NVD002), the desiccated biomaterial (NVD002 lyo) and the desiccated and sterilized biomaterial (NVDX2) were processed for protein and miRNA extraction.

### a) Protein Extraction

Proteins were extracted from non-irradiated and irradiated samples in Guanidine HCL [4 M], Benzamidine [5 mM], N-ethylmaleimide [10 mM], PMSF [1 mM] at 4° C. during 24h, then addition of tris HCL [50 mM] at pH 7.4 for 5 h (all from Sigma-Aldrich®, Saint Louis, USA) and purified through desalting columns (PD10 de GE Healthcare®, Chicago, USA). Quantification of growth factors VEGF, IGF-1 and SDF-1α content was performed by ELISA (Human VEGF quantikine ELISA Kit, Human SDF-1α quantikine ELISA Kit, Human IGF-1 quantikine ELISA Kit; R&D Systems®, Minneapolis, Minnesota, USA).

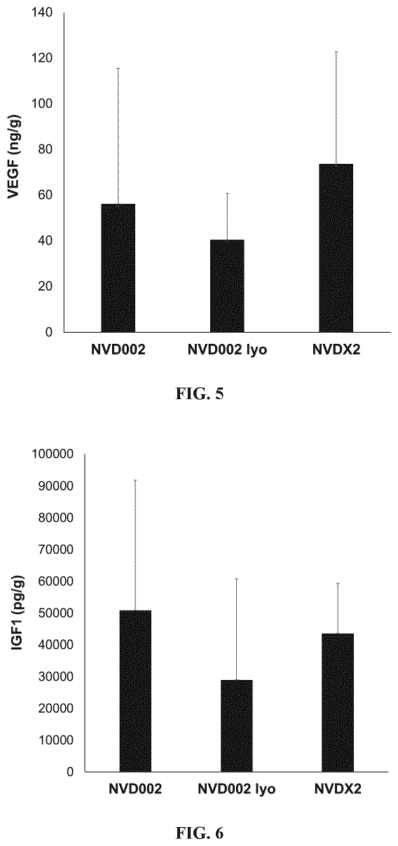

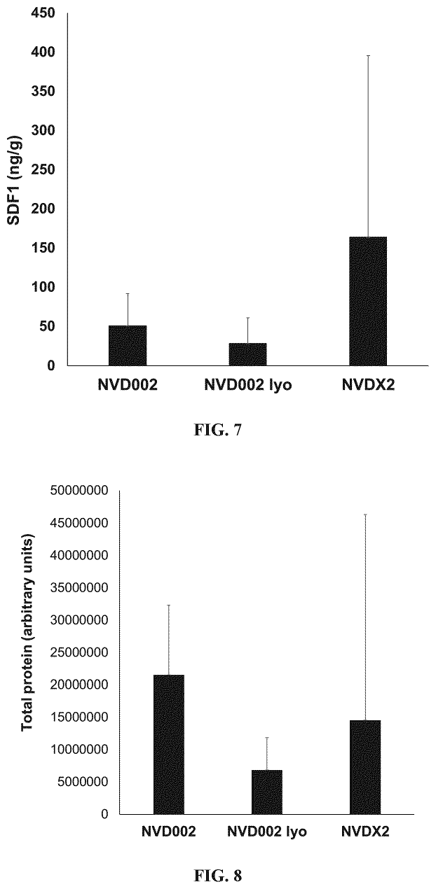

The protein levels of VEGF and SDF-1 were found upregulated in the NVDX2 biomaterial as compared to both the fresh NVD002 biomaterial and the desiccated NVD002 lyo biomaterial (see FIG. 5 and FIG. 7, respectively). On the contrary, the protein levels of IGF-1 in NVD002 and NVDX2 biomaterials were comparable, and upregulated as compared to the NVD002 lyo biomaterial (see FIG. 6). Finally, the total protein levels in all three biomaterials were globally similar (see FIG. 8).

b) miRNA Extraction and RT-PCR

miRNA was extracted using miRNeasy kit Mastermix (Qiagen®, Hilden, Germany) following the manufacturer's protocol. RNA concentration was determined by Nanodrop (ThermoFisher®, Waltham, Massachusetts, USA).

For quantification of miRNA expression, 50 ng RNA was reverse transcribed into cDNA using qScript miRNA cDNA Synthesis kit (Quanta Biosciences®), and qRT-PCR was conducted in triplicate using Perfecta SYBR® Green Super Mix (Quanta Biosciences®). Thermal cycling was performed on an Applied Biosystems® 7900 HT detection system (Applied Biosystems®). Data was normalized to miR-16-5p and U6 small nuclear RNA using the Delta-Delta Ct method.

### 2. Results

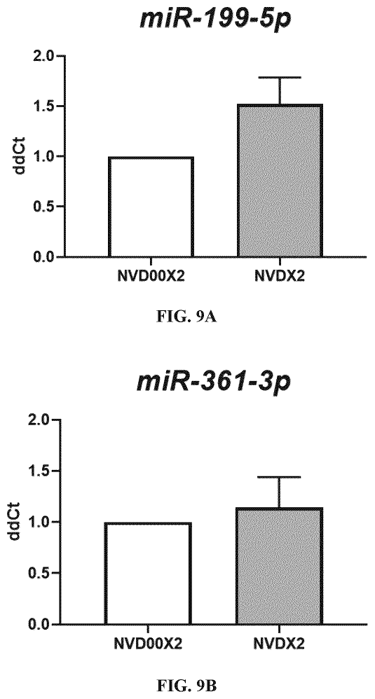

As shown in FIG. 9A-B, no significant differences in miR-199-5p and miR-361-3p expression, respectively, was observed between NVD00X2 (lyophilized NVD002) and NVDX2 (lyophilized and gamma-irradiated NVD002).

### Example 6: Efficacy of NVDX2 Biomaterial in a Hyperglycemic and Ischemic Model

The aim of this study was to assess the efficacy of a biological powder used as a wound dressing, NVDX2 (from human origin), in the treatment of ischemic/hyperglycemic wounds in a Wistar rat model. NVDX2 is the freeze-dried, gamma-irradiated version of NVD002 which is a 3D-graft, scaffold-free, composed of a mix of human adipose tissue-derived stem cells (ASCs), porcine gelatin beads (Cultispher® S, Percell Biolytica®, Sweden) embedded in an extra-cellular matrix produced by the ASCs.

### 1. Materials and Methods

### a) Experimental Design

The efficacy of NVDX2 was evaluated in a xenogenic (human to rat) model of ischemic (vs. non-ischemic) wound in hyperglycemic Wistar rats (n=13). The 13 hyperglycemic rats formed 1 experimental group subdivided into two subgroups according to the number of NVDX2 applications on the wounds (only one application at day 1 (×1) versus two applications at days 1 and 7 respectively (×2)).

### b) Surgical Procedure

This in vivo study was approved by the ethic committee of the CER-Groupe, Biotechnology Department, B6900 AYE, Belgium. The protocol is inspired by the model developed by the Division of Plastic, Reconstructive & Aesthetic Surgery, “H6pitaux Universitaires de Geneve”, University of Geneva, Faculty of Medicine, Geneva, Switzerland (Andre-Levigne et al., Wound Repair and Regeneration. 2016—Alizadeh et al., Wound Repair and Regeneration. 2007).

13 male Wistar rats of approximately 250-300 g were used for this study. Hyperglycemia was induced by intraperitoneal injection of Streptozotocin (STZ) [50 mg/kg] on rats presenting a minimum weight of 250 g. Animals presenting a glycemia over 9 mM were considered as hyperglycemic and were enrolled in the study. Of the 13 originated rats, all were enrolled in the study. Seven to ten days after the STZ injection, hyperglycemic rats were surgically operated to induce a unilateral ischemic model. This model was obtained by the resection of a portion of the femoral artery (from the inguinal area to the knee area) of the left posterior limb of the rat. This model allows, in one individual rat, the presence of ischemic (posterior-left) and non-ischemic (posterior-right) limbs in a hyperglycemic context. Once the artery's portion resected, the incision was stitched and an ALZET 2ML2 pump delivering 5 l/hour of Buprenorphine [0.3 mg/ml] was placed subcutaneously in the rat's back region. After that, a wound of 1 cm² was performed on the back side of the feet by resection of the skin till the tendons and a picture was taken with a measurement tool aside as reference. Test item was then applied on the wound. One rat died during surgery.

The wounds were covered by pouring NVDX2 right on the wound in the case of the non-ischemic limb. For ischemic limb, some sterile physiological solution was dropped (2-3 drops) to allow the “gluing” of NVDX2 (powder) on the “dry wound”. Once the test-item placed on the wound, a bandage was realized constituted of one layer of Tegaderm® followed by the apposition of another layer of sticking plaster and a specific collar was placed around the neck of the rat.

### c) Follow-Up

The animals were subjects to a daily clinical follow-up and according to the wound healing progress, the rats were euthanized, pictures of both posterior limbs were taken, and these feet were cut and placed in formol 10% for histological analyses. For almost all rats, intermediary pictures were taken every three times a week between day 13 to day 37 post-surgery.

### d) Terminal Procedures

Animals were sacrificed by lethal intraperitoneal injection of pentobarbital.

### e) Macroscopic Evaluation of Wound Healing

A kinetic of wound healing was established by measuring the wound area on the pictures of the feet taken from day 0 to day 37.

To quantify the wound closure, the wound area was measured by image analysis using Image J software by two independent operators. The remaining area of the wound was calculated on the wound area measured at each time point between D0 and D37 and was expressed in comparison to the wound area at the implantation time (D0), fixed at 100%.

To describe the contraction and the epithelialization of the wound, wound area was fractionated into different and constitutive parts of the wound: 1) initial wound (blue area), 2) hairless closed wound (white; epithelialized wound). The wound closed by contraction was calculated by the subtraction of the hairless closed wound and the unclosed wound areas to the total wound area.

### f) Histopathologic and 2D Histo-Morphometric Analyses

Legs were dissected to remove the wound tissue and this latest was oriented transversally to have histological slides of the entire thickness of the tissue. Histological slides of 5 m were prepared and stained with HE, TM, CD3, CD68, KU80 and α-SMA.

CD3 (T lymphocytes) and CD68 (macrophages) immunostainings were performed for the evaluation of the immune and inflammatory responses. The number of CD3 and CD68 positive cells was manually counted using NDPview2 software. A region of interest was manually delineated to define the area of the “implant site” on the section.

To evaluate the pro-angiogenic properties of implanted tissues, quantification of area occupied by blood vessels (Masson's Trichrome staining) were performed: a region of interest was manually delineated on the basis of the tissue features to define the area of the “implant site” on the section. Each blood vessel was manually delineated to quantify the area occupied by blood vessels in the region of interest. The surface corresponding to and the number of blood vessels were reported to the total area of the “implant site”. KU80 staining was performed to highlight the presence of human cells at the implantation site. aSMA immunostaining was done to quantify the presence of smooth muscles fibers, responsible of the contraction of the wound. Smooth muscles fibers were quantified by point counting at magnification ×10 on 3 non-overlapping areas of about 2.9 mm². To assess the formation of hypertrophic scars, the wound thickness was measured on histological slides.

### g) Images Analysis

Histological slides were examined using NDPView.2, Hamamatsu Photonics. Image analysis was performed using ImageJ2, NIH.

### 2. Results

On the 13 rats who received streptozotocin injection, all were hyperglycemic (blood glucose >9 mM) and were selected for the study, but only one developed surgical complication, died during the surgery and was therefore excluded from the study.

The evolution of both glycemia and weight were tightly followed to see the maintain of the hyperglycemic status and to evaluate the probable weight loss due to this hyperglycemic status. By the clinical glycemia follow-up, it could be observed that all the rats were presenting a glycemia ≥9 mM during all the in vivo phase of the study (till their euthanasia).

It was observed a slight decrease in the body weight of all rats after the STZ injection (first or second) leading to reach the critical body weight loss of 20% in comparison to the body weight measured before the first STZ injection.

### a) Macroscopic Evaluation of Wound Healing

Macroscopic pictures of wounds were taken during the follow-up and at the endpoint day. A better wound healing can be observed from day 15 after surgery (D15) in the NVDX2-treated non-ischemic limb with a total wound closure estimated around days 22 and 23 post-surgery (mean of all surviving rats). A delay is observed in the wound healing process from day 15 after surgery (D15) in the NVDX2-treated ischemic limb with a total wound closure estimated at day 31 post-surgery (mean of all surviving rats).

The kinetic of wound closure was measured on the wound's pictures. While the surface of the wounds remained constant during the 7-8 first days after NVDX2-treatment, wound healing associated with a reduction of the wound's surface was observed from days 13-16. A complete and irreversible wound closure of the non-ischemic limb was estimated between days 22 and 23 after NVDX2-treatment (1 or 2 NVDX2 applications). In the ischemic limb, a complete and irreversible wound closure was estimated at day 31 after NVDX2-treatment (1 or 2 NVDX2 applications) (Table 17).

TABLE 17
Time required for complete wound closure
via macroscopic observations

| | Time for complete wound closure (days) | |
|---|---|---|
| | Non-ischemic limb | Ischemic limb |
| Mean 1 × NVDX2 (n = 6) | 22 ± 3.4 | 30.8 ± 7.1 |
| Mean 2 × NVDX2 (n = 4) | 23.8 ± 5.0 | 31.3 ± 4.4 |
| Global mean | 22.7 | 31 |

### b) Microscopic Evaluation of Wound Healing

Histological slides after hematoxylin-eosin staining were observed for each animal, at the day of its sacrifice. In two early sacrificed rats, the full thickness wound could be observed at day 2 and the development of a granulation tissue was found at day 15. The test item was clearly visible until day 15, but only some particles could be observed at later time-point.

Each wound was found to be completely healed with a complete epithelial layer at maximum days 36/37 (exception of the ischemic limb of one rat (treated 2×); no data for one rat (treated 1×) sacrificed later, at day 49).

In the both ischemic and non-ischemic limbs, a complete epithelial layer was observed at day 28 post-surgery in one rat (treated 2×) and on day 29 post-surgery (one rat treated 1×). The sub-epithelial layer was not totally reorganized at day 28 in the ischemic limb of one rat (treated 2×) in comparison to the sub-epithelial layer of the ischemic limb of one rat (treated 1×1) at day 29.

### c) Lymphocytes CD3 and Macrophages CD68 Recruitment

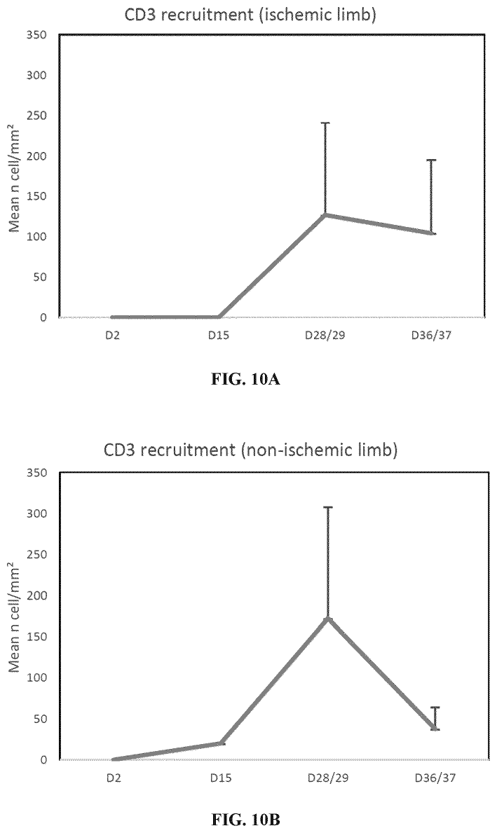

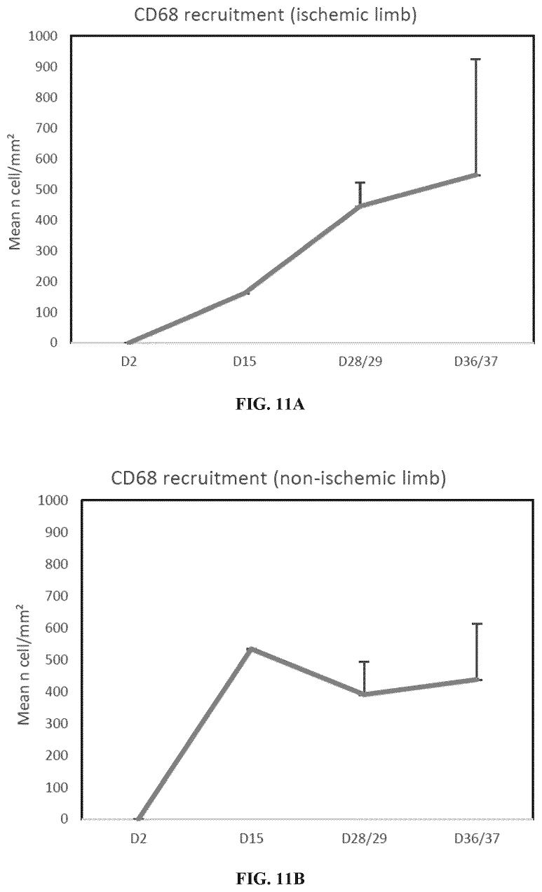

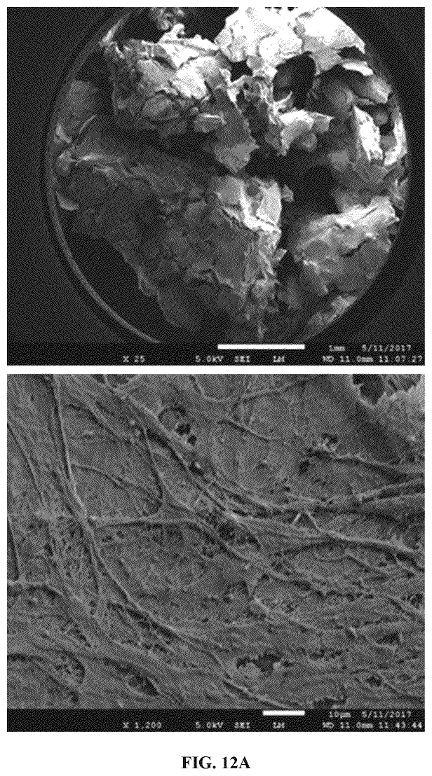

CD3 and CD68 recruitments in non-ischemic and ischemic wounds at each sacrifice time point are represented in FIG. 10A-B and FIG. 11A-B, respectively. Note that different number of rats at each “time-point”: 1 rat for day 2, 1 rat for day 15, 2 rats for days 28/29 and 7 rats for days 36/37 were analyzed.

For both treated limbs, we observed a light and transient CD3+ recruitment, beginning at day 15 for non-ischemic leg (FIG. 10A), with a peak at days 28/29 followed by a significant decreased at days 36/37. For the ischemic limb (FIG. 10B), it began with the peak at day 28/29 followed by a slight decrease at days 36/37.

The different treatment groups were discriminated and results are depicted in Table 18 and Table 19. Mean±SD with the discrimination between 1-time and 2-times NVDX2 treated rats. These means were obtained by several counts, on HE stained histological slides, performed at the periphery and at the core of the wound area and mixed together (CD3+ cells/mm²).

TABLE 18
CD3 recruitment in ischemic legs.

| Ischemic limb | |
|---|---|
| Mean | SD |

| | | | | |
|---|---|---|---|---|
| CD3+ cells/mm² | D28/29 | D36/37 | D28/29 | D36/37 |
| 1 × NVDX2 | 29.4 | 98.8 | NA | 69.9 |
| 2 × NVDX2 | 224.0 | 86.3 | NA | 48.9 |

TABLE 19
CD3 recruitment in non-ischemic legs.

| Non-ischemic limb | |
|---|---|
| Mean | SD |

| | | | | |
|---|---|---|---|---|
| CD3+ cells/mm² | D28/29 | D36/37 | D28/29 | D36/37 |
| 1 × NVDX2 | 59.7 | 51.8 | NA | 25.3 |
| 2 × NVDX2 | 284.2 | 47.6 | NA | 18.1 |

From Table 19 and Table 20, it was observed, in both treated limbs, a difference in CD3+ cells recruitment at days 28/29 between the rat treated one time with NVDX2 and the rat treated two times with NVDX2: 29.4 CD3+ cells/mm² vs 224.0 CD3+ cells/mm² and 59.7 CD3+ cells/mm² vs 284.2 CD3+ cells/mm² for ischemic and non-ischemic limbs respectively.

This difference could be explained by the immune reaction against NVDX2 during the second application occurring 1 week after the first one. The elicitation of the immune response was caused by this second NVDX2 application, leading to a classical graft rejection response.

This difference observed at days 28/29 secmed to be cleared at days 36/37 were it was observed a similar number of CD3+ cells/mm² in both treated groups with 98.8±69.9 (1×NVDX2) vs 86.3±48.9 (2×NVDX2) and 51.8±25.3 (1×NVDX2) vs 47.6±18.1 (2×NVDX2) for ischemic and non-ischemic limbs respectively. The data for days 36/37 were obtained from 5 and 3 rats for 1×NVDX2 and 2×NVDX2 applications respectively.

For both treated limbs, it was observed an increased CD68+ recruitment from day 15 for both treated legs (FIG. 11A-B). This increase seemed to continue or to be maintained in the following days to reach 546.9±376.9 CD68+ cells/mm² and 437.2±174.6 CD68+ cells/mm² for ischemic and non-ischemic limbs respectively.

Table 20 and Table 21 summarize the results obtained after discrimination of the different treatment groups. Mean±SD with the discrimination between 1-time and 2-times NVDX2 treated rats. These means were obtained by several counts, on HE stained histological slides, performed at the periphery and at the core of the wound area and mixed together (CD68+ cells/mm²).

TABLE 20
CD68 recruitment in ischemic legs.

| Ischemic limb | |
|---|---|
| Mean | SD |

| | | | | |
|---|---|---|---|---|
| CD3+ cells/mm² | D28/29 | D36/37 | D28/29 | D36/37 |
| 1 × NVDX2 | 477.1 | 582.3 | NA | 430.1 |
| 2 × NVDX2 | 413.2 | 546.6 | NA | 203.9 |

TABLE 21
CD68 recruitment in non-ischemic legs.

| Non-ischemic limb | |
|---|---|
| Mean | SD |

| | | | | |
|---|---|---|---|---|
| CD3+ cells/mm² | D28/29 | D36/37 | D28/29 | D36/37 |
| 1 × NVDX2 | 404.0 | 402.8 | NA | 123.3 |
| 2 × NVDX2 | 375.9 | 393.8 | NA | 50.0 |

From Table 20 and Table 21, it was observed, in both treated limbs, a similarity in CD68+ cells recruitment at days 28/29 between the rat treated one time with NVDX2 and the rat treated two times with NVDX2: 477.1 CD68+ cells/mm² vs 413.2 CD68+ cells/mm² and 404.0 CD68+ cells/mm² vs 375.9 CD68+ cells/mm² for ischemic and non-ischemic limbs respectively. This similarity in terms of CD68+ cells/mm² was also observed at days 36/37 with 582.3±430.1 (1×NVDX2) vs 546.6±203.9 (2×NVDX2) and 582.3±430.1 (1×NVDX2) vs 546.6±203.9 (2×NVDX2) for ischemic and non-ischemic legs respectively.

This CD68 recruitment could be explained by a classical reaction against foreign bodies, without any difference between the two treatment groups (1×NVDX2 vs 2×NVDX2).

### 3. Conclusion and Discussion

The aim of this study was to assess the efficacy of a biological lyophilized and gamma-irradiated bandage, NVDX2 (from human origin), in the treatment of ischemic/hyperglycemic wounds in a Wistar rat model.

This acknowledged model of deep-thickness ischemic/hyperglycemic wound was selected as it is a very stringent model of hypoxic wound associated with impaired angiogenesis as can be found in diabetic patients. Indeed, in most cases, chronic wounds are the consequence of severe tissue ischemia, which is particularly common among patients with diabetes mellitus or with smokers. Ischemia has been shown to decrease fibroblast replication, collagen production, to increase collagen degradation and to decrease wound contraction (Hunt et al., Surg Gynecol Obstet 1972; Steinbrech et al., J Surg Res 1999; Yamanaka et al., J Dermatol Sci 2000; Alizadeh et al., Wound Repair and Regeneration. 2007).

In addition, hyperglycemia exponentially exacerbates the negative effects of ischemia on wound repair, especially on wound contraction and myofibroblast differentiation (Tobalem et al., Plast Reconstr Surg Glob Open 2015). Similarly, in vitro hypoxia suppresses myofibroblast differentiation (Modarressi et al., J Invest Dermatol 2010).

For this study with NVDX2, 13 male Wistar rats were injected intraperitoneally with [50 mg/kg] of STZ. The hyperglycemic status of animal was confirmed by a blood glucose over 9 mM and ischemia was induced by ligation of the femoral artery of one posterior limb before implantation of NVDX2.

The kinetic of wound closure, based on the macroscopic evaluation of the wounds, was measured on the wound's pictures. While the surface of the wounds remained constant during the 7-8 first days after NVDX2-treatment, wound healing associated with a reduction of the wound's surface was observed from days 13-16. A complete and irreversible wound closure of the non-ischemic limb was estimated between days 22 and 23 after NVDX2-treatment (1 or 2 NVDX2 applications). In the ischemic limb, a complete and irreversible wound closure was estimated at day 31 after NVDX2-treatment (1 or 2 NVDX2 applications).

Each wound was found to be completely healed with a complete epithelial layer at maximum days 36/37. In the both ischemic and non-ischemic limbs, a complete epithelial layer was observed at day 28 post-surgery and on day 29 post-surgery.

A macrophages infiltration was found in NVDX2 groups (1×NVDX2 and 2×NVDX2) from day 15 post-implantation till the total wound closure time illustrated at days 36/37. This macrophages recruitment, known as a typical reaction against foreign bodies, was associated with a transient T-lymphocytes recruitment, indicated an immune response against the implanted product which were found in the dermis up to day 37 in some cases. The T-lymphocytes peak of recruitment was observed at days 28/29 with a quite different profile according to one-time or two-times NVDX2 applications. This difference could be explained by the immune reaction against NVDX2 during the second application occurring 1 week after the first one. The elicitation of the immune response could be caused by this second NVDX2 application, leading to a classical graft rejection response.

These data show that NVDX2 demonstrated efficacy in stringent xenogeneic model of hyperglycemic and ischemic deep-thickness wound.

### Example 7: Comparison Analysis by Scanning Electron Microscopy of the Fresh (NVD003) and Freeze-Dried, Non-Irradiated (NVD0031yo) Biomaterials Obtained in Example 1 with the HA//β-TCP Particles

Samples of NVD003 and freeze-dried NVD003 (NVD0031yo) biomaterials were fixed in glutaraldehyde 2% during 2h followed by 3 washing in PBS (3×10 min). Samples were dehydrated in baths with increasing concentrations of ethanol (10%, 30%, 50%, 60%, 70%, 80% et 100%); during 15 minutes in each bath. Samples were dried using CPD critical soit dryer and mineralized with gold. Finally, samples were observed using a SEM-FEG JEOL7600F (JEOL®, Japan).

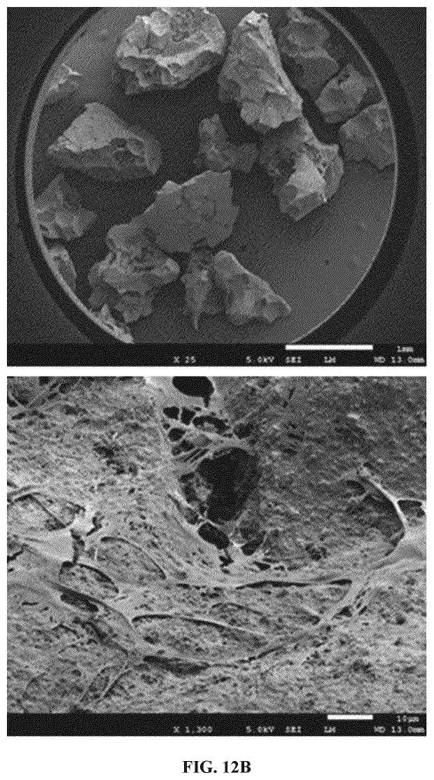

FIG. 12A shows the structure of the NVD003 biomaterial, whereas FIG. 12B shows the structure of the NVD0031yo biomaterial. The NVD0031yo has a similar microscopic structure as NVD003, although the average size of the particles is significantly lower, when observed with a ×25 zoom. In addition, both materials appear like a cohesive particle, wherein the cohesion would be performed by the extracellular matrix and the ceramic material. In the NVD003 biomaterial, all particles appear to be linked by the ECM. In contrast, the NVD0031yo biomaterial appears to be composed of groups of multiple particles linked by the ECM.

### Example 8: Impact of NVD003 and NVD0031yo on Genes Expression of Bone Marrow MSCs In Vitro

The objective of this study was to evaluate the capacity of NVD003 and NVD0031yo biomaterials to induce osteoblast differentiation.

### 1. Materials and Methods

Bone marrow mesenchymal stem cells (BMSC) were cultivated in basal medium (DMEM medium supplemented with 5% FBS) with or without 100 mg or 500 mg HA/bTCP; 100 mg or 500 mg NVD003; or 100 mg or 500 mg NVD0031yo. A positive control of osteoblast differentiation (MD) was performed by cultivating MSCs in osteoblast differentiation medium (DMEM medium supplemented with 5% FBS, BMP-2 (100 ng/mL), ascorbic acid (50 μg/mL) and β-glycerophosphate (10 mM)).

Treatments were carried out in 6-well plates and applied over 7 or 14 days in triplicates. At day 7 and day 14, cells were detached, frozen in FBS 10% DMSO. Supernatants were also collected during the medium refreshments and at the end of the treatments, stored at −20° C.

RNA extraction was performed on one out of the three replicates that were collected for each treatment for further analysis.

A gene expression profiling on 92 osteodifferentiation markers (using the Taqman® osteopanel array) was first performed in the positive and negative control samples. From this screening, 13 genes appeared to be more than 2-fold induced in the BMSCs in the presence of osteodifferentiation medium mostly at day 7 and less at day 14 (see Table 22, Table 23 and Table 24 below). The mRNA expression levels for each sample were normalized to the respective expression levels measured in the negative control.

### 2. Results

More globally, three genes' expression patterns have been identified. The first pattern included genes that are induced upon the treatment of both NVD003 and NVD0031yo biomaterials (Table 22). The second pattern included genes that are induced upon the treatment of NVD003 biomaterial but not NVD0031yo biomaterial (Table 23). Finally, the third pattern included genes that are induced upon the treatment of NVD0031yo biomaterial but not NVD003 biomaterial (Table 24).

TABLE 22
Genes induced upon the treatment of both NVD003 and
NVD003lyo biomaterials (MD: differentiation medium)

| | | HA/TCP- | NVD003 | NVD003lyo | NVD003 | NVD003lyo |
|---|---|---|---|---|---|---|
| | MD | 500 mg | 100 mg | 100 mg | 500 mg | 500 mg |
| MMP8 | 19.7 | 0.3 | 14.6 | 54.05 | 7.6 | 25.65 |
| MMP13 | 8.8 | 0.4 | 2.3 | 183.30 | 11.3 | 24.5 |
| COL11A1 | 2.3 | 0.3 | 6.8 | 2.45 | 4.7 | 0.75 |
| TGFB3 | 1.65 | 1.6 | 2.5 | 0.75 | 2.2 | 2.6 |
| BMP2 | 8.65 | 12.2 | 0.9 | 46.65 | 3.6 | 60.3 |
| BMP6 | 3.6 | 3.2 | 1.6 | 1.60 | 3.9 | 2.65 |
| MSX1 | 2.35 | 3.0 | 1.2 | 1.40 | 0.4 | 3.15 |
| COL4A4 | 1.05 | 0.4 | 36.5 | 1.95 | 94.9 | 0.7 |
| COL3A1 | 1.1 | 0.6 | 2.4 | 1.20 | 1.4 | 2.25 |
| COL18A1 | 0.5 | 1.7 | 2.2 | 1.00 | 1.3 | 2.35 |
| TWIST2 | 1.4 | 5.2 | 1.1 | 2.40 | 2 | 4.05 |
| VDR | 1.25 | 2.8 | 1.2 | 5.60 | 1.6 | 5.85 |
| BMP1 | 1 | 1.3 | 1.9 | 1.55 | 1.3 | 2.9 |
| COL5A1 | 1.45 | 0.5 | 1.5 | 2.00 | 1.1 | 1.9 |
| COL7A1 | 0.85 | 1.1 | 0.7 | 5.30 | 0.0 | 9.9 |

TABLE 23
Genes induced upon the treatment of NVD003
biomaterial but not NVD003lyo biomaterial

| | | HA/TCP- | NVD003 | NVD003lyo | NVD003 | NVD003lyo |
|---|---|---|---|---|---|---|
| | MD | 500 mg | 100 mg | 100 mg | 500 mg | 500 mg |
| IBSP | 24.85 | 0.3 | 1.7 | 0.20 | 6.6 | >LOD |
| IGF1 | 6.35 | 11.5 | 11.5 | 0.50 | 1.3 | >LOD |
| IGF2 | 2.85 | 0.5 | 15.0 | 1.25 | 6.3 | 0.9 |
| BMP5 | 6.85 | >LOD | 9.6 | 2.10 | 6.1 | >LOD |
| ALPL | 1.85 | 0.3 | 6.4 | 1.15 | 10.1 | 2.25 |
| MGP | 0.95 | — | 4.7 | 0.10 | 1.2 | 0.1 |
| TGFB2 | 1.6 | — | 0.9 | 0.75 | 2.5 | 0.8 |
| FGFR2 | 1.75 | — | 2.0 | 0.30 | 0.1 | 1.1 |

TABLE 24
Genes induced upon the treatment of NVD003lyo
biomaterial but not NVD003 biomaterial

| | | HA/TCP- | NVD003 | NVD003lyo | NVD003 | NVD003lyo |
|---|---|---|---|---|---|---|
| | MD | 500 mg | 100 mg | 100 mg | 500 mg | 500 mg |
| ENAM | 2.4 | >LOD | >LOD | 0.00 | >LOD | 4.5 |
| COMP | 8.25 | 1.0 | 1.2 | 6.80 | 0.0 | 1.9 |
| COL10A1 | 0.85 | 0.7 | 0.8 | 107.40 | 1.4 | 48.65 |
| SPP1 | 0.85 | 4.2 | 0.0 | 0.80 | 0.1 | 2.7 |
| SMAD1 | 1.35 | 1.1 | 1.1 | 1.20 | 0.8 | 2.3 |
| VEGFA | 0.8 | 2.6 | 0.5 | 1.85 | 1.3 | 1.9 |
| MINPP1 | 1.3 | 1.2 | 1.0 | 1.35 | 1.1 | 1.85 |

These results confirm that the genes expression's profiles induced by NVD003 and NVD0031yo biomaterials are not equivalent.

### Example 9: Impact of the Freeze-Drying of NVD003 Biomaterial on In Vivo Bioactivity

### 1. Materials and Methods

Implantation in a femoral critical-sized bone-defect of (NVD003) was performed on 56 male nude rats whom only 42 enrolled in the study. Analyses performed: histology, μCT scans and q-RT-PCR (rat primers).

At 1-month post-implantation, the total RNAs was extracted from explants using the Qiazol lysis reagent (Qiagen®, Hilden, Germany) and a Precellys homogenizer (Bertin Instruments®, Montigny-le-Bretonneux, France). RNAs were purified using Rneasy mini kit (Qiagen®, Hilden, Germany) with an additional on column DNase digestion according to the manufacturer's instruction. Quality and quantity of RNAs were determined using a spectrophotometer (Spectramax 190, Molecular Devices®, California, USA). cDNA was synthesized from 0.5 μg of total RNA using RT² RNA first strand kit (Qiagen®, Hilden, Germany) for osteogenic and angiogenic genes expression profiles commercially available PCR arrays (Human RT² Profiler Assay—Angiogenesis; Human RT² Profiler Assay—Osteogenesis, Qiagen®). The ABI Quantstudio 5 system (Applied Biosystems®) and SYBR Green ROX Mastermix (Qiagen®, Hilden, Germany) were used for detection of the amplification product. Quantification was obtained according to the ΔΔCT method. The final result of each sample was normalized to the means of expression level of three housekeeping genes (ACTB, B2M and GAPDH).

The osteogenic genes expression was compared between the explants obtained from biomaterial of the invention at 1-month post-implantation. Eighty-four osteogenic genes were then tested for the explant.

For quantification of miRNA expression, 50 ng RNA was reverse transcribed into cDNA using qScript miRNA cDNA Synthesis kit (Quanta Biosciences®), and qRT-PCR was conducted in triplicate using Perfecta SYBR Green Super Mix (Quanta Biosciences®). Thermal cycling was performed on an Applied Biosystems 7900 HT detection system (Applied Biosystems®). Data was normalized to miR-16-5p and U6 small nuclear RNA using the Delta-Delta Ct method.

Grafts were explanted at 1-month post-transplantation for osteo-induction at molecular level. Biomarkers of osteo-induction were the following: VEGFA, VEGFB, IGF-1; SMAD2, SMAD3, SMAD4, SMAD5; ITGAV, ITGB1, VCAM1.

### 2. Results

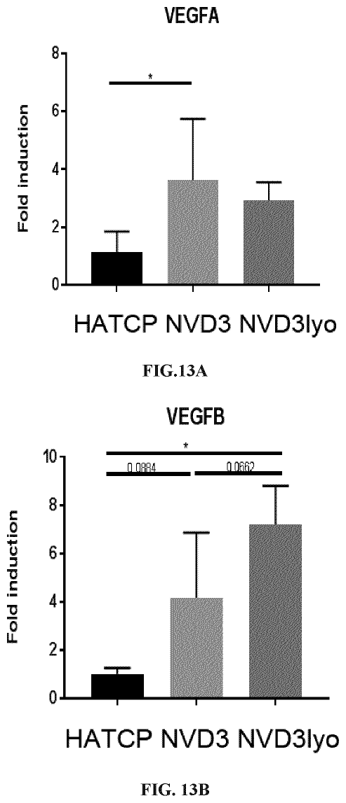

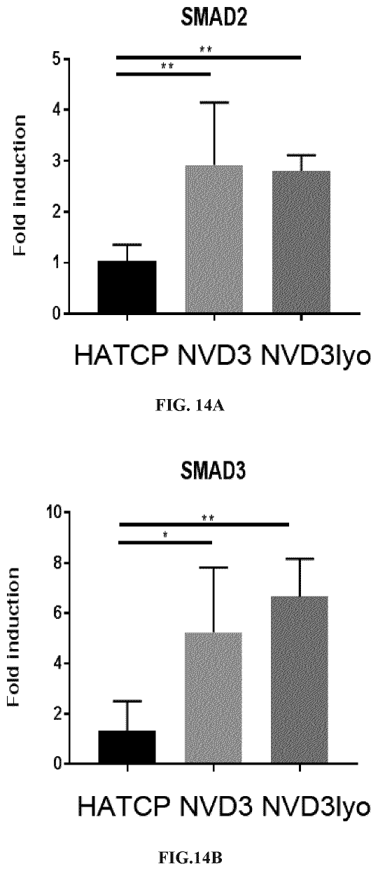

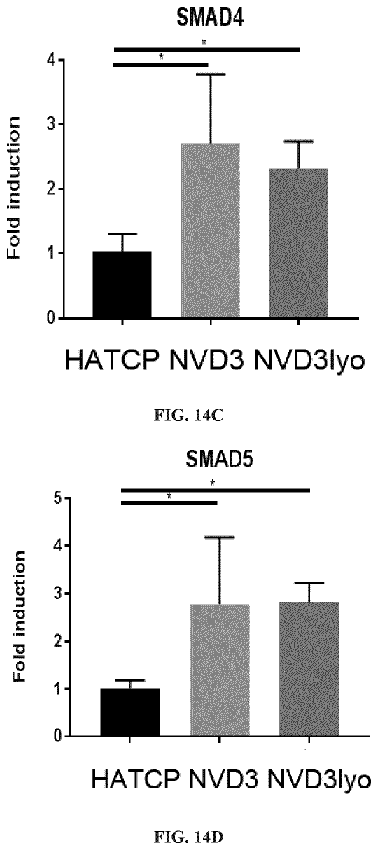

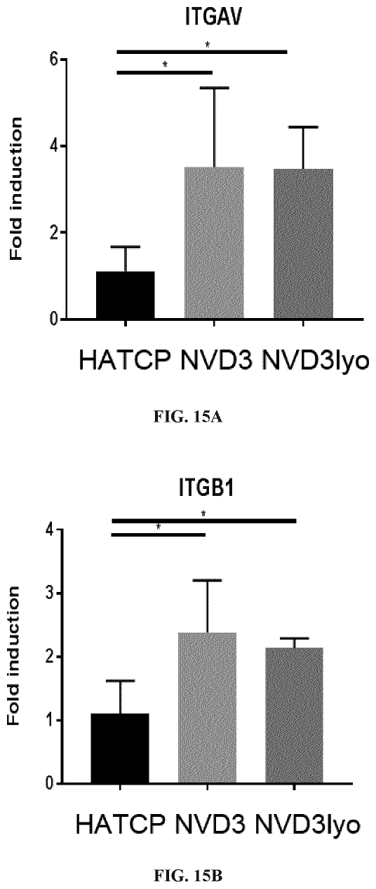

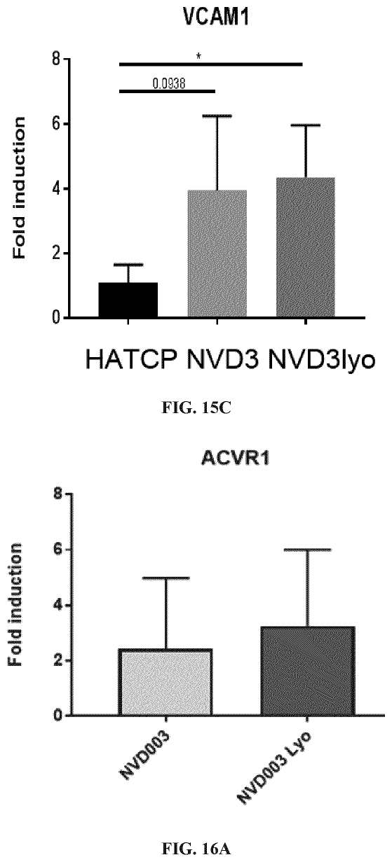

A globally similar profile of osteo-induction was demonstrated for fresh NVD003 and NVD0031yo at molecular level with a superiority in comparison to HA/βTCP particles alone when VEGF-A (FIG. 13A), SMAD-2 to SMAD-5 (FIG. 14A-D), ITGAV, ITGB-1 and VCAM-1 (FIG. 15A-C) were assayed. On the other hand, the relative amount of VEGF-B in the NVD0031yo biomaterial was significantly increased as compared to the NVD003 biomaterial (FIG. 13B), whereas the relative amount of IGF-1 was significantly decreased in the NVD0031yo biomaterial as compared to the NVD003 biomaterial.

The osteoinduction was confirmed histologically by a staining of alcian blue for endochondral ossification (*).

### Example 10: Impact of the Freeze-Drying of Fresh NVD003 Biomaterial on Osteogenic Genes Stability and the miR Cellular Content as Compared to Fresh NVD003 Biomaterial

### 1. Osteogenic Genes Stability

Total RNAs were extracted as disclosed in example 3.

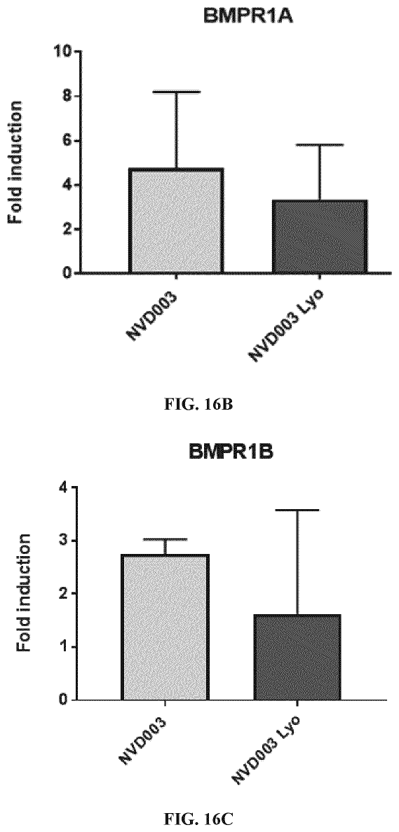

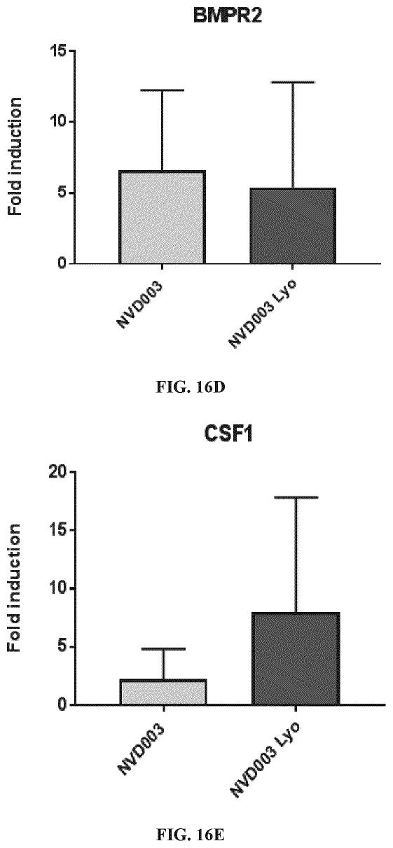

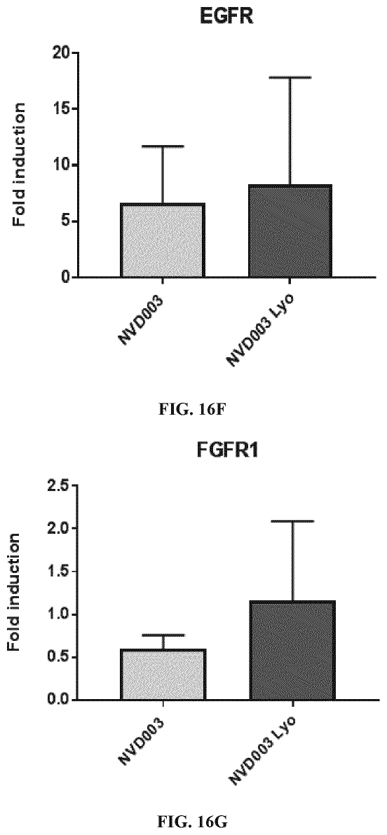

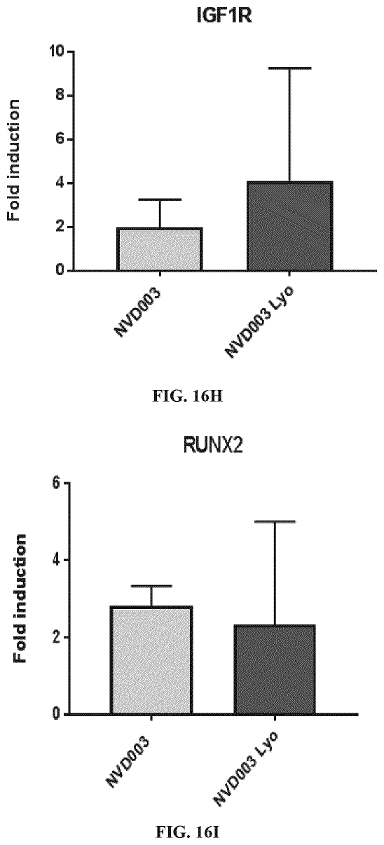

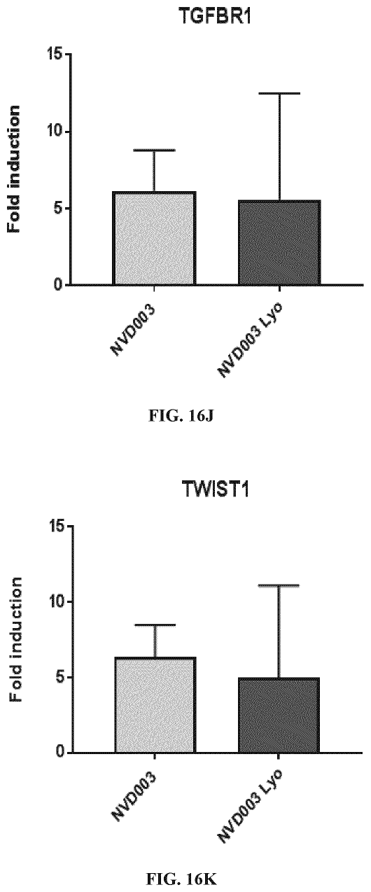

When the biomarker for skeletal development was assessed in the NVD003 and the NVD0031yo biomaterials, the levels of these biomarker were globally altered (FIG. 16A-K). For example, the average relative amount of factors ACVR-1, CSF-1, EGFR, FGFR-1, IGF-1R was globally increased in the NVD0031yo biomaterial as compared to the NVD003 biomaterial. On the other hand, the average relative amount of factors BMPR-1A, BMPR-1B, BMPR-2, RUNX2, TGFBR-2 and TWIST-1 was globally decreased in the NVD0031yo biomaterial as compared to the NVD003 biomaterial These data suggest that the freeze-drying of the fresh biomaterial NVD003 had a global impact with respect to the factors' content expressed by the ASCs.

### 2. miRNAs Content

### 2.1. RNAs Extraction

mRNAs isolation was performed from biopsies of NVD003, freeze-dried NVD003 (NVD0031yo) and freeze-dried/irradiated NVD003 (NVDX3). mRNA was extracted using miRNeasy kit Mastermix (Qiagen®, Hilden, Germany) following the manufacturer's protocol. RNA concentration was determined by Nanodrop (ThermoFisher®, Waltham, Massachusetts, USA).

2.2. Quantitative RT-PCR (gRT-PCR)

Quantification of miRNAs expression was performed as disclosed in example 3.

### 2.3. Exosomes Purification

Exosomes have been isolated by differential centrifugation from culture medium whereby larger “contaminants” are first excluded by pelleting out through increasing speeds of centrifugation before exosomes, small extracellular vesicles and even protein aggregates are pelleted at very high speeds (˜100,000×g).

### 2.4. Results

TABLE 25
identification of miRNAs from purified exosomes

| | | | |
|---|---|---|---|
| hsa-let-7a-5p | hsa-let-7i-5p | hsa-miR-660-5p | hsa-miR-6832-3p |
| hsa-let-7b-5p | hsa-miR-409-3p | hsa-miR-664a-3p | hsa-miR-146a-5p |
| hsa-miR-24-3p | hsa-miR-210-3p | hsa-miR-185-5p | hsa-miR-16-2-3p |
| hsa-miR-21-5p | hsa-miR-199a-3p | hsa-miR-3651 | hsa-miR-181b-5p |
| hsa-let-7f-5p | hsa-miR-30a-3p | hsa-miR-495-3p | hsa-miR-26a-2-3p |
| hsa-miR-574-3p | hsa-miR-320b | hsa-let-7a-3p | hsa-miR-376a-3p |
| hsa-miR-23b-3p | hsa-miR-193a-5p | hsa-miR-28-5p | hsa-miR-539-5p |
| hsa-miR-1273g-3p | hsa-miR-382-5p | hsa-miR-99b-3p | hsa-miR-708-5p |
| hsa-miR-25-3p | hsa-miR-423-3p | hsa-miR-103a-3p | hsa-miR-98-3p |
| hsa-miR-199a-5p | hsa-miR-17-5p | hsa-miR-19a-3p | hsa-miR-1237-5p |
| hsa-miR-196a-5p | hsa-miR-19b-3p | hsa-miR-126-5p | hsa-miR-223-3p |
| hsa-miR-214-3p | hsa-miR-92b-3p | hsa-miR-2053 | hsa-miR-532-5p |
| hsa-miR-125a-5p | hsa-miR-320a | hsa-miR-29b-1-5p | hsa-miR-542-3p |
| hsa-miR-221-3p | hsa-miR-3074-5p | hsa-miR-3648 | hsa-miR-663a |
| hsa-miR-222-3p | hsa-miR-376c-3p | hsa-miR-374a-3p | hsa-miR-101-3p |
| hsa-let-7e-5p | hsa-let-7b-3p | hsa-miR-454-3p | hsa-miR-143-3p |
| hsa-miR-191-5p | hsa-miR-625-3p | hsa-miR-532-3p | hsa-miR-21-3p |
| hsa-miR-199b-3p | hsa-miR-99b-5p | hsa-miR-136-3p | hsa-miR-224-5p |
| hsa-miR-342-3p | hsa-miR-34a-5p | hsa-miR-361-3p | hsa-miR-26a-5p |
| hsa-miR-23a-3p | hsa-miR-5096 | hsa-miR-1246 | hsa-miR-27a-5p |
| hsa-miR-424-3p | hsa-miR-30e-3p | hsa-miR-130b-3p | hsa-miR-324-5p |
| hsa-miR-28-3p | hsa-miR-22-3p | hsa-miR-134-5p | hsa-miR-340-3p |
| hsa-let-7g-5p | hsa-miR-151a-3p | hsa-miR-154-5p | hsa-miR-379-5p |
| hsa-miR-92a-3p | hsa-miR-186-5p | hsa-miR-34a-3p | hsa-miR-409-5p |
| hsa-miR-424-5p | hsa-miR-193b-5p | hsa-miR-576-5p | hsa-miR-543 |
| hsa-let-7d-3p | hsa-miR-328-3p | hsa-miR-874-3p | hsa-miR-5787 |
| hsa-miR-4454 | hsa-miR-4449 | hsa-miR-100-5p | hsa-miR-6089 |
| hsa-miR-146b-5p | hsa-miR-27a-3p | hsa-miR-103b | hsa-miR-127-3p |
| hsa-miR-423-5p | hsa-miR-30c-5p | hsa-miR-1273a | hsa-miR-149-5p |
| hsa-miR-29a-3p | hsa-miR-494-3p | hsa-miR-1306-5p | hsa-miR-181c-5p |
| hsa-miR-574-5p | hsa-miR-98-5p | hsa-miR-138-5p | hsa-miR-193b-3p |
| hsa-miR-199b-5p | hsa-miR-10a-5p | hsa-miR-15b-3p | hsa-miR-222-5p |
| hsa-miR-125b-5p | hsa-miR-29b-3p | hsa-miR-26b-3p | hsa-miR-3613-5p |
| hsa-miR-3184-3p | hsa-miR-374b-5p | hsa-miR-10b-5p | hsa-miR-365b-3p |
| hsa-let-7c-5p | hsa-miR-335-5p | hsa-miR-22-5p | hsa-miR-3960 |
| hsa-miR-337-3p | hsa-miR-374c-3p | hsa-miR-3613-3p | hsa-miR-485-3p |
| hsa-let-7d-5p | hsa-miR-425-5p | hsa-miR-655-3p | hsa-miR-6087 |
| hsa-miR-145-5p | hsa-miR-181a-5p | hsa-miR-7-1-3p | hsa-miR-92a-1-5p |
| hsa-miR-93-5p | hsa-miR-196b-5p | hsa-miR-23a-5p | hsa-miR-4668-5p |
| hsa-miR-619-5p | hsa-let-7f-1-3p | hsa-miR-24-2-5p | hsa-miR-3605-3p |
| hsa-miR-130a-3p | | | |

TABLE 26
identification of cellular miRNAs

| | | | |
|---|---|---|---|
| hsa-let-7a-5p | hsa-miR-3653-5p | hsa-miR-98-5p | hsa-miR-28-5p |
| hsa-let-7b-5p | hsa-miR-342-3p | hsa-miR-664a-3p | hsa-miR-10a-5p |
| hsa-miR-24-3p | hsa-miR-28-3p | hsa-miR-92b-3p | hsa-miR-151a-3p |
| hsa-let-7f-5p | hsa-miR-23b-3p | hsa-miR-4449 | hsa-miR-30e-3p |
| hsa-miR-199a-5p | hsa-let-7c-5p | hsa-miR-320a | hsa-miR-324-5p |
| hsa-miR-214-3p | hsa-miR-222-3p | hsa-miR-181a-5p | hsa-miR-495-3p |
| hsa-miR-3607-5p | hsa-miR-29a-3p | hsa-miR-3651 | hsa-miR-576-5p |
| hsa-miR-125a-5p | hsa-miR-92a-3p | hsa-miR-185-5p | hsa-miR-625-3p |
| hsa-miR-199b-3p | hsa-miR-30a-3p | hsa-miR-664b-5p | hsa-miR-671-5p |
| hsa-miR-125b-5p | hsa-miR-424-3p | hsa-miR-196b-5p | hsa-miR-1271-5p |
| hsa-miR-21-5p | hsa-miR-423-3p | hsa-miR-27a-3p | hsa-miR-186-5p |
| hsa-let-7e-5p | hsa-miR-34a-5p | hsa-miR-29b-3p | hsa-miR-23a-5p |
| hsa-let-7i-5p | hsa-miR-424-5p | hsa-miR-664b-3p | hsa-miR-3613-5p |
| hsa-let-7g-5p | hsa-miR-145-5p | hsa-miR-99b-5p | hsa-miR-376c-3p |
| hsa-miR-574-3p | hsa-miR-328-3p | hsa-miR-103a-3p | hsa-miR-409-3p |
| hsa-miR-574-5p | hsa-miR-3074-5p | hsa-miR-6516-3p | hsa-miR-4461 |
| hsa-miR-191-5p | hsa-let-7d-3p | hsa-miR-22-3p | hsa-miR-454-3p |
| hsa-miR-196a-5p | hsa-miR-93-5p | hsa-miR-26a-5p | hsa-miR-6724-5p |
| hsa-miR-221-3p | hsa-miR-23a-3p | hsa-miR-103b | hsa-let-7b-3p |
| hsa-miR-25-3p | hsa-miR-19b-3p | hsa-miR-1291 | hsa-miR-190a-5p |
| hsa-miR-423-5p | hsa-miR-146b-5p | hsa-miR-425-5p | hsa-miR-26b-3p |
| hsa-miR-210-3p | hsa-miR-320b | hsa-miR-22-5p | hsa-miR-3609 |
| hsa-miR-1273g-3p | hsa-miR-337-3p | hsa-miR-374c-3p | hsa-miR-411-5p |
| hsa-let-7d-5p | hsa-miR-17-5p | hsa-let-7i-3p | hsa-miR-425-3p |
| hsa-miR-199b-5p | hsa-miR-130a-3p | hsa-miR-374b-5p | hsa-miR-4485-3p |
| hsa-miR-199a-3p | hsa-miR-193b-5p | hsa-miR-455-3p | hsa-miR-30c-5p |
| hsa-miR-193a-5p | hsa-miR-382-5p | hsa-miR-532-3p | hsa-miR-619-5p |
| hsa-miR-3184-3p | | | |

TABLE 27
Level of exosomal miRNAs in the NVD003
biomaterial as compared to a 2D culture

| miRNA | logFC | FC | logCPM | PValue | FDR |
|---|---|---|---|---|---|
| hsa-miR-210-3p | 3.8329 | 14.2502 | 11.0073987 | 5.86E−07 | 0.00016341 |
| hsa-miR-3687 | −4.2961 | −19.6462 | 10.1302138 | 4.12E−07 | 0.00016341 |
| hsa-miR-4454 | 3.3561 | 10.2401 | 12.1672668 | 1.14E−06 | 0.00021296 |
| hsa-miR-3653-5p | −4.4413 | −21.7264 | 9.79288414 | 3.76E−06 | 0.00052506 |
| hsa-miR-664b-5p | −4.2939 | −19.6152 | 9.71861657 | 9.08E−06 | 0.00101337 |
| hsa-miR-619-5p | 2.7545 | 6.7485 | 11.3607639 | 0.00016339 | 0.0130247 |
| hsa-miR-664b-3p | −4.9289 | −30.4622 | 10.1914599 | 0.00014999 | 0.0130247 |
| hsa-miR-3613-3p | −3.0938 | −8.5377 | 10.8210184 | 0.00105987 | 0.07392561 |
| hsa-miR-409-3p | −1.5541 | −2.9365 | 12.8917856 | 0.00139377 | 0.08641401 |
| hsa-miR-3607-5p | −2.9445 | −7.6981 | 9.62722759 | 0.00178514 | 0.0996108 |
| hsa-miR-1246 | 5.1208 | 34.7953 | 8.66637826 | 0.00292854 | 0.14855699 |
| hsa-miR-3609 | −4.9838 | −31.6427 | 8.47677701 | 0.00415237 | 0.19308534 |
| hsa-miR-222-3p | 1.4242 | 2.6837 | 13.2032086 | 0.0053805 | 0.23094758 |
| hsa-miR-181a-5p | 2.3137 | 4.9717 | 9.35881731 | 0.00612351 | 0.24406574 |
| hsa-miR-6832-3p | 4.7957 | 27.7766 | 8.50241186 | 0.01014361 | 0.37734231 |
| hsa-miR-335-5p | 3.3247 | 10.0198 | 9.35699942 | 0.01119602 | 0.39046125 |
| hsa-let-7a-3p | 5.8907 | 59.3317 | 9.07299564 | 0.01316075 | 0.40798328 |
| hsa-miR-28-3p | 1.2784 | 2.4258 | 12.7300164 | 0.01298217 | 0.40798328 |
| hsa-miR-663a | −3.4819 | −11.1732 | 10.9716677 | 0.01441164 | 0.42324701 |
| hsa-miR-19a-3p | −1.6552 | −3.1497 | 10.3578129 | 0.01740016 | 0.46234701 |
| hsa-miR-3651 | −2.7857 | −6.8957 | 11.5575736 | 0.01725774 | 0.46234701 |
| hsa-miR-125a-5p | 1.2160 | 2.3231 | 13.546949 | 0.0201696 | 0.51157428 |
| hsa-miR-374c-3p | 2.1030 | 4.2963 | 9.5512217 | 0.02120255 | 0.51439235 |
| hsa-miR-4668-5p | −1.6714 | −3.1853 | 10.651278 | 0.02283107 | 0.53082232 |
| hsa-miR-301a-3p | −2.8147 | −7.0361 | 8.96350949 | 0.02430933 | 0.54258424 |
| hsa-miR-664a-3p | −1.7910 | −3.4606 | 10.6389527 | 0.02619156 | 0.56211116 |
| hsa-miR-485-5p | −2.0114 | −4.0317 | 9.0528546 | 0.03141568 | 0.64925748 |
| hsa-miR-382-5p | −1.0946 | −2.1355 | 12.0209678 | 0.03304208 | 0.65848152 |
| hsa-miR-4449 | −1.5489 | −2.9260 | 11.4255575 | 0.03601783 | 0.68391665 |
| hsa-miR-138-5p | −1.5886 | −3.0076 | 9.89864899 | 0.03676971 | 0.68391665 |
| hsa-miR-181c-5p | 4.1112 | 17.2823 | 8.20638387 | 0.04528724 | 0.81517026 |
| hsa-miR-374b-5p | 1.7817 | 3.4383 | 9.69392048 | 0.04793018 | 0.83578254 |

TABLE 28
Level of cellular miRNAs in the NVD003
biomaterial as compared to a 2D culture

| miRNA | logFC | FC | logCPM | PValue | FDR |
|---|---|---|---|---|---|
| hsa-miR-210-3p | 6.4907 | 89.9288 | 12.5959151 | 3.65E−17 | 1.29E−14 |
| hsa-miR-4485-3p | −3.7281 | −13.2516 | 11.4563949 | 3.29E−09 | 5.82E−07 |
| hsa-miR-409-3p | −3.3163 | −9.9616 | 11.3106568 | 3.00E−06 | 0.00035375 |
| hsa-miR-382-5p | −1.6105 | −3.0536 | 11.8932813 | 0.00020693 | 0.01831299 |
| hsa-miR-24-3p | 0.9484 | 1.9298 | 16.020164 | 0.00036741 | 0.0260129 |
| hsa-miR-93-5p | −1.5223 | −2.8725 | 12.3200694 | 0.00054198 | 0.03197659 |
| hsa-let-7i-5p | 1.1389 | 2.2021 | 13.7506601 | 0.00119986 | 0.06067875 |
| hsa-miR-3074-5p | 1.2618 | 2.3980 | 11.4482089 | 0.00741164 | 0.32796499 |
| hsa-let-7c-5p | −0.9293 | −1.9044 | 13.0382042 | 0.01316331 | 0.47049582 |
| hsa-miR-154-5p | −2.0724 | −4.2061 | 10.1415627 | 0.01329084 | 0.47049582 |
| hsa-miR-6723-5p | −4.7630 | −27.1540 | 9.31652789 | 0.01570223 | 0.50532623 |
| hsa-miR-671-5p | 4.2840 | 19.4815 | 9.3994702 | 0.03498742 | 1 |

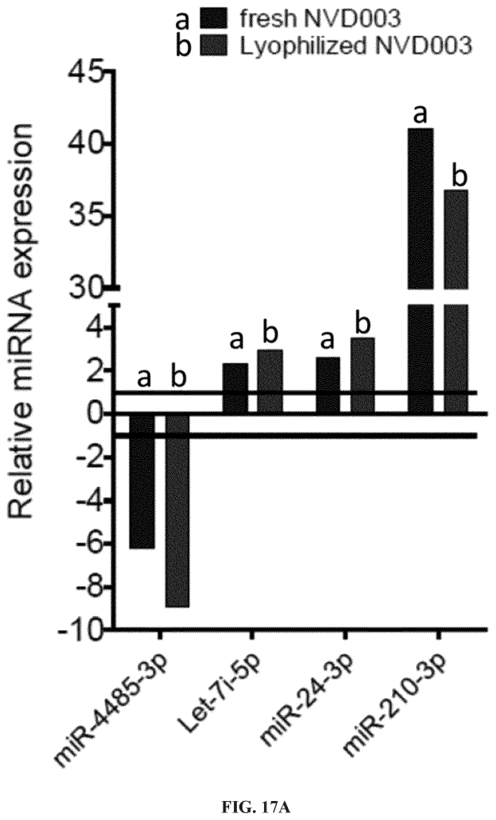

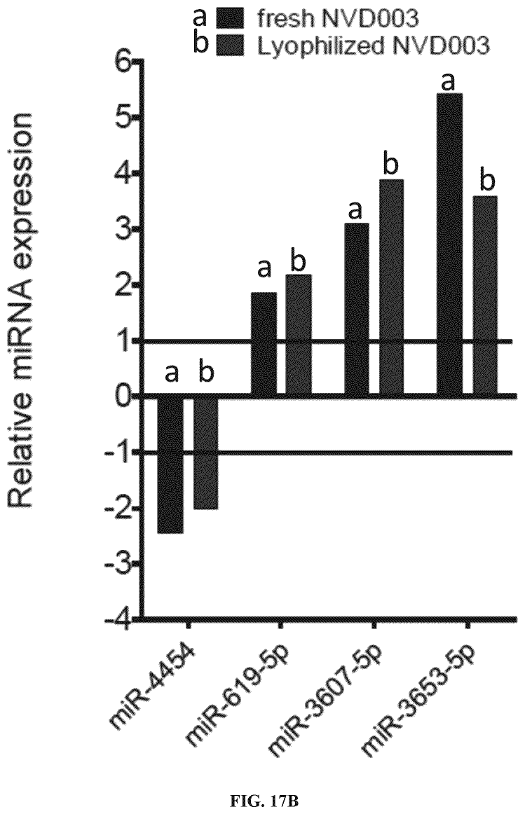

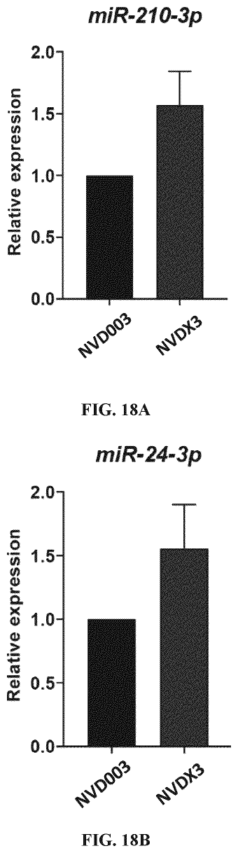

Comparison between the NVD003 and the NVDX3 biomaterials showed that the cellular miRNA content was globally altered, with either an increase in the average amount of individual miRNAs or a decrease in the average amount of individual miRNAs (FIG. 17A-B and FIG. 18A-B). These data suggest that the freeze-drying of the fresh NVD003 biomaterial had a global impact with respect to the miRNAs cellular and exosomal content synthesized and secreted, respectively, by the ASCs.

### Example 11: Impact of Freeze-Drying and Gamma-Irradiation on NVD003 on Osteogenic Genes Stability and Cellular miR Profiles Between Fresh NVD003 and Freeze-Dried NVD003 (NVD0031yo)

Biopsies of lyophilized NVD003 derived from 3 donors were gamma-irradiated in 4 different conditions (dose (12 kGy vs 25 kGy) and temperature (20° C. vs −80° C.)) and compared to non-irradiated tissues in terms of growth factors and mRNA expression. samples of about 350 mg of NVD0031yo biomaterial from each donor was weighted and placed in Glass bottles. Three samples were used as negative control, non-irradiated samples, and 12 samples were sent to Sterigenics for irradiation. Negative control samples were sent also to Sterigenics and were stored in the same conditions as the irradiated samples.

After the sterilization procedure, samples were processed for protein extraction and growth factors quantification by ELISA as well as for mRNA expression by q-RT-PCR and cellular miR-210-3p expression.

Proteins were extracted from non-irradiated and irradiated samples in Guanidine HCL [4 M], Benzamidine [5 mM], N-ethylmaleimide [10 mM], PMSF [1 mM] at 4° C. during 24h, then addition of tris HCL [50 mM] at pH 7.4 for 5 h (all from Sigma-Aldrich®, Saint Louis, USA) and purified through desalting columns (PD10 de GE Healthcare®, Chicago, USA). Quantification of growth factors VEGF, IGF1, SDF 1a, OPG content was performed by ELISA (Human VEGF quantikine ELISA Kit, Human SDF 1a quantikine ELISA Kit, Human IGF1 quantikine ELISA Kit, OPG Duo Set Elisa, R&D Systems®, Minneapolis, Minnesota, USA).

Total RNAs were extracted and quantified as disclosed in example 3.

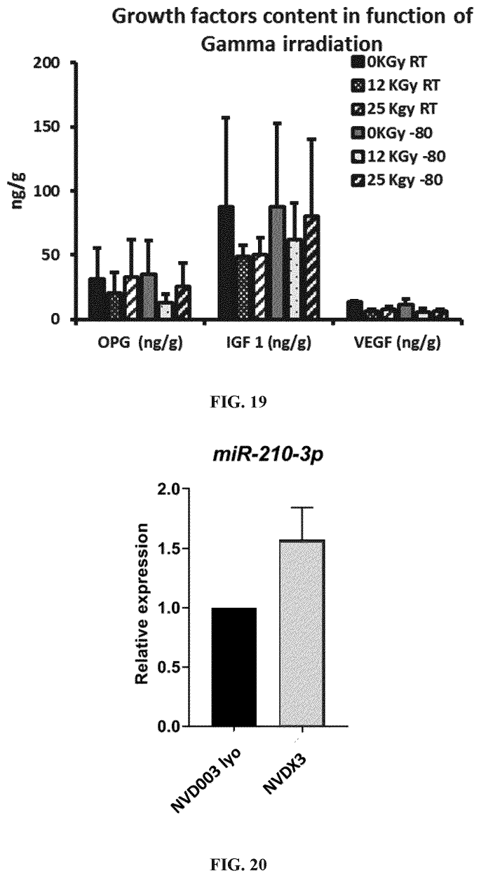

The OPG, IGF-1 and VEGF factors were globally preserved upon irradiation at the 2 tested doses (12 kGy and 25 kGy) either at room temperature or at −80° C. (FIG. 19).

Similarly, irradiation of the biomaterial had no negative impact with respect to the content of hsa-miR-210-3p (FIG. 20).

Although gamma-irradiation is known to have significant negative impact on the structure of polypeptides and nucleic acids in general, the experimental data suggest that the both the factors' content (polypeptides) and the miRNAs' cellular content from the fresh NVD003 biomaterial appear to be preserved from degradation upon gamma-irradiation.

### Example 12: In Vitro Evaluation of the Inhibition of Osteoclastogenesis

### 1. Impact of Lypholization and Gamma Irradiation on NVD003 on the Inhibition of Osteoclasts Maturation and Activity

### 1.1. Impact of NVDX3 on the Differentiation of Osteoclasts Precursors

Human CD14+ monocytes were isolated from peripheral blood of healthy volunteers, obtained in agreement with the “Etablissement Frangais du Sang”.

Following isolation of peripheral blood mononuclear cells, freshly isolated precursors were seeded and incubated for 2 hours (minimum time for cell attachment) in medium supplemented with 1% FBS, 25 ng/mL human MCSF+/−100 ng/mL human RANKL in 24-well culture plates. NVD003 (n=3 donors), HA/β-TCP (n=4 different batches) and NVDX3 (n=3 donors) at 5, 20 and 100 mg/well were added in transwells. Medium was changed at day 4.

A TRAP staining was performed after 5 at 6 days (depending on the donor of CD14+ cells), when multinucleated cells were present. The number of TRAP-positive cells containing more than three nuclei was determined in each well.

### 1.2. Impact of NVDX3 on the Mature Osteoclasts (Cytotoxicity)

Osteoclast precursor cells were isolated from the peripheral blood. Following isolation of peripheral blood mononuclear cells by Ficoll-Hypaque centrifugation, monocytes (CD14+ cells) were sorted (MACS®, MiltenyiBiotec). Freshly isolated precursors were differentiated into osteoclasts in the presence of M-CSF and RANKL (“plus RANKL” control) for 5 to 6 days (depending on the donor of CD14+ cells). Cells in medium without RANKL served as a negative control (“no RANKL” control). When the multinucleated cells were observed in the positive control, NVD003 (n=3 donors), HA/βTCP (n=4 different batches) and NVDX3 (n=3 donors) at 5, 20 and 100 mg/well were added in transwells.

48 hours after the addition of NVD003, HA/β-TCP or NVDX3, a TRAP staining was performed. The number of TRAP-positive cells containing more than three nuclei was determined in each well.

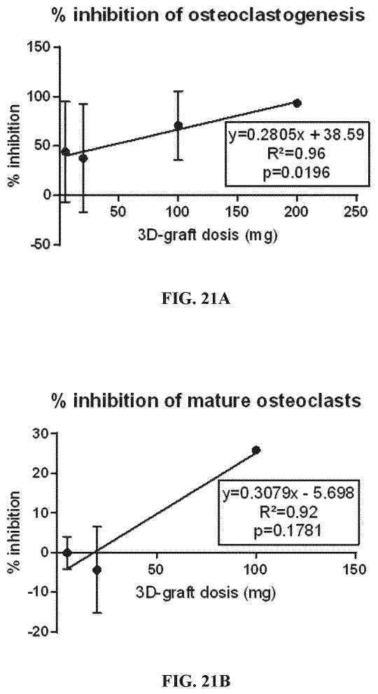

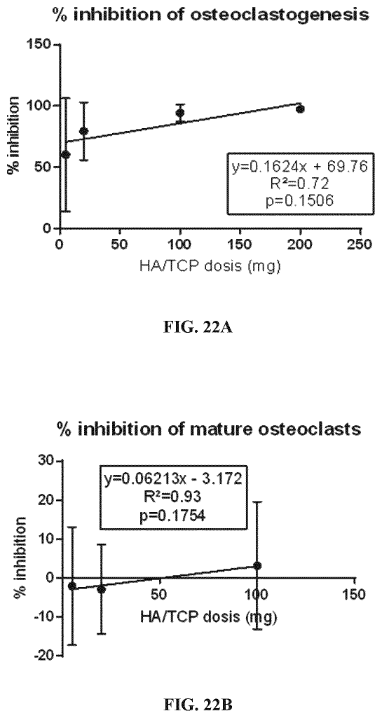

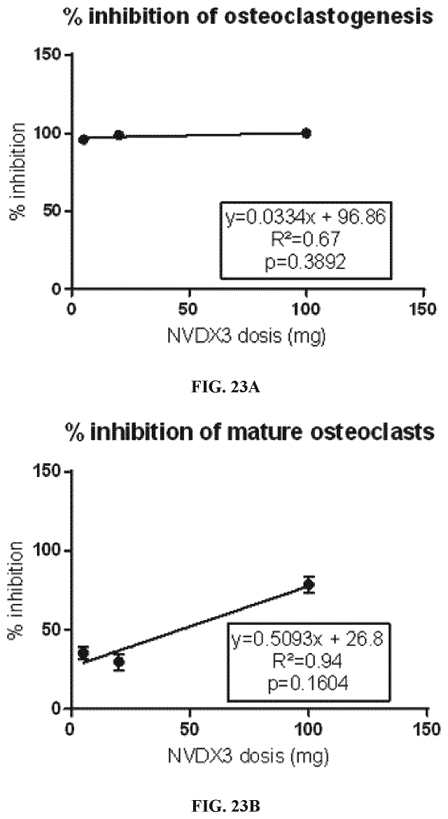

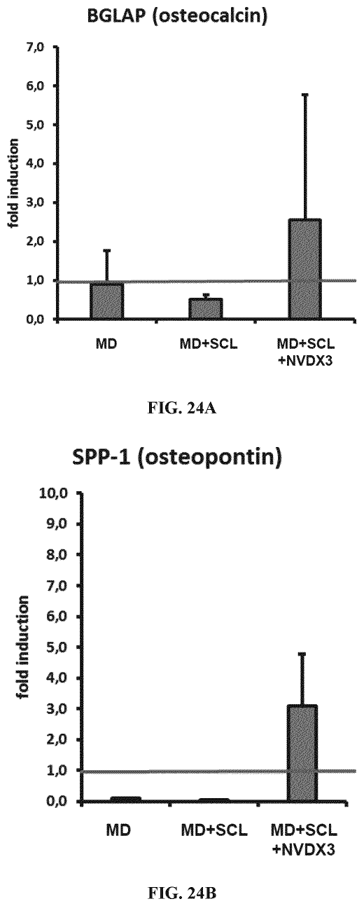

The dose-response in terms of inhibition of osteoclasts maturation and inhibition of osteoclasts activity were maintained between fresh NVD003 (FIG. 21A-B) and lyophilized/irradiated NVD003 (NVDX3; FIG. 23A-B) with a significant better inhibition of matured osteoclast (the functional test assays the impact of the biomaterials onto osteoclasts viability) in comparison to HA/β-TCP particles alone FIG. 22A-B). No significant difference of inhibition of osteoclastogenesis and mature osteoclasts was found when comparing each product by doses.

### 2. Impact of Lyophilization and Gamma-Irradiation on NVD003 on the Promotion of Osteogenesis

### 2.1. Counteracting the Negative Effect of Sclerotin

Adipose stem cells (ASCs) at passage 4 were placed in 12 wells plates for 2 days before incubation in osteo-differentiation medium (MD=positive control), MD+sclerostin (SCL) 100 mg/ml or MD+sclerostin (SCL) 100 mg/ml+NVDX3 100 mg/well for 10 days with medium change after 5 days. In addition, cells were placed in proliferation medium (MP) as negative control.

After 10 days of culture, cells were placed in Qiazol lysis reagent (Qiagen®, Hilden, Germany) for RNA isolation, extraction and quantification as disclosed in example 3.

In another experiment, adipose stem cells at passage 4 were placed in 12 wells in osteo-differentiation medium at 0.5% HPL (MD=positive control), M D et 0.5% HPL+sclerostin (SCL) 10 or 100 mg/ml or MD at 0.5% HPL+sclerostin (SCL) 10 or 100 mg/ml+NVDX3 400 mg/well for 11 days. In addition, cells were placed in osteo-differentiation medium at 5% HPL. Viability was followed at 9 days during 48h by RealTime Glo MT Cell Viability assay (Promega® G9711). Experiments were performed in duplicate.

The miRNAs content in NVDX3 (after lyophilization and gamma-irradiation) demonstrated in vitro promotion of osteogenesis of mesenchymal stem cells (derived from adipose stem cells).

As shown in FIG. 24A-B, the NVDX3 biomaterial counteracts the negative effect of sclerotin with respect to the osteogenic properties of osteocalcin (BGLAP) and osteopontin (SPP-1).

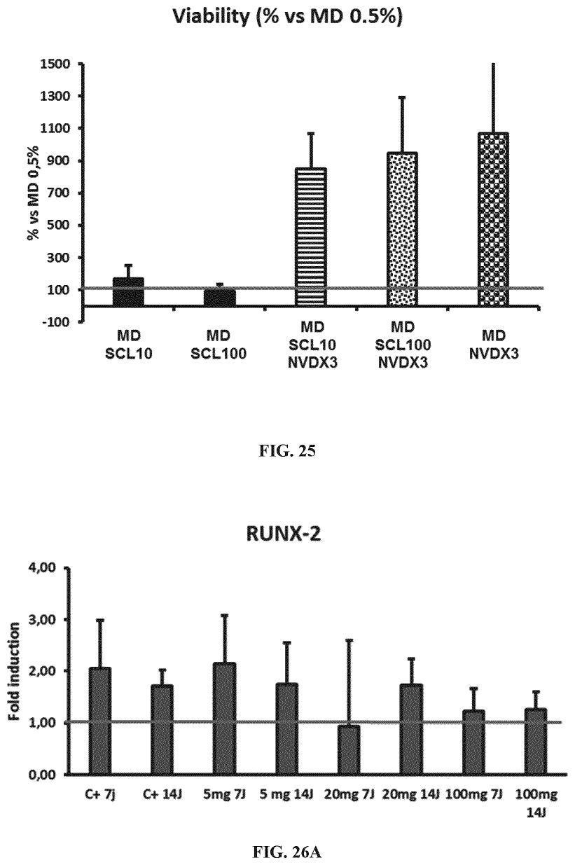

In addition, NVDX3 (after lyophilization and irradiation) demonstrated the capacity to antagonize the effect of sclerostin on adipose stem cells in term of viability (FIG. 25).

### 2.2. Impact of NVDX3 on the Differentiation of Osteoblasts Precursors

The osteo-inductive effect of NVDX3 was evaluated. A model of osteoblastogenesis from human mesenchymal stem cells was used. Mesenchymal stem cells were thawed according to the recommendations of the supplier. Cells were seeded and cultured in flask in the medium recommended by the supplier for cell proliferation (RoosterBio, KT-001). Four days after thawing, human MSCs were detached with trypsin-EDTA and counted. The cells were seeded at 2.10⁴ cells per well and cultured in monolayer in 24-well plates in DMEM medium supplemented with 1% FBS for 4 days (the day of seeding is designated day-4). After 4 days of culture in DMEM medium, cells were placed in basal medium (DMEM 1% FBS+ascorbic acid (50 μg/mL) and b-glycerophosphate (10 mM), differentiation medium (positive control) (DMEM 1% FBS+ascorbic acid (50 μg/mL) and b-glycerophosphate (10 mM), dexamethasone (10-8 M) and a vitamin D3 (10-8 M)) or basal medium and NVDX3 (n=2 donors) at 5, 20 and 100 mg/well in transwells (the first day of treatment is “Day 0”). Medium and treatments were changed at day 4, day 7 and day 11. All treatments and controls were carried out in duplicate.

Cells were lysed at day 7 and day 14 using Qiazol lysis reagent (Qiagen®, Hilden, Germany). Total RNAs were extracted from cell lysates and quantified as disclosed in example 8.

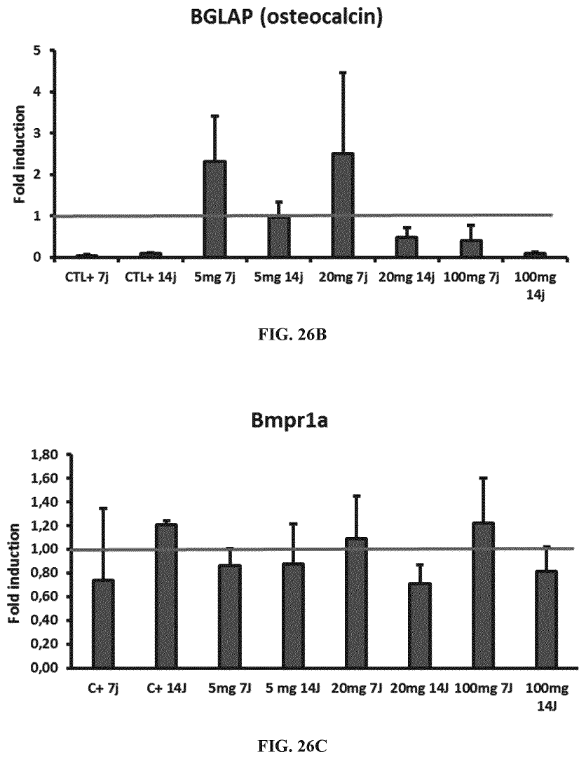

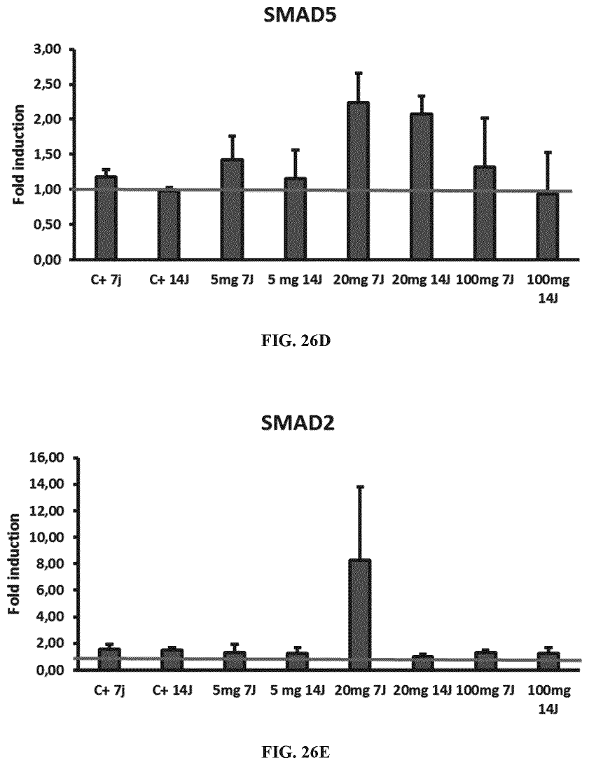

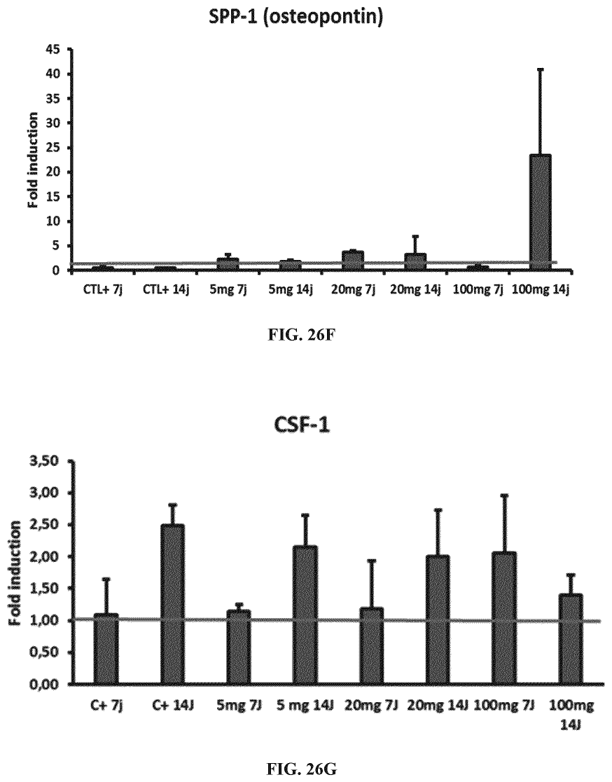

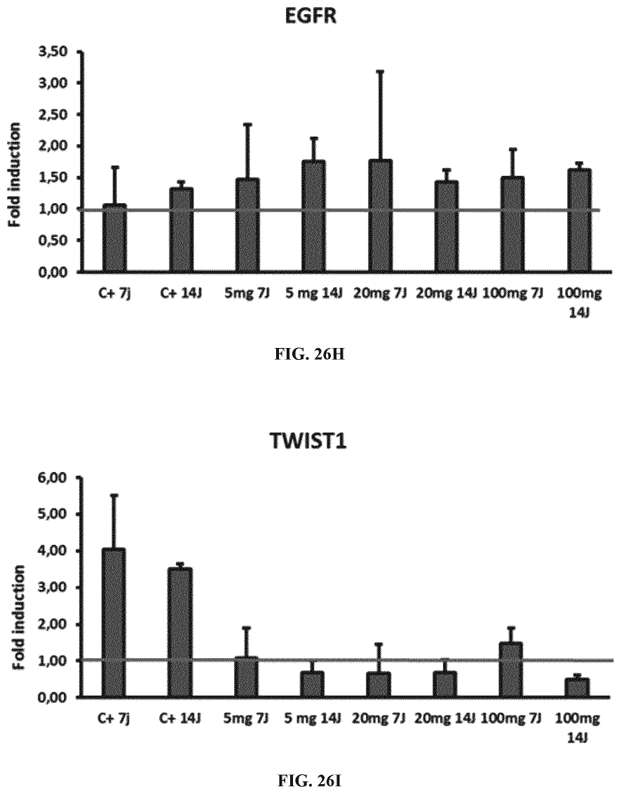

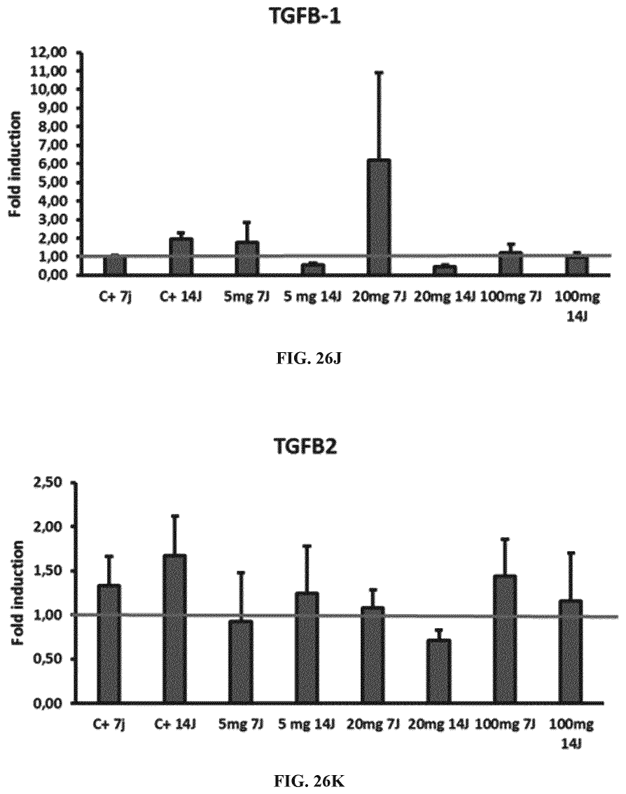

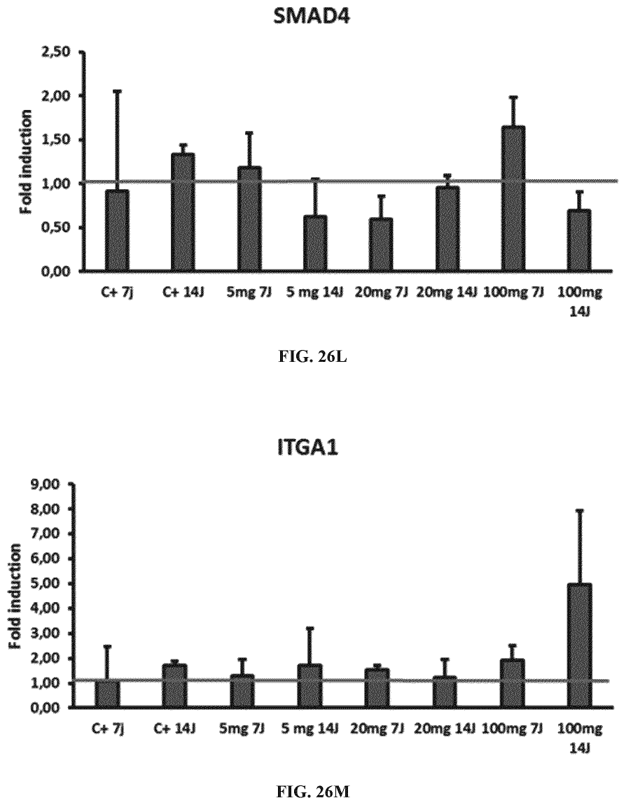

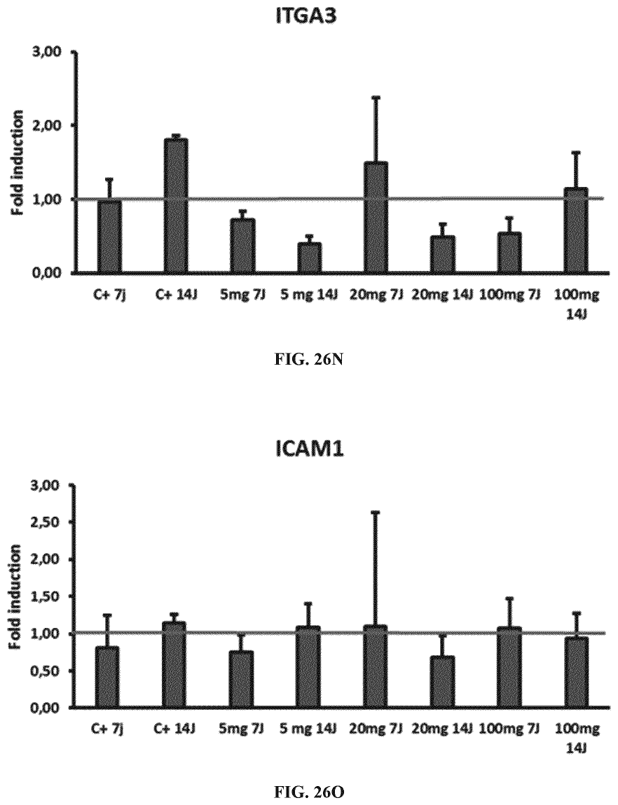

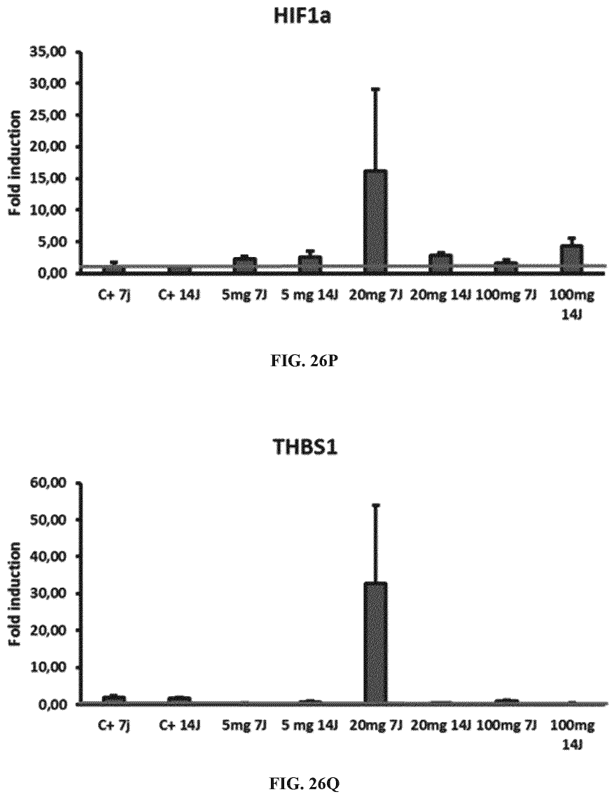

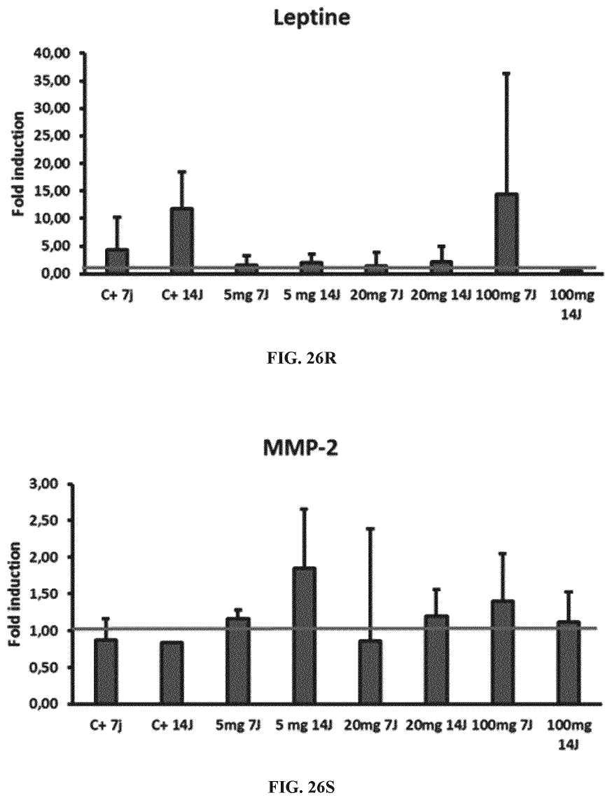

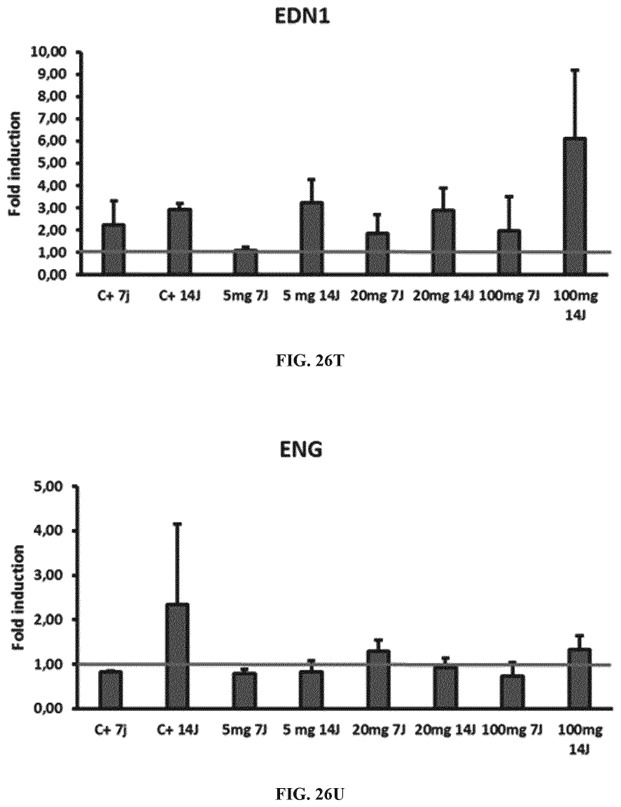

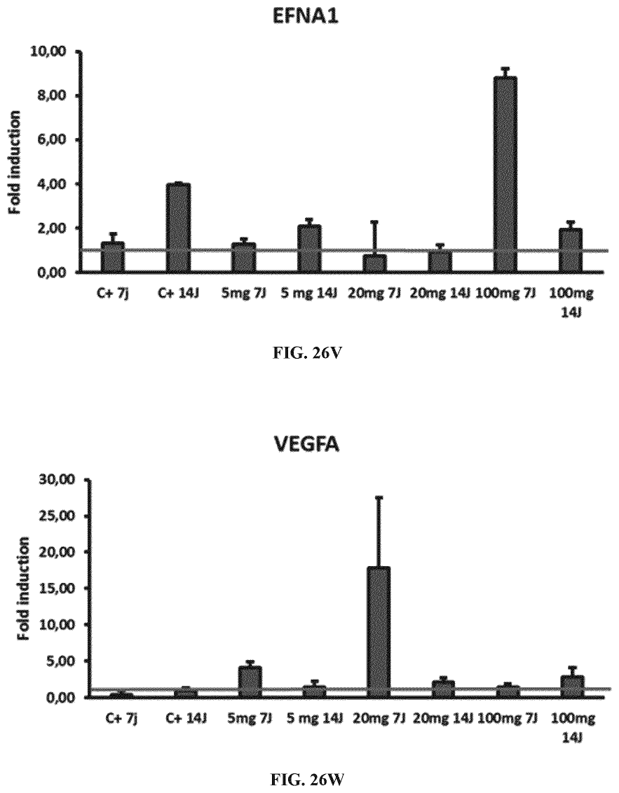

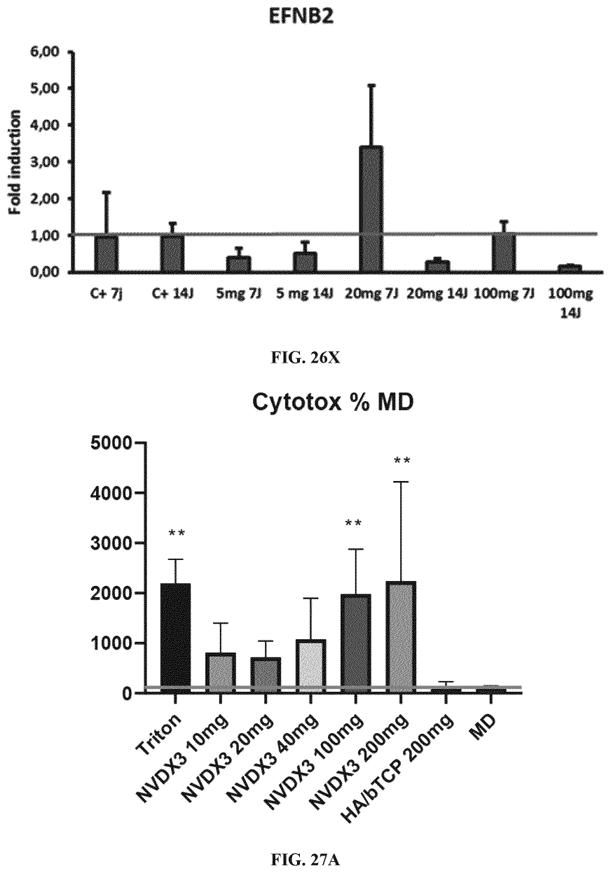

Osteogenic and angiogenic genes expression are varying depending on the time of assessment (7 vs 14 days) and the dose of test item applied. However, the majority of tested genes were, at least transiently, overexpressed after NVDX3 treatment at each tested dose in comparison to the negative control (basal medium, set at 1), as found in the positive control (differentiation medium). These results show that NVDX3 possesses osteo-inductive properties and can promote the osteo-differentiation of osteoblasts precursors (FIG. 26A-X). In addition, these results demonstrate that the osteogenic/angiogenic effect of the NVDX3 biomaterial is contact-independent, and is mediated by soluble factors.

### 2.3. Impact of NVDX3 on the Mature Osteoblasts (Cytotoxicity)

SaOS₂ cells were plated in 12-wells plates in Mc Coy's medium+10% FBS, 1% P/S and 0.1% amphotericin B for 2-3 days then placed in Mc Coy's 1% FBS and NVDX3 (n=3) or HA/βTCP were added in transwells (12 mm Transwell with 0.4 m pore polyester membrane insert). The following conditions were carried out in triplicate:
Cells+medium with 1% FBS+2% Triton “control”
Cells+medium with 1% FBS “control”
Cells+medium with 1% FBS+10 mg NVDX3
Cells+medium with 1% FBS+20 mg NVDX3
Cells+medium with 1% FBS+40 mg NVDX3
Cells+medium with 1% FBS+100 mg NVDX3
Cells+medium with 1% FBS+200 mg NVDX3
Cells+medium with 1% FBS+200 mg HA/TCP (Biomatlante®)

The cytotoxicity was evaluated at 24h by a Viability assay a cytotoxicity assay and the LDH production was assessed.

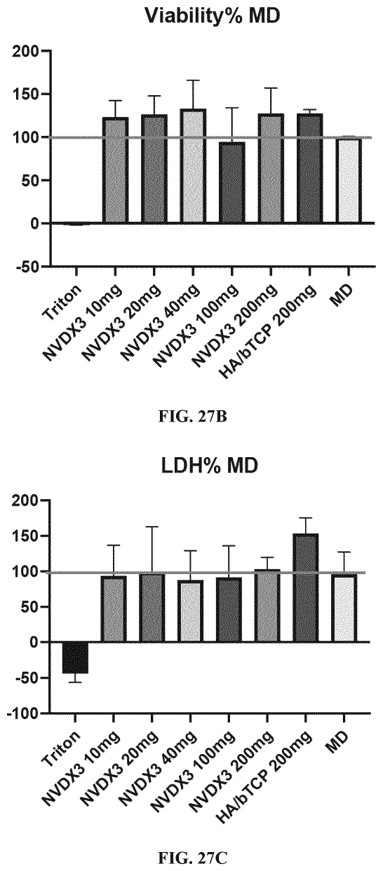

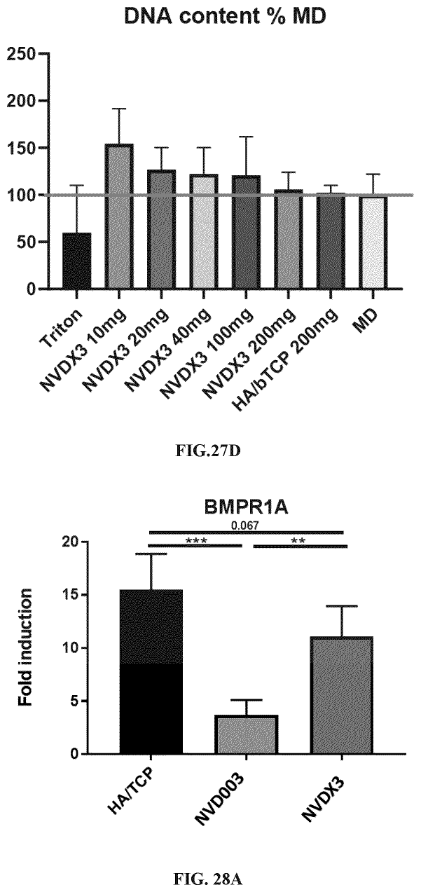

The positive control (Triton) showed a rapid cytotoxicity (FIG. 27A) with no viability (FIG. 27B), no cellular turnover associated with a low DNA content, a low level of “free-DNA” staining (cytotoxicity; FIG. 27D), and no LDH production (FIG. 27C).

The basal condition (MD) showed low cytotoxicity (FIG. 27A) and therefore a low cellular turnover. This was associated with a low cumulative mitochondrial activity on 24h (low viability; FIG. 27B), high DNA content (FIG. 27D) and moderate LDH activity (FIG. 27C).

The negative control (HA/βTCP) showed a low cytotoxicity (FIG. 27A), a high viability (FIG. 27B) associated with high DNA content (FIG. 27D) and moderate LDH activity (FIG. 27C).

A dose-response cytotoxic effect was found with increasing doses of NVDX3 from 10 mg/well (12 well plates; FIG. 27A). A significant higher cytotoxicity was found at 100 and 200 mg NVDX3 in comparison with cells in MD (p<0.01; FIG. 27A) In contrast, no cytotoxicity was noted at 200 mg HA/βTCP (FIG. 27A). However, similar viability (FIG. 27B), DNA content (FIG. 27D) and LDH production (FIG. 27C) were measured for doses up to 200 mg/well of NVDX3 tested and HA/BTCP.

### 3. Conclusion and Discussion

The aim of this study was to describe the anti-resorptive and osteogenic properties of NVDX3. The impact of these products on human osteoclast formation and viability using an in vitro model of human osteoclastogenesis was evaluated. In addition, the effect of NVDX3 on human osteoblast formation and viability using an in vitro model of human osteoblastogenesis was studied.

In this study, NVDX3 showed a total inhibitory effect on osteoclasts differentiation at the three tested doses (5 mg, 20 mg and 100 mg). This inhibitory effect was more important at low doses (5 mg and 20 mg) with NVDX3 treatment in comparison with NVD00X, NVD003 and HA/TCP.

NVDX3 showed also an inhibitory effect on mature osteoclasts inhibiting by 35% the osteoclasts viability in the presence of 5 mg of the compound, by 30% in the presence of 20 mg and by 80% in the presence of 100 mg.

These results indicate that the anti-resorptive properties of NVD003 are maintained after freeze-drying and gamma-irradiation.

In addition, NVDX3 was shown to promote osteo-differentiation of osteoblasts precursors and, while a cytotoxic effect of mature osteoblasts was noted, it was associated with a rapid cellular turnover with no impact on cell content or cell viability.

In conclusion, NVDX3 presents anti-resorptive and osteogenic properties in vitro associated to the absence of cytotoxicity on osteoblasts.

### Example 13: In Vivo Promotion of Osteogenesis of NVDX3

### 1. Osteo-Induction

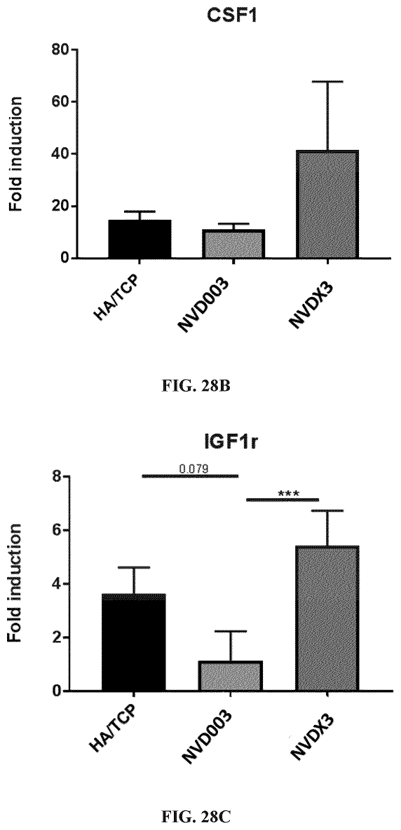

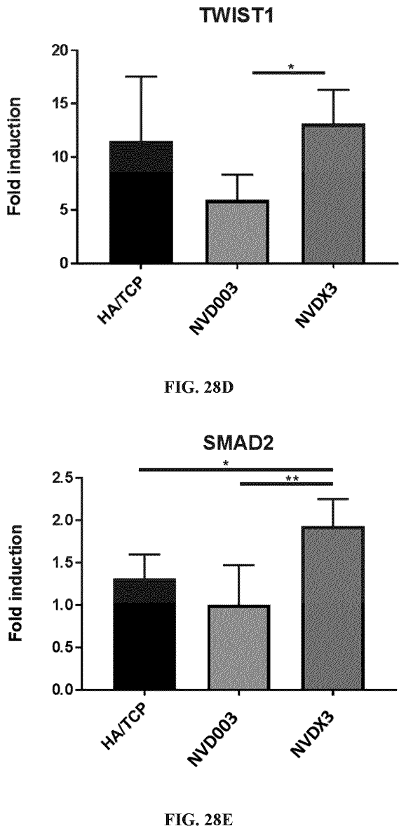

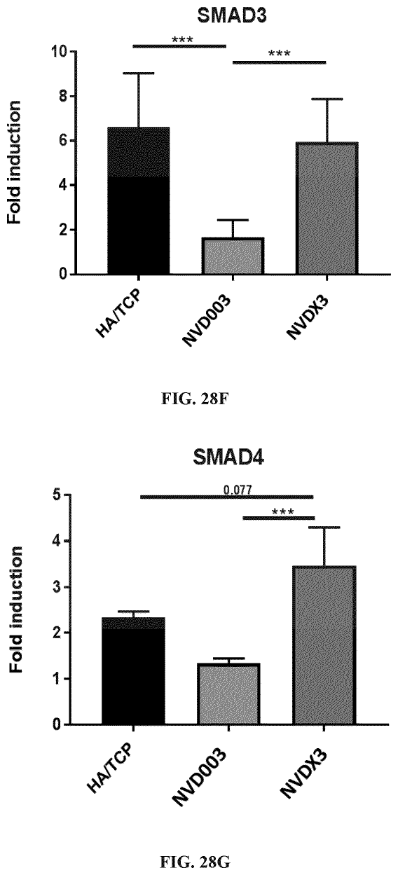

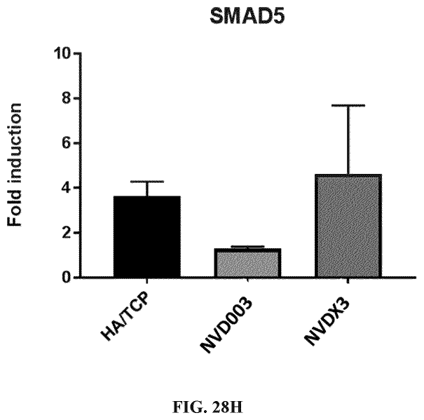

NVDX3 (after lyophilization and gamma irradiation) demonstrated a significant higher osteo-induction at molecular level after 1-month post-transplantation in comparison to HA/βTCP alone and fresh NVD003 (FIG. 28A-H).

### 2. Immune Response—Anti-HLA1 (Anti-Human Leucocyte Antigen 1) Antibodies Detection

This significant higher osteo-induction (at molecular level) is associated to a significant lower immune response in term of antibody production (anti-HLA) demonstrating the tolerance of lyophilized-irradiated NVD003 (NVDX3).

To evaluate the humoral response following NVD003 (or HA/β-TCP or NVDX3) implantation in the case of a critical-sized bone-defect in Wistar rats, we use the FlowPRA™ Class I Screening Test technology. The protocol is quite different according to the type of Ig we investigate.

### a) Anti-HLA1 IgG Detection

In an appropriate tube, the serum sample is mixed with FlowPRA™ Class I Screening Test Beads (One Lambda®, USA—FL1-30) according to the manufacturer's instructions. Incubation of 30 minutes at 20° C. under darkness. Two washes with PBS-BSA 0.5% are performed with centrifugation steps of 2 minutes at 9,000×g. Addition of biotinylated anti-IgG (BioLegend®, USA—405428) followed by an incubation of 30 minutes at 20° C. under darkness. Two washes with PBS-BSA 0.5% are performed with centrifugation steps of 2 minutes at 9,000×g. Addition of PE-streptavidin (BD Biosciences®, USA—554061) followed by an incubation of 30 minutes at 20° C. under darkness. Two washes with PBS-BSA 0.5% are performed with centrifugation steps of 2 minutes at 9,000×g. Addition of PFA 0.5%, transfer in the reading plate and acquisition of 5,000 to 10,000 beads with a cytometer (Beckman Coulter).

### b) Anti-HLA1 IgM Detection

Prior any mix with the specific beads, the sera samples must be depleted in IgG. The depletion is performed by the addition, to the sample, of biotinylated anti-IgG (BioLegend®, USA—405428) followed by an incubation of 20 minutes at 4° C. and under darkness. Addition of magnetic beads coupled with streptavidin (Streptavidin Particles Plus—DM—BD Biosciences®, USA—557812) and positioning on a magnetic bench during 7 minutes at 20° C. Transfer of the supernatant in a new tube. The serum sample is mixed with FlowPRA™ Class I Screening Test Beads (One Lambda®, USA—FL1-30) according to the manufacturer's instructions. Incubation of 60 minutes at 20° C. under darkness. Two washes with PBS-BSA 0.5% are performed with centrifugation steps of 2 minutes at 9,000×g. Addition of biotinylated anti-IgM (BioLegend®), USA—408903) followed by an incubation of 30 minutes at 20° C. under darkness. Two washes with PBS-BSA 0.5% are performed with centrifugation steps of 2 minutes at 9000×g. Addition of PE-streptavidin (BD Biosciences®, USA—554061) followed by an incubation of 30 minutes at 20° C. under darkness. Two washes with PBS, 0.5% BSA are performed with centrifugation steps of 2 minutes at 9,000×g. Addition of 0.5% paraformaldehyde, transfer in the reading plate and acquisition of 5,000 to 10,000 beads with a cytometer (Beckman Coulter®).

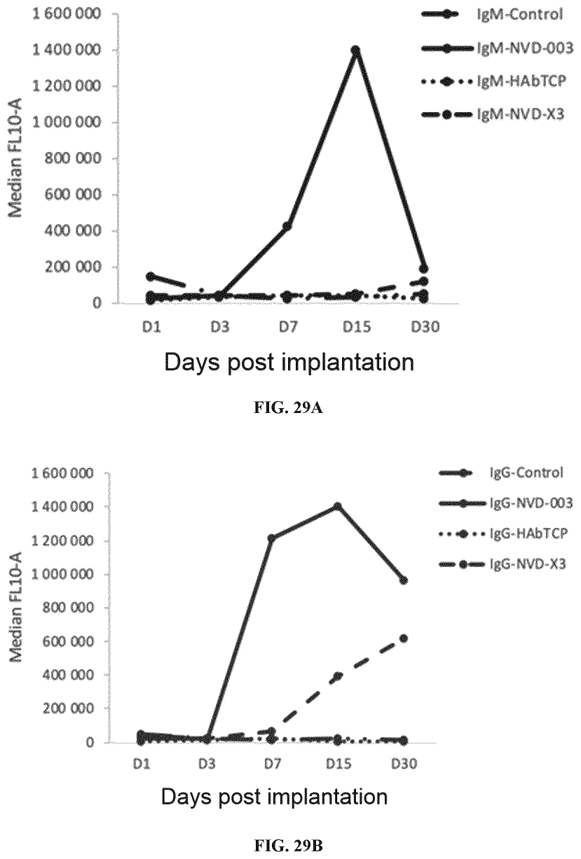

As shown in FIG. 29A-B, the transplantation of the NVDX3 biomaterial does not result in the elicitation of an anti-HLA IgM immune response (FIG. 29A) and results in a poor elicitation of an anti-HLA IgG immune response (FIG. 29B). These experimental data suggest that the NVDX3 biomaterial may be adapted to allogenic transplantation. By comparison, the NVD003 biomaterial promotes both an anti-HLA IgM and IgG immune response upon transplantation, which confirm that only autologous transplantation may be performed with said biomaterial.

### Example 14: Impact of NVDX2 on Glucocorticoids-Exposed ASCs and HDFa

### 1. Materials and Methods

NVDX2 biomaterials were produced accordingly to example 1 (n=2, from 2 different donors). Dexamethasone (Aacidexam®, 5 mg/mL solution for injection, lot 09169TB24) was diluted to a final concentration of 10 μM, both in DMEM+1% FBS and DMEM+1% hPL.

Human dermal fibroblasts (HDFa; Cell Applications®, cat: 106-05a) were seeded in 12-well plates at 12,000 cells/cm² and cultured in DMEM+10% FBS. ASCs (at P5) were also seeded in 12-well plates at 12,000 cells/cm² and cultured in DMEM+10% hPL. After 24h of culture, the medium was changed to DMEM+1% FBS for HDFa and DMEM+1% hPL for ASCs and exposed to 0 or 10 μM of dexamethasone, in the presence of NVDX2 (20 mg or 50 mg) placed in transwells and without NVDX2. The metabolic activity of proliferating cells was measured 48h after exposure by CCK-8 (Sigma Aldrich®, Cell counting Kit—8). Subsequently, the DNA extraction was performed by lysing the cells with TE buffer (1×) and centrifugating the lysate at 12,000×g for 5 min. The dsDNA content was determined on the supernatant by the Quant iT PicoGreen® dsDNA Kit according to the manufacturer's instructions.

### 2. Results

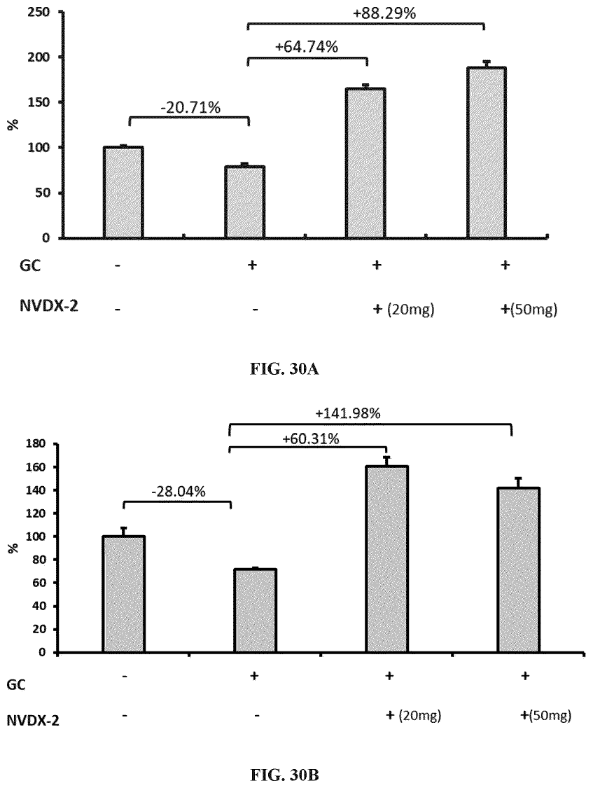

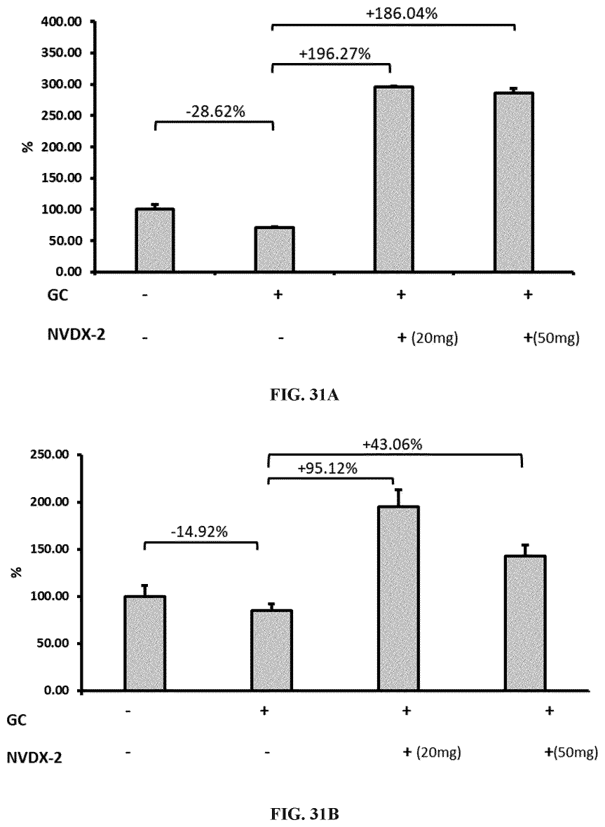

The exposure of HDFa and ASCs to glucocorticoids (dexamethasone) decreases the cell viability of the cells after the 48h exposure based on the metabolic activity (FIG. 30A-B) and DNA quantitation (FIG. 31A-B). However, the presence of NVDX2 increases the cell viability of the HDFa and ASCs during the exposure time, demonstrating that NVDX2 compensates for the inhibition of cell proliferation by the glucocorticoids. The effect can be observed for both doses (20 mg and 50 mg) and is confirmed by the DNA quantitation. Few reduction of DNA quantity in the presence of GC was observed for ASCs (FIG. 31B). However, the presence of NVDX2 increases the DNA quantity in the ASCs culture during the exposure time and both doses.

### 3. Conclusion

These data demonstrated the compensation of the side effect of glucocorticoids (dexamethasone) by NVDX2 on HDFa and ASCs.

### Example 15: Antiproliferative Properties of NVDX2-Derived and NVDX3-Derived Exosomes

### 1. Material and Methods

### 1.1. Cancer Line Cells

Three tumoral cell lines were used as target cells: H143B human osteosarcoma cells were obtained from ATCC® (CRL-8303™); A375 human melanoma cells were obtained from ATCC® (CRL-1619™); U87 human glioblastoma cells were obtained from ATCC® (HTB-14™).

### 1.2. Methods

### a) Preparation of ANVD002 Biomaterial

Human subcutaneous adipose tissues are harvested by lipo-aspiration (following the Coleman technique in the abdominal region) after informed consent and serologic screening.

Then, the lipoaspirate is digested by a collagenase solution (Serva Electrophoresis® GmbH, Heidelberg, Germany) in Hanks' Balanced Salt Solution during 50-70 min at 37° C.±1° C. The digestion is stopped by the addition of MP medium (proliferative medium consisting of Dulbecco's modified Eagle's medium, 4.5 g/L Glucose/Ala-Gln (UltraGlutamine (Lonza®) or Glutamax® (Gibco®), supplemented with 5% Human Platelet Lysate, 1% of penicillin/streptomycin. The digested adipose tissue is centrifuged (500×g, 10 min, at room temperature) and the supernatant is discarded. The pelleted Stromal Vascular Fraction (SVF) is re-suspended into MP medium and passed through a 200-500 m mesh filter. The filtered cell suspension is centrifuged (500×g, 10 min, 20° C.). The pellet containing the hASC is resuspended into MP medium. A sample of the cell suspension is used to seed one 75 cm² T-flask (Passage P0).

Between P0 and the fourth passage (P3/P4), cells are cultivated on T-flasks and fed with fresh proliferative medium (MP). This medium is composed of DMEM medium (4.5 g/L glucose and 4 mM Ala-Gln) supplemented with 5% hPL (v/v), pH (7.2-7.4). Cells are passaged when reaching a confluence of about 80-90%. At each passage, cells are detached from their culture vessel with TrypLE® (Select 1×). TrypLE digestion is performed for 5-15 min at 37° C.±2° C. and stopped by the addition of MP medium (proliferative medium). Cells are then centrifuged (500×g, 5 min, room temperature), and re-suspended in MP medium (proliferative medium).

At the fourth passage (P3/P4), cells are seeded in re-closable cell culture flasks (150 cm²) and fed with osteogenic differentiation medium (MD). This medium is composed of proliferative medium (DMEM, Ala-Gln, hPL 5%) supplemented with dexamethasone (I M), ascorbic acid (0.25 mM) and sodium phosphate (2.93 mM). The volume of cell suspension is standardized to 70 ml per 150 cm² for the differentiation phase (MD medium).

When cells reach a confluence and if a morphologic change appears and if at least one osteoid nodule (un-mineralized, organic portion of the bone matrix that forms prior to the maturation of bone tissue) is observed in each flask, the 3-D induction can be started.

The culture vessels containing the confluent monolayer of adherent osteogenic cells are sprinkled with Cultispher particles (1.5 cc for a 150 cm² vessel). Few days after the addition of the Cultispher, the osteogenic cells and the particles dispersed become progressively entombed in mineralizing extracellular matrix.

Few days after, the osteogenic cells and the Cultispher particles start forming a large 3-dimensional patch (or few smaller patches) of partially mineralized translucid and malleable membrane detaching from each culture vessels. Regular medium exchanges are performed every 3 to 4 days during the 3D induction. Those medium exchanges are performed by carefully preventing removal of Cultispher particles and developing structure(s).

After about 15 days, the scaffold-free 3D culture (NVD-002) is developed and detached from the T-flasks. Cultures are maintained during 5 to 8 weeks after the addition of particles with medium change every 3-4 days.

### b) Preparation of ANVD003 Biomaterial

The protocol is identical to the preparation of NVD002 biomaterial (see section a) above), except for the 3-D induction of cells.

After being exposed to the osteogenic differentiation medium (MD), the culture vessels containing the confluent monolayer of adherent osteogenic cells are sprinkled with HA/β-TCP particles (3 cc for a 150 cm² vessel).

Few days after the addition of the HA/β-TCP, the osteogenic cells and the particles dispersed become progressively entombed in mineralizing extracellular matrix. Few days after, the osteogenic cells and HA/β-TCP particles start forming a large 3-dimensional patch (or few smaller patches) of partially mineralized brownish-yellow moldable putty detaching from each culture vessels. Regular medium exchanges are performed every 3 to 4 days during the 3D induction. Those medium exchanges are performed by carefully preventing removal of HA/β-TCP particles and developing structure(s).

After about 15 days, the scaffold-free 3D structure (NVD003 biomaterial) is developed and detached from the T-flasks. Cultures are maintained during 5 to 8 weeks after the addition of particles with medium change every 3-4 days.

### c) Isolation of Exosomes

8 weeks after the addition of particles, NVD002 and NVD003 biomaterials were rinsed 3 times with PBS were placed in MD without hPL+5% FBS depleted in exosomes for 72h. Supernatant was then harvested and centrifuged at 400×g for 5 minutes followed by 20 minutes at 2,000×g at 4° C. Supernatant was kept at 2/8° C. for direct exosomes isolation. Isolated exosomes were then stored at −80 C.°.

Exosomes have been isolated by differential centrifugation from culture medium whereby larger “contaminants” are first excluded by pelleting out through increasing speeds of centrifugation before exosomes, small extracellular vesicles and even protein aggregates are pelleted at very high speeds (˜100,000×g).

### d) Proliferation Assay

NVD003 and NVD002-derived exosomes from 3 donors were co-incubated in 96-wells plates with those three cell lines at 2.5 and 25 μg/ml for up to 72h at 37° C., 5% CO₂. A cell viability test (using the CellTiter-Glo® Cell viability Assay from PROMEGA®) was performed after 30 minutes to 48h of co-incubation, at minimum 5 different time points, to evaluate the proliferation of targeted cells. The CellTiter-Glo® Luminescent Cell Viability Assay from PROMEGA® is a homogeneous method to determine the number of viable cells in culture based on quantitation of the ATP present, which signals the presence of metabolically active cells). Experiments were performed in triplicate.

### e) Statistical Analysis

Statistically significant differences between groups (with normal distribution) were tested by paired t-test and one-way analysis of variance with the Bonferroni post hoc test. Non-normal distributions of data were analyzed using the Kruskal-Wallis test. Statistical tests were performed with Prism GraphPad 2 (NIH). Statistical significance are as follows: *:p<0.05; **: p<0.01; ***: p<0.005; ****: p<0.0001.

### 2. Results

### 2.1. In Vitro Effect of Exosomes on Human Osteosarcoma Cells (H143B)

### a) Effect of NVD002-Exosomes

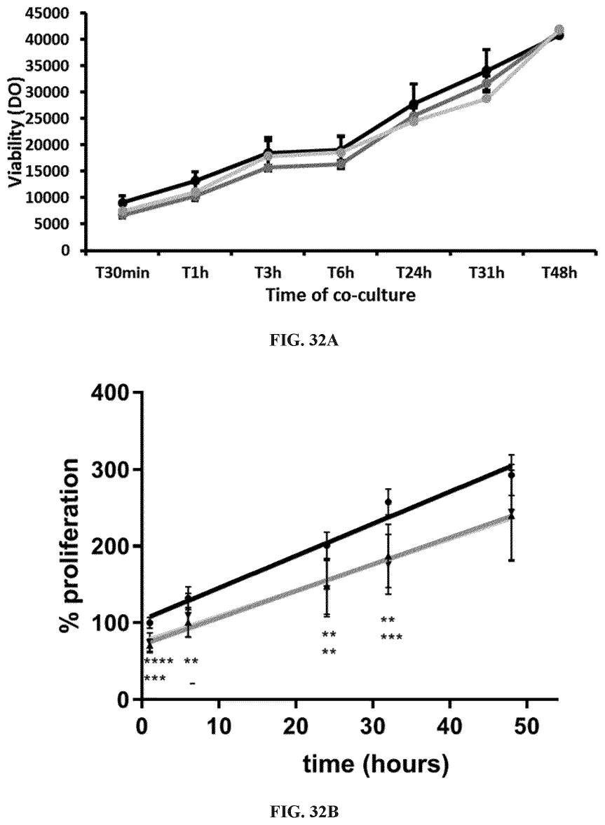

Proliferation curves of H143B cells cultured with exosomes showed a slightly lower level of viability than the control cells cultured without exosomes. In addition, a more marked effect was noted with the highest dose of exosomes (25 μg/ml vs 2.5 μg/ml) (FIG. 32A). Linear regression of the proliferation curves was calculated. Lower proliferation rates were found for cells cultured with exosomes, at both 2.5 and 25 μg/ml. A significant lower viability signal was found in cells co-cultured with exosomes at 2.5 and 25 μg/ml at 1, 24 and 32h of incubation and 2.5 μg/ml at 1, 6, 24 and 32h of incubation (p<0.01). (FIG. 32B).

Although a higher slope was found for cells cultured without exosomes, it was associated with a higher viability level.

### b) Effect of NVD003-Exosomes

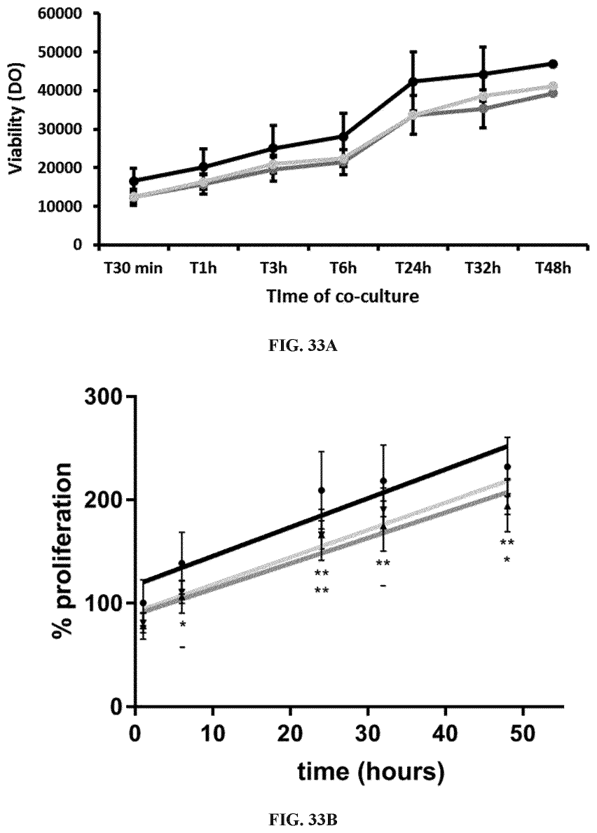

Proliferation curves of H143B cells cultured with exosomes showed a slightly lower level of viability than the control cells cultured without exosomes (FIG. 33A).

Linear regression of the proliferation curves was calculated. Lower proliferation rates were found for cells cultured with exosomes, at both 2.5 and 25 μg/ml. A significant lower viability signal was found in cells co-cultured with exosomes at 2.5 at 6, 24, 32 and 48h of incubation and 25 μg/ml only at 24h and 48h of incubation p<0.01). (FIG. 33B) Although a higher slope was found for cells cultured without exosomes, it was associated with a higher viability level.

### c) Conclusion

In conclusion, NVD002- and NVD003-derived exosomes can reduce the proliferation of human osteosarcoma cell lines in vitro. A dose-response effect was observed.

### 2.2. In Vitro Effect of Exosomes on Human Melanoma Cells (A375)

### a) Effect of NVD002-Exosomes

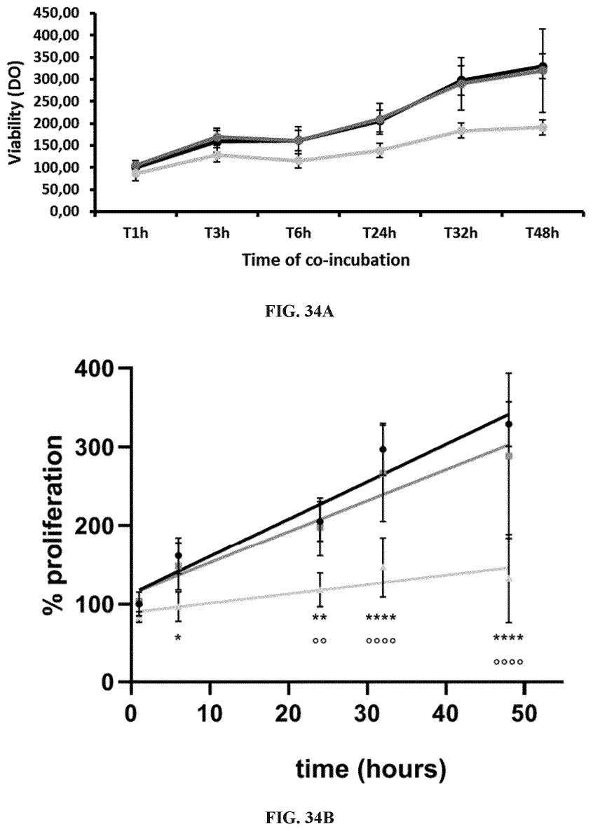

Although similar profile was found between cells cultured without exosomes and 2.5 μg/ml exosomes, proliferation curves of A375 cells cultured with 25 μg/ml exosomes showed a lower level of viability than the control cells cultured without exosomes (FIG. 34A).

Linear regression of the proliferation curves was calculated. Lower proliferation rates were found for cells cultured with exosomes, at both 2.5 and 25 μg/ml. A significant lower viability signal was found in cells co-cultured with exosomes at 25 μg/ml at 1, 24, 32 and 48h of incubation (p<0.01). In addition, a significant lower viability signal was found at 24, 32 and 48h in cells treated with 25 μg/ml NVD002-Exo vs 2.5 μg/ml (p<0.01) (FIG. 34B).

Although a higher slope was found for cells cultured without exosomes, it was associated with a higher viability level.

### b) Effect of NVD003-Exosomes

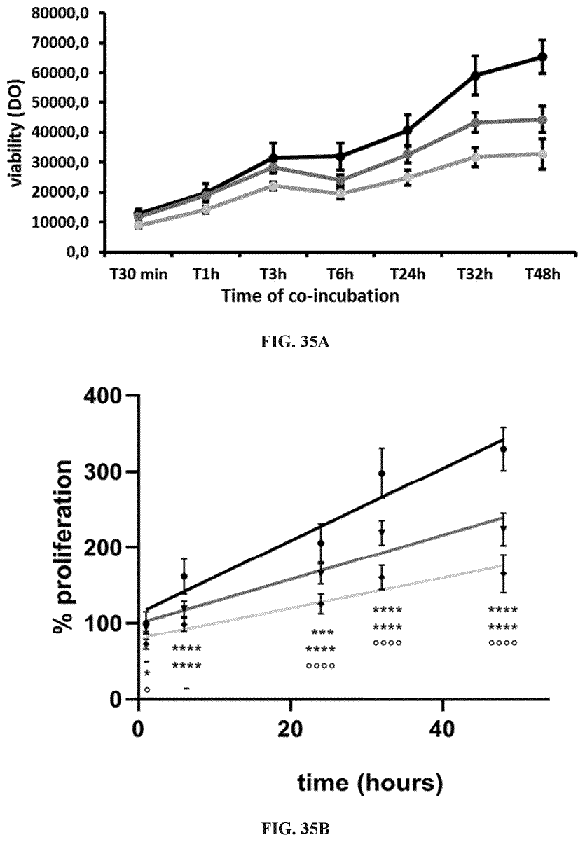

Proliferation curves of A375 cells cultured with 2.5 and 25 μg/ml exosomes showed a lower level of viability than the control cells cultured without exosomes. This effect was more marked at 25 μg/ml exosomes than 2.5 μg/ml (FIG. 35A).

Linear regression of the proliferation curves was calculated. Lower proliferation rates were found for cells cultured with exosomes, at both 2.5 and 25 μg/ml. A significant lower viability signal was found in cells co-cultured with exosomes at 25 μg/ml at each time point of incubation (p<0.01). A significant lower viability signal was found in cells co-cultured with exosomes at 2.5 μg/ml at 6, 24, 32 and 48h (p<0.05). A significant lower viability signal was found in cells co-cultured with exosomes at 25 μg/ml vs 2.5 μg/ml NVD003-Exo at 1, 24, 32, 48h (p<0.05) (FIG. 35B).

Although a higher slope was found for cells cultured without exosomes, it was associated with a higher viability level.

### c) Conclusion

In conclusion, NVD002- and NVD003-derived exosomes can reduce the proliferation of human melanoma cell lines in vitro. A dose-response effect was observed.

### 2.3. In Vitro Effect of Exosomes on Human Glioblastoma Cells (U87)

### a) Effect of NVD002-Exosomes

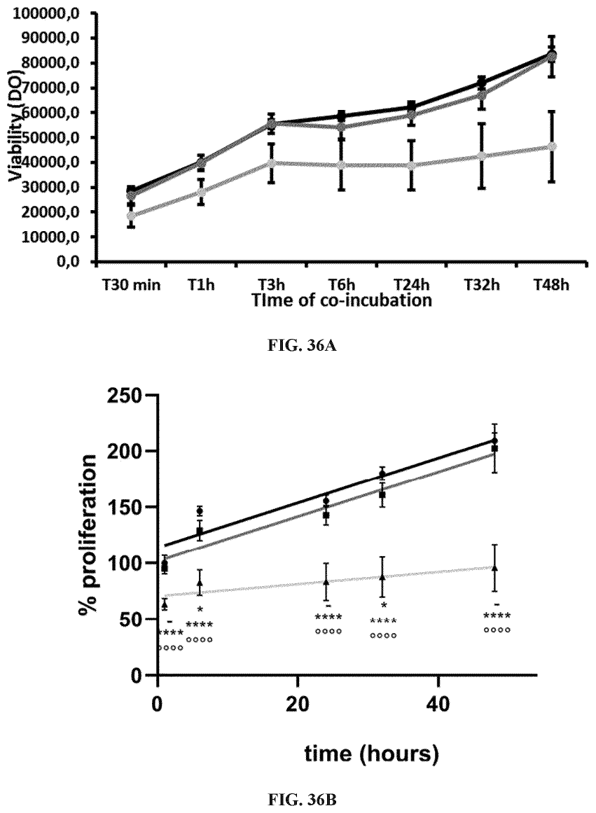

Although similar profile was found between cells cultured without exosomes and 2.5 μg/ml exosomes, proliferation curves of U87 cells cultured with 25 μg/ml exosomes showed a lower level of viability than the control cells cultured without exosomes (FIG. 36A).

Linear regression of the proliferation curves was calculated. Lower proliferation rates were found for cells cultured with exosomes, at both 2.5 and 25 μg/ml, with a more marked effect at 25 than 2.5 μg/ml. A significant lower viability signal was found in cells co-cultured with exosomes at 2.5 only at 6 and 32h (p<0.05) and 25 μg/ml at each time of incubation (p<0.0001). In addition, a significant lower viability signal was found for U87 cultured with 25 μg/ml NVD002-Exo vs 2.5 μg/ml at each tested timepoint (p<0.0001) (FIG. 36B).

Although a higher slope was found for cells cultured without exosomes, it was associated with a higher viability level.

### b) Effect of NVD003-Exosomes

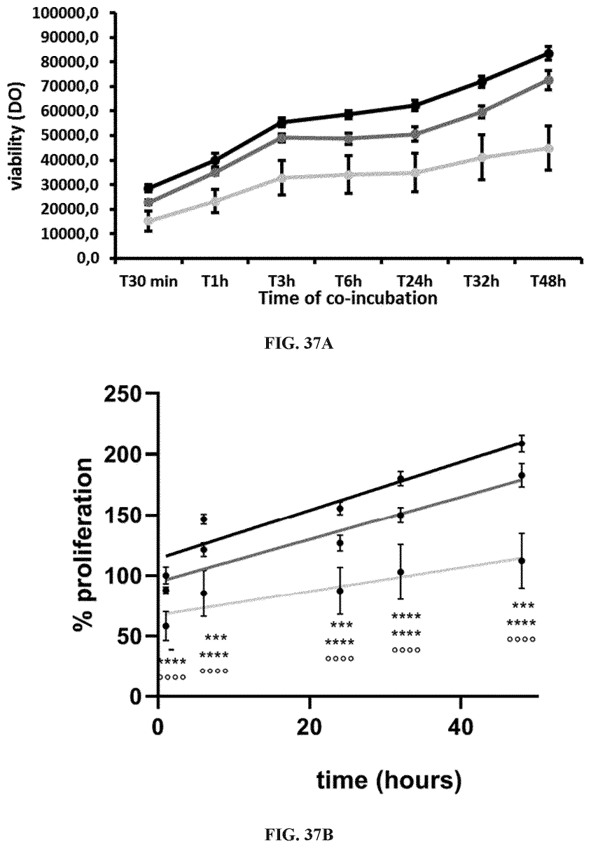

Proliferation curves of U87 cells cultured with 2.5 and 25 μg/ml exosomes showed a lower level of viability than the control cells cultured without exosomes. This effect was more marked at 25 μg/ml exosomes than 2.5 μg/ml (FIG. 37A).

Linear regression of the proliferation curves was calculated. Lower proliferation rates were found for cells cultured with exosomes, at both 2.5 and 25 μg/ml. A significant lower viability signal was found in cells co-cultured with exosomes at 2.5 μg/ml at 6, 24, 32 and 48h (p<0.0001). In addition, a significant lower viability signal was found for U87 cultured with 25 μg/ml NVD003-Exo vs 2.5 μg/ml at each tested timepoint (p<0.0001). (FIG. 37B).

Although a higher slope was found for cells cultured without exosomes, it was associated with a higher viability level.

### c) Conclusion

In conclusion, NVD002- and NVD003-derived exosomes can reduce the proliferation of human glioblastoma cell lines in vitro.

1. A sterile and desiccated biomaterial comprising devitalized differentiated cells having tissue regenerating and/or repairing properties, and a particulate material, the cells and the particulate material being embedded in an extracellular matrix.

2. The biomaterial according to claim 1, wherein said cells are selected in a group comprising primary cells, stem cells, genetically modified cells, and a combination thereof.

3. The biomaterial according to claim 1 or 2, wherein at most 10% of said cells are viable, preferably at most 1%.

4. The biomaterial according to any one of claims 1 to 3, wherein said particulate material is selected from the group comprising or consisting of:
an organic material, including demineralized bone matrix (DBM), gelatin, agar/agarose, alginates chitosan, chondroitin sulfate, collagen, elastin or elastin-like peptides (ELP), fibrinogen, fibrin, fibronectin, proteoglycans, heparan sulfate proteoglycans, hyaluronic acid, polysaccharides, laminins and cellulose derivatives;
a ceramic material, including particles of calcium phosphate (CaP), calcium carbonate (CaCO₃), calcium sulfate (CaSO₄), or calcium hydroxide (Ca(OH)₂), or combinations thereof,
a polymer, including polyanhydrides, polylactic acid (PLA), poly(lactic-co-glycolic acid) (PLGA), polyethylene oxide/polyethylene glycol (PEO/PEG), poly(vinyl alcohol) (PVA), fumarate-based polymers such as, for example poly(propylene fumarate) (PPF) or poly(propylene fumarate-co-ethylene glycol) (P(PF-co-EG)), oligo(poly(ethylene glycol) fumarate) (OPF), poly (n-isopropylacrylamide) (PNIPPAAm), poly(aldehyde guluronate) (PAG), poly(n-vinyl pyrrolidone) (PNVP), or combinations thereof;
a gel, including a self-assembling oligopeptide gel, a microgel, a nanogel, a particulate gel, a hydrogel, a thixotropic gel, a xerogel, a responsive gel, or combinations thereof,
a creamer;
and any combination thereof.

5. The biomaterial according to any one of claims 1 to 4, wherein said biomaterial comprises an altered factors content as compared to the factors content obtained from a corresponding fresh, non-sterile, non-desiccated biomaterial.

6. The biomaterial according to claim 5, wherein the factors content includes growth factors and/or transcription factors.

7. The biomaterial according to claim 5 or 6, wherein the factors content comprises IGF-1 and/or VEGF and/or SDF-1α and/or OPG.

8. The biomaterial according to claim 5, wherein said factors content includes a RNAs content.

9. The biomaterial according to claim 8, wherein the RNAs content comprises at least one miRNA selected in any one of Table 1, Table 2, Table 3, Table 4, Table 5, Table 6, Table 7, Table 8, Table 9, Table 10, Table 11 or Table 12.

10. The biomaterial according to any one of claims 1 to 9, wherein said desiccated biomaterial is obtained by freeze-drying.

11. The biomaterial according to any one of claims 1 to 10, wherein said sterile biomaterial is obtained by gamma-irradiation, preferably at a dose of about 7 kGy to about 45 kGy, more preferably at room temperature.

12. A method for generating a sterile and desiccated biomaterial comprising devitalized differentiated cells and a particulate material, the cells and the particulate material being embedded in an extracellular matrix, said method comprising the steps of:
(1) contacting (i) viable cells capable to undergo differentiation with (ii) a particulate material, so as to obtain a first combination;
(2) culturing the first combination obtained in step (1) in a culture medium such that the cells secrete an extracellular matrix and synthesize a factors content, so as to acquire tissue regenerating and/or repairing properties, and wherein the cells and the particulate material are embedded in the extracellular matrix, so as to form a multidimensional structure;
(3) submitting the multidimensional structure obtained at step (2) to desiccation, so as to obtain a desiccated biomaterial;
(4) submitting the desiccated biomaterial obtained at step (3) to sterilization, preferably by gamma-irradiation, so as to obtain a sterile, desiccated biomaterial.

13. The method according to claim 12, wherein the viable cells capable to undergo differentiation are selected in a group comprising primary cells; stem cells, in particular stems cells from adipose tissue, bone marrow, or umbilical cord blood; genetically modified cells; and a mixture thereof.

14. A desiccated and sterile biomaterial obtainable by the method according to any one of claims 12 to 13.

15. A pharmaceutical composition comprising a biomaterial according to any one of claims 1 to 11, or according to claim 14, and a pharmaceutically acceptable vehicle.

16. The pharmaceutical composition according to claim 15, wherein the composition is in the form of a paste or a film.

17. A medical device comprising a biomaterial according to any one of claims 1 to 11, according to claim 14, or a pharmaceutical composition according to claim 15 or 16.

18. A biomaterial according to any one of claims 1 to 11, according to claim 14, or a pharmaceutical composition according to claim 15 or 16, for use as a medicament.

19. The biomaterial or the pharmaceutical composition for use according to claim 18, for preventing and/or treating a tissue disorder.

20. The biomaterial or the pharmaceutical composition for use according to claim 19, wherein said tissue is selected from the group comprising bone tissue, cartilage tissue, skin tissue, muscular tissue, epithelial tissue, endothelial tissue, neural tissue, connective tissue and adipose tissue.

21. The biomaterial or the pharmaceutical composition for use according to claim 19, wherein the tissue disorder is selected in a group comprising aplasia cutis congenita; a burn; a cancer, including a breast cancer, a skin cancer and a bone cancer; a Compartment syndrome (CS); epidermolysis bulbosa; giant congenital nevi; an ischemic muscular injury of lower limbs; a muscle contusion, rupture or strain; a post-radiation lesion; and an ulcer, including a diabetic ulcer, preferably a diabetic foot ulcer; arthritis; bone fracture; bone frailty; Caffey's disease; congenital pseudarthrosis; cranial deformation; cranial malformation; delayed union; infiltrative disorders of bone; hyperostosis; loss of bone mineral density; metabolic bone loss; osteogenesis imperfecta; osteomalacia; osteonecrosis; osteopenia; osteoporosis; Paget's disease; pseudarthrosis; sclerotic lesions; spina bifida; spondylolisthesis; spondylolysis; chondrodysplasia; costochondritis; enchondroma; hallux rigidus; hip labral tear; osteochondritis dissecans; osteochondrodysplasia; polychondritis; and the likes.

22. The biomaterial or the pharmaceutical composition for use according to claim 18 or 19, for preventing and/or treating a bone disorder and/or a cartilage disorder.

23. The biomaterial or the pharmaceutical composition for use according to claim 18, for tissue reconstruction.

24. The biomaterial or the pharmaceutical composition for use according to claim 18 or 19, for compensating the side effects of a primary treatment of a tissue disorder, and/or for strengthening a primary treatment of a tissue disorder.

25. The biomaterial or the pharmaceutical composition for use according to claim 18 or 19, for compensating the side effects of a therapeutic treatment known to have a deleterious effect on tissues, in particular bone tissue, cartilage tissue, skin tissue, muscular tissue, epithelial tissue, endothelial tissue, neural tissue, connective tissue and adipose tissue.
